(19) United States
(12) Patent Application Publication
(10) Pub. No.: US 2023/0285549 A1
(43) Pub. Date: Sep. 14, 2023

(54) CD4+HELPER EPITOPES AND USES TO ENHANCE ANTIGEN-SPECIFIC IMMUNE RESPONSES

(71) Applicant: THE WISTAR INSTITUTE OF ANATOMY & BIOLOGY, Philadelphia, PA (US)

(72) Inventors: Ziyang XU, Philadelphia, PA (US); Daniel W. KULP, Morristown, NJ (US); David B. WEINER, Merion, PA (US)

(73) Assignee: THE WISTAR INSTITUTE OF ANATOMY & BIOLOGY, Philadelphia, PA (US)

(21) Appl. No.: 18/013,542

(22) PCT Filed: Jun. 30, 2021
(86) PCT No.: PCT/US21/40008

§ 371 Date: Dec. 28, 2022

Related U.S. Application Data
(60) Provisional application No. 63/046,687, filed on Jun. 30, 2020.

(51) Int. Cl. A61K 39/39; A61K 39/21; A61P 31/18

(52) U.S. Cl. CPC A61K 39/39; A61K 39/21; A61P 31/18; A61K 2039/55516

(57) ABSTRACT

Disclosed are compositions comprising an expressible nucleic acid sequence comprising a first nucleic acid sequence comprising a sequence that encodes an adjuvant polypeptide or a pharmaceutically acceptable salt thereof and a second nucleic acid sequence comprising a sequence that encodes an antigen from a virus or from a cancer. In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker.

### BACKGROUND

Vaccination is an approach where antigenic materials are introduced into the hosts to elicit adaptive immune responses that may confer them with protection from subsequent pathogen exposure (Clem, 2011). Humoral immunity is an important branch of the adaptive immune system, in which antibodies produced by B cells serve either to directly neutralize targets on the pathogens through paratope-epitope interactions (Corti and Lanzavecchia, 2013; Kwong et al., 2013), or to indirectly mediate inactivation of the pathogens by engaging the complement system or effector cells such as macrophages and natural killer cells through Fc-dependent mechanisms (Kurdi et al., 2018; Seidel et al., 2013; van Erp et al., 2019). Antibody responses serve as an important correlate for protection for many emerging and re-emerging infectious diseases, including but not limited to HIV-1 (Burton and Hangartner, 2016), influenza (Laursen et al., 2018), and coronaviruses (Jiang et al., 2020). A strategy to enhance humoral responses induced by vaccination is, therefore, of great significance.

CD4+ T cells, particularly T-follicular helper (Tfh) cells, play a critical role in the maturation of antibody responses (Crotty, 2014). In the germinal center, immunological synapses are formed between Tfh and Germinal Center B (GCB) cells through interactions of pairs of adhesion molecules such as LFA1-ICAM-1 and SAP-Ly108 to enable transfer of soluble cytokines, such as IL-4 and IL-21, from Tfh to GCB cells and promote ligand-receptor interaction, such as CD40L-CD40 binding, to enhance survival, differentiation, somatic hypermutation, and class switching in the GCB cells (Carrasco et al., 2004; Elgueta et al., 2009; Flynn et al., 1998; Kageyama et al., 2012). Provision of T-cell help, however, is contingent upon Tfh activation by GCB cells through T-cell receptor (TCR) peptide-MHC II interaction (Zhang et al., 2013). As such, robust germinal center B-cell responses are dependent on presentation of MHC II-restricted epitope, derived from the antigen, by GCB to Tfh cells. However, different epitopes have varying affinity for binding to MHC-II receptors depending on the hosts' haplotype such that peptide vaccines as well as smaller protein domains may not intrinsically contain a potent CD4+ helper epitope to drive germinal center responses (Elbahnasawy et al., 2018; Falugi et al., 2001; Pichichero, 2013). Such is the rationale for conjugating peptide and carbohydrate vaccines to protein carriers, like Keyhole limpet hemocyanin (KLH) (Ragupathi et al., 2002), tetanus toxin (Diethelm-Okita et al., 2000), or hepatitis B-surface antigen (HbsAg) (Collins et al., 2017). However, these large protein carriers may contain irrelevant immunodominant surfaces which may skew induced antibody responses away from the desired epitopes, creating additional uncertainties and challenges to this approach (Ghosh et al., 2013; Valea et al., 2018; Xu and Kulp, 2019).

Direct incorporation and fusion of a potent CD4+ helper epitope with the target antigen may be a simpler and more effective strategy to enhance the induced humoral immunity. Several important epitopes have been identified in this manner. Incorporation of Pan DR epitope (PADRE), for example, has demonstrated to improve immunogenicity of peptide and protein vaccines in animal studies and it has also been explored in several clinical studies (Alexander et al., 2000; Ghaffari-Nazari et al., 2015; Snook et al., 2019). Identification of additional potent CD4-helper epitopes can create new tools to be used in conjunction with, or as alternative to, these established CD4-helper epitopes as molecular adjuvants to various vaccine antigens.

### SUMMARY OF EMBODIMENTS

The present invention relates to a novel CD4+ helper epitope. In particular, the present invention relates to a composition comprising an expressible nucleic acid sequence encoding an adjuvant peptide comprising an HLA I-Ab epitope from Aquifex aeolicus or a functional fragment thereof, wherein the adjuvant peptide is no more than 20 amino acids, or in alternate embodiments, no more than about 15 amino acids in length. In certain embodiments, the present invention provides for an adjuvant peptide capable of binding HLA-DRB1*07:01, HLA-DRB1*15:01 and HLA-DRB5*01:01; or an adjuvant peptide comprising about 70%, 75%, 80%, 85%, 86%, 87%, 90%, 91%, 92, 93%, 94%, 95%, 96%, 97%, 98%, 99%, or 100% sequence identity to SEQ ID NO:1. In some embodiments, the disclosure relates to a pharmaceutical composition comprising a plasmid comprising a nucleic acid sequence encoding SEQ ID NO:1 or a variant thereof that is 70%, 75%, 80%, 85%, 86%, 87%, 90%, 91%, 92, 93%, 94%, 95%, 96%, 97%, 98%, 99%, or 100% sequence identity to SEQ ID NO: 1; and a pharmaceutically acceptable carrier.

In additional embodiments, the expressible nucleic acid can comprise a nucleic acid sequence that encodes a viral antigen or a cancer antigen, and the viral antigen, in some embodiments, can in turn comprise a Coronaviridae antigen, Respiratory syncytial virus (RSV) antigen, or Influenza antigen. In certain embodiments, the Coronaviridae antigen can be from SARS-Cov-2 or a functional fragment thereof that comprises at least about 70%, 75%, 80%, 85%, 86%, 87%, 88%, 89%, 90%, 91%, 92, 93%, 94%, 95%, 96%, 97%, 98%, 99% sequence identity to any disclosed SARS-Cov-2 antigen disclosed herein. Likewise, in certain embodiments the Influenza antigen can be HA or a functional fragment thereof that comprises at least about 70%, 75%, 80%, 85%, 86%, 87%, 90%, 91%, 92, 93%, 94%, 95%, 96%, 97%, 98%, 99% to any disclosed HA antigen disclosed herein. Finally, in certain embodiments the RSV antigen can be an amino acid sequence or a functional fragment thereof that comprises at least about 70%, 75%, 80%, 85%, 86%, 87%, 90%, 91%, 92, 93%, 94%, 95%, 96%, 97%, 98%, 99% to any disclosed RSV antigen disclosed herein.

In additional embodiments, the cancer antigen can comprise a breast cancer antigen, prostate cancer antigen, or a skin cancer antigen. In certain embodiments, the breast cancer antigen can be a HER2 or a functional fragment thereof that comprises at least about 70%, 75%, 80%, 85%, 86%, 87%, 90%, 91%, 92, 93%, 94%, 95%, 96%, 97%, 98%, 99%, 100% sequence identity to any disclosed breast cancer antigen disclosed herein. Likewise, in certain embodiments the prostate cancer antigen can be PSA or a functional fragment thereof that comprises at least about 70%, 75%, 80%, 85%, 86%, 87%, 90%, 91%, 92, 93%, 94%, 95%, 96%, 97%, 98%, 99% to any disclosed prostate antigen disclosed herein. Finally, in certain embodiments the skin cancer antigen can be an amino acid sequence or a functional fragment thereof that comprises at least about 70%, 75%, 80%, 85%, 86%, 87%, 90%, 91%, 92, 93%, 94%, 95%, 96%, 97%, 98%, 99% to any disclosed skin cancer antigen disclosed herein.

The present invention also relates to a composition comprising an amino acid sequence comprising an adjuvant peptide comprising an HLA I-Ab epitope from Aquifex aeolicus or a functional fragment thereof, wherein the adjuvant peptide can be no more than 20 amino acids, or in certain embodiments, no more than about 15 amino acids in length. In alternate embodiments, the adjuvant peptide can be capable of binding HLA-DRB1*07:01, HLA-DRB1*15:01 and HLA-DRB5*01:01, and in other embodiments, can comprise 70%, 75%, 80%, 85%, 86%, 87%, 90%, 91%, 92, 93%, 94%, 95%, 96%, 97%, 98%, 99% or 100% sequence identity to SEQ ID NO:1.

In alternate embodiments, the composition's amino acid sequence can further comprises a viral antigen and/or cancer antigen. In further embodiments, the amino acid sequence can comprise, from amino terminal to carboxy terminal orientation, the adjuvant peptide, a linker domain, and a viral and/or cancer antigen.

The present invention also relates to a pharmaceutical composition comprising: (i) any one or plurality of the nucleic acid sequences of described herein; and (ii) a pharmaceutically acceptable carrier.

In some embodiments, the disclosure relates methods of 1) inducing an immune response in a subject, 2) treating and/or preventing a viral infection or hyperproliferative disorder in a subject in need thereof, and 3) vaccinating a subject in need thereof, each by administering a therapeutically effective amount of the nucleic acid sequences or amino acid sequences as described herein. In some embodiments, the disclosed vaccine comprises a nucleic acid encoding an adjuvant disclosed herein, such as SEQ ID NO:1.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A-1G show evaluation of CD4+ T-cell responses to the nanoparticle structural domains induced by DNA vaccines. Mice received 25 μg DNA vaccination with EP twice three weeks apart and were euthanized two weeks post the second vaccination for cellular analysis. FIG. 1A: CD4+ T-cell IFNγ responses induced to the 3BVE, LS and PfV domains by DLnano_3BVE_GT8, DLnano_LS_GT8, and DLnano_PfV_GT8 vaccinations in BALB/c mice. FIG. 1B: Comparison of CD4+ cytokine responses to the LS domain induced by DLnano_LS_GT8 in BALB/c versus C57BL/6 mice. FIG. 1C: Comparison of polyfunctional CD4+ T-cell responses to the LS domain induced by DLnano_LS_GT8 in BALB/c versus C57BL/6 mice. FIG. 1D and FIG. 1E: Matrix mapping by IFNγ ELISpot assays (FIG. 1D) and ICS (FIG. 1E) to determine HLA I-Ad CD4+ T-cell epitopes in the LS domain in BALB/c mice immunized with DLnano_LS_GT8. FIG. 1F and FIG. 1G: Matrix mapping by IFNγ ELISpot assays (FIG. 1F) and ICS (FIG. 1G) to determine HLA I-Ab CD4+ T-cell epitopes in the LS domain in C57BL/6 mice immunized with DLnano_LS_GT8. Each group includes five mice; each dot represents an animal; error bar represents standard deviation; two-tailed Mann-Whitney Rank Test used to compare groups; p-values were adjusted for multiple comparison where appropriate; *, p-value<0.05.

FIG. 2A-2B shows in silico analysis using the SMM-align and NN-align to predict binding affinity, in terms of IC₅₀ value (nM), of the identified LS-3 (FIG. 2A), LS-13 and LS-15 (FIG. 2B) epitopes to common human and murine HLA alleles.

FIG. 3A-3F show analysis of the contributions of the identified LS-3 CD4-helper epitope to the antibody responses induced by DLnano_LS_GT8 in C57BL/6 mice. Mice received 25 μg DNA vaccination with EP twice three weeks apart and were euthanized two weeks post the second vaccination for cellular analysis. FIG. 3A: Engineering of CD4MutLS_GT8 mutants by selected mutations of the LS-3 epitope (in dark gray) that knocked out C57BL/6 HLA-IAb binding but still preserve assembly of the nanoparticle using structure-guided design, the remaining LS domain is shown in gray, the GT8 domain (light gray) is not shown. FIG. 3B: SEC-trace of lectin-column purified transfection supernatant of CD4MutLS_GT8 to determine the assembly status of designed CD4MutLS_GT8. FIG. 3C: Characterization of binding of recombinantly produced CD4MutLS_GT8, eOD-GT8-60mer and GT8-mono to VRC01 by ELISA. FIG. 3D and FIG. 3E: Cytokine expression by the ICS assay in C57BL/6 mice immunized with either DLnano_LS_GT8 or DLnano_CD4MutLS_GT8 to confirm knockout of the dominant LS-3 CD4+ helper epitope in CD4MutLS_GT8. FIG. 3F: Humoral responses to GT8 for mice immunized with DLnano_CD4MutLS_GT8, DLnano_LS_GT8 or DLmono_GT8 seven d.p.i. Each group includes five mice; each dot represents a mouse; error bar represents standard deviation; two-tailed Mann-Whitney Rank Test used to compare groups; p-values were adjusted for multiple comparison where appropriate; *, p-value<0.05.

FIG. 4A-4J show determination of whether the identified LS-3 epitope can enhance induced humoral responses to a model CA09 influenza HA immunogen (HA-RBD) through engineered genetic fusion of the identified epitopes with CA09 HA-RBD. C57BL/6 mice received either 25 μg DNA vaccination with EP twice four weeks apart and were euthanized one weeks post the second vaccination or 10 μg RIBI-adjuvanted protein vaccinations three times four weeks apart and were euthanized one week post the third vaccination. FIG. 4A: Layouts of the engineered LS3-CA09, LS3KO-CA09, and PADRE-CA09 fusion constructs. FIG. 4B and FIG. 4C: Flow plots (FIG. 4B) and groups statistics (FIG. 4C) to compare CD4+ T-cell cytokine responses induced by either DNA-encoded LS3-CA09 or LS3KO-CA09 immunizations in mice to LS3 and LS3KO peptides respectively. FIG. 4D and FIG. 4E: Flow plots (FIG. 4D) and groups statistics (FIG. 4E) to compare CD4+ T-cell cytokine responses induced by either DNA-encoded LS3-CA09 or PADRE-CA09 immunizations in mice to LS3 and PADRE peptides respectively. FIG. 4F: Comparison of poly-functional IFNγ+TNFα+IL-2+CD4+ T-cell responses to either LS3 or PADRE peptides in mice immunized as described in FIG. 4D and FIG. 4E. FIG. 4G and FIG. 4H: Comparison of anti-HA binding antibody responses (FIG. 4G) and HAI titers (FIG. 4H) in mice immunized with DNA-encoded LS3KO-CA09, LS3-CA09 or PADRE-CA09. FIG. 4I and FIG. 4J: Comparison of anti-HA binding antibody responses (FIG. 4I) and HAI titers (FIG. 4J) in mice immunized with RIBI-adjuvanted protein LS3KO-CA09, LS3-CA09 or PADRE-CA09. Each group includes five mice; each dot represents a mouse; error bar represents standard deviation; two-tailed Mann-Whitney Rank Test used to compare groups; p-values were adjusted for multiple comparison where appropriate; *, p-value<0.05.

FIG. 5A-5D show evaluation of T-cell responses to either the LS or GT8 domains induced by DLnano_LS_GT8 vaccination in BALB/c or C57BL/6 mice. Mice received 25 μg DNA vaccination with EP twice three weeks apart and were euthanized two weeks post the second vaccination for cellular analysis. FIG. 5A and FIG. 5B: Matrix mapping by IFNγ ELISpot assays in the GT8 domain to determine the dominant T-cell epitopes in BALB/c (FIG. 5A) or C57BL/6 (FIG. 5B) mice. FIG. 5C and FIG. 5D: Identification of the dominant CD8+ T-cell epitope by ICS in the GT8 domain for BALB/c mice (FIG. 5C) and in the LS domain for C57BL/6 mice (FIG. 5D). Each group includes five mice; error bar represents standard deviation; arrow above the bar graph represents the dominant peptide pool identified.

FIG. 6A-6B show analysis of the contributions of the identified LS-3 CD4-helper epitope to the antibody responses induced by DLnano_LS_GT8 in C57BL/6 mice. Mice were immunized in the same manner as described in FIG. 3. FIG. 6A: SEC-MAL trace of SEC-purified CD4MutLS_GT8; the molecular weight was determined to be around 2 MDa for CD4MutLS_GT8. FIG. 6B Humoral responses induced to GT8 by two doses of DLnano_CD4MutLS_GT8 in comparison to DLnano_LS_GT8 and DLmono_GT8, as assessed by ELISA; p-values compare differences between DLnano_CD4MutLS_GT8 and DLnano_LS_GT8 at each timepoint. Each group includes five mice; error bar represents standard deviation; two-tailed Mann-Whitney Rank Test used to compare groups; *, p-value<0.05.

FIG. 7A-7G show determination of whether the identified LS-3 epitope can enhance induced humoral responses to a model CA09 influenza HA immunogen. C57BL/6 mice received either DNA or protein vaccinations and were euthanized as described in FIG. 4. FIG. 7A and FIG. 7B: IFNγ+ ELIspot assays comparing T-cell responses induced by either DNA-encoded LS3-CA09 or LS3KO-CA09 immunizations in mice to LS3 and LS3KO peptides respectively. FIG. 7C and FIG. 7D: IFNγ+ ELIspot assays comparing T-cell responses induced by either DNA-encoded LS3-CA09 or PADRE-CA09 immunizations in mice to LS3 and PADRE peptides respectively. FIG. 7E: ICS analysis of CD4+ IFNγ+ responses induced by protein LS3KO-CA09, LS3-CA09, or PADRE-CA09 vaccinations in mice to LS3KO, LS3, and PADRE peptides respectively. FIG. 7F and FIG. 7G: IFNγ+ ELIspot assays comparing T-cell responses induced by protein LS3KO-CA09, LS3-CA09, or PADRE-CA09 vaccinations in mice to LS3KO, LS3, and PADRE peptides respectively. Each group includes five mice; each dot represents a mouse; error bar represents standard deviation; two-tailed Mann-Whitney Rank Test used to compare groups; p-values were adjusted for multiple comparison where appropriate; *, p-value<0.05.

### DETAILED DESCRIPTION

The disclosed method and compositions may be understood more readily by reference to the following detailed description of particular embodiments and the examples included therein and to the figures and their previous and following description. It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

It must be noted that as used herein and in the appended claims, the singular forms “a”, “an,” and “the” include plural reference unless the context clearly dictates otherwise. Thus, for example, reference to “a nucleic acid sequence” includes a plurality of nucleotides that are formed, reference to “the nucleic acid sequence” is a reference to one or more nucleic acid sequences and equivalents thereof known to those skilled in the art, and so forth.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD4+ T cells” or “CD4+ T cell activation” refer to a process (e.g., a signaling event) causing or resulting in one or more cellular responses of a CD4+ T cell (CTL), selected from: proliferation, differentiation, cytokine secretion, cytotoxic effector molecule release, cytotoxic activity, and expression of activation markers. As used herein, an “activated CD4+ T cell” refers to a CD4+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD4+ T cell activation are known in the art and are described herein.

The term “combination therapy” as used herein is meant to refer to administration of one or more therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dose having a fixed ratio of each therapeutic agent or in multiple, individual doses for each of the therapeutic agents. For example, one combination of the present disclosure may comprise a pooled sample of one or more nucleic acid molecules comprising one or a plurality of expressible nucleic acid sequences and an adjuvant and/or an anti-viral agent administered at the same or different times. In some embodiments, the pharmaceutical composition of the disclosure can be formulated as a single, co-formulated pharmaceutical composition comprising one or more nucleic acid molecules comprising one or a plurality of expressible nucleic acid sequences and one or more adjuvants and/or one or more anti-viral agents. As another example, a combination of the present disclosure (e.g., DNA or RNA vaccines and anti-viral agent) may be formulated as separate pharmaceutical compositions that can be administered at the same or different time. As used herein, the term “simultaneously” is meant to refer to administration of one or more agents at the same time. For example, in certain embodiments, antiviral vaccine or immunogenic composition and antiviral agents are administered simultaneously). Simultaneously includes administration contemporaneously or immediately sequentially, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered intramuscularly only. The components may be administered in any therapeutically effective sequence. A “combination” embraces groups of compounds or non-small chemical compound therapies useful as part of a combination therapy. In some embodiments, the therapeutic agent is an anti-retroviral therapy, (such as one or a combination of efavirenz, lamivudine and tenofovir disoproxil fumarate) or anti-flu therapy (such as TamiFlu®). In some embodiments, the therapeutic agent is one or a combiantion of: abacavir/dolutegravir/lamivudine (Triumeq), dolutegravir/rilpivirine (Juluca), elvitegravir/cobicistat/emtricitabine/tenofovir disoproxil fumarate (Stribild), elvitegravir/cobicistat/emtricitabine/tenofovir alafenamide (Genvoya), efavirenz/emtricitabine/tenofovir disoproxil fumarate (Atripla), emtricitabine/rilpivirine/tenofovir disoproxil fumarate (Complera), emtricitabine/rilpivirine/tenofovir alafenamide (Odefsey), bictegravir, emtricitabine, and tenofovir alafenamide (Biktarvy). In some embodiments, the therapeutic agent is one or a combination of a reverse transcrioptase inhibitor of a retrovirus such as efavirenz (Sustiva), etravirine (Intelence), nevirapine (Viramune), nevirapine extended-release (Viramune XR), rilpivirine (Edurant), delavirdine mesylate (Rescriptor). In some embodiments, the therapeutic agent is one or a combination of a protease inhibitor of a retrovirus, such as: atazanavir/cobicistat (Evotaz), darunavir/cobicistat (Prezcobix), lopinavir/ritonavir (Kaletra), ritonavir (Norvir), atazanavir (Reyataz), darunavir (Prezista), fosamprenavir (Lexiva), tipranavir (Aptivus).

As used herein, “expression” refers to the process by which a polynucleotide is transcribed from a DNA template (such as into and mRNA or other RNA transcript) and/or the process by which a transcribed mRNA (or administered mRNA) is translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. In some embodiments, the at least one expressible nucleic acid sequence comprises only DNA nucleotides, RNA nucleotides or comprises both RNA and DNA nucleotides. In some embodiments, the at least one expressible nucleic acid consist of RNA. In some embodiments, the at least one expressible nucleic acid consist of DNA.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based (such wild-type or full length sequences “reference sequences” or each individually a “reference sequence”). In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type human sequence upon which the sequence is derived. In some embodiments, the functional fragment may retain about 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, 100, 200, 300, 400, 500, 600, 700, 800, 900, 1000 or more nucleotides or amino acids.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

As used herein in the specification and in the claims, “or” should he understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein an “antigen” is meant to refer to any substance that elicits an immune response.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other. In some of the disclosed methods of treatment or prevention, the method comprises a step of electroporation of a subject's tissue for a sufficient time and with a sufficient electrical field capable of inducing uptake of the pharmaceutical compositions disclosed herein into the antigen-presenting cells. In some embodiments, the cells are antigen presenting cells.

The term “pharmaceutically acceptable excipient,” “pharmaceutically acceptable carrier” or “pharmaceutically acceptable diluent” as used herein is meant to refer to an excipient, carrier or diluent that can be administered to a subject, together with an agent or the pharmaceutical compositions disclosed herein, and which is inert or fails to eliminate the pharmacological activity of the active agent of the pharmaceutical composition. In some embodiments, the pharmaceutically acceptable carrier does fails to destroy or is incapable of eliminating the pharmacological activity of an active agent/vaccine and is nontoxic when administered in doses sufficient to deliver a therapeutic amount of the active agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the present disclosure. That is, e.g., a purified polypeptide of the present disclosure is a polypeptide that is at least from about 70 to 100% pure, i.e., the polypeptide is present in a composition wherein the polypeptide constitutes from about 70 to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure.

The terms “subject,” “individual,” and “patient” are used interchangeably herein to refer to a vertebrate, preferably a mammal, more preferably a human. Mammals include, but are not limited to, murine, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the subject is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal. The term “mammal” encompasses both humans and non-humans and includes but is not limited to humans, non-human primates, canines, felines, murine, bovines, equines, caprine, and porcines.

The term “therapeutic effect” as used herein is meant to refer to some extent of relief of one or more of the symptoms of a disorder (e.g., SARS-CoV-2 infection) or its associated pathology. A “therapeutically effective amount” as used herein is meant to refer to an amount of an agent which is effective, upon single or multiple dose administration (such as a first, second and/or third booster) to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the present disclosure employed in a pharmaceutical composition at levels lower than that required in order to achieve the desired therapeutic effect and gradually increase the dosage until the desired effect is achieved.

The terms “treat,” “treated,” “treating,” “treatment,” and the like as used herein are meant to refer to reducing or ameliorating a disorder and/or symptoms associated therewith (e.g., a viral infection). “Treating” can refer to administration of the DNA and/or RNA vaccines described herein to a subject after the onset, or suspected onset, of a viral infection. “Treating” includes the concepts of “alleviating,” which refers to lessening the frequency of occurrence or recurrence, or the severity, of any symptoms or other ill effects related to a virus and/or the side effects associated with viral therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

For any therapeutic agent described herein the therapeutically effective amount may be initially determined from preliminary in vitro studies and/or animal models. A therapeutically effective dose may also be determined from human data. The applied dose can be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties.

Nucleic acids containing one or more non-naturally occurring or modified nucleotides are also included within one definition of nucleic acids. The modified nucleotide analog may he located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NH₂, NHR, N₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of viral antigens. In some embodiments, the disclosure relates to a pharmaceutical composition comprising a first, second, third or more nucleic acid molecule, each of which encoding one or a plurality of viral antigens and at least one of each plasmid comprising one or more of the compositions disclosed herein.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Usually two sequences are sufficiently complementary when at least about 90% (preferably at least about 95%) of the nucleotides share base pair organization over a defined length of the molecule.

By “substantially identical” is meant nucleic acid molecule (or polypeptide) exhibiting at least about 50% identity to a reference amino acid sequence (for example, any one of the amino acid sequences described herein) or nucleic acid sequence (for example, any one of the nucleic acid sequences described herein). In some embodiments, such a sequence is at least about 60%, 70%, 80% or 85%, 90%, 95% or even 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), comprising additional, exogenous DNA, RNA or hybrid DNA or RNA molecules that can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

The term “vaccine” as used herein is meant to refer to a composition for generating immunity for the prophylaxis and/or treatment of diseases (e.g., viral infections). Accordingly, vaccines are medicaments which comprise antigens in protein and/or nucleic acid forms and are in animals for generating specific defense and protective substance by vaccination. A “vaccine composition” or a “DNA vaccine composition” can include a pharmaceutically acceptable excipient, carrier or diluent. A “DNA vaccine composition” as used herein can comprise a DNA vaccine, a RNA vaccine or a combination thereof.

“Variants” are intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring or endogenous nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly. In some embodiments, the nucleic acid molecules or the nucleic acid sequences comprise conservative mutations of 1, 2, 3, 4, 5, 6, 7, 8, 9, 10 or more nucleotides.

Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

In one aspect of the invention, it is desired that the LS-3 constructs provides for improved transcription and translation, including having one or more of the following: low GC content leader sequence to increase transcription; mRNA stability and codon optimization; eliminating to the extent possible cis-acting sequence motifs (i.e., internal TATA-boxes).

In some aspects of the invention, it is desired to incorporate the LS-3 constructs into a vaccine regimen, either as part of the vaccine composition or as a separate composition delivered in a coordinated fashion with the vaccine in order to generate a broad immune against vaccine immunogens. In some aspects of the invention, it is desired to provide the LS-3 constructs as an immunotherapeutic which can be used to modulate immune responses in an individual. In some aspects of the invention, it is desired to provide the improved LS-3 constructs in order to provide expression vectors which can be used to obtain high levels of LS-3 expression.

a. Antibody

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

b. Coding Sequence

“Coding sequence” or “encoding nucleic acid” as used herein may mean refers to the nucleic acid (RNA or DNA molecule) that comprise a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

1. Hyperproliferative

As used herein, the term “hyperproliferative diseases” is meant to refer to those diseases and disorders characterized by hyperproliferation of cells, senescence of cells or failure to clear, disruption of the cell cycle or disruption of apoptosis of cells and the term “hyperproliferative-associated protein” is meant to refer to proteins that are associated with a hyperproliferative disease. In some embodiments, hyperproliferative cells are those that are oncogenic, neoplastic, cancerous, tumor-forming or metastasizing.

m. Identical

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

n. Impedance

“Impedance” as used herein may be used when discussing the feedback mechanism and can be converted to a current value according to Ohm's law, thus enabling comparisons with the preset current.

o. Immune Response

“Immune response” as used herein may mean the activation of a host's immune system, e.g., that of a mammal, in response to the introduction of one or more RSV consensus antigen via the provided DNA plasmid vaccines. The immune response can be in the form of a cellular or humoral response, or both.

p. Intracellular Pathogen

“Intracellular pathogen” as used herein, is meant to refer to a virus or pathogenic organism that, at least part of its reproductive or life cycle, exists within a host cell and therein produces or causes to be produced, pathogen proteins.

q. Nucleic Acid

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

r. Operably Linked

“Operably linked” as used herein when referring to a gene operably linked to a promoter refers to the linkage of the two components such that expression of the gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. When referring to a signal peptide operable linked to a protein, the term refers to the protein having the signal peptide incorporated as part of the protein in a manner that it can function as a signal peptide. When referring to coding sequence that encodes a signal peptide operable linked to coding sequence that encodes a protein, the term refers to the coding sequences arranged such that the translation of the coding sequence produces a protein having the signal peptide incorporated as part of the protein in a manner that it can function as a signal peptide

s. Promoter

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

t. Stringent Hybridization Conditions

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50%>formamide, 5×SSC, and 1% SDS, incubating at 42° C., or, 5×SSC, 1% SDS, incubating at 65° C., with wash in 0.2×SSC, and 0.1% SDS at 65° C.

u. Substantially Complementary

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

v. Substantially Identical

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

w. Target Protein

“Target protein” as used herein is meant to refer to peptides and protein which are part of vaccines or which are encoded by gene constructs of DNA vaccines that act as target proteins for an immune response. The terms “target protein” and “immunogen” are used interchangeably and refer to a protein against which an immune response can be elicited. The target protein is an immunogenic protein that shares at least an epitope with a protein from the pathogen or undesirable cell-type such as a cancer cell or a cell involved in autoimmune disease against which an immune response is desired. The immune response directed against the target protein will protect the individual against and/or treat the individual for the specific infection or disease with which the target protein is associated

x. Variant

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties

y. Vector

“Vector” used herein may mean a nucleic acid sequence containing an origin of replication. A vector may be a plasmid, bacteriophage, bacterial artificial chromosome or yeast artificial chromosome. A vector may be a DNA or R A vector. A vector may be either a self-replicating extrachromosomal vector or a vector which integrates into a host genome.

### 2. LS-3 Epitope

Provided herein is LS-3 construct which encodes a HLA-IAb helper epitope LS-3 from Aquifex aeolicus. The LS-3 epitope can comprise the sequence: LRFGIVASRANHALV (SEQ ID NO: 1) and a LS-3 construct can comprise a nucleic acid sequence comprising the sequence CTGAGGTTCGGCATCGTGGCCAGCAGGGCCAACCACGCCCTGGTG (SEQ ID NO: 2). In some embodiments, the LS-3 epitope is encoded by a construct comprising a coding sequence on one plasmid. In some embodiments, the construct comprises promoter.

The LS-3 nucleic acid sequence (SEQ ID NO: 2) can optimized for human expression. The sequence have lower homology with the host genome to change the RNA structure and avoid cryptic regulation sequences. The sequences provide improved mRNA stability and expression.

Provided herein is a vector that is capable of expressing the LS-3 constructs in the cell of a mammal in a quantity effective to modulate an immune response in the mammal. Each vector may comprise heterologous nucleic acid encoding the one or both subunits. The vector may be a plasmid. The plasmid may be useful for transfecting cells with nucleic acid encoding the LS-3 epitope, which the transformed host cell is cultured and maintained under conditions wherein expression of the LS-3 epitope takes place.

The plasmid may comprise a nucleic acid encoding one or more antigens. The plasmid may further comprise an initiation codon, which may be upstream of the coding sequence, and a stop codon, which may be downstream of the coding sequence. The initiation and termination codon may be in frame with the coding sequence.

The plasmid may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040 175727, the contents of which are incorporated by reference herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference in their entireties.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may also comprise a regulatory sequence, which may be well suited for gene expression in a cell into which the plasmid is administered. The coding sequence may comprise a codon that may allow more efficient transcription of the coding sequence in the host cell.

The coding sequence may also comprise an Ig leader sequence. The leader sequence may be 5′ of the coding sequence. The consensus antigens encoded by this sequence may comprise an N-terminal Ig leader followed by a consensus antigen protein. The N-terminal Ig leader may be IgE or IgG.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNA I or pcDNA3 (Invitrogen, San Diego, Calif.), which maybe used for protein production in mammalian cells such as Chinese hamster ovary (CHO) cells.

4. Vaccine

According to some embodiments of the invention, the delivery of a nucleic acid sequence that encodes the LS-3 epitope or functional fragments thereof, in combination with a nucleic acid sequence that encodes an immunogen to an individual enhances the immune response against the immunogen. When the nucleic acid molecules that encode the immunogens and LS-3 are taken up by cells of the individual, the immunogen and LS-3 are expressed in cells and the proteins are thereby delivered to the individual. Aspects of the invention provide methods of delivering the coding sequences of the immunogen and LS-3 on a single nucleic acid molecule, methods of delivering the coding sequences of the immunogen and LS-3 on different nucleic acid molecules and methods of delivering the coding sequences of the proteins as part of recombinant vaccines and as part of attenuated vaccines.

According to some aspects of the present invention, compositions and methods are provided which prophylactically and/or therapeutically immunize an individual against a pathogen or abnormal, disease-related cells. The vaccine may be any type of vaccine such as, a live attenuated vaccine, a recombinant vaccine or a nucleic acid or DNA vaccine. By delivering nucleic acid molecules that encode an immunogen and LS-3 epitope or functional fragments thereof the immune response induced by the vaccine may be modulated. The LS-3 constructs are particularly useful when delivered in combination with a nucleic acid molecule that encodes an immunogen such as for example as part of a plasmid or the genome of a recombinant vector or attenuated pathogen or cell. The LS-3 constructs may be used in vaccines prophylactically in order to induce a protective immune response in an uninfected or disease free individual. LS-3 constructs are particularly useful when delivered to induce a protective immune response in humans. The LS-3 constructs may be used in vaccines therapeutically in order to induce a immune response in an infected or diseased individual. The LS-3 constructs are particularly useful when delivered to induce a therapeutic immune response in humans. In some embodiments, nucleic acid molecules comprising the LS-3 constructs are delivered in a cell free composition. In some embodiments, nucleic acid molecules comprising the LS-3 constructs are delivered in a composition free of cancer cells. In some embodiments, comprising the LS-3 constructs are administered free of any other cytokine.

Provided herein are vaccine capable of generating in a mammal an immune response against pathogens, immunogens expressed on cells associated with disease and other immunogens against which an immune response is desired. The vaccine may comprise each plasmid as discussed above. The vaccine may comprise a plurality of the plasmids, or combinations thereof. The vaccine may be provided to induce a therapeutic or prophylactic immune response.

Genetic constructs may comprise a nucleotide sequence that encodes a target protein or an immunomodulating protein operably linked to regulatory elements needed for gene expression. According to the invention, combinations of gene constructs that include one construct that comprises an expressible form of the nucleotide sequence that encodes a target protein and one construct that includes an expressible form of the nucleotide sequence that encodes an immunomodulating protein are provided. Delivery into a living cell of the DNA or RNA molecule(s) that include the combination of gene constructs results in the expression of the DNA or RNA and production of the target protein and one or more immunomodulating proteins. An enhanced immune response against the target protein results.

The present invention may be used to immunize an individual against pathogens such as viruses, prokaryote and pathogenic eukaryotic organisms such as unicellular pathogenic organisms and multicellular parasites. The present invention is particularly useful to immunize an individual or subject against those pathogens which infect cells and which are not encapsulated such as viruses, and prokaryote such as gonorrhea, listeria and shigella. In addition, the present invention is also useful to immunize an individual against protozoan pathogens that include a stage in the life cycle where they are intracellular pathogens. Table 1 provides a listing of some of the viral families and genera for which vaccines according to the present invention can be made. DNA constructs that comprise DNA sequences that encode the peptides that comprise at least an epitope identical or substantially similar to an epitope displayed on a pathogen antigen such as those antigens listed on the tables are useful in vaccines. Moreover, the present invention is also useful to immunize an individual against other pathogens including prokaryotic and eukaryotic protozoan pathogens as well as multicellular parasites such as those listed on Table 2.

### Table 1—Viruses

### Picornavirus Family

Genera:

Pvhinoviruses: (Medical) responsible for −50% cases of the common cold.

Ethero viruses: (Medical) includes polioviruses, coxsackieviruses, echoviruses, and human enteroviruses such as hepatitis A virus.

Apthoviruses: (Veterinary) these are the foot and mouth disease viruses.

Target antigens: VP1, VP2, VP3, VP4, VPG

### Calcivirus Family

Genera:

Norwalk Group of Viruses: (Medical) these viruses are an important causative agent of epidemic gastroenteritis.

### Togavirus Family

Genera:
Alphaviruses: (Medical and Veterinary) examples include Sindbis virus, RossRiver virus
   and Venezuelan Eastern & Western Equine encephalitis viruses.

Reovirus: (Medical) Rubella virus.

### Flariviridae Family

Examples include: (Medical) dengue, yellow fever, Japanese encephalitis, St. Louis encephalitis and tick borne encephalitis viruses. West Nile virus (Genbank NC001563, AF533540, AF404757, AF404756, AF404755, AF404754, AF404753, AF481864, M12294, AF317203, AF196835, AF260969, AF260968, AF260967, AF206518 and AF202541)

Representative Target antigens: E NS5 C

Hepatitis C Virus: (Medical) these viruses are not placed in a family yet but are believed to be
either a togavirus or a flavivirus. Most similarity is with togavirus family.

### Coronavirus Family: (Medical and Veterinary)

Infectious bronchitis virus (poultry)

Porcine transmissible gastroenteric virus (pig)
Porcine hemagglutinating encephalomyelitis virus (pig)
Feline infectious peritonitis virus (cats)
Feline enteric coronavirus (cat)
Canine coronavirus (dog)
SARS associated coronavirus
The human respiratory coronaviruses cause about 40% of cases of common cold. EX. 224E, OC43 Note—coronaviruses may cause non-A, B or C hepatitis
Target antigens: E1—also called M or matrix protein E2—also called S or Spike protein
   E3—also called BE or hemagglutin-elterose glycoprotein (not present in all coronaviruses)

### N—

nucleocapsid

### Rhabdovirus Family

Genera:

Vesiculovirus, Lyssavirus: (medical and veterinary) rabies

Target antigen: G protein, N protein

### Filoviridae Family: (Medical)

Hemorrhagic fever viruses such as Marburg and Ebola virus

### Paramyxovirus Family:

Genera:

Paramyxovirus: (Medical and Veterinary) Mumps virus, New Castle disease virus (important pathogen in chickens)

Morbillivirus: (Medical and Veterinary) Measles, canine distemper

Pneumovirus: (Medical and Veterinary) Respiratory syncytial virus

Orthomyxovirus Family (Medical) The Influenza virus

### Bunyavirus Family

Genera:

Bunyavirus: (Medical) California encephalitis, La Crosse

Phlebovirus: (Medical) Rift Valley Fever

Hantavirus: Puremala is a hemahagin fever virus

Nairvirus (Veterinary) Nairobi sheep disease

Also many unassigned bungaviruses

Arenavirus Family (Medical) LCM, Lassa fever virus

### Reovirus Family

Genera:

Reovirus: a possible human pathogen

Rotavirus: acute gastroenteritis in children

Orbiviruses: (Medical and Veterinary) Colorado Tick fever,

Lebombo (humans) equine encephalosis, blue tongue

### Retroyirus Family

Sub-Family:

Oncorivirinal: (Veterinary) (Medical) feline leukemia virus, HTLVI and HTLVII

Lentivirinal: (Medical and Veterinary) HIV, feline immunodeficiency virus, equine infections, anemia virus

### Spumavirinal Papovavirus Family

Sub-Family:

Polyomaviruses: (Medical) BKU and JCU viruses

Sub-Family:

Papillomavirus: (Medical) many viral types associated with cancers or malignant progression of papilloma.

Adenovirus (Medical) EX AD7, ARD., O.B.—cause respiratory disease—some adenoviruses such as 275 cause enteritis

### Parvovirus Family (Veterinary)

Feline parvovirus: causes feline enteritis

Feline panleucopeniavirus

Canine parvovirus

Porcine parvovirus

### Herpesvirus Family

Sub-Family:

alphaherpesviridue

Genera:

Simplexvirus (Medical)

HSVI (Genbank X141 12, NC001806),

HSVII (NC001798)

Varicella zoster: (Medical Veterinary)

Pseudorabies

varicella zoster

Sub-Family

betaherpesviridae

Genera:

Cytomegalovirus (Medical)

HCMV

Muromegalovirus

Sub-Family.

Gammaherpesviridae

Genera:

Lymphocryptovirus (Medical)

EBV—(Burkitt's lymphoma)

### Poxvirus Family

Sub-Family:

Chordopoxviridae (Medical—Veterinary)

Genera:

Variola (Smallpox)

Vaccinia (Cowpox)

Parapoxivirus—Veterinary

Auipoxvirus—Veterinary

Capripoxvirus

Leporipoxvirus

Suipoxviru's

Sub-Family:

Entemopoxviridue

### Hepadnavirus Family

Hepatitis B virus

Unclassified Hepatitis delta virus

### Table 2

Bacterial pathogens

Pathogenic gram-positive cocci include: pneumococcal; staphylococcal;

and streptococcal.

Pathogenic gram-negative cocci include: meningococcal; and gonococcal.

Pathogenic enteric gram-negative bacilli include: enterobacteriaceae; pseudomonas, acinetobacteria and eikenella, melioidosis; salmonella; shigellosis; haemophilus; chancroid; brucellosis; tularemia; yersinia (pasteurella); streptobaciUus mortiliformis and spirillum; listeria monocytogenes; erysipelothrix rhusiopathiae; diphtheria, cholera, anthrax; donovanosis (granuloma inguinale); and bartonellosis.

Pathogenic anaerobic bacteria include: tetanus; botulism; other Clostridia; tuberculosis; leprosy; and other mycobacteria.

Pathogenic spirochetal diseases include: syphilis;—treponematoses: yaws, pinta and endemic syphilis; and leptospirosis.

Other infections caused by higher pathogen bacteria and pathogenic fungi include: actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma, and chromomycosis; and dermatophytosis.

Rickettsial infections include rickettsial and rickettsioses.

Examples of mycoplasma and chlamydial infections include: Mycoplasma pneumoniae; lymphogranuloma venereum; psittacosis; and perinatal chlamydial infections.

Pathogenic eukaryotes

Pathogenic protozoans and helminths and infections thereby include: amebiasis; malaria; leishmaniasis; trypanosomiasis; toxoplasmosis; Pneumocystis carinii; babesiosis; giardiasis; trichinosis; filariasis; schistosomiasis; nematodes; trematodes or flukes; and cestode (tapeworm) infections.

In order to produce a genetic vaccine to protect against pathogen infection, genetic material that encodes immunogenic proteins against which a protective immune response can be mounted must be included in a genetic construct as the coding sequence for the target. Because DNA and RNA are both relatively small and can be produced relatively easily, the present invention provides the additional advantage of allowing for vaccination with multiple pathogen antigens. The genetic construct used in the genetic vaccine can include genetic material that encodes many pathogen antigens. For example, several viral genes may be included in a single construct thereby providing multiple targets.

Tables 1 and 2 include lists of some of the pathogenic agents and organisms for which genetic vaccines can be prepared to protect an individual from infection by them.

In some embodiments, vaccines comprise the optimized LS-3 nucleic acid sequence in combination with one or more DNA vaccine constructs set forth in the following patent documents which are each incorporated herein by reference. In some embodiments, vaccines comprise the optimized LS-3 in combination with (human immunodeficiency virus) an HIV vaccine, an (hepatitis C virus) HCV vaccine, a human papilloma virus (HPV) vaccine, an influenza vaccine or an hTERT-targeted cancer vaccines as disclosed in PCT application PCT/US07/74769 and corresponding U.S. patent application Ser. No. 12/375,518. In some embodiments, vaccines comprise the optimized IL-12 in combination with an Influenza vaccines disclosed in PCT application PCT/US08/83281 and corresponding U.S. patent application Ser. No. 12/269,824 or PCT application PCT/US11/22642 and corresponding U.S. patent application Ser. No. 12/694,238. In some embodiments, vaccines comprise the optimized IL-12 in combination with an HCV vaccines disclosed in PCT application PCT/US08/081627 and corresponding U.S. patent application Ser. No. 13/127,008. In some embodiments, vaccines comprise the optimized LS-3 in combination with an HPV vaccines disclosed in PCT application.

PCT/US10/21869 and corresponding U.S. patent application Ser. No. 12/691,588 or U.S. provisional application Ser. No. 61/442,162. In some embodiments, vaccines comprise the optimized LS-3 in combination with an Smallpox vaccines disclosed in PCT application PCT/US09/045420 and corresponding U.S. patent application Ser. No. 12/473,634. In some embodiments, vaccines comprise the optimized LS-3 in combination with an Chikungunya vaccines disclosed in PCT application PCT/US09/039656 and corresponding U.S. patent application Ser. No. 12/936,186. In some embodiments, vaccines comprise the optimized LS-3 in combination with an foot and mouth disease virus (FMDV) vaccines disclosed in PCT application PCT/US10/55187. In some embodiments, vaccines comprise the optimized LS-3 in combination with an Malaria vaccines disclosed in U.S. provisional application Ser. No. 61/386,973. In some embodiments, vaccines comprise the optimized LS-3 in combination with an prostate cancer vaccines disclosed in U.S. provisional application Ser. No. 61/413,176 or U.S. provisional application Ser. No. 61/417,817. In some embodiments, vaccines comprise the optimized LS-3 in combination with an human cytomegalovirus (CMV) vaccines disclosed in U.S. provisional application Ser. No. 61/438,089. In some embodiments, vaccines comprise the optimized LS-3 in combination with Methicillin-Resistant Staphylococcus aureus (MRSA) vaccines disclosed in U.S. Provisional Application Ser. No. 61/569,727, filed on Dec. 12, 2011, entitled “PROTEINS COMPRISING MRSA PBP2A AND FRAGMENTS THEREOF, NUCLEIC ACIDS ENCODING THE SAME, AND COMPOSITIONS AND THEIR USE TO PREVENT AND TREAT MRSA INFECTIONS” and designated attorney docket number 133172.04000 (X5709) and its corresponding PCT Application claiming priority to U.S. Provisional Application Ser. No. 61/569,727, filed on the same day as the application filed herewith, each of which incorporate by reference in their entireties. All patents and patent applications disclosed herein are incorporated by reference in their entireties.

Another aspect of the present invention provides a method of conferring a protective immune response against hyperproliferating cells that are characteristic in hyperproliferative diseases and to a method of treating individuals suffering from hyperproliferative diseases. Examples of hyperproliferative diseases include all forms of cancer and psoriasis.

It has been discovered that introduction of a genetic construct that includes a nucleotide sequence which encodes an immunogenic “hyperproliferating cell”-associated protein into the cells of an individual results in the production of those proteins in the vaccinated cells of an individual. To immunize against hyperproliferative diseases, a genetic construct that includes a nucleotide sequence that encodes a protein that is associated with a hyperproliferative disease is administered to an individual. In some embodiments, the hyperproliferative disease is cancer.

In order for the hyperproliferative-associated protein to be an effective immunogenic target, it must be a protein that is produced exclusively or at higher levels in hyperproliferative cells as compared to normal cells. Target antigens include such proteins, fragments thereof and peptides; which comprise at least an epitope found on such proteins. In some cases, a hyperproliferative-associated protein is the product of a mutation of a gene that encodes a protein. The mutated gene encodes a protein that is nearly identical to the normal protein except it has a slightly different amino acid sequence which results in a different epitope not found on the normal protein. Such target proteins include those which are proteins encoded by oncogenes such as myb, myc, fyn, and the translocation gene bcr/abl, ras, src, P53, neu, trk and EGRF. In addition to oncogene products as target antigens, target proteins for anti-cancer treatments and protective regimens include variable regions of antibodies made by B cell lymphomas and variable regions of T cell receptors of T cell lymphomas which, in some embodiments, are also used target antigens for autoimmune disease. Other tumor-associated proteins can be used as target proteins such as proteins that are found at higher levels in tumor cells including the protein recognized by monoclonal antibody 17-IA and folate binding proteins or PSA.

While the present invention may be used to immunize an individual against one or more of several forms of cancer, the present invention is particularly useful to prophylactically immunize an individual who is predisposed to develop a particular cancer or who has had cancer and is therefore susceptible to a relapse. Developments in genetics and technology as well as epidemiology allow for the determination of probability and risk assessment for the development of cancer in individual. Using genetic screening and/or family health histories, it is possible to predict the probability a particular individual has for developing any one of several types of cancer.

Similarly, those individuals who have already developed cancer and who have been treated to remove the cancer or are otherwise in remission are particularly susceptible to relapse and reoccurrence. As part of a treatment regimen, such individuals can be immunized against the cancer that they have been diagnosed as having had in order to combat a recurrence. Thus, once it is known that an individual has had a type of cancer and is at risk of a relapse, they can be immunized in order to prepare their immune system to combat any future appearance of the cancer.

The present invention provides a method of treating individuals suffering from hyperproliferative diseases. In such methods, the introduction of genetic constructs serves as an immunotherapeutic, directing and promoting the immune system of the individual to combat hyperproliferative cells that produce the target protein. In treating or preventing cancer, embodiments which are free of cells are particularly useful.

The present invention provides a method of treating individuals suffering from autoimmune diseases and disorders by conferring a broad based protective immune response against targets that are associated with autoimmunity including cell receptors and cells which produce “self-directed antibodies.

T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors that bind to endogenous antigens and initiate the inflammatory cascade associated with autoimmune diseases.

Vaccination against the variable region of the T cells would elicit an immune response including CTLs to eliminate those T cells.

In RA, several specific variable regions of T cell receptors (TCRs) that are involved in the disease have been characterized. These TCRs include ν β-3, ν β-14, 20 ν β-17 and Va-17. Thus, vaccination with a DNA construct that encodes at least one of these proteins will elicit an immune response that will target T cells involved in RA. See: Howell, M. D., et al., 1991 Proc. Nat. Acad. Sci. USA 88:10921-10925; Piliard, X., et al, 1991 Science 253:325-329; Williams, W. V., et al, 1992 J Clin. Invest. 90:326-333; each of which is incorporated herein by reference. In MS, several specific variable regions of TCRs that are involved in the disease have been characterized. These TCRs include ν β-7, and Va-10. Thus, vaccination with a DNA construct that encodes LS-3 epitope and at least one of these proteins will elicit an immune response that will target T cells involved in MS. See: Wucherpfennig, K. W., et al, 1990 Science 248:1016-1019; Oksenberg, J. R., et al, 1990 Nature 345:344-346; each of which is incorporated herein by reference.

In scleroderma, several specific variable regions of TCRs that are involved in the disease have been characterized. These TCRs include ν β-6, ν β-8, V β-14 and Va-16, Va-3C, Va-7, Va-14, Va-15, Va-16, Va-28 and Va-12. Thus, vaccination with a DNA construct that encodes LS-3 epitope and at least one of these proteins will elicit an immune response that will target T cells involved in scleroderma.

In order to treat patients suffering from a T cell mediated autoimmune disease, particularly those for which the variable region of the TCR has yet to be characterized, a synovial biopsy can be performed. Samples of the T cells present can be taken and the variable region of those TCRs identified using standard techniques. Genetic vaccines can be prepared using this information.

B cell mediated autoimmune diseases include Lupus (SLE), Grave's disease, myasthenia gravis, autoimmune hemolytic anemia, autoimmune thrombocytopenia, asthma, cryoglobulinemia, primary biliary sclerosis and pernicious anemia. Each of these diseases is characterized by antibodies that bind to endogenous antigens and initiate the inflammatory cascade associated with autoimmune diseases. Vaccination against the variable region of antibodies would elicit an immune response including CTLs to eliminate those B cells that produce the antibody.

In order to treat patients suffering from a B cell mediated autoimmune disease, the variable region of the antibodies involved in the autoimmune activity must be identified. A biopsy can be performed and samples of the antibodies present at a site of inflammation can be taken. The variable region of those antibodies can be identified using standard techniques. Genetic vaccines can be prepared using this information.

In the case of SLE, one antigen is believed to be DNA. Thus, in patients to be immunized against SLE, their sera can be screened for anti-DNA antibodies and a vaccine can be prepared which includes DNA constructs that encode the variable region of such anti-DNA antibodies found in the sera.

Common structural features among the variable regions of both TCRs and antibodies are well known. The DNA sequence encoding a particular TCR or antibody can generally be found following well known methods such as those described in Kabat, et al 1987 Sequence of Proteins of Immunological Interest U.S. Department of Health and Human Services, Bethesda Md., which is incorporated herein by reference. In addition, a general method for cloning functional variable regions from antibodies can be found in Chaudhary, V. K., et al, 1990 Proc. Natl. Acad Sci. USA 87:1066, which is incorporated herein by reference.

In addition to using expressible forms of immunomodulating protein coding sequences to improve genetic vaccines, the present invention relates to improved attenuated live vaccines and improved vaccines that use recombinant vectors to deliver foreign genes that encode antigens. Examples of attenuated live vaccines and those using recombinant vectors to deliver foreign antigens are described in U.S. Pat. Nos. 4,722,848; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; and 5,482,713, which are each incorporated herein by reference. Gene constructs are provided which include the nucleotide sequence of the LS-3 constructs or functional fragments thereof, wherein the nucleotide sequence is operably linked to regulatory sequences that can function in the vaccine to effect expression. The gene constructs are incorporated in the attenuated live vaccines and recombinant vaccines to produce improved vaccines according to the invention.

The vaccine may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be one or more additional adjuvants. An adjuvant may be other genes that are expressed from the same or from an alternative plasmid or are delivered as proteins in combination with the plasmid above in the vaccine. The one or more adjuvants may be proteins and/or nucleic acid molecules that encode proteins selected from the group consisting of: PADRE, a-interferon (IFN-a), β-interferon (IFN-β), γ-interferon, platelet derived growth factor (PDGF), TNFa, TNFp, GM-CSF, epidermal growth factor (EGF), cutaneous T cell-attracting chemokine (CTACK), epithelial thymus-expressed chemokine (TECK), mucosae-associated epithelial chemokine (MEC), IL-15 including IL-15 having the signal sequence or coding sequence that encodes the signal sequence deleted and optionally including a different signal peptide such as that from IgE or coding sequence that encodes a difference signal peptide such as that from IgE, IL-28, MHC, CD80, CD86, IL-1, IL-2, IL-4, IL-5, IL-6, IL-10, IL-18, IL-12, MCP-1, MIP-1α, MIP-Iβ, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof. In some embodiments, an additional adjuvant may be one or more proteins and/or nucleic acid molecules that encode proteins selected from the group consisting of: IL-15, IL-28, CTACK, TECK, MEC or RANTES. Examples of IL-15 constructs and sequences are disclosed in PCT application no. PCT/US04/18962 and corresponding U.S. application Ser. No. 10/560,650, and in PCT application no. PCT/US07/00886 and corresponding U.S. application Ser. No. 12/160,766, and in PCT application no. PCT/US Ser. No. 10/048,827. Examples of IL-28 constructs and sequences are disclosed in PCT application no. PCT/US09/039648 and corresponding U.S. application Ser. No. 12/936,192. Examples of RANTES and other constructs and sequences are disclosed in PCT application no. PCT/US 1999/004332 and corresponding U.S. application Ser. No. 09/622,452. Other examples of RANTES constructs and sequences are disclosed in PCT application no. PCT/US11/024098. Examples of RANTES and other constructs and sequences are disclosed in PCT application no. PCT/US 1999/004332 and corresponding U.S. application Ser. No. 09/622,452. Other examples of RANTES constructs and sequences are disclosed in PCT application no. PCT/US11/024098. Examples of chemokines CTACK, TECK and MEC constructs and sequences are disclosed in PCT application no. PCT/US2005/042231 and corresponding U.S. application Ser. No. 11/719,646. Examples of OX40 and other immunomodulators are disclosed in U.S. application Ser. No. 10/560,653. Examples of DR5 and other immunomodulators are disclosed in U.S. application Ser. No. 09/622,452.

The vaccine may further comprise a genetic vaccine facilitator agent as described in U.S. Pat. No. 5,962,428, which is fully incorporated by reference in its entirety.

The vaccine may comprise the consensus antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some embodiments, the pharmaceutical compositions contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of the consensus antigen or plasmid thereof.

The vaccine may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

### 5. Methods of Delivery the Vaccine

Provided herein is a method for delivering a vaccine including the LS-3 epitope to produce immune responses effective against the vaccine immunogens. The method of delivering the vaccine or vaccination may be provided to induce a therapeutic and prophylactic immune response. The vaccination process may generate an immune response against immunogens in a subject. The vaccine may be delivered to an individual to modulate the activity of the mammal's immune system and enhance the immune response. The delivery of the vaccine may be the transfection of sequences encoding the immunogen and the LS-3 epitope on one or more nucleic acid molecules. The coding sequences are expressed in cells and delivered to the surface of the cell upon which the immune system recognized and induces a cellular, humoral, or cellular and humoral response. The delivery of the vaccine may be use to induce or elicit and immune response in mammals against the immunogen by administering to the mammals the vaccine as discussed above. The inclusion of the LS-3 epitope results in a more effective immune response.

Upon delivery of the vaccine and plasmid into the cells of the mammal, the transfected cells will express and secrete immunogens and LS-3 epitope encoded by the plasmids injected from the vaccine. These immunogens will be recognized as foreign by the immune system and antibodies will be made against them. These antibodies will be maintained by the immune system and allow for an effective response to subsequent infections. The presence of the LS-3 epitope encoded by the LS-3 epitope constructs results in a greater immune response.

The vaccine may be administered to a mammal to elicit an immune response in a mammal. The mammal may be human, primate, non-human primate, cow, cattle, sheep, goat, antelope, bison, water buffalo, bison, bovids, deer, hedgehogs, elephants, llama, alpaca, mice, rats, and chicken.

a. Combination Treatments

The LS-3 epitope may be administered in combination with other proteins or genes encoding one or more of alpha-interferon, γ-interferon, platelet derived growth factor (PDGF), TNFa, TNFp, GM-CSF, epidermal growth factor (EGF), cutaneous T cell-attracting chemokine (CTACK), epithelial thymus-expressed chemokine (TECK), mucosae-associated epithelial chemokine (MEC), IL-15 (including IL-15 having the signal sequence deleted and optionally including the signal peptide from IgE), MHC, CD80, CD86, IL-1, IL-2, IL-4, IL-5, IL-6, IL-10, IL-12, IL-18, IL-28, MCP-1, MIP-1α, MIP-Iβ, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, N K, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or combinations thereof.

The vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns”, or other physical methods such as electroporation (“EP”), “hydrodynamic method”, or ultrasound.

The plasmid of the vaccine may be delivered to the mammal by several well known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The consensus antigen may be delivered via DNA injection and along with in vivo electroporation.

### 6. Immunomodulating Compositions and Methods

In some embodiments, the nucleic acid sequences that encode the LS-3 eptiope are delivered without the addition of nucleic acid sequences that encode an immunogen. In some embodiments, the method is free of delivery of a nucleic acid that encodes an immunogen. In such methods, the nucleic acid sequences that encode the LS-3 epitope subunits are used as immunotherapeutics which, when expressed to produce functional LS-3, impart a desired immunomodulatory effect on the individual. The nucleic acid sequences that encode the LS-3 epitope are provided and delivered as described above except for the exclusion of nucleic acid sequences that encode an immunogen. In such methods, the nucleic acid sequences that encode the LS-3 epitope may used as immunotherapeutics alone or in combination with other immunomodulatory proteins such as those described above in the section entitled combination treatments.

### A. Nucleic Acid Compositions

Disclosed are compositions comprising one or plurality of expressible nucleic acid sequences. In some embodiments, the expressible nucleic acid sequence is a DNA. In other embodiments, the expressible nucleic acid sequence is a RNA. In some embodiments, the expressible nucleic acid is operably linked to one or a plurality of regulatory sequences. In some embodiments, the expressible nucleic acid sequence is comprised and forms a part of a nucleic acid molecule, such as a vector or plasmid.

In one aspect, the expressible nucleic acid sequence of the disclosure comprises a first nucleic acid sequence encoding a scaffold domain comprising a self-assembling polypeptide or a pharmaceutically acceptable salt thereof, and a second nucleic acid sequence encoding an antigen domain comprising a viral antigen or a pharmaceutically acceptable salt thereof. The self-assembling polypeptide is a self-assembling peptide that is expressed to envelope the viral antigen. Transformed or transfected cells exposed to such expressible nucleic acid sequences can produce the self-assembling peptide which envelopes the viral antigens, thereby stimulating the viral antigen-specific immune response against the antigen. In some embodiments, the antigen-specific immune response is a therapeutically effective immune response against the virus from which the antigen amino acid sequence is obtained. In some embodiments, the viral antigen encoded by the expressible nucleic acid of the disclosure comprises a coronaviral antigen. In some embodiments, the expressible nucleic acid sequence further comprises a third nucleic acid sequence encoding a leader sequence or a pharmaceutically acceptable salt thereof. In some embodiments, the leader sequence is an IgE or IgG leader sequence. In some embodiments, the expressible nucleic acid sequence further comprises a fourth nucleic acid sequence encoding a linker peptide or a pharmaceutically acceptable salt thereof, wherein the fourth nucleic acid sequence is positioned between the first nucleic acid sequence and the second nucleic acid sequence in the 5′ to 3′ orientation. In some embodiments, the expressible nucleic acid sequence comprises a first nucleic acid sequence encoding a scaffold domain comprising a self-assembling polypeptide or a pharmaceutically acceptable salt thereof, a second nucleic acid sequence encoding an antigen domain comprising a viral antigen or a pharmaceutically acceptable salt thereof, and a third nucleic acid sequence encoding a leader sequence or a pharmaceutically acceptable salt thereof. In some embodiments, the expressible nucleic acid sequence comprises a first nucleic acid sequence encoding a scaffold domain comprising a self-assembling polypeptide or a pharmaceutically acceptable salt thereof, a second nucleic acid sequence encoding an antigen domain comprising a viral antigen or a pharmaceutically acceptable salt thereof, a third nucleic acid sequence encoding a leader sequence or a pharmaceutically acceptable salt thereof, and a fourth nucleic acid sequence encoding a linker peptide or a pharmaceutically acceptable salt thereof, wherein the fourth nucleic acid sequence is positioned between the first nucleic acid sequence and the second nucleic acid sequence in the 5′ to 3′ orientation.

In some embodiments, the expressible nucleic acid sequence of the disclosure comprises a nucleic acid sequence encoding a viral trimer polypeptide, a functional fragment thereof or a pharmaceutically acceptable salt thereof. In some embodiments, the expressible nucleic acid sequence comprises, in a 5′ to 3′ orientation, a first nucleic acid sequence encoding a leader sequence or a pharmaceutically acceptable salt thereof, and a second nucleic acid sequence encoding a viral trimer polypeptide, a functional fragment or variant thereof or a pharmaceutically acceptable salt thereof. In some embodiments, the leader sequence is an IgE or IgG leader sequence. In some embodiments, the expressible nucleic acid sequence comprises, in a 5′ to 3′ orientation, a first nucleic acid sequence encoding a leader sequence or a pharmaceutically acceptable salt thereof, and a second nucleic acid sequence encoding a viral polypeptide that is a component of a viral trimer, a functional fragment or variant thereof or a pharmaceutically acceptable salt thereof. In some embodiments, the viral polypeptide that is a component of a viral trimer is a monomer of a viral trimer, such that, upon expression, the monomers spontaneously aggregate to form a trimeric viral polypeptide. In some embodiments, the viral trimer encoded by the expressible nucleic acid of the disclosure comprises a coronaviral trimer. In some embodiments, the viral trimer comprises the spike protein of SARS-CoV-2, a functional fragment or variant thereof or a pharmaceutically acceptable salt thereof.

In some embodiments, the nucleic acid sequences encoding the viral antigens or viral trimers comprised in the expressible nucleic acid of the disclosure comprise one or a plurality of mutations so to tailor the vaccine induced responses. Such mutations result in creating glycan sites in the encoded polypeptide so that glycosylation events can be obtained. In some embodiments, such glycan modifications or mutations decrease the bottom reactivity. In some embodiments, such glycan modifications or mutations increase antigen activity.

1. Leader Sequence

The expressible nucleic acid sequence of the present disclosure optionally comprises a nucleic acid sequence encoding a leader sequence, a functional fragment or variant thereof or a pharmaceutically acceptable salt thereof. A “leader sequence” may from time to time refer to a “signal peptide” and thus, the terms “leader sequence” and “signal peptide” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences, when present, are linked at the N terminus of the protein. The presence of a leader sequence may be required for proper secretion of the viral antigen or trimer encoded by the expressible nucleic acid sequence of the disclosure.

A non-limiting example of the leader sequence is the IgE leader sequence comprising the amino acid sequence of MDWTWILFLVAAATRVHS (SEQ ID NO: 1; also named “MD39”) encoded by one of the following nucleic acid sequences:

(SEQ ID NO: 2; “MD39”)
atggactggacatggattctgttcctggtcgctgccgctacaagagtgcat
tcc;
(SEQ ID NO: 3; “CPG9.2”)
atggattggacttggattctgttcctggtcgcagcagccacacgagtgcat
agc;
and
(SEQ ID NO: 4)
atggactggacctggattctgttcctggtggccgccgccacaagggtgcac
agc

Another non-limiting example of the leader sequence is the amino acid sequence of MDWTWRILFLVAAATGTHA (SEQ ID NO: 5) encoded by the nucleic acid sequence of atggactggacctggagaatcctgttcctggtggccgccgccaccggcacacacgccgatacacacttccccatctgcatcttttgctg tggctgttgccataggtccaagtgtgggatgtgctgcaaaact (SEQ ID NO: 6).

Thus, in some embodiments when the leader sequence is present, the leader sequence may comprise at least about 60%, 65%, 70%, 75%, 80%, 85%, 90%, 95%, or 99% sequence identity to SEQ ID NO: 1 or SEQ ID NO: 5, or a functional fragment or variant thereof. In some embodiments when the leader sequence is present, the leader sequence may comprise the amino acid sequence of SEQ ID NO: 1 or SEQ ID NO: 5, or a functional fragment or variant thereof. In some embodiments when the leader sequence is present, the leader sequence may be encoded by a nucleic acid sequence comprising at least about 60%, 65%, 70%, 75%, 80%, 85%, 90%, 95%, or 99% sequence identity to SEQ ID NO: 2, SEQ ID NO: 3, SEQ ID NO: 4 or SEQ ID NO: 6, or a functional fragment or variant thereof. In some embodiments when the leader sequence is present, the leader sequence may be encoded by the nucleic acid sequence of SEQ ID NO: 2, SEQ ID NO: 3, SEQ ID NO: 4 or SEQ ID NO: 6, or a functional fragment or variant thereof.

2. Self-Assembling Polypeptide

The disclosure relates to an expressible nucleic acid sequence comprising at least one nucleic acid sequence encoding a scaffold domain comprising a self-assembling polypeptide, a functional fragment or variant thereof or a pharmaceutically acceptable salt thereof. Self-assembling polypeptide are polypeptides capable of undergoing spontaneous assembling into ordered nanostructures. Effectively self-assembling polypeptides can act as building blocks to form the scaffold domain of the present disclosure. In some embodiments, the self-assembling polypeptides encoded by the expressible nucleic acid sequence of the disclosure are monomeric forms of viral trimers or variants thereof. In some embodiments, the self-assembling polypeptides are monomers of nanoparticle structural proteins that self-assemble into nanoparticles upon expression.

The self-assembling peptide is a scaffold of the lumazine synthase of hyperthermophilic bacterium Aquifex aeolicus having the amino acid sequence of SEQ ID NO: 8 (LS-3 scaffold) encoded by the nucleic acid sequence of SEQ ID NO: 7.

(SEQ ID NO: 7)
atgcagatctacgaaggaaaactgaccgctgagggactgaggttcggaatt
gtcgcaagccgcgcgaatcacgcactggtggataggctggtggaaggcgct
atcgacgcaattgtccggcacggcgggagagaggaagacatcacactggtg
agagtctgcggcagctgggagattcccgtggcagctggagaactggctcga
aaggaggacatcgatgccgtgatcgctattggggtcctgtgccgaggagca
actcccagcttcgactacatcgcctcagaagtgagcaaggggctggctgat
ctgtccctggagctgaggaaacctatcacttttggcgtgattactgccgac
accctggaacaggcaatcgaggcggccggcacctgccatggaaacaaaggc
tgggaagcagccctgtgcgctattgagatggcaaatctgttcaaatctctg
cga
(SEQ ID NO: 8)
MQIYEGKLTAEGLRFGIVASRANHALVDRLVEGAIDAIVRHGGREEDITLV
RVCGSWEIPVAAGELARKEDIDAVIAIGVLCRGATPSFDYIASEVSKGLAD
LSLELRKPITFGVITADTLEQAIEAAGTCHGNKGWEAALCAIEMANLFKSL
R

3. Linker

The expressible nucleic acid sequence of the present disclosure optionally comprises a nucleic acid sequence encoding a linker peptide, a functional fragment or variant thereof or a pharmaceutically acceptable salt thereof. Any type of linker or linker peptide can be used. The term “linker” or “linker peptide” is used interchangeable herein.

In some embodiments, each linker or linker peptide is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length.

In some embodiments, each linker or linker peptide is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, each linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, each linker or linker peptide is about 21 natural or non-natural amino acids in length.

In some embodiments, the length of each linker or linker peptide is different. For example, in some embodiments, the length of a first linker or linker peptide is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length, where the length of the first linker is different from the length of the second linker. Various configurations can be envisioned by the present disclosure, where the linker domain comprises 1, 2, 3, 4, 5, 6, 7, 8, 9, 10 or more linkers or linker peptides wherein the linkers or linker peptides are of similar or different lengths. In some embodiments, two linkers or linker peptides can be used together. Accordingly, in some embodiments, the first linker or linker peptide is independently selectable from about 0 to about 25 natural or non-natural amino acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length. In some embodiments, the second linker or linker peptide is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural amino acids in length. In some embodiments, the first linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length. In some embodiments, the second linker or linker peptide is independently selectable from a linker or linker peptide that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural amino acids in length.

A non-limiting example of a linker peptide may comprise the amino acid sequence of GGSGGSGGSGGSGGG (SEQ ID NO: 22) encoded by the nucleic acid sequence of ggaggctccggaggatctggagggagtggaggctcaggaggaggc (SEQ ID NO: 21).

A linker or linker peptide can be either flexible or rigid or a combination thereof. An example of a flexible linker is a GGS repeat. In some embodiments, the GGS can be repeated about 1, 2, 3, 4, 5, 6, 7, 8, 9, or 10 times. Non-limiting examples of such linker peptides may comprise the amino acid sequence of GGSGGSGGS (SEQ ID NO: 23), GGSGGSGGSGGS (SEQ ID NO: 24), or GGSGGSGGSGGSGGGGSGGGSGGG (SEQ ID NO: 25). An example of a rigid linker is 4QTL-115 Angstroms, single chain 3-helix bundle represented by the sequence:

(SEQ ID NO: 26)
NEDDMKKLYKQMVQELEKARDRMEKLYKEMVELIQKAIELMRKIFQEVKQE
VEKAIEEMKKLYDEAKKKIEQMIQQIKQGGDKQKMEELLKRAKEEMKKVKD
KMEKLLEKLKQIMQEAKQKMEKLLKQLKEEMKKMKEKMEKLLKEMKQRMEE
VKKKMDGDDELLEKIKKNIDDLKKIAEDLIKKAEENIKEAKKIAEQLVKRA
KQLIEKAKQVAEELIKKILQLIEKAKEIAEKVLKGLE

Other non-limiting examples of linker peptides may be encoded by the nucleic acid sequence of

(SEQ ID NO: 27)
ggcggctctggcggaagtggcggaagtgggggaagtggaggcggcggaagc
gggggaggcagcgggggaggg,
(SEQ ID NO: 28)
ggcggaagcggcggaagcggcgggtct,
(SEQ ID NO: 29)
ggcggcagcggcggcagcggcgggagcggaggaagt,
or
(SEQ ID NO: 30)
ggcggctctggcggaagtggcggaagtgggggaagtggaggcggcggaagc
gggggaggcagcgggggaggg.

Additional non-limiting examples of linker peptides include Link 14 linker (SEQ ID NO: 32) encoded by the nucleic acid sequence of SEQ ID NO: 31;

(SEQ ID NO: 31)
tctcacagcggctccggcggctctggcagcggcggccacgcc
(SEQ ID NO: 32)
SHSGSGGSGSGGHA

CPG9.2 linker 1 (SEQ ID NO: 34) encoded by the nucleic acid sequence of SEQ ID NO: 33;

(SEQ ID NO: 33)
gggggaaatagtagcggc
(SEQ ID NO: 34)
GGNSSG

CPG9.2 linker 2 (SEQ ID NO: 36) encoded by the nucleic acid sequence of SEQ ID NO: 35;

(SEQ ID NO: 35)
ggcggcaacggcagcggcggcggcagcggctccggcggcaacggctctagc
ggc
(SEQ ID NO: 36)
GGNGSGGGSGSGGNGSSG

PDGFR linker (between trimer or TS1 and PDGFR; SEQ ID NO: 38) encoded by the nucleic acid sequence of SEQ ID NO: 37;

(SEQ ID NO: 37)
ggaggaggaagcgggggaagcgggggaagcggaggaagcgggggaagcggg
ggaagc
(SEQ ID NO: 38)
GGGSGGSGGSGGSGGSGGS

Foldon PDGFR linker 1 (SEQ ID NO: 40) encoded by the nucleic acid sequence of SEQ ID NO: 39;

(SEQ ID NO: 39)
ggaggaggaagcgggggaagcggcggcggc
(SEQ ID NO: 40)
GGGSGGSGGG

Foldon PDGFR linker 2 (SEQ ID NO: 42) encoded by the nucleic acid sequence of SEQ ID NO: 41;

(SEQ ID NO: 41)
gggggaagcggaggaagcgggggaagcgggggaagc
(SEQ ID NO: 42)
GGSGGSGGSGGS

3BVE linker (SEQ ID NO: 44) encoded by the nucleic acid sequence of SEQ ID NO: 43;

(SEQ ID NO: 43)
ggaagcggc
(SEQ ID NO: 44)
GSG

I3_1 linker (SEQ ID NO: 46) encoded by the nucleic acid sequence of SEQ ID NO: 45;

(SEQ ID NO: 45)
ggcggcagcggcagcggcgggagcggagga
(SEQ ID NO: 46)
GGSGSGGSGG

I3_2 linker (SEQ ID NO: 48) encoded by the nucleic acid sequence of SEQ ID NO: 47;

(SEQ ID NO: 47)
ggagggagcgatatgagaaaggacgccgagagacggtttgataagttcgt
ggaggctgctaagaataagtttgacaagtttaaggctgccctgcggaagg
gcgacatcaaggaggagaggagaaaggatatgaagaagctggcaaggaag
gaggcagagcaggcaaggagggccgtgaggaacagactgagcgagctgct
gtccaagatcaacgacatgcccatcaccaatgatcagaagaagctgatgt
ctaatgacgtgctgaagttcgccgcagaagccgaaaagaagattgaagcc
ctggcagcagacgccgaaggaggaagcgggagc
(SEQ ID NO: 48)
GGSDMRKDAERRFDKFVEAAKNKFDKFKAALRKGDIKEERRKDMKKLARK
EAEQARRAVRN-RLSELLSKINDMPITNDQKKLMSNDVLKFAAEAEKKIE
ALAADAEGGSGS

LS_1 linker (SEQ ID NO: 50) encoded by the nucleic acid sequence of SEQ ID NO: 49;

(SEQ ID NO: 49)
gggggctctagcgggaaaagtctggtggataccgtctatgctctgaaaga
tgaggtgcaggaactgaggcaggacaacaaaaagatgaagaagagcctgg
aggaggagcagagggccagaaaggacctggaaaaactggtgcggaaagtg
ctgaaaaacatgaatgacggagggagtagcggg
(SEQ ID NO: 50)
GGSSGKSLVDTVYALKDEVQELRQDNKKMKKSLEEEQRARKDLEKLVRKV
LKNMNDGGSSG

LS_2 linker (SEQ ID NO: 52) encoded by the nucleic acid sequence of SEQ ID NO: 51;

(SEQ ID NO: 51)
gggggctctagcggggcagacccaaagaaagtgctggataaggcaaagga
tcaggcagagaatagagtgagagaactgaaacagaaactggaggaactgt
ataaggaggcccggaagctggacctgacccaggagatgaggagaaagctg
gagctgcgctacatcgccgccatgctgatggccatcggcgacatctataa
cgccatcaggcaggccaagcaggaggccgataagctgaagaaggccggcc
tggtgaatagccagcagctggacgagctgaagcggcgcctggaggagctg
aaggaggaggcctccaggaaggccagagattatgggcgggaatttcagct
gaaactggagtatggcggcggaagcggaagcgggagcggg
(SEQ ID NO: 52)
GGSSGADPKKVLDKAKDQAENRVRELKQKLEELYKEARKLDLTQEMRRKL
ELRYIAAMLMAIGDIYNAIRQAKQEADKLKKAGLVNSQQLDELKRRLEEL
KEEASRKARDYGREFQLKLEYGGGSGSGSG

QB_1 linker (SEQ ID NO: 54) encoded by the nucleic acid sequence of SEQ ID NO: 53;

(SEQ ID NO: 53)
ggaggctcttcaggcggcacagacgtgggggcaatcgctggaaaggctaa
cgaggctggacagggggcttatgatgctcaggtcaaaaacgacgagcagg
atgtggagctggccgaccacgaggccaggatcaagcagctgagaatcgat
gtggacgatcacgagtctcggatcaccgccaacacaaaggccatcacagc
cctgaatgtgcgcgtgaccacagcagagggagagatcgcatccctgcaga
ccaacgtgagcgccctggacggaagggtgaccacagcagagaacaatatc
tccgccctgcaggcagattacgtgagcggcggcagctccggctccgga
(SEQ ID NO: 54)
GGSSGGTDVGAIAGKANEAGQGAYDAQVKNDEQDVELADHEARIKQLRID
VDDHESRITANTKAITALNVRVTTAEGEIASLQTNVSALDGRVTTAENNI
SALQADYVSGGSSGSG

QB_2 linker (SEQ ID NO: 56) encoded by the nucleic acid sequence of SEQ ID NO: 55; and

(SEQ ID NO: 55)
ggaggctctggaagcgggggaagtagcggacctcacatgattgctccagg
acatcgggacgagtttgaccctaagctgccaacaggcgagaaagaagagg
tgccaggcaagcccggcatcaagaaccctgagacaggcgacgtggtgagg
ccccctgtggattctgtgacaaagtacggcccagtgaagggcgacagcat
cgtggagaaggaggagatccccttcgagaaggagaggaagtttaaccctg
atctggccccaggcaccgagaaggtgacaagagagggccagaagggcgag
aagaccatcaccacacccacactgaagaatcctctgaccggcgagatcat
cagcaagggcgagtccaaggaggagatcacaaaggaccccatcaacgaac
tgaccgaatggggaccagagacaggaggaagcggcagcggcggaagcagc
(SEQ ID NO: 56)
GGSGSGGSSGPHMIAPGHRDEFDPKLPTGEKEEVPGKPGIKNPETGDVVR
PPVDSVTKYGPVKGDSIVEKEEIPFEKERKFNPDLAPGTEKVTREGQKGE
KTITTPTLKNPLTGEIISKGESKEEITKDPINELTEWGPETGGSGSGGSS

IC1/IC2 linker (SEQ ID NO: 58) encoded by the nucleic acid sequence of SEQ ID NO: 57.

(SEQ ID NO: 57)
ggaggcagcggcagcggcagcggg
(SEQ ID NO: 58)
GGSGSGSG

Accordingly, in some embodiments, the linker peptide encoded by the expressible nucleic acid sequence of the present disclosure comprises at least about 60%, 65%, 70%, 75%, 80%, 85%, 90%, 95%, or 99% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the linker peptide comprises the amino acid sequence of SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the nucleic acid sequence encoding the linker peptide comprises at least about 60%, 65%, 70%, 75%, 80%, 85%, 90%, 95%, or 99% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof. In some embodiments, the nucleic acid sequence encoding the linker peptide comprises the nucleotide sequence of SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

4. Viral Antigens

The expressible nucleic acid sequence of the present disclosure comprises a nucleic acid sequence encoding an antigen domain comprising a viral antigen, a functional fragment or variant thereof or a pharmaceutically acceptable salt thereof. In some embodiments, the viral antigen comprises an antigen from a virus from the family of Coronaviridae. In some embodiments, the viral antigen comprises an antigen from a coronavirus. In some embodiments, the viral antigen comprises an antigen from SARS-CoV. In some embodiments, the viral antigen comprises an antigen from SARS-CoV-2. In some embodiments, the viral antigen comprises the spike protein of SARS-CoV-2, a functional fragment or variant thereof or a pharmaceutically acceptable salt thereof. In some embodiments, the viral antigen comprises a viral trimer polypeptide, a functional fragment or variant thereof or a pharmaceutically acceptable salt thereof. In some embodiments, the viral trimer comprises a trimer from a virus from the family of Coronaviridae. In some embodiments, the viral trimer comprises a trimer from a coronavirus. In some embodiments, the viral trimer comprises a trimer from SARS-CoV. In some embodiments, the viral trimer comprises a trimer from SARS-CoV-2. In some embodiments, the viral trimer comprises the spike protein of SARS-CoV-2, a functional fragment or variant thereof or a pharmaceutically acceptable salt thereof.

A non-limiting example of a viral antigen is a fragment of the surface glycoprotein (or spike protein or S protein) of SARS-CoV-2 having the amino acid sequence of SEQ ID NO: 60 encoded by the nucleic acid sequence of SEQ ID NO: 59 (GenBank Accession No. QHD43416).

(SEQ ID NO: 59)
atgtttgtttttcttgttttattgccactagtctctagtcagtgtgttaa
tcttacaaccagaactcaattaccccctgcatacactaattctttcacac
gtggtgtttattaccctgacaaagttttcagatcctcagttttacattca
actcaggacttgttcttacctttcttttccaatgttacttggttccatgc
tatacatgtctctgggaccaatggtactaagaggtttgataaccctgtcc
taccatttaatgatggtgtttattttgcttccactgagaagtctaacata
ataagaggctggatttttggtactactttagattcgaagacccagtccct
acttattgttaataacgctactaatgttgttattaaagtctgtgaatttc
aattttgtaatgatccatttttgggtgtttattaccacaaaaacaacaaa
agttggatggaaagtgagttcagagtttattctagtgcgaataattgcac
ttttgaatatgtctctcagccttttcttatggaccttgaaggaaaacagg
gtaatttcaaaaatcttagggaatttgtgtttaagaatattgatggttat
tttaaaatatattctaagcacacgcctattaatttagtgcgtgatctccc
tcagggtttttcggctttagaaccattggtagatttgccaataggtatta
acatcactaggtttcaaactttacttgctttacatagaagttatttgact
cctggtgattcttcttcaggttggacagctggtgctgcagcttattatgt
gggttatcttcaacctaggacttttctattaaaatataatgaaaatggaa
ccattacagatgctgtagactgtgcacttgaccctctctcagaaacaaag
tgtacgttgaaatccttcactgtagaaaaaggaatctatcaaacttctaa
ctttagagtccaaccaacagaatctattgttagatttcctaatattacaa
acttgtgcccttttggtgaagtttttaacgccaccagatttgcatctgtt
tatgcttggaacaggaagagaatcagcaactgtgttgctgattattctgt
cctatataattccgcatcattttccacttttaagtgttatggagtgtctc
ctactaaattaaatgatctctgctttactaatgtctatgcagattcattt
gtaattagaggtgatgaagtcagacaaatcgctccagggcaaactggaaa
gattgctgattataattataaattaccagatgattttacaggctgcgtta
tagcttggaattctaacaatcttgattctaaggttggtggtaattataat
tacctgtatagattgtttaggaagtctaatctcaaaccttttgagagaga
tatttcaactgaaatctatcaggccggtagcacaccttgtaatggtgttg
aaggttttaattgttactttcctttacaatcatatggtttccaacccact
aatggtgttggttaccaaccatacagagtagtagtactttcttttgaact
tctacatgcaccagcaactgtttgtggacctaaaaagtctactaatttgg
ttaaaaacaaatgtgtcaatttcaacttcaatggtttaacaggcacaggt
gttcttactgagtctaacaaaaagtttctgcctttccaacaatttggcag
agacattgctgacactactgatgctgtccgtgatccacagacacttgaga
ttcttgacattacaccatgttcttttggtggtgtcagtgttataacacca
ggaacaaatacttctaaccaggttgctgttctttatcaggatgttaactg
cacagaagtccctgttgctattcatgcagatcaacttactcctacttggc
gtgtttattctacaggttctaatgtttttcaaacacgtgcaggctgttta
ataggggctgaacatgtcaacaactcatatgagtgtgacatacccattgg
tgcaggtatatgcgctagttatcagactcagactaattctcctcggcggg
cacgtagtgtagctagtcaatccatcattgcctacactatgtcacttggt
gcagaaaattcagttgcttactctaataactctattgccatacccacaaa
ttttactattagtgttaccacagaaattctaccagtgtctatgaccaaga
catcagtagattgtacaatgtacatttgtggtgattcaactgaatgcagc
aatcttttgttgcaatatggcagtttttgtacacaattaaaccgtgcttt
aactggaatagctgttgaacaagacaaaaacacccaagaagtttttgcac
aagtcaaacaaatttacaaaacaccaccaattaaagattttggtggtttt
aatttttcacaaatattaccagatccatcaaaaccaagcaagaggtcatt
tattgaagatctacttttcaacaaagtgacacttgcagatgctggcttca
tcaaacaatatggtgattgccttggtgatattgctgctagagacctcatt
tgtgcacaaaagtttaacggccttactgttttgccacctttgctcacaga
tgaaatgattgctcaatacacttctgcactgttagcgggtacaatcactt
ctggttggacctttggtgcaggtgctgcattacaaataccatttgctatg
caaatggcttataggtttaatggtattggagttacacagaatgttctcta
tgagaaccaaaaattgattgccaaccaatttaatagtgctattggcaaaa
ttcaagactcactttcttccacagcaagtgcacttggaaaacttcaagat
gtggtcaaccaaaatgcacaagctttaaacacgcttgttaaacaacttag
ctccaattttggtgcaatttcaagtgttttaaatgatatcctttcacgtc
ttgacaaagttgaggctgaagtgcaaattgataggttgatcacaggcaga
cttcaaagtttgcagacatatgtgactcaacaattaattagagctgcaga
aatcagagcttctgctaatcttgctgctactaaaatgtcagagtgtgtac
ttggacaatcaaaaagagttgatttttgtggaaagggctatcatcttatg
tccttccctcagtcagcacctcatggtgtagtcttcttgcatgtgactta
tgtccctgcacaagaaaagaacttcacaactgctcctgccatttgtcatg
atggaaaagcacactttcctcgtgaaggtgtctttgtttcaaatggcaca
cactggtttgtaacacaaaggaatttttatgaaccacaaatcattactac
agacaacacatttgtgtctggtaactgtgatgttgtaataggaattgtca
acaacacagtttatgatcctttgcaacctgaattagactcattcaaggag
gagttagataaatattttaagaatcatacatcaccagatgttgatttagg
tgacatctctggcattaatgcttcagttgtaaacattcaaaaagaaattg
accgcctcaatgaggttgccaagaatttaaatgaatctctcatcgatctc
caagaacttggaaagtatgagcagtatataaaatggccatggtacatttg
gctaggttttatagctggcttgattgccatagtaatggtgacaattatgc
tttgctgtatgaccagttgctgtagttgtctcaagggctgttgttcttgt
ggatcctgctgcaaatttgatgaagacgactctgagccagtgctcaaagg
agtcaaattacattacacataa
(SEQ ID NO: 60)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Non-limiting examples of fragments of the S protein of SARS-CoV-2 comprises the following sequences:

(SEQ ID NO: 171)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
KYFKNHTSPDVDLGDISGINASVVNIQKEIDRLNEVAKNLNESLIDLQELGKYEQYIK
WPW
(SEQ ID NO: 172)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQUITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WP
(SEQ ID NO: 173)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQUITTDNTFVSGNCDVVIGIVNNTVYDPLQPELDSFKEELD
KYFKNHTSPDVDLGDISGINASVVNIQKEIDRLNEVAKNLNESLIDLQELGKYEQ
(SEQ ID NO: 174)
SQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQGN
FKNLREFVFKNIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGSNVFQTRA
GCLIGAEHVNNSYECDIPIGAGICASYQTQTNSPRRARSVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSFIEDLLFN
KVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLS
STASALGKLQDVVNQNAQALNTLVKQLSSNFGAISSVLNDILSRLDKVEAEVQIDRLI
TGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNF
YEPQUITTDNTFVSGNCDVVIGIVNNTVYDPLQPELDSFKEELDKYFKNHTSPDVDLG
DISGINASVVNIQKEIDRLNEVAKNLNESLIDLQELGKYEQ
(SEQ ID NO: 175)
NLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGG
NYNYLYRLFRKSNLKPFERDIST
(SEQ ID NO: 176)
SFIEDLLFNKVTLADAGFIKQYGDCLGDIAARDLICAQKF
(SEQ ID NO: 177)
PSKRSFIEDLLFNKV

A further non-limiting example of a viral antigen is a fragment of the envelop protein (or E protein) of SARS-CoV-2 having the amino acid sequence of SEQ ID NO: 62 encoded by the nucleic acid sequence of SEQ ID NO: 61 (GenBank Accession No. QHD43418).

(SEQ ID NO: 61)
atgtactcattcgtttcggaagagacaggtacgttaatagttaatagcgt
acttctttttcttgctttcgtggtattcttgctagttacactagccatcc
ttactgcgcttcgattgtgtgcgtactgctgcaatattgttaacgtgagt
cttgtaaaaccttctttttacgtttactctcgtgttaaaaatctgaattc
ttctagagttcctgatcttctggtctaa
(SEQ ID NO: 62)
MYSFVSEETGTLIVNSVLLFLAFVVFLLVTLAILTALRLCAYCCNIVNVS
LVKPSFYVYSRVKNLNSSRVPDLLV

Another non-limiting example of a viral antigen is a fragment of the membrane glycoprotein (or M protein) of SARS-CoV-2 having the amino acid sequence of SEQ ID NO: 62 encoded by the nucleic acid sequence of SEQ ID NO: 61 (GenBank Accession No. QHD43419).

(SEQ ID NO: 63)
ccatggcagattccaacggtactattaccgttgaagagcttaaaaagctc
cttgaacaatggaacctagtaataggtttcctattccttacatggatttg
tcttctacaatttgcctatgccaacaggaataggtttttgtatataatta
agttaattttcctctggctgttatggccagtaactttagcttgttttgtg
cttgctgctgtttacagaataaattggatcaccggtggaattgctatcgc
aatggcttgtcttgtaggcttgatgtggctcagctacttcattgcttctt
tcagactgtttgcgcgtacgcgttccatgtggtcattcaatccagaaact
aacattcttctcaacgtgccactccatggcactattctgaccagaccgct
tctagaaagtgaactcgtaatcggagctgtgatccttcgtggacatcttc
gtattgctggacaccatctaggacgctgtgacatcaaggacctgcctaaa
gaaatcactgttgctacatcacgaacgctttcttattacaaattgggagc
ttcgcagcgtgtagcaggtgactcaggttttgctgcatacagtcgctaca
ggattggcaactataaattaaacacagaccattccagtagcagtgacaat
attgctttgcttgtacagtaa
(SEQ ID NO: 64)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ

Yet another non-limiting example of a viral antigen is a fragment of the nucleocapsid phosphoprotein (or N protein) of SARS-CoV-2 having the amino acid sequence of SEQ ID NO: 66 encoded by the nucleic acid sequence of SEQ ID NO: 65 (GenBank Accession No. QHD43423), or a variant thereof:

(SEQ ID NO: 65)
atgtctgataatggaccccaaaatcagcgaaatgcaccccgcattacgtt
tggtggaccctcagattcaactggcagtaaccagaatggagaacgcagtg
gggcgcgatcaaaacaacgtcggccccaaggtttacccaataatactgcg
tcttggttcaccgctctcactcaacatggcaaggaagaccttaaattccc
tcgaggacaaggcgttccaattaacaccaatagcagtccagatgaccaaa
ttggctactaccgaagagctaccagacgaattcgtggtggtgacggtaaa
atgaaagatctcagtccaagatggtatttctactacctaggaactgggcc
agaagctggacttccctatggtgctaacaaagacggcatcatatgggttg
caactgagggagccttgaatacaccaaaagatcacattggcacccgcaat
cctgctaacaatgctgcaatcgtgctacaacttcctcaaggaacaacatt
gccaaaaggcttctacgcagaagggagcagaggcggcagtcaagcctctt
ctcgttcctcatcacgtagtcgcaacagttcaagaaattcaactccaggc
agcagtaggggaacttctcctgctagaatggctggcaatggcggtgatgc
tgctcttgctttgctgctgcttgacagattgaaccagcttgagagcaaaa
tgtctggtaaaggccaacaacaacaaggccaaactgtcactaagaaatct
gctgctgaggcttctaagaagcctcggcaaaaacgtactgccactaaagc
atacaatgtaacacaagctttcggcagacgtggtccagaacaaacccaag
gaaattttggggaccaggaactaatcagacaaggaactgattacaaacat
tggccgcaaattgcacaatttgcccccagcgcttcagcgttcttcggaat
gtcgcgcattggcatggaagtcacaccttcgggaacgtggttgacctaca
caggtgccatcaaattggatgacaaagatccaaatttcaaagatcaagtc
attttgctgaataagcatattgacgcatacaaaacattcccaccaacaga
gcctaaaaaggacaaaaagaagaaggctgatgaaactcaagccttaccgc
agagacagaagaaacagcaaactgtgactcttcttcctgctgcagatttg
gatgatttctccaaacaattgcaacaatccatgagcagtgctgactcaac
tcaggcctaa
(SEQ ID NO: 66)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

Accordingly, in some embodiments, the viral antigen encoded by the expressible nucleic acid sequence of the present disclosure comprises at least about 60%, 65%, 70%, 75%, 80%, 85%, 90%, 95%, or 99% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof or a pharmaceutically acceptable salt thereof. In some embodiments, the viral antigen comprises the amino acid sequence of SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof or a pharmaceutically acceptable salt thereof. In some embodiments, the nucleic acid sequence encoding the viral antigen comprises at least about 60%, 65%, 70%, 75%, 80%, 85%, 90%, 95%, or 99% sequence identity to SEQ ID NO: 59, SEQ ID NO: 61, SEQ ID NO: 63 or SEQ ID NO: 65, or a functional fragment or variant thereof or a pharmaceutically acceptable salt thereof. In some embodiments, the nucleic acid sequence encoding the viral antigen comprises the nucleotide sequence of SEQ ID NO: 59, SEQ ID NO: 61, SEQ ID NO: 63 or SEQ ID NO: 65, or a functional fragment or variant thereof or a pharmaceutically acceptable salt thereof.

In some embodiments, the expressible nucleic acid sequence encodes a fusion protein comprising one or a plurality of coronaviral envelope polypeptides or functional fragments thereof. In some embodiments, the fusion protein comprise a furin cleavage site. In some embodiments, the expressible nucleic acid sequence comprises a first nucleic acid sequence encoding, in a 5′ to 3′ orientation, at least three monomers of coronaviral envelope proteins. In some embodiments, the at least three monomers of coronaviral envelope proteins are separated by a furin cleavage site. In some embodiments, the furin cleavage site comprises at least about 60%, 65%, 70%, 75%, 80%, 85%, 90%, 95%, or 99% sequence identity to RRRRRR (SEQ ID NO: 67), or a functional fragment or variant thereof or a pharmaceutically acceptable salt thereof. In some embodiments, the furin cleavage site comprises the amino acid sequence of SEQ ID NO: 67, or a functional fragment or variant thereof or a pharmaceutically acceptable salt thereof. In some embodiments, the expressible nucleic acid sequence encodes a polypeptide free of carbohydrate proximate to at least 30 amino acids from the carboxy end of the polypeptide. In some embodiments, the expressible nucleic acid sequence encodes a polypeptide free of carbohydrate proximate to at least 20 amino acids from the carboxy end of the polypeptide. In some embodiments, the expressible nucleic acid sequence encodes a polypeptide free of carbohydrate proximate to at least 10 amino acids from the carboxy end of the polypeptide. In some embodiments, the expressible nucleic acid sequence encodes a polypeptide free of carbohydrate proximate to at least 50 amino acids from the carboxy end of the polypeptide.

In some embodiments, the expressible nucleic acid sequence of the disclosure comprises at least a first nucleic acid sequence encoding a first, a second and/or a third polypeptides, each first, second or third polypeptide comprising a viral antigen. In some embodiments, the expressible nucleic acid sequence encodes one or a plurality of fusion proteins, each fusion protein comprising at least a first, a second, and/or a third polypeptide contiguously linked by a linker sequence. In some embodiments, the expressible nucleic acid sequence of the disclosure comprises at least a first nucleic acid sequence encoding at least one self-assembling polypeptide. In some embodiments, the self-assembling polypeptide is at least one self-assembling component of a nanoparticle or at least one coronaviral monomer, the coronaviral monomer capable of assembling into a coronaviral trimer upon expression in a cell. In some embodiments, the expressible nucleic acid sequence comprises a nucleic acid sequence encoding a coronaviral antigen, but free of a nucleic acid sequence encoding a self-assembling polypeptide. In some embodiments, the expressible nucleic acid sequence of the disclosure comprises a nucleic acid sequence operably linked to a regulatory sequence and encodes a fusion peptide comprising one or a plurality of self-assembling polypeptides, wherein at least one of the self-assembling polypeptides is a self-assembling coronaviral antigen.

In some embodiments, upon administration to a subject a composition comprising the expressible nucleic acid sequence of the disclosure, the expressible nucleic acid sequence is transfected or transduced into an antigen presenting cell. After a plurality of expressible nucleic acid sequences are expressed, the self-assembling polypeptides assemble with into a non-native form of a viral antigen. In some embodiments, the non-native form of a viral antigen comprises a coronaviral trimer exposing an amino acid sequence that is not naturally exposed or free of carbohydrate as compared to its corresponding native form or variants thereof. Expression and presentation of the one or plurality of self-assembling polypeptides elicits an immune response against an epitope. In some embodiments, the epitope comprises a non-native secondary structure of the one or plurality of self-assembling polypeptides. In some embodiments, the compositions comprise a nucleic acid sequence encoding any combination of nucleic acid sequences disclosed herein or variants thereof. In some embodiments, the compositions comprise a viral particle that comprises an expressible nucleic acid sequence encoding any combination of nucleic acid sequences disclosed herein or variants thereof. The component of the self-assembling peptide can be any monomer that, upon expression, self-assembles into a particle comprising 7, 14, 27 or 60 peptides sided particle, each peptide side fused to at least one antigen from the Coronoviridae family. In some embodiments, the composition comprises a particle comprising 7, 14, 27 or 60 peptides sided particle, each peptide side is fused to at least one antigen from the Coronoviridae family, wherein the antigen is positioned in an energetically stable state as compared to the unassociated energy state. In some embodiments, the energetically stable state is identified by association of the peptide to an antibody through surface plasmon resonance (SPR). In some embodiments, the energetically stable state is measured by absorbance units when either a ligand for the antigen or the antigen is immobilized to a surface, and the other binding partner is then passed over the surface as analyte. In some embodiments, the association can be measured through SPR on a BIACORE® system.

A detailed discussion of the technical aspects of the BIACORE® instruments and the phenomenon of SPR may be found in U.S. Pat. No. 5,313,264 (the full disclosure of which is incorporated by reference herein in its entirety). In the BIACORE® system, the SPR response values are expressed in resonance units (RU). One RU represents a change of 0.0001° in the angle of minimum reflected light intensity. For an SPR based sensor system like the BIACORE® system, a difference in refractive index between the two guiding fluids of, say, about 100 RU may be convenient, and the fluid interface position may be determined by means of per se conventional sensorgrams.

In some embodiments, it may be preferred to keep the total flow rate constant when introducing the sample flow. In such a case, the flow rates of the two guiding fluids are reduced while maintaining the flow rate ratio between them. Assume, for example, that the flow rate of one guiding fluid is 70 μl/min and the flow rate of the other guiding fluid is 30 μl/min, the total flow rate being 100 μl/min, and that a sample fluid flow of 20 μl/min is introduced between the guiding fluids. To maintain the total fluid flow rate at 100 μl/min, the flow rates of the guiding fluids will have to be reduced to 60 and 20 μl/min, respectively. The position of a sample fluid flow on a surface may be presented in various ways. A non-limiting example of a experiment indicating the relative responses obtained at different detector rows as the sample flow is guided laterally across the sensing surface of a flow cell by two guiding buffers in a BIACORE® system equipped with a W-cell (BIACORE® S51 is a SPR-based biosensor instrument, normally equipped with two Y-type flow cells, each allowing a dual flow over the a sensor surface for hydrodynamic addressing; Biacore AB, Uppsala, Sweden). Total buffer flow can be set to 100 μl/min, and the flow rates of the two buffer flows can be changed in steps of 2 μl/min, starting with 2 μl/min for one buffer and 98 μl/min for the other. Sample fluid flow can be 20 μl/min all the time. Relative responses >0.1 (i.e. 10% coverage of the detector row) are represented are measured as absorbance over time. This approach thus permits convenient visual monitoring of the sample fluid flow.

In some embodiments, the stability of an antigen secondary structure with an elevated stability as compared to a native antigen or antigen not fused to self-assembling peptide is from about 10 to about 10,000 RU more than the RU from a control as measured by SPR. In some embodiments, the stability of an antigen secondary structure with an elevated stability as compared to a native antigen or antigen not fused to self-assembling peptide is from about 5 to about 1,000 RU more than the RU from a control as measured by SPR. In some embodiments, the stability of an antigen secondary structure with an elevated stability as compared to a native antigen or antigen not fused to self-assembling peptide is from about 100 to about 10,000 RU more than the RU from a control as measured by SPR. In some embodiments, the stability of an antigen secondary structure with an elevated stability as compared to a native antigen or antigen not fused to self-assembling peptide is from about 100 to about 500 RU more than the RU from a control as measured by SPR. In some embodiments, the stability of an antigen secondary structure with an elevated stability as compared to a native antigen or antigen not fused to self-assembling peptide is from about 100 to about 200 RU more than the RU from a control as measured by SPR.

5. Regulatory Sequences

In some embodiments, the expressible nucleic acid sequence can be operably linked to one or a plurality of regulatory sequences. The term “regulatory sequence” as used herein refer to DNA sequences which are necessary to effect expression of sequences to which they are ligated. The term “regulatory sequence” is intended to include, as a minimum, all components necessary for expression and optionally additional advantageous components. Examples of regulatory sequences include, but not limited to, promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. In some embodiments, the regulatory sequence is a promoter sequence. As used herein, a “promoter” means a region of DNA upstream from the transcription start and which is involved in binding RNA polymerase and other proteins to start transcription. Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. Consequently, a repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions. The term “promoter” also includes the transcriptional regulatory sequences of a classical prokaryotic gene, in which case it may include a −35 box sequence and/or a −10 box transcriptional regulatory sequences. The term “promoter” is also used to describe a synthetic or fusion molecule, or derivative which confers, activates or enhances expression of a nucleic acid molecule in a cell, tissue or organ.

6. Expressible Nucleic Acid Sequences

The expressible nucleic acid sequence comprised in the composition of the present disclosure can be in form of a DNA molecule, a RNA molecule or transcript, or a DNA/RNA hybrid. In some embodiments, the expressible nucleic acid sequence is in form of a DNA molecule. In some embodiments, the expressible nucleic acid sequence is in form of a RNA molecule or transcript. In some embodiments, the expressible nucleic acid sequence is in form of a DNA/RNA hybrid.

In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 8, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 10, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 12, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 14, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 16, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 18, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 20, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 8, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 175. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 8, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 176. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 8, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 177. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 12, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 175. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 12, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 176. In some embodiments, the expressible nucleic acid sequence comprises a first nucleic acid sequence encoding a self-assembling polypeptide comprising at least about 70%, 75%, 80%, 85%, 90%, 95%, 96%, 97%, 98%, 99% or 100% sequence identity to SEQ ID NO: 12, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 177.

In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof.

In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 171. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 172. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 173. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 174. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 175. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 176. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 1, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 177. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 171. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 172. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 173. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 174. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 175. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 176. In some embodiments, the expressible nucleic acid sequence comprises a first nucleic acid sequence encoding a leader sequence comprising at least about 70%, 75%, 80%, 85%, 90%, 95%, 96%, 97%, 98%, 99% or 100% sequence identity to SEQ ID NO: 5, or a functional fragment or variant thereof, and a second nucleic acid sequence encoding a viral antigen comprising at least about 70%, 75%, 80%, 85%, 90%, 95%, 96%, 97%, 98%, 99% or 100% sequence identity to SEQ ID NO: 177.

Exemplary expressible nucleic acid sequences include, but not limited to those provided in TABLE X. In some embodiments, a nucleic acid molecule of the disclosure comprises one or more expressible nucleic acid sequences below:

TABLE X
Exemplary Expressible Nucleic Acid Sequences (DNA and RNA) of the
Disclosure and the corresponding coding polypeptide sequences (underlined amino acid
residues are glycan sites).

I. CoV2 Nanoparticle Constructs
WuhanS_FP12_L9GT60_pVax
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcgacaccatcacactgccatgccgccctgcac
cacctccacattgtagctccaacatcaccggcctgattctgacaagacaggggggatatagtaacgataataccgtgattttcaggccct
caggaggggactggagggacatcgcacgatgccagattgctggaacagtggtctctactcagctgtttctgaacggcagtctggctga
ggaagaggtggtcatccgatctgaagactggcgggataatgcaaagtcaatttgtgtgcagctgaacacaagcgtcgagatcaattgc
actggcgcagggcactgtaacatttctcgggccaaatgggataataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcgatagcacttggtttgattccaccggtgggagcggaagtggcggttccggatcattcattgaagaccttctcttt
aacaaggtgaccctcgccgatgcaggtttcattaagcaatatggtgattgcctgggagacatcgcggctcgtgatcttatttgtgcgcag
aaattttaatga (SEQ ID NO: 68)
ggauccgccaccauggacuggaccuggauucuguuccugguggccgccgccacaagggugcacagcaugcagaucuacg
aaggaaaacugaccgcugagggacugagguucggaauugucgcaagccgcgcgaaucacgcacugguggauaggcuggu
ggaaggcgcuaucgacgcaauuguccggcacggcgggagagaggaagacaucacacuggugagagucugcggcagcugg
gagauucccguggcagcuggagaacuggcucgaaaggaggacaucgaugccgugaucgcuauugggguccugugccgag
gagcaacucccagcuucgacuacaucgccucagaagugagcaaggggcuggcugaucugucccuggagcugaggaaacc
uaucacuuuuggcgugauuacugccgacacccuggaacaggcaaucgaggcggccggcaccugccauggaaacaaaggcu
gggaagcagcccugugcgcuauugagauggcaaaucuguucaaaucucugcgaggaggcuccggaggaucuggagggag
uggaggcucaggaggaggcgacaccaucacacugccaugccgcccugcaccaccuccacauuguagcuccaacaucaccg
gccugauucugacaagacaggggggauauaguaacgauaauaccgugauuuucaggcccucaggaggggacuggaggga
caucgcacgaugccagauugcuggaacaguggucucuacucagcuguuucugaacggcagucuggcugaggaagaggug
gucauccgaucugaagacuggcgggauaaugcaaagucaauuugugugcagcugaacacaagcgucgagaucaauugca
cuggcgcagggcacuguaacauuucucgggccaaaugggauaauacccugaagcagaucgccaguaaacugagagagcag
uacggcaauaagacaaucaucuucaagccuucuaguggaggcgacccagaguucgugaaccauagcuuuaauugcgggg
gagaguuuuuuauugugauuccacacagcuguucgauagcacuugguuugauuccaccggugggagcggaaguggcg
uugccugggagacaucgcggcucgugaucuuauuuguggcagaaauuuuaauga (SEQ ID NO: 69)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGDTITLPCRPAPPPHCSSNITGLILTRQGGYSNDNTVIFRPSGGD
WRDIARCQIAGTVVSTQLFLNGSLAEEEVVIRSEDWRDNAKSICVQLNTSVEINCTGA

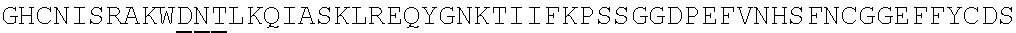

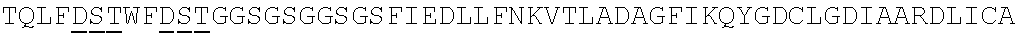

QKF** (SEQ ID NO: 70)
WuhanS_FP_L9GT60_pVax
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcgacaccatcacactgccatgccgccctgcac
cacctccacattgtagctccaacatcaccggcctgattctgacaagacaggggggatatagtaacgataataccgtgattttcaggccct
caggaggggactggagggacatcgcacgatgccagattgctggaacagtggtctctactcagctgtttctgaacggcagtctggctga
ggaagaggtggtcatccgatctgaagactggcgggataatgcaaagtcaatttgtgtgcagctgaacacaagcgtcgagatcaattgc
actggcgcagggcactgtaacatttctcgggccaaatgggataataccctgaagcagatcgccagtaaactgagagagcagtacggc
aataagacaatcatcttcaagccttctagtggaggcgacccagagttcgtgaaccatagctttaattgcgggggagagttcttttattgtg
attccacacagctgttcgatagcacttggtttgattccaccggtgggagcggaagtggcggttccggaccttcaaagagatctttcattg
aagacctgcttttcaacaaggtctaatga (SEQ ID NO: 71)
ggauccgccaccauggacuggaccuggauucuguuccugguggccgccgccacaagggugcacagcaugcagaucuacg
aaggaaaacugaccgcugagggacugagguucggaauugucgcaagccgcgcgaaucacgcacugguggauaggcuggu
ggaaggcgcuaucgacgcaauuguccggcacggcgggagagaggaagacaucacacuggugagagucugcggcagcugg
gagauucccguggcagcuggagaacuggcucgaaaggaggacaucgaugccgugaucgcuauugggguccugugccgag
gagcaacucccagcuucgacuacaucgccucagaagugagcaaggggcuggcugaucugucccuggagcugaggaaacc
uaucacuuuuggcgugauuacugccgacacccuggaacaggcaaucgaggcggccggcaccugccauggaaacaaaggcu
gggaagcagcccugugcgcuauugagauggcaaaucuguucaaaucucugcgaggaggcuccggaggaucuggagggag
uggaggcucaggaggaggcgacaccaucacacugccaugccgcccugcaccaccuccacauuguagcuccaacaucaccg
gccugauucugacaagacaggggggauauaguaacgauaauaccgugauuuucaggcccucaggaggggacuggaggga
caucgcacgaugccagauugcuggaacaguggucucuacucagcuguuucugaacggcagucuggcugaggaagaggug
gucauccgaucugaagacugggggauaaugcaaagucaauuugugugcagcugaacacaagcgucgagaucaauugca
cuggcgcagggcacuguaacauuucucgggccaaaugggauaauacccugaagcagaucgccaguaaacugagagagcag
uacggcaauaagacaaucaucuucaagccuucuaguggaggcgacccagaguucgugaaccauagcuuuaauugcgggg
gagaguucuuuuauugugauuccacacagcuguucgauagcacuugguuugauuccaccggugggagcggaaguggcg
guuccggaccuucaaagagaucuuucauugaagaccugcuuuucaacaaggucuaauga (SEQ ID NO: 72)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGDTITLPCRPAPPPHCSSNITGLILTRQGGYSNDNTVIFRPSGGD
WRDIARCQIAGTVVSTQLFLNGSLAEEEVVIRSEDWRDNAKSICVQLNTSVEINCTGA

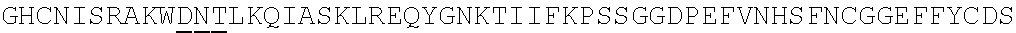

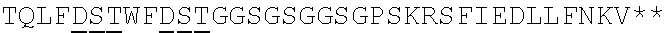

(SEQ ID NO: 73)
WuhanS_RBD_gmax_180_pVax
ggatccgccaccatggactggacctggattctgttcctggtggccgccgccacaagggtgcacagcctctcaattgccccaacgttga
tcaaccgggacaagccatacacgaaagaggaacttatggagatattgcggttggccattatagctgaactcgatgcaattaatctctatg
aacaaatggcccgctatagcgaagacgaaaatgtgagaaagatcttgttggacgtcgctagggaagagaaagcacacgtaggagag
ttcatggctttgttgcttaacctcgaccctgagcaagtcacagagctgaagggcgggttcgaggaagttaaagaattgaccggtataga
agctcacattaatgacaacaagaaagaggaaagtaatgtagagtatttcgagaagctcagatctgccttgttggatggagtcaacaagg
gtcgcagcttgctcaaacatctgcccgttacaagaatagaagggcagtcttttcgagtagacatcatcaaatttgaggacggcgtccga
gtggttaaacaagagtataagcctataccccttcttaagaagaagttctacgtcggcattcgagaactgaatgacgggacatatgatgtc
agcattgctactaaagccggtgagctgctggttaaagacgaagaaagtcttgtgatccgggaaattttgtcaacggaaggcatcaagaa
aatgaaattgtcatcctgggacaatccagaagaagccctgaatgatttgatgaatgcgctccaagaagctagcaatgctagtgctggcc
ccttcggccttattatcaatccaaagcggtacgccaaactgctgaagatctatgaaaagtcaggtaagatgctcgtagaagtactcaagg
aaatcttccggggtggaataatcgtaactcttaatatcgacgaaaacaaagtgattatcttcgctaatacgcccgccgttctggacgtggt
ggtgggtcaagacgttacgctccaggagcttggtccggaaggggatgatgtcgcattcctggtcagtgaagccattggtataagaatc
aagaacccggaagctatagttgttctcgaaggcgggtctggtgggagcggtggtagtggtggttctggtggtggtgggtcaggtggc
ggctcaggcggcggcaatctgtgccctttcggtgaggtctttaatgcaacaagatttgcaagtgtttacgcctggaaccgtaagcgcatt
agcaactgcactgccgattactctgtgctgtacaacagcacaagcttttccacatttaaatgttacggggtttcccctaccaacctcagcg
acctctgctttactaatgtttacgcagattccttcgttatccgaggcgatgaagtccggcagatagctcccggacagaccggcaaaatcg
ctgactacaactataaactgccgaacgacagcacagggtgtgtaattgcttggaacagcaataacctcgattcaaaggttggcggaaat
tacaattatctttaccgtctgttccggaaaagcaatctgaaaccctttgagagagacatcagcacggaaatttatcaagccggttcaacac
catgtaacggagttgaaggctttaattgctattttcccctgcaatcttacggatttcaacctacgaacggggtcggttaccaaccttaccgg
gtggtcgtgctgagcttcgaattgcttcatgccccagccaccgtgtgtgggccataatga (SEQ ID NO: 74)
ggauccgccaccauggacuggaccuggauucuguuccugguggccgccgccacaagggugcacagccucucaauugccc
gaugcaauuaaucucuaugaacaaauggcccgcuauagcgaagacgaaaaugugagaaagaucuuguuggacgucgcua
gggaagagaaagcacacguaggagaguucauggcuuuguugcuuaaccucgacccugagcaagucacagagcugaaggg
ggguucgaggaaguuaaagaauugaccgguauagaagcucacauuaaugacaacaagaaagaggaaaguaauguagag
uauuucgagaagcucagaucugccuuguuggauggagucaacaagggucgcagcuugcucaaacaucugcccguuacaa
gaauagaagggcagucuuuucgaguagacaucaucaaauuugaggacggcguccgagugguuaaacaagaguauaagcc
uauaccccuucuuaagaagaaguucuacgucggcauucgagaacugaaugacgggacauaugaugucagcauugcuacu
ugaaauugucauccugggacaauccagaagaagcccugaaugauuugaugaaugcgcuccaagaagcuagcaaugcuag
cgcuaauacgcccgccguucuggacgugguggugggucaagacguuacgcuccaggagcuugguccggaaggggauga
ugucgcauuccuggucagugaagccauugguauaagaaucaagaacccggaagcuauaguuguucucgaaggcgggucu
ggugggagcggugguaguggugguucuggugguggugggucagguggcggcucaggcggcggcaaucugugcccuuu
cggugaggucuuuaaugcaacaagauuugcaaguguuuacgccuggaaccguaagcgcauuagcaacugcacugccgau
cuuuacuaauguuuacgcagauuccuucguuauccgaggcgaugaaguccggcagauagcucccggacagaccggcaaa
aucgcugacuacaacuauaaacugccgaacgacagcacaggguguguaauugcuuggaacagcaauaaccucgauucaaa
uucaaccuacgaacggggucgguuaccaaccuuaccggguggucgugcugagcuucgaauugcuucaugccccagccac
cgugugugggccauaauga (SEQ ID NO: 75)
MDWTWILFLVAAATRVHSLSIAPTLINRDKPYTKEELMEILRLAIIAELDAINLYEQM
ARYSEDENVRKILLDVAREEKAHVGEFMALLLNLDPEQVTELKGGFEEVKELTGIEA
HINDNKKEESNVEYFEKLRSALLDGVNKGRSLLKHLPVTRIEGQSFRVDIIKFEDGVR
VVKQEYKPIPLLKKKFYVGIRELNDGTYDVSIATKAGELLVKDEESLVIREILSTEGIK
KMKLSSWDNPEEALNDLMNALQEASNASAGPFGLIINPKRYAKLLKIYEKSGKMLV
EVLKEIFRGGIIVTLNIDENKVIIFANTPAVLDVVVGQDVTLQELGPEGDDVAFLVSEA
IGIRIKNPEAIVVLEGGSGGSGGSGGSGGGGSGGGSGGGNLCPFGEVFNATRFASVYA
WNRKRISNCTADYSVLYNSTSFSTFKCYGVSPTNLSDLCFTNVYADSFVIRGDEVRQI
APGQTGKIADYNYKLPNDSTGCVIAWNSNNLDSKVGGNYNYLYRLFRKSNLKPFER
DISTEIYQAGSTPCNGVEGFNCYFPLQSYGFQPTNGVGYQPYRVVVLSFELLHAPATV
CGP** (SEQ ID NO: 76)
WuhanS_RBD_gmax_LS_pVax
ggatccgccaccatggactggacctggattctgttcctggtggccgccgccacaagggtgcacagcatgcagatctacgaaggaaaa
ctgaccgctgagggactgaggttcggaattgtcgcaagccgcgcgaatcacgcactggtggataggctggtggaaggcgctatcga
cgcaattgtccggcacggcgggagagaggaagacatcacactggtgagagtctgcggcagctgggagattcccgtggcagctgga
gaactggctcgaaaggaggacatcgatgccgtgatcgctattggggtcctgtgccgaggagcaactcccagcttcgactacatcgcct
cagaagtgagcaaggggctggctgatctgtccctggagctgaggaaacctatcacttttggcgtgattactgccgacaccctggaaca
ggcaatcgaggcggccggcacctgccatggaaacaaaggctgggaagcagccctgtgcgctattgagatggcaaatctgttcaaat
ctctgcgaggaggctccggaggatctggagggagtggaggctcaggaggaggcaatctgtgccctttcggtgaggtctttaatgcaa
caagatttgcaagtgtttacgcctggaaccgtaagcgcattagcaactgcactgccgattactctgtgctgtacaacagcacaagcttttc
cacatttaaatgttacggggtttcccctaccaacctcagcgacctctgctttactaatgtttacgcagattccttcgttatccgaggcgatga
agtccggcagatagctcccggacagaccggcaaaatcgctgactacaactataaactgccgaacgacagcacagggtgtgtaattgc
ttggaacagcaataacctcgattcaaaggttggcggaaattacaattatctttaccgtctgttccggaaaagcaatctgaaaccctttgag
agagacatcagcacggaaatttatcaagccggttcaacaccatgtaacggagttgaaggctttaattgctattttcccctgcaatcttacg
gatttcaacctacgaacggggtcggttaccaaccttaccgggtggtcgtgctgagcttcgaattgcttcatgccccagccaccgtgtgtg
ggccataatga (SEQ ID NO: 77)
ggauccgccaccauggacuggaccuggauucuguuccugguggccgccgccacaagggugcacagcaugcagaucuacg
aaggaaaacugaccgcugagggacugagguucggaauugucgcaagccgcgcgaaucacgcacugguggauaggcuggu
ggaaggcgcuaucgacgcaauuguccggcacggcgggagagaggaagacaucacacuggugagagucugcggcagcugg
gagauucccguggcagcuggagaacuggcucgaaaggaggacaucgaugccgugaucgcuauugggguccugugccgag
gagcaacucccagcuucgacuacaucgccucagaagugagcaaggggcuggcugaucugucccuggagcugaggaaacc
uaucacuuuuggcgugauuacugccgacacccuggaacaggcaaucgaggcggccggcaccugccauggaaacaaaggcu
gggaagcagcccugugcgcuauugagauggcaaaucuguucaaaucucugcgaggaggcuccggaggaucuggagggag
uggaggcucaggaggaggcaaucugugcccuuucggugaggucuuuaaugcaacaagauuugcaaguguuuacgccug
gaaccguaagcgcauuagcaacugcacugccgauuacucugugcuguacaacagcacaagcuuuuccacauuuaaauguu
acgggguuuccccuaccaaccucagcgaccucugcuuuacuaauguuuacgcagauuccuucguuauccgaggcgauga
aguccggcagauagcucccggacagaccggcaaaaucgcugacuacaacuauaaacugccgaacgacagcacagggugug
uaauugcuuggaacagcaauaaccucgauucaaagguuggcggaaauuacaauuaucuuuaccgucuguuccggaaaag
caaucugaaacccuuugagagagacaucagcacggaaauuuaucaagccgguucaacaccauguaacggaguugaaggcu
uuaauugcuauuuuccccugcaaucuuacggauuucaaccuacgaacggggucgguuaccaaccuuaccggguggucgu
gcugagcuucgaauugcuucaugccccagccaccgugugugggccauaauga (SEQ ID NO: 78)
MDWTWILFLVAAATRVHSMQIYEGKLTAEGLRFGIVASRANHALVDRLVEGAIDAI
VRHGGREEDITLVRVCGSWEIPVAAGELARKEDIDAVIAIGVLCRGATPSFDYIASEV
SKGLADLSLELRKPITFGVITADTLEQAIEAAGTCHGNKGWEAALCAIEMANLFKSLR
GGSGGSGGSGGSGGGNLCPFGEVFNATRFASVYAWNRKRISNCTADYSVLYNSTSFS
TFKCYGVSPTNLSDLCFTNVYADSFVIRGDEVRQIAPGQTGKIADYNYKLPNDSTGC
VIAWNSNNLDSKVGGNYNYLYRLFRKSNLKPFERDISTEIYQAGSTPCNGVEGFNCY
FPLQSYGFQPTNGVGYQPYRVVVLSFELLHAPATVCGP** (SEQ ID NO: 79)
CoV2-RBD_7mer_pVax
ggatccgccaccatggactggacctggattctgttcctggtggccgccgccacaagggtgcacagcaatctctgtccattcggagagg
ttttcaacgcgacgagattcgcctcagtttatgcctggaaccgtaaacggatatcaaactgcgtggctgactactctgttttatacaactcc
gcctctttcagtaccttcaagtgttacggtgtcagccctaccaaattgaatgatctctgctttacaaatgtttacgcagattcttttgtcataag
gggcgatgaggttcggcaaatcgcccccgggcagacaggcaaaattgcggactataattataagttgccagacgatttcacgggctg
cgtcatcgcctggaacagtaataatctcgattcaaaagtgggtgggaactacaattatctctacaggttattccggaagtcaaatctgaag
cccttcgaacgcgacatcagtacggagatttaccaggctggaagcactccgtgcaacggggtggaggggttcaactgttattttcctct
gcagtcttatgggtttcagcccactaatggtgtgggataccagccgtacagagtcgtggtgctgtccttcgaacttctccacgctcccgc
caccgtctgtggtcccgggggatctggcggatcagggggtagtggaggtagcggcggcgggaagaaacagggagacgctgacgt
ctgtggggaagtggcttacatccagagcgtggtgtctgattgccatgtaccaaccgcggagctcaggactcttttagagattcggaaac
tgtttctggagatccaaaagctgaaggtcgaactccagggcctgtcaaaagaatgataa (SEQ ID NO: 80)
ggauccgccaccauggacuggaccuggauucuguuccugguggccgccgccacaagggugcacagcaaucucuguccau
ucggagagguuuucaacgcgacgagauucgccucaguuuaugccuggaaccguaaacggauaucaaacugcguggcuga
cuacucuguuuuauacaacuccgccucuuucaguaccuucaaguguuacggugucagcccuaccaaauugaaugaucuc
ugcuuuacaaauguuuacgcagauucuuuugucauaaggggcgaugagguucggcaaaucgcccccgggcagacaggca
aaauugcggacuauaauuauaaguugccagacgauuucacgggcugcgucaucgccuggaacaguaauaaucucgauuc
aaaagugggugggaacuacaauuaucucuacagguuauuccggaagucaaaucugaagcccuucgaacgcgacaucagua
cggagauuuaccaggcuggaagcacuccgugcaacgggguggagggguucaacuguuauuuuccucugcagucuuaug
gguuucagcccacuaauggugugggauaccagccguacagagucguggugcuguccuucgaacuucuccacgcucccgc
caccgucuguggucccgggggaucuggcggaucaggggguaguggagguagcggcggcgggaagaaacagggagacgc
ugacgucuguggggaaguggcuuacauccagagcguggugucugauugccauguaccaaccgcggagcucaggacucuu
uuagagauucggaaacuguuucuggagauccaaaagcugaaggucgaacuccagggccugucaaaagaaugauaa
(SEQ ID NO: 81)
NLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGKKQGDADVCGEVAYIQSVV
SDCHVPTAELRTLLEIRKLFLEIQKLKVELQGLSKE** (SEQ ID NO: 82)
CoV2-RBD_gmax_14mer_pVax
ggatccgccaccatggactggacctggattctgttcctggtggccgccgccacaagggtgcacagcaatctgtgtcccttcggggag
gttttcaatgctaccagatttgccagtgtgtatgcttggaatcggaagagaatctccaattgcacagcagattattcagttctctacaactct
acatcttttagtacctttaagtgttacggggtgagtcccactaacctttcagatttatgtttcaccaatgtctacgctgactccttcgtgatccg
gggggatgaggtgagacagattgcacctggacaaactggcaaaatcgccgactacaattacaaacttccaaacgactctacagggtg
tgtaatcgcttggaacagcaataatctggatagcaaagtaggcggcaattataattacctctacagactgtttaggaagtccaacctgaa
accatttgagagggacatcagcactgaaatctaccaggggggagcaccccttgtaatggagtcgagggtttcaactgttacttcccac
tgcagagctacgggttccagcctaccaatggtgtcggttaccagccctatcgagttgtggtgttgtcattcgaactgttacatgcacctgc
aacggtctgtggacccgggggttcagggggtagtggggggtccggtgggagcggtgggggcaagaaacaggggatgaatccgct
catcgccgccgcctctgtgatagctgctggcctggccgtgggcctggcatcaatcgggcccggggtgggccaaggcaccgccgcc
ggccaggccgtcgagggtattgcaaggcagccggaggcagaaggcaaaattagagggaccctgttgttgtctttagcgttcatggaa
gccctcactatttacggactggttgtggccttagcccttctgtttgccaatcctttcgtgtaatga (SEQ ID NO: 83)
ggauccgccaccauggacuggaccuggauucuguuccugguggccgccgccacaagggugcacagcaaucugugucccu
ucggggagguuuucaaugcuaccagauuugccaguguguaugcuuggaaucggaagagaaucuccaauugcacagcaga
uuauucaguucucuacaacucuacaucuuuuaguaccuuuaaguguuacggggugagucccacuaaccuuucagauuua
uguuucaccaaugucuacgcugacuccuucgugauccggggggaugaggugagacagauugcaccuggacaaacuggca
aaaucgccgacuacaauuacaaacuuccaaacgacucuacaggguguguaaucgcuuggaacagcaauaaucuggauagc
aaaguaggcggcaauuauaauuaccucuacagacuguuuaggaaguccaaccugaaaccauuugagagggacaucagcac
ugaaaucuaccaggggggagcaccccuuguaauggagucgaggguuucaacuguuacuucccacugcagagcuacggg
uuccagccuaccaauggugucgguuaccagcccuaucgaguugugguguugucauucgaacuguuacaugcaccugcaa
cggucuguggacccggggguucaggggguagugggggguccggugggagcggugggggcaagaaacaggggaugaauc
cgcucaucgccgccgccucugugauagcugcuggccuggccgugggccuggcaucaaucgggcccggggugggccaagg
caccgccgccggccaggccgucgaggguauugcaaggcagccggaggcagaaggcaaaauuagagggacccuguuguug
ucuuuagcguucauggaagcccucacuauuuacggacugguuguggccuuagcccuucuguuugccaauccuuucgug
uaauga (SEQ ID NO: 84)
NLCPFGEVFNATRFASVYAWNRKRISNCTADYSVLYNSTSFSTFKCYGVSPTNLSDL
CFTNVYADSFVIRGDEVRQIAPGQTGKIADYNYKLPNDSTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGKKQGMNPLIAAASVIAAGL
AVGLASIGPGVGQGTAAGQAVEGIARQPEAEGKIRGTLLLSLAFMEALTIYGLVVAL
ALLFANPFV** (SEQ ID NO: 85)
CoV2-RBD_gmax_24mer_pVax
ggatccgccaccatggactggacctggattctgttcctggtggccgccgccacaagggtgcacagcaacctgtgccccttcggggaa
gtttttaatgccactcggtttgcctccgtttacgcctggaacaggaagcgcatttccaactgcaccgccgactatagcgtcctttataacag
cacatccttttcaactttcaagtgttacggggtttcccctacaaatctctctgacctgtgttttacaaatgtgtatgcagactctttcgtgattag
gggagatgaggtgcgccagatcgcccctggacagaccggaaaaatcgccgattataattataagcttcccaacgactccacaggctg
tgtaattgcctggaattctaataacctggactctaaagtgggcggtaactacaattatctgtatagactcttcagaaagtctaacctcaaac
catttgaacgggacatctcaaccgagatctaccaagccgggtccaccccctgtaacggcgtggaaggcttcaactgttatttccccctc
cagtcctatggcttccaacccacaaatggagtcggctaccagccttacagggtggttgtgctgtcatttgagctcctccacgctcctgcc
actgtatgtgggccaggcgggtccggaggttcaggcggtagcggcggctcaggtggaggaggactgtctaaagatattataaaactg
ctgaacgaacaagtgaacaaggagatgcagagcagcaacctttacatgtctatgagcagttggtgttacactcactctctcgacggcgc
cggcctgttcctgtttgatcacgccgcggaggagtatgaacatgctaaaaagcttatcatcttcctcaacgaaaataacgtgccagtgca
gttgacctctatttccgctcccgaacataagttcgaaggcctcacacagatctttcagaaggcttacgagcatgaacaacacatttcaga
gagcatcaacaacatcgtggaccatgcgatcaagtctaaggaccacgcgacttttaacttcctccagtggtatgtcgccgaacagcatg
aggaggaagtgttgttcaaagacatcctggacaagattgaacttattggcaacgaaaaccacggcctctacctggccgatcagtacgt
gaaaggtatcgcgaagtcacgaaagagttaatga (SEQ ID NO: 86)
ggauccgccaccauggacuggaccuggauucuguuccugguggccgccgccacaagggugcacagcaaccugugccccu
ucggggaaguuuuuaaugccacucgguuugccuccguuuacgccuggaacaggaagcgcauuuccaacugcaccgccga
cuauagcguccuuuauaacagcacauccuuuucaacuuucaaguguuacgggguuuccccuacaaaucucucugaccug
uguuuuacaaauguguaugcagacucuuucgugauuaggggagaugaggugcgccagaucgccccuggacagaccggaa
aaaucgccgauuauaauuauaagcuucccaacgacuccacaggcuguguaauugccuggaauucuaauaaccuggacucu
aaagugggcgguaacuacaauuaucuguauagacucuucagaaagucuaaccucaaaccauuugaacgggacaucucaac
cgagaucuaccaagccggguccacccccuguaacggcguggaaggcuucaacuguuauuucccccuccaguccuauggcu
uccaacccacaaauggagucggcuaccagccuuacagggugguugugcugucauuugagcuccuccacgcuccugccac
uguaugugggccaggcggguccggagguucaggcgguagcggcggcucagguggaggaggacugucuaaagauauuau
aaaacugcugaacgaacaagugaacaaggagaugcagagcagcaaccuuuacaugucuaugagcaguugguguuacacuc
acucucucgacggcgccggccuguuccuguuugaucacgccgcggaggaguaugaacaugcuaaaaagcuuaucaucuu
ccucaacgaaaaaacgugccagugcaguugaccucuauuuccgcucccgaacauaaguucgaaggccucacacagaucu
uucagaaggcuuacgagcaugaacaacacauuucagagagcaucaacaacaucguggaccaugcgaucaagucuaaggac
cacgcgacuuuuaacuuccuccagugguaugucgccgaacagcaugaggaggaaguguuguucaaagacauccuggaca
agauugaacuuauuggcaacgaaaaccacggccucuaccuggccgaucaguacgugaaagguaucgcgaagucacgaaag
aguuaauga (SEQ ID NO: 87)
NLCPFGEVFNATRFASVYAWNRKRISNCTADYSVLYNSTSFSTFKCYGVSPTNLSDL
CFTNVYADSFVIRGDEVRQIAPGQTGKIADYNYKLPNDSTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGGLSKDIIKLLNEQVNKEMQS
SNLYMSMSSWCYTHSLDGAGLFLFDHAAEEYEHAKKLIIFLNENNVPVQLTSISAPE
HKFEGLTQIFQKAYEHEQHISESINNIVDHAIKSKDHATFNFLQWYVAEQHEEEVLFK
DILDKIELIGNENHGLYLADQYVKGIAKSRKS** (SEQ ID NO: 88)
CoV2-RBD_gmax_7mer_pVax
ggatccgccaccatggactggacctggattctgttcctggtggccgccgccacaagggtgcacagcaatctttgtccattcggggaag
tgtttaacgccactaggttcgctagtgtgtacgcctggaatcggaagcggatttcaaattgtaccgccgattattctgtcctttacaacagt
accagcttttccacttttaaatgctacggagtatctcctacaaacttgagtgacctgtgttttacgaacgtctacgctgactctttcgttattag
gggagacgaagttagacaaatcgctccaggccagactggcaaaatagccgactataactataaactcccaaacgattccacaggctg
cgttattgcctggaacagcaataacctggactctaaagtcggaggtaactataactacttgtacaggctcttccgcaagagcaaccttaa
gccatttgagcgagatatctccaccgagatttatcaggcagggagcaccccatgcaacggagtggaggggtttaattgctattttccact
gcagtcctatggctttcaaccaacaaacggagtaggctaccaaccgtatcgcgttgtcgtcctgagtttcgaactgttgcacgcccctgc
gaccgtatgtggccccggcggctcaggggggagtggtgggagcgggggctctgggggggggaaaaaacagggggacgccgat
gtttgcggcgaggtggcctatatacagtcagtggtctccgactgtcatgtaccaactgccgaactcaggactcttctggagataaggaa
gttgttcctggagatacagaagctcaaggtcgagttacagggtctctcaaaggaatgatga (SEQ ID NO: 89)
ggauccgccaccauggacuggaccuggauucuguuccugguggccgccgccacaagggugcacagcaaucuuuguccau
ucggggaaguguuuaacgccacuagguucgcuaguguguacgccuggaaucggaagcggauuucaaauuguaccgccga
uuauucuguccuuuacaacaguaccagcuuuuccacuuuuaaaugcuacggaguaucuccuacaaacuugagugaccug
uguuuuacgaacgucuacgcugacucuuucguuauuaggggagacgaaguuagacaaaucgcuccaggccagacuggca
aaauagccgacuauaacuauaaacucccaaacgauuccacaggcugcguuauugccuggaacagcaauaaccuggacucu
aaagucggagguaacuauaacuacuuguacaggcucuuccgcaagagcaaccuuaagccauuugagcgagauaucucac
cgagauuuaucaggcagggagcaccccaugcaacggaguggagggguuuaauugcuauuuuccacugcaguccuauggc
uuucaaccaacaaacggaguaggcuaccaaccguaucgcguugucguccugaguuucgaacuguugcacgccccugcgac
cguauguggccccggcggcucaggggggaguggugggagcgggggcucugggggggggaaaaaacagggggacgccga
uguuugcggcgagguggccuauauacagucaguggucuccgacugucauguaccaacugccgaacucaggacucuucug
gagauaaggaaguuguuccuggagauacagaagcucaaggucgaguuacagggucucucaaaggaaugauga (SEQ
ID NO: 90)
NLCPFGEVFNATRFASVYAWNRKRISNCTADYSVLYNSTSFSTFKCYGVSPTNLSDL
CFTNVYADSFVIRGDEVRQIAPGQTGKIADYNYKLPNDSTGCVIAWNSNNLDSKVGG
NYNYLYRLFRKSNLKPFERDISTEIYQAGSTPCNGVEGFNCYFPLQSYGFQPTNGVGY
QPYRVVVLSFELLHAPATVCGPGGSGGSGGSGGSGGGKKQGDADVCGEVAYIQSVV
SDCHVPTAELRTLLEIRKLFLEIQKLKVELQGLSKE** (SEQ ID NO: 91)
II. CoV2 Trimer Constructs
WuhanS_SolTri_FS1_pVax
ggatccgccaccatggactggacctggattctgttcctggtggccgccgccacaagggtgcacagcatgtttgttttcctcgtcctcttg
cccctcgtctctagtcaatgtgttaatttgaccacacgaacccaactgccacctgcctacaccaacagttttaccagaggagtttattaccc
cgacaaagtattcaggtcatcagtgctgcatagtacccaagacttgtttctccccttctttagtaacgttacatggttccacgccattcacgt
gagtgggacaaatggaacaaaacgcttcgacaaccctgtgctccccttcaacgatggtgtatactttgctagtaccgagaagagcgga
attatccgcgggtggatctttggaacaacactggacagcaaaacccaaagcctgcttatcgttaacaatgctactaacgttgtgatcaaa
gtgtgtgaattccaattttgtaatgatccgtttctcggagtttactaccacaagaacaacaaaagttggatggaaagcgaattccgggtgta
ctcctcagcaaataattgtacctttgagtacgtgagtcaaccctttctcatggacctggaaggaaaacaaggcaatttcaagaacctgcg
ggagtttgtgttcaagaatattgatggctattttaaaatttattctaagcatactccaatcaacctggtaagggacctgccccaaggcttttca
gccctcgaaccgcttgtagatttgcctatcgggataaacattacgcgatttcaaacgctgttggcgctccaccggagctacttgactcctg
gcgatagcagctccggttggaccgctggagcggccgcttattacgtcggctatctgcaacccaggacgttcctgctcaagtataatga
gaacgggacgattacagatgcagtggattgtgcgcttgatcctctctctgaaaccaagtgcactctcaagtctttcacggtggagaaag
gcatttatcaaactagtaactttcgagtacagcctactgagagtatcgttaggttcccaaacattacgaacctctgtccctttggagaagtat
tcaatgctactcgctttgcaagcgtttatgcctggaatcgcaaacgcatcagcaattgcgtcgccgattattctgtcctttataatagcgcat
cattttcaacatttaagtgttatggggtgagtccgactaagctcaatgatttgtgcttcacaaacgtctacgcggacagctttgtgataagg
ggcgacgaagttcgccaaatcgctcccggccaaactgggaaaatcgcggattacaactataaattgcccgatgacttcaccggctgtg
tcattgcctggaactctaataacctcgatagcaaggtggggggaactataattatttgtaccgcctgtttcgaaagtccaatctcaaacc
ctttgagcgggacatttccactgagatctatcaggcagggagtacaccttgtaacggcgtggaaggctttaactgttattttcccctgcaa
agttacggttttcaacctaccaacggagttggctatcaaccttatcgagtcgtcgtgctgagttttgagttgctgcatgccccagccaccgt
ctgtggacctaagaaatccaccaacctcgtgaagaacaagtgcgtcaattttaattttaacggcctgactgggaccggtgtcctcaccga
atctaataagaagttcctgccatttcaacaattcggacgggacatcgctggaacgacagatgctgtccgtgatcctcagacactggaga
ttctggacatcactccttgcagctttggcggagtctctgttattactcccggaactaacacttctaaccaagttgctgtcctctatcaggacg
tgaactgcactgaagtgcccgtggcaatccatgcaggccaactgacccccacttggagagtctacagcacggggagcaatgtcttcc
aaacaagggccggatgccttattggagcggagcacgttaataactcatacgagtgtgatataccaattggagcaggaatttgtgcttcct
accagacccaaactaacagtcccagggggctaggagtgtcgctagccagagcatcatcgcgtacacaatgtctctcggcgcagaa
aactcagtcgcctatagcaacaactcaattgccattcccaccaacttcacaatttccgtaaccactgaaattctgcctgttagcatgacaaa
gacatcagttgattgtacaatgtacatatgtggagacagcaccgaatgcagcaaccttttgcttcaatatggctccttttgtacccaactca
acagggcactcactgggatagcagtcgaacaagataagaacacccaagaggtgtttgcacaagtcaaacaaatctataaaacgccgc
ccataaaagactttggcggattcaatttcagccagatcttgcctgacccatccaagccttcaaagaggagctttattgaggatcttctcttc
aataaagtgacactggcggacgccggttttatcaagcaatatggtgattgtctcggtgacatagcagctagagatctgatttgcgctcag
aaatttaatggccttactgtgcttcccccactgctgaccgatgaaatgattgcacaatatacaagcgcccttttggccgggactattacttc
cgggtggaccttcggcgccggcgccgctctgcaaattcctttcgcaatgcagatggcctaccggttcaatggcataggtgtcactcag
aacgttctttatgagaatcagaaactcatcgcgaaccagtttaattcagcgatcggcaagattcaggactccttgtcctcaactgcgtcag
ctttgggaaaacttcaagacgtcgtgaaccagaatgctcaggcgctcaataccctggtgaaacaacttagcagtaactttggggctattt
ctagcggtccaaacgatatactgtcccgactcccgaaagtcgaggccgaagtccaaattgatcgtcttattacagggagactccaatct
cttcaaacatatgtcactcaacagctcattagggctgcggagatccgggcttccgcaaatcttgccgcgacaaagatgagtgaatgcgt
cttgggacaatctaagagggtggacttttgtggaaaaggttaccatctcatgtccttccctcagtcagcgccccacggagtcgttttcctg
cacgtaacgtatgtcccggctcaagagaagaacttcactactgcaccagcgatttgccatgacggtaaagcccattttccccgcgagg
gcgtatttgtgtccaacggtacccactggttcgtaacccaacggaatttctatgagccccaaatcattacaacagataatacagatgtttcc
gggaattgcgacgttgttattggcatcgttaacaacaccgtttacgatcccttgcaaccggaactggactcctttaaagaagaactcgac
aagtattttaagaaccacacatcaccagatgtcgatcttggcgacatttccggcattaacgcttcagttgtaaatattcagaaagagatag
atcgcctgaatgaggtggctaagaacctgaacgaatctctcattgatctccaagagctgggaaagtacgaacaatacatcaaatggcct
tctgggcgtcgccgaagacgacgagggtccggcggctcagggagcggctatatccctgaggcgcctcgggacggacaagcttatg
tgaggaaagatggagaatgggtattgctgtcaaccttcctgggataatga (SEQ ID NO: 92)
ggauccgccaccauggacuggaccuggauucuguuccugguggccgccgccacaagggugcacagcauguuuguuuucc
ucguccucuugccccucgucucuagucaauguguuaauuugaccacacgaacccaacugccaccugccuacaccaacagu
uuuaccagaggaguuuauuaccccgacaaaguauucaggucaucagugcugcauaguacccaagacuuguuucuccccu
ucuuuaguaacguuacaugguuccacgccauucacgugagugggacaaauggaacaaaacgcuucgacaacccugugcuc
cccuucaacgaugguguauacuuugcuaguaccgagaagagcggaauuauccgcggguggaucuuuggaacaacacugg
acagcaaaacccaaagccugcuuaucguuaacaaugcuacuaacguugugaucaaagugugugaauuccaauuuuguaau
gauccguuucucggaguuuacuaccacaagaacaacaaaaguuggauggaaagcgaauuccggguguacuccucagcaaa
uaauuguaccuuugaguacgugagucaacccuuucucauggaccuggaaggaaaacaaggcaauuucaagaaccugcgg
gaguuuguguucaagaauauugauggcuauuuuaaaauuuauucuaagcauacuccaaucaaccugguaagggaccugc
cccaaggcuuuucagcccucgaaccgcuuguagauuugccuaucgggauaaacauuacgcgauuucaaacgcuguuggc
gcuccaccggagcuacuugacuccuggcgauagcagcuccgguuggaccgcuggagcggccgcuuauuacgucggcuau
cugcaacccaggacguuccugcucaaguauaaugagaacgggacgauuacagaugcaguggauugugcgcuugauccuc
ucucugaaaccaagugcacucucaagucuuucacgguggagaaaggcauuuaucaaacuaguaacuuucgaguacagccu
acugagaguaucguuagguucccaaacauuacgaaccucugucccuuuggagaaguauucaaugcuacucgcuuugcaa
gcguuuaugccuggaaucgcaaacgcaucagcaauugcgucgccgauuauucuguccuuuauaauagcgcaucauuuuc
aacauuuaaguguuauggggugaguccgacuaagcucaaugauuugugcuucacaaacgucuacgcggacagcuuugug
auaaggggcgacgaaguucgccaaaucgcucccggccaaacugggaaaaucgcggauuacaacuauaaauugcccgauga
cuucaccggcugugucauugccuggaacucuaauaaccucgauagcaaggugggcgggaacuauaauuauuuguaccgc
cuguuucgaaaguccaaucucaaacccuuugagcgggacauuuccacugagaucuaucaggcagggaguacaccuugua
acggcguggaaggcuuuaacuguuauuuuccccugcaaaguuacgguuuucaaccuaccaacggaguuggcuaucaacc
uuaucgagucgucgugcugaguuuugaguugcugcaugccccagccaccgucuguggaccuaagaaauccaccaaccuc
gugaagaacaagugcgucaauuuuaauuuuaacggccugacugggaccgguguccucaccgaaucuaauaagaaguucc
ugccauuucaacaauucggacgggacaucgcuggaacgacagaugcuguccgugauccucagacacuggagauucugga
caucacuccuugcagcuuuggcggagucucuguuauuacucccggaacuaacacuucuaaccaaguugcuguccuuau
caggacgugaacugcacugaagugcccguggcaauccaugcaggccaacugacccccacuuggagagucuacagcacggg
gagcaaugucuuccaaacaagggccggaugccuuauuggagcggagcacguuaauaacucauacgagugugauauacca
auuggagcaggaauuugugcuuccuaccagacccaaacuaacagucccagggggcuaggagugucgcuagccagagca
ucaucgcguacacaaugucucucggcgcagaaaacucagucgccuauagcaacaacucaauugccauucccaccaacuuca
caauuuccguaaccacugaaauucugccuguuagcaugacaaagacaucaguugauuguacaauguacauauguggaga
cagcaccgaaugcagcaaccuuuugcuucaauauggcuccuuuuguacccaacucaacagggcacucacugggauagcag
ucgaacaagauaagaacacccaagagguguuugcacaagucaaacaaaucuauaaaacgccgcccauaaaagacuuuggcg
gauucaauuucagccagaucuugccugacccauccaagccuucaaagaggagcuuuauugaggaucuucucuucaauaaa
gugacacuggcggacgccgguuuuaucaagcaauauggugauugucucggugacauagcagcuagagaucugauuugc
gcucagaaauuuaauggccuuacugugcuucccccacugcugaccgaugaaaugauugcacaauauacaagcgcccuuuu
ggccgggacuauuacuuccggguggaccuucggcgccggcgccgcucugcaaauuccuuucgcaaugcagauggccuac
cgguucaauggcauaggugucacucagaacguucuuuaugagaaucagaaacucaucgcgaaccaguuuaauucagcga
ucggcaagauucaggacuccuuguccucaacugcgucagcuuugggaaaacuucaagacgucgugaaccagaaugcuca
ggcgcucaauacccuggugaaacaacuuagcaguaacuuuggggcuauuucuagcgguccaaacgauauacugucccga
cucccgaaagucgaggccgaaguccaaauugaucgucuuauuacagggagacuccaaucucuucaaacauaugucacuca
acagcucauuagggcugcggagauccgggcuuccgcaaaucuugccgcgacaaagaugagugaaugcgucuugggacaa
ucuaagaggguggacuuuuguggaaaagguuaccaucucauguccuucccucagucagcgccccacggagucguuuucc
ugcacguaacguaugucccggcucaagagaagaacuucacuacugcaccagcgauuugccaugacgguaaagcccauuuu
ccccgcgagggcguauuuguguccaacgguacccacugguucguaacccaacggaauuucuaugagccccaaaucauuac
aacagauaauacagauguuuccgggaauugcgacguuguuauuggcaucguuaacaacaccguuuacgaucccuugcaa
ccggaacuggacuccuuuaaagaagaacucgacaaguauuuuaagaaccacacaucaccagaugucgaucuuggcgacau
uuccggcauuaacgcuucaguuguaaauauucagaaagagauagaucgccugaaugagguggcuaagaaccugaacgaa
ucucucauugaucuccaagagcugggaaaguacgaacaauacaucaaauggccuucugggcgucgccgaagacgacgagg
guccggcggcucagggagcggcuauaucccugaggcgccucgggacggacaagcuuaugugaggaaagauggagaaugg
guauugcugucaaccuuccugggauaauga (SEQ ID NO: 93)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSGIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIAGTTDAVRDPQTLEILDITPCSFGGVSVITPGTNTSNQVAVLYQDV
NCTEVPVAIHAGQLTPTWRVYSTGSNVFQTRAGCLIGAEHVNNSYECDIPIGAGICAS
YQTQTNSPRRARSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTDVSGNCDVVIGIVNNTV
YDPLQPELDSFKEELDKYFKNHTSPDVDLGDISGINASVVNIQKEIDRLNEVAKNLNE
SLIDLQELGKYEQYIKWPSGRRRRRRGSGGSGSGYIPEAPRDGQAYVRKDGEWVLLS
TFLG** (SEQ ID NO: 94)
WuS_IgE_StrepHis_pVax
gccaccatggactggacctggatactctttctcgtagcagcagccacacgagtgcattcaagccaatgcgtgaatctgactacaagga
ctcagctgccccctgcatacacgaacagtttcacccgcggtgtatattatccggacaaagtattcaggtctagtgtgctgcactcaaccc
aggatttgtttctgcccttcttctctaacgtgacatggttccacgccatccatgtgtcaggtacgaacggtaccaagagatttgataacccg
gtactgccatttaatgacggcgtctattttgcttccactgagaagagcaacatcataagaggctggatctttggaactactctggacagca
aaacccagagcttgctgatcgtgaacaacgcgacaaatgtagtgatcaaagtatgtgagtttcaattctgtaacgatcccttccttggggt
ttattaccataagaataataagagttggatggagtccgaatttagagtttactcctcagctaataactgtacgttcgagtatgtctcccaacc
ttttcttatggatctcgaagggaaacagggtaactttaagaatcttcgagaatttgtgttcaagaacatcgacggttattttaagatctacagt
aagcatactcccataaatctggttagagatctcccgcaaggattttccgcactggagccccttgtagaccttcccattggaataaacataa
cacgtttccagacactcctcgctctgcataggtcatatctcaccccgggcgattcttccagcggatggaccgctggagctgctgcttact
acgtaggatacctgcaaccccggacatttctgctcaagtataacgaaaatgggactattacggacgctgtggactgtgctcttgaccca
cttagcgagacaaaatgcacgctgaaaagttttaccgtggagaaggggatctatcaaacgagcaattttagggttcagcctaccgaatc
aatcgtcagatttcccaatatcactaacctgtgccctttcggggaagttttcaacgcaacccggtttgcgagcgtatacgcttggaatcgc
aaaaggataagcaattgcgttgccgattactccgttctttacaattcagcatcattttctacttttaaatgctacggcgtgtctcccacaaaac
tgaatgacctgtgttttacgaacgtgtatgcagacagctttgtgattaggggtgatgaggttagacaaatcgcaccaggtcagaccggta
agatcgctgattacaactacaaactgcccgatgacttcacaggatgcgtgattgcctggaattccaacaatctggattctaaggttggcg
gcaattacaattacctgtataggttgtttcggaagtcaaacctgaaacccttcgaaagagacatttctaccgagatttatcaagcgggttca
actccttgtaatggagttgaaggcttcaattgttactttccccttcaatcatacggattccaaccaaccaatggggtcggataccagccata
tagggttgttgtcctgtcattcgaacttctccacgcaccagccaccgtatgtggacccaagaagtctactaatctggtgaagaacaaatg
cgtcaatttcaactttaatgggttgaccggcactggggtgctgactgaatccaacaagaagtttctgccgttccaacaattcggacgcga
tatcgctgatacaaccgatgccgttagagatccccaaacattggagattctggatattacgccttgttcattcggtggtgtttccgtgattac
ccctggcaccaatacgagtaaccaagtggcggtgctgtatcaagatgtgaactgtactgaagtgccggtggctatacatgccgaccaa
ctcacaccaacatggagagtatatagcacgggttccaatgtgtttcaaactagggctggctgtttgattggcgctgaacatgttaataattc
ctatgaatgcgatattcccatcggtgccgggatttgcgcaagttatcaaacgcaaactaactcccccgggtcagcatcctctgtcgcttc
ccaatcaatcatcgcctataccatgagtcttggggcagaaaattccgttgcttattctaacaattccattgcaattcctacgaacttcaccat
ctcagttactacagaaatacttcccgtgtcaatgacgaagacatccgtagattgcacaatgtatatatgtggggactcaactgaatgctca
aacctgctcctgcaatacggatcattttgcacccaactgaacagagcattgaccggtatagccgtggagcaagataagaacactcaag
aagtattcgcccaggtcaaacaaatctataaaactccgcctataaaagattttggcggctttaacttttcccaaatactgcctgacccaagt
aagccctcaaaacgtagctttatagaggacctcttgtttaataaggtgacactcgctgacgctggattcattaagcaatatggtgactgctt
gggagatattgccgcccgcgatctcatttgtgcacaaaagttcaacggcctcacagtcctgccccctctgctgacggatgaaatgatcg
ctcaatacacctcagctctcctggcaggcaccataacaagcgggtggacatttggtgccggggcagcactgcaaatcccattcgcaat
gcaaatggcttataggttcaatgggatcggcgtaactcaaaatgtcctctacgagaaccagaaactcatagctaaccaattcaattctgc
aatcgggaaaatccaggactccctgagctcaacggccagcgcactgggcaagctccaagatgtggtcaaccaaaacgcacaagca
ctgaatactcttgtgaaacaactcagctccaatttcggggcaatatcaagtgtcctcaatgatattcttagcaggcttgatccacccgaag
ccgaggtgcagatcgacaggctcataacaggcaggctccagtcccttcaaacgtatgtaactcagcaactgattcgggctgccgagat
tcgagcttcagctaatttggcagctacgaagatgagcgaatgcgtcctgggacagtctaaaagagtagacttttgcggcaaagggtatc
atctgatgagcttcccacaaagtgctccacatggcgtggttttcctgcatgtcacttatgttcccgcacaagagaagaacttcactaccgc
accagcgatctgtcacgatggtaaagcacatttcccgcgggaaggcgtattcgtatctaacggcacccactggttcgttactcaacgca
acttttatgaaccacaaatcattacaaccgataacacttttgtttcaggcaattgcgatgttgtcatcggcattgtgaataacactgtgtacg
atccacttcaaccagaattggacagctttaaagaggagcttgataagtatttcaagaatcatacctctcccgacgtggacctcggggaca
tctctggaataaatgctagcgtcgttaatatacagaaagagattgatcgtctgaacgaagtggctaagaatctgaatgaaagccttatcga
tctgcaagaactggggaagtacgaacagggatacataccggaagccccacgcgacggtcaggcttatgttaggaaggatggagaat
gggttttgctctccacgtttctcgggcttgaagttttgttccaaggaccctggtcacacccccaatttgagaaacaccatcaccaccatca
ccaccactgataa (SEQ ID NO: 95)
gccaccauggacuggaccuggauacucuuucucguagcagcagccacacgagugcauucaagccaaugcgugaaucugac
uacaaggacucagcugcccccugcauacacgaacaguuucacccgcgguguauauuauccggacaaaguauucaggucua
gugugcugcacucaacccaggauuuguuucugcccuucuucucuaacgugacaugguuccacgccauccaugugucagg
uacgaacgguaccaagagauuugauaacccgguacugccauuuaaugacggcgucuauuuugcuuccacugagaagagc
aacaucauaagaggcuggaucuuuggaacuacucuggacagcaaaacccagagcuugcugaucgugaacaacgcgacaaa
uguagugaucaaaguaugugaguuucaauucuguaacgaucccuuccuugggguuuauuaccauaagaauaauaagagu
uggauggaguccgaauuuagaguuuacuccucagcuaauaacuguacguucgaguaugucucccaaccuuuucuuaugg
aucucgaagggaaacaggguaacuuuaagaaucuucgagaauuuguguucaagaacaucgacgguuauuuuaagaucua
caguaagcauacucccauaaaucugguuagagaucucccgcaaggauuuuccgcacuggagccccuuguagaccuuccca
uuggaauaaacauaacacguuuccagacacuccucgcucugcauaggucauaucucaccccgggcgauucuuccagcgga
uggaccgcuggagcugcugcuuacuacguaggauaccugcaaccccggacauuucugcucaaguauaacgaaaauggga
cuauuacggacgcuguggacugugcucuugacccacuuagcgagacaaaaugcacgcugaaaaguuuuaccguggagaa
ggggaucuaucaaacgagcaauuuuaggguucagccuaccgaaucaaucgucagauuucccaauaucacuaaccugugcc
cuuucggggaaguuuucaacgcaacccgguuugcgagcguauacgcuuggaaucgcaaaaggauaagcaauugcguugc
cgauuacuccguucuuuacaauucagcaucauuuucuacuuuuaaaugcuacggcgugucucccacaaaacugaaugacc
uguguuuuacgaacguguaugcagacagcuuugugauuaggggugaugagguuagacaaaucgcaccaggucagaccg
guaagaucgcugauuacaacuacaaacugcccgaugacuucacaggaugcgugauugccuggaauuccaacaaucuggau
ucuaagguuggcggcaauuacaauuaccuguauagguuguuucggaagucaaaccugaaacccuucgaaagagacauuu
cuaccgagauuuaucaagcggguucaacuccuuguaauggaguugaaggcuucaauuguuacuuuccccuucaaucaua
cggauuccaaccaaccaauggggucggauaccagccauauaggguuguuguccugucauucgaacuucuccacgcaccag
ccaccguauguggacccaagaagucuacuaaucuggugaagaacaaaugcgucaauuucaacuuuaauggguugaccgg
cacuggggugcugacugaauccaacaagaaguuucugccguuccaacaauucggacgcgauaucgcugauacaaccgaug
ccguuagagauccccaaacauuggagauucuggauauuacgccuuguucauucggugguguuuccgugauuaccccugg
caccaauacgaguaaccaaguggcggugcuguaucaagaugugaacuguacugaagugccgguggcuauacaugccgac
caacucacaccaacauggagaguauauagcacggguuccaauguguuucaaacuagggcuggcuguuugauuggcgcug
aacauguuaauaauuccuaugaaugcgauauucccaucggugccgggauuugcgcaaguuaucaaacgcaaacuaacucc
cccgggucagcauccucugucgcuucccaaucaaucaucgccuauaccaugagucuuggggcagaaaauuccguugcuu
auucuaacaauuccauugcaauuccuacgaacuucaccaucucaguuacuacagaaauacuucccgugucaaugacgaag
acauccguagauugcacaauguauauauguggggacucaacugaaugcucaaaccugcuccugcaauacggaucauuuu
gcacccaacugaacagagcauugaccgguauagccguggagcaagauaagaacacucaagaaguauucgcccaggucaaa
caaaucuauaaaacuccgccuauaaaagauuuuggcggcuuuaacuuuucccaaauacugccugacccaaguaagcccuc
aaaacguagcuuuauagaggaccucuuguuuaauaaggugacacucgcugacgcuggauucauuaagcaauauggugac
ugcuugggagauauugccgcccgcgaucucauuugugcacaaaaguucaacggccucacaguccugcccccucugcuga
cggaugaaaugaucgcucaauacaccucagcucuccuggcaggcaccauaacaagcggguggacauuuggugccggggc
agcacugcaaaucccauucgcaaugcaaauggcuuauagguucaaugggaucggcguaacucaaaauguccucuacgaga
accagaaacucauagcuaaccaauucaauucugcaaucgggaaaauccaggacucccugagcucaacggccagcgcacug
ggcaagcuccaagauguggucaaccaaaacgcacaagcacugaauacucuugugaaacaacucagcuccaauuucggggc
aauaucaaguguccucaaugauauucuuagcaggcuugauccacccgaagccgaggugcagaucgacaggcucauaacag
gcaggcuccagucccuucaaacguauguaacucagcaacugauucgggcugccgagauucgagcuucagcuaauuuggc
agcuacgaagaugagcgaaugcguccugggacagucuaaaagaguagacuuuugcggcaaaggguaucaucugaugagc
uucccacaaagugcuccacauggcgugguuuuccugcaugucacuuauguucccgcacaagagaagaacuucacuaccgc
accagcgaucugucacgaugguaaagcacauuucccgcgggaaggcguauucguaucuaacggcacccacugguucguu
acucaacgcaacuuuuaugaaccacaaaucauuacaaccgauaacacuuuuguuucaggcaauugcgauguugucaucgg
cauugugaauaacacuguguacgauccacuucaaccagaauuggacagcuuuaaagaggagcuugauaaguauuucaag
aaucauaccucucccgacguggaccucggggacaucucuggaauaaaugcuagcgucguuaauauacagaaagagauuga
ucgucugaacgaaguggcuaagaaucugaaugaaagccuuaucgaucugcaagaacuggggaaguacgaacagggauac
auaccggaagccccacgcgacggucaggcuuauguuaggaaggauggagaauggguuuugcucuccacguuucucgggc
uugaaguuuuguuccaaggaccuggucacacccccaauuugagaaacaccaucaccaccaucaccaccacugauaa
(SEQ ID NO: 96)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYL
YRLFRKSNLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH

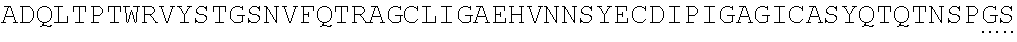

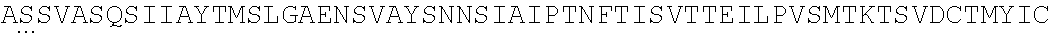

GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS

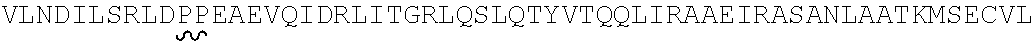

GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQGYIPEAPRDGQAYVRKDGEWVLLSTFLGLEVLFQGPWSHPQFEKHHHHHHHH**
(SEQ ID NO: 97)
WuS_IgE_DownDS2_2P_pVax
gccaccatggactggacctggatactctttctcgtagcagcagccacacgagtgcattcatcacagtgcgttaatctgaccacccgtac
acaactcccacccgcatacacaaatagctttacacgcggagtgtattaccccgataaagtctttcggagctcagtgctccattctactcaa
gatcttttcctgccgttctttagtaacgttacttggtttcatgcaatacatgtgtctggcacaaacggaaccaaacgttttgataatccggtgtt
gccatttaatgatggtgtatattttgcttccacggaaaagtcaaacatcatccgtgggtggatctttggcaccactcttgatagcaaatgtca
aagccttctgattgttaataacgctacaaacgtcgtaattaaagtgtgtgaattccagttctgtaatgaccccttcctcggagtatattacca
caagaataacaaatcttggatggagagcgaatttagagtttacagttcagccaataactgtacatttgaatatgtcagtcagcctttcctcat
ggacctcgaaggtaaacaaggtaattttaagaacttgagagagttcgtgtttaagaacatcgatggctatttcaaaatttactctaagcaca
caccaatcaacctggttcgagacctgccccagggtttctcagctttggaaccattggtggacctgccaatcggcattaacattaccagat
ttcaaactttgttggcactccaccggtcatatcttacccccggagacagttcctcaggctggacggcaggcgccgccgcgtactatgttg
ggtatctccaaccccgaaccttccttctcaaatacaatgaaaacgggacgattacagatgcagtcgattgcgccctggaccccttgtcc
gaaactaaatgcactctgaagagtttcacggtagagaagggaatctatcaaacgagcaattttcgagtccaaccaacggaatctattgtg
cggtttcccaatatcacaaacctctgtccattcggagaagtctttaatgctaccaggtttgcgtctgtatatgcatggaaccgaaagagga
tttccaattgcgtagcggactacagtgtcctttataacagcgcttcattttccacgtttaagtgttatggtgtttctccaacgaaactcaacga
cctctgttttactaacgtttacgctgacagctttgttatacgtggggacgaagtcaggcaaattgctcctggacagactggaaagatcgct
gattataattataaacttcctgacgatttcaccggctgcgttattgcatggaactccaacaatctggattcaaaagtgggtggaaattataat
tatctgtataggttgtttcggaagagcaatcttaagccctttgagcgggacatatgtaccgaaatttaccaagcaggctccaccccatgca
atggagtagaagggttcaattgctattttcctctgcaaagttatggctttcaacccaccaacggagttgggtatcaaccttacagggttgtc
gtgctgagtttcgaattgctccacgcacccgctacagtatgtggccccaagaagtccactaatcttgttaagaataaatgcgtgaacttca
acttcaatggacttacaggtactggagtactcacggaatcaaacaagaaatttctcccatttcaacagtttggccgagatatagctgacac
cacagatgctgttcgcgacccccagacgttggaaatacttgatatcactccctgcagcttcggcggcgtgagcgtgatcactccaggta
ctaatacgagcaatcaagttgccgttctgtaccaagatgtgaactgcaccgaggttccagtggcaattcacgccgaccaacttactccc
acctggcgggtctattccaccggatcaaacgtcttccaaactcgcgctggttgccttatcggtgcagagcacgttaataattcctatgaat
gtgacattcccataggagcaggcatctgtgcatcttatcaaacccagactaattcccctggttccgcttcctctgttgcatcccagtccata
attgcctacactatgagtctcggggctgaaaattccgtggcctattctaataattcaatcgccatcccaaccaattttaccatatccgtaacg
actgaaatacttcctgtcagtatgaccaagacctcagtggactgcaccatgtacatctgcggcgattctactgaatgttccaatctgctttt
gcaatatggttcattctgcacccaactcaacagggctcttacagggatcgccgtcgaacaggataagaatacccaggaagtgttcgcc
caagttaagcaaatttacaagacaccacccatcaaggacttcgggggttcaacttcagccaaattctgcccgacccgtctaagccttct
aagcgctctttcattgaggatcttttgttcaataaggttacgcttgccgatgcagggtttatcaaacagtatggcgactgtcttggggatatc
gcagctagggatcttatttgtgcacagaaatttaatggcctgactgttcttccccctttgctcactgacgagatgattgcccagtacacttca
gctctcctggccgggactataacttctggttggaccttcggagctggcgccgccctgcaaattccatttgcaatgcagatggcttatcgct
tcaacggaattggggtgacccaaaatgttctctacgagaaccagaaactcattgcaaaccagttcaattctgcgatcgggaagatccag
gattccctgtctagtacggctagtgccctcggtaagctccaagacgtcgtcaaccaaaacgcccaggccttgaacacccttgtcaaaca
actgagctccaattttggggctattagcagtgtgctgaatgatatcctgtcccgccttgacccaccggaagcggaagtccaaattgatcg
actgatcactgggcgtctccaatcccttcaaacttacgtgacccaacaactcatccgagcagctgagattagggctagcgctaaccttg
ctgctactaagatgtcagagtgtgtcctcggccagtctaagagagtggacttttgtgggaaagggtaccacttgatgtcattcccacaaa
gcgccccacacggcgtggtgtttctccacgtcacttacgttccagctcaggaaaagaactttaccaccgcccccgctatatgtcatgatg
ggaaggcccactttcctcgtgaaggtgtctttgtcagcaatggcacacactggtttgtgacccaacggaatttctatgagcctcagattatt
accacggataacactttcgtatcagggaattgtgatgtggttatcggcatcgttaataatacagtgtatgacccactgcagccagagcttg
acagcttcaaagaagagctcgataagtactttaagaatcatacaagtcctgacgttgatcttggggatattagtgggattaacgccagcg
tcgtcaatattcagaaagagattgacaggttgaacgaagtagctaagaatcttaatgaaagcctgatagatttgcaagaacttggtaagta
tgagcaggggtacatacccgaggctcctcgggatgggcaggcctatgtacgcaaagacggtgaatgggtattgctcagcacttttctc
ggctgataa (SEQ ID NO: 98)
gccaccauggacuggaccuggauacucuuucucguagcagcagccacacgagugcauucaucacagugcguuaaucugac
cacccguacacaacucccacccgcauacacaaauagcuuuacacgcggaguguauuaccccgauaaagucuuucggagcu
cagugcuccauucuacucaagaucuuuuccugccguucuuuaguaacguuacuugguuucaugcaauacaugugucugg
cacaaacggaaccaaacguuuugauaauccgguguugccauuuaaugaugguguauauuuugcuuccacggaaaaguca
aacaucauccguggguggaucuuuggcaccacucuugauagcaaaugucaaagccuucugauuguuaauaacgcuacaa
acgucguaauuaaagugugugaauuccaguucuguaaugaccccuuccucggaguauauuaccacaagaauaacaaaucu
uggauggagagcgaauuuagaguuuacaguucagccaauaacuguacauuugaauaugucagucagccuuuccucaugg
accucgaagguaaacaagguaauuuuaagaacuugagagaguucguguuuaagaacaucgauggcuauuucaaaauuua
cucuaagcacacaccaaucaaccugguucgagaccugccccaggguuucucagcuuuggaaccauugguggaccugccaa
ucggcauuaacauuaccagauuucaaacuuuguuggcacuccaccggucauaucuuacccccggagacaguuccucaggc
uggacggcaggcgccgcgcguacuauguuggguaucuccaaccccgaaccuuccuucucaaauacaaugaaaacgggac
gauuacagaugcagucgauugcgcccuggaccccuuguccgaaacuaaaugcacucugaagaguuucacgguagagaag
ggaaucuaucaaacgagcaauuuucgaguccaaccaacggaaucuauugugcgguuucccaauaucacaaaccucugucc
auucggagaagucuuuaaugcuaccagguuugcgucuguauaugcauggaaccgaaagaggauuuccaauugcguagcg
gacuacaguguccuuuauaacagcgcuucauuuuccacguuuaaguguuaugguguuucuccaacgaaacucaacgacc
ucuguuuuacuaacguuuacgcugacagcuuuguuauacguggggacgaagucaggcaaauugcuccuggacagacugg
aaagaucgcugauuauaauuauaaacuuccugacgauuucaccggcugcguuauugcauggaacuccaacaaucuggau
ucaaaaguggguggaaauuauaauuaucuguauagguuguuucggaagagcaaucuuaagcccuuugagcgggacauau
guaccgaaauuuaccaagcaggcuccaccccaugcaauggaguagaaggguucaauugcuauuuuccucugcaaaguua
uggcuuucaacccaccaacggaguuggguaucaaccuuacaggguugucgugcugaguuucgaauugcuccacgcaccc
gcuacaguauguggccccaagaaguccacuaaucuuguuaagaauaaaugcgugaacuucaacuucaauggacuuacagg
uacuggaguacucacggaaucaaacaagaaauuucucccauuucaacaguuuggccgagauauagcugacaccacagaug
cuguucgcgacccccagacguuggaaauacuugauaucacucccugcagcuucggcggcgugagcgugaucacuccagg
uacuaauacgagcaaucaaguugccguucuguaccaagaugugaacugcaccgagguuccaguggcaauucacgccgacc
aacuuacucccaccugggggucuauuccaccggaucaaacgucuuccaaacucgcgcugguugccuuaucggugcaga
gcacguuaauaauuccuaugaaugugacauucccauaggagcaggcaucugugcaucuuaucaaacccagacuaauuccc
cugguuccgcuuccucuguugcaucccaguccauaauugccuacacuaugagucucggggcugaaaauuccguggccua
uucuaauaauucaaucgccaucccaaccaauuuuaccauauccguaacgacugaaauacuuccugucaguaugaccaaga
ccucaguggacugcaccauguacaucugcggcgauucuacugaauguuccaaucugcuuuugcaauaugguucauucg
cacccaacucaacagggcucuuacagggaucgccgucgaacaggauaagaauacccaggaaguguucgcccaaguuaagc
aaauuuacaagacaccacccaucaaggacuucggggguucaacuucagccaaauucugcccgacccgucuaagccuucu
aagcgcucuuucauugaggaucuuuuguucaauaagguuacgcuugccgaugcaggguuuaucaaacaguauggcgacu
gucuuggggauaucgcagcuagggaucuuauuugugcacagaaauuuaauggccugacuguucuucccccuuugcucac
ugacgagaugauugcccaguacacuucagcucuccuggccgggacuauaacuucugguuggaccuucggagcuggcgcc
gcccugcaaauuccauuugcaaugcagauggcuuaucgcuucaacggaauuggggugacccaaaauguucucuacgaga
accagaaacucauugcaaaccaguucaauucugcgaucgggaagauccaggauucccugucuaguacggcuagugcccuc
gguaagcuccaagacgucgucaaccaaaacgcccaggccuugaacacccuugucaaacaacugagcuccaauuuuggggc
uauuagcagugugcugaaugauauccugucccgccuugacccaccggaagcggaaguccaaauugaucgacugaucacu
gggcgucuccaaucccuucaaacuuacgugacccaacaacucauccgagcagcugagauuagggcuagcgcuaaccuugc
ugcuacuaagaugucagaguguguccucggccagucuaagagaguggacuuuugugggaaaggguaccacuugauguc
auucccacaaagcgccccacacggcgugguguuucuccacgucacuuacguuccagcucaggaaaagaacuuuaccaccg
cccccgcuauaugucaugaugggaaggcccacuuuccucgugaaggugucuuugucagcaauggcacacacugguuugu
gacccaacggaauuucuaugagccucagauuauuaccacggauaacacuuucguaucagggaauugugaugugguuauc
ggcaucguuaauaauacaguguaugacccacugcagccagagcuugacagcuucaaagaagagcucgauaaguacuuuaa
gaaucauacaaguccugacguugaucuuggggauauuagugggauuaacgccagcgucgucaauauucagaaagagauu
gacagguugaacgaaguagcuaagaaucuuaaugaaagccugauagauuugcaagaacuugguaaguaugagcaggggu
acauacccgaggcuccucgggaugggcaggccuauguacgcaaagacggugaauggguauugcucagcacuuuucucgg
cugauaa (SEQ ID NO: 99)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT

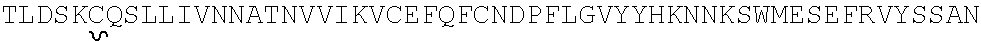

NCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYL

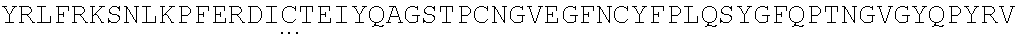

VVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS

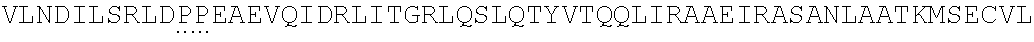

GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQGYIPEAPRDGQAYVRKDGEWVLLSTFLG** (SEQ ID NO: 100)
WuS_IgE_DownDS1_2P_pVax
gccaccatggactggacctggatactctttctcgtagcagcagccacacgagtgcattcatcccagtgcgtgaacctgaccacccgaa
ctcaactcccaccagcatacaccaactcatttacaagaggagtttattacccggacaaggtatttcgaagttcagttcttcacagcaccca
agacctgtttctgccattcttcagtaatgtcacttggtttcacgcgatacatgtcagcggtacaaacgggacaaagcgattcgataaccca
gtactcccattcaacgacggagtgtattttgcatctacagagaaatccaacattatacgcgggtggatctttggaactactctggactcca
agacacagagcctgctcattgtgaacaatgcaacgaatgtcgtcataaaagtctgtgaatttcaattttgcaacgatcctttcctcggagtc
tattaccataagaacaataagagttggatggagagtgagtttcgcgtctattcttccgcgaacaattgtacatttgaatatgtatcacaacc
ctttcttatggatttggaaggcaaacaaggtaacttcaagaacttgcgcgagttcgtgttcaagaacatagactgttattttaagatctatag
taagcatacgccaatcaatctggtgcgagatttgcctcagggcttttctgctcttgaacccttggttgatctgcccatcgggatcaacataa
ccagatttcaaacgttgctcgcactccaccgcagctatctcactcctggcgattcctcatctgggtggaccgccggagctgctgcttatta
cgtcggctatctccagccgcgtactttcctgctcaagtataatgagaatggcaccattaccgatgctgtggattgtgctcttgatccactct
ctgaaaccaaatgcactctcaagtcttttaccgtggaaaagggtatttatcagacatctaattttcgggtgcaacctactgagtcaattgtac
ggtttcctaacataactaacctttgtccatttggggaagtcttcaatgccacgcggttcgcatcagtctatgcatggaacagaaaacgtatc
tccaactgcgtcgccgattattccgtcctttacaatagcgctagcttttccacattcaaatgttatggcgtatcaccaaccaaacttaacgat
ctctgctttactaatgtctacgctgactctttcgttattcgaggtgacgaggtgcgccaaattgcgcctggtcaaaccggaaagattgccg
attataactacaagctccccgacgactttacgggttgtgtgatcgcctggaatagcaataacctcgattctaaagttggcggtaattataac
tatctgtacagactctttaggaaaagtaatctcaagcccttttgcagggatatctcaaccgaaatctaccaagccggcagcactccttgca
atggtgtcgaggggtttaattgttatttcccactgcaatcttacggctttcaaccgactaatggagtcggttatcaaccctatagggtggtg
gtactctcctttgaacttttgcacgctccggcaacagtttgtggaccaaagaaaagtacgaaccttgttaagaataagtgtgttaatttcaat
tttaacggcctcactggaacaggtgtcctcacagaaagcaacaagaagtttctccctttccaacagtttggacgggatatcgccgacact
actgacgccgtcagagatcctcaaactctcgaaatcttggatatcacaccatgttctttcggtggtgtctccgtcataacaccaggaacta
acacctctaatcaagtggccgtgctctatcaggacgtcaattgcacagaagtgcctgtcgcaatccatgctgatcagctcactcccacct
ggcgtgtgtattccactggctctaatgtctttcagacacgggcaggttgccttattggggcagagcatgtgaacaattcctacgaatgcg
atatacccattggggcaggcatttgcgccagctaccaaacccaaactaacagccccgggagtgccagcagcgtggcatctcagtcca
ttattgcctatacgatgagcctgggtgctgaaaatagcgtggcttatagtaataactctatcgccatacccacaaacttcaccatttcagtg
accaccgaaatccttcctgtttctatgaccaaaacgtccgtcgattgtacaatgtacatttgcggcgatagcactgaatgttcaaacctgct
cctgcaatacggctctttctgtactcagctcaaccgggcactcaccggcatagccgtcgaacaagacaagaatacccaggaagtctttg
cgcaggtgaaacaaatctataagaccccaccaataaaagatttcggcggttttaatttcagccaaatcttgcctgatcccagcaagccat
ctaaacggtctttcattgaagatctcctgttcaacaaggttacgctggctgacgccgggtttattaagcaatatggcgattgccttgggga
cattgccgcacgagacctcatttgtgcccagaaattcaacgggctcaccgtattgcccccgctcctcacagacgaaatgatcgcccaat
atacaagcgccctgcttgcgggaaccattacaagcggttggacctttggtgccggcgcagctctgcaaatacccttcgcaatgcaaat
ggcatatcggtttaatggaattggcgtaacccaaaacgtgctgtatgaaaaccagaaactgatcgcaaatcaattcaatagtgctatagg
aaagatccaagacagtctgtcttccactgctagcgcgctggggaagctccaagacgttgtgaaccaaaacgcgcaggccctgaatac
cctggtgaagcaactttcaagcaatttcggtgctatatcttctgtcctcaatgacattctctctcggctcgatcccccggaagccgaagttc
agatagaccgtttgatcacaggccgcttgcaatccctgcaaacctacgttacacaacaactgattcgcgccgccgaaattcgggcatcc
gccaatctggccgcaaccaaaatgtccgagtgtgttctcggtcaatccaaacgcgtggatttctgcggaaaaggataccatttgatgtca
tttccacaatcagctccacacggtgttgtattcctgcacgtgacctacgtgccagcccaggagaagaattttactactgcgcccgccattt
gtcatgacgggaaggctcattttcctcgggaaggggttttcgtctcaaacggtacccattggttcgtgactcagaggaacttttatgaacc
tcaaatcataacgaccgataacacgtttgtaagtggcaattgcgacgtggtcatcgggattgtaaacaatactgtctatgaccctctccaa
ccagagcttgacagctttaaagaagagcttgataaatactttaagaaccatacctcaccagacgtcgatttgggagatatcagtggcatt
aatgcctctgtcgtcaatatccagaaagagattgaccgcttgaacgaagttgccaagaatcttaatgagtctctgattgacttgcaagaatt
gggaaaatatgaacaaggatatattccagaagcccctcgcgatgggcaagcatatgttcgaaaggatggggaatgggtgctgctcag
cacctttctcggttgataa (SEQ ID NO: 101)
gccaccauggacuggaccuggauacucuuucucguagcagcagccacacgagugcauucaucccagugcgugaaccugac
cacccgaacucaacucccaccagcauacaccaacucauuuacaagaggaguuuauuacccggacaagguauuucgaaguu
caguucuucacagcacccaagaccuguuucugccauucuucaguaaugucacuugguuucacgcgauacaugucagcgg
uacaaacgggacaaagcgauucgauaacccaguacucccauucaacgacggaguguauuuugcaucuacagagaaaucca
acauuauacgcggguggaucuuuggaacuacucuggacuccaagacacagagccugcucauugugaacaaugcaacgaau
gucgucauaaaagucugugaauuucaauuuugcaacgauccuuuccucggagucuauuaccauaagaacaauaagaguu
ggauggagagugaguuucgcgucuauucuuccgcgaacaauuguacauuugaauauguaucacaacccuuucuuaugga
uuuggaaggcaaacaagguaacuucaagaacuugcgcgaguucguguucaagaacauagacuguuauuuuaagaucuau
aguaagcauacgccaaucaaucuggugcgagauuugccucagggcuuuucugcucuugaacccuugguugaucugccca
ucgggaucaacauaaccagauuucaaacguugcucgcacuccaccgcagcuaucucacuccuggcgauuccucaucuggg
uggaccgccggagcugcugcuuauuacgucggcuaucuccagccgcguacuuuccugcucaaguauaaugagaauggca
ccauuaccgaugcuguggauugugcucuugauccacucucugaaaccaaaugcacucucaagucuuuuaccguggaaaa
ggguauuuaucagacaucuaauuuucgggugcaaccuacugagucaauuguacgguuuccuaacauaacuaaccuuugu
ccauuuggggaagucuucaaugccacgcgguucgcaucagucuaugcauggaacagaaaacguaucuccaacugcgucg
ggguauuuaucagacaucuaauuuucgggugcaaccuacugagucaauuguacgguuuccuaacauaacuaaccuuugu
ccauuuggggaagucuucaaugccacgcgguucgcaucagucuaugcauggaacagaaaacguaucuccaacugcgucg
ccgauuauuccguccuuuacaauagcgcuagcuuuuccacauucaaauguuauggcguaucaccaaccaaacuuaacgau
cucugcuuuacuaaugucuacgcugacucuuucguuauucgaggugacgaggugcgccaaauugcgccuggucaaaccg
gaaagauugccgauuauaacuacaagcuccccgacgacuuuacggguugugugaucgccuggaauagcaauaaccucga
uucuaaaguuggcgguaauuauaacuaucuguacagacucuuuaggaaaaguaaucucaagcccuuuugcagggauauc
ucaaccgaaaucuaccaagccggcagcacuccuugcaauggugucgagggguuuaauuguuauuucccacugcaaucuu
acggcuuucaaccgacuaauggagucgguuaucaacccuauaggguggugguacucuccuuugaacuuuugcacgcucc
ggcaacaguuuguggaccaaagaaaaguacgaaccuuguuaagaauaaguguguuaauuucaauuuuaacggccucacu
ggaacagguguccucacagaaagcaacaagaaguuucucccuuuccaacaguuuggacgggauaucgccgacacuacuga
cgccgucagagauccucaaacucucgaaaucuuggauaucacaccauguucuuucgguggugucuccgucauaacaccag
gaacuaacaccucuaaucaaguggccgugcucuaucaggacgucaauugcacagaagugccugucgcaauccaugcugau
cagcucacucccaccuggcguguguauuccacuggcucuaaugucuuucagacacgggcagguugccuuauuggggcag
agcaugugaacaauuccuacgaaugcgauauacccauuggggcaggcauuugcgccagcuaccaaacccaaacuaacagc
cccgggagugccagcagcguggcaucucaguccauuauugccuauacgaugagccugggugcugaaaauagcguggcuu
auaguaauaacucuaucgccauacccacaaacuucaccauuucagugaccaccgaaauccuuccuguuucuaugaccaaaa
cguccgucgauuguacaauguacauuugcggcgauagcacugaauguucaaaccugcuccugcaauacggcucuuucug
uacucagcucaaccgggcacucaccggcauagccgucgaacaagacaagaauacccaggaagucuuugcgcaggugaaac
aaaucuauaagaccccaccaauaaaagauuucggcgguuuuaauuucagccaaaucuugccugaucccagcaagccaucu
aaacggucuuucauugaagaucuccuguucaacaagguuacgcuggcugacgccggguuuauuaagcaauauggcgauu
gccuuggggacauugccgcacgagaccucauuugugcccagaaauucaacgggcucaccguauugcccccgcuccucaca
gacgaaaugaucgcccaauauacaagcgcccugcuugcgggaaccauuacaagcgguuggaccuuuggugccggcgcag
cucugcaaauacccuucgcaaugcaaauggcauaucgguuuaauggaauuggcguaacccaaaacgugcuguaugaaaac
cagaaacugaucgcaaaucaauucaauagugcuauaggaaagauccaagacagucugucuuccacugcuagcgcgcuggg
gaagcuccaagacguugugaaccaaaacgcgcaggcccugaauacccuggugaagcaacuuucaagcaauuucggugcua
uaucuucuguccucaaugacauucucucucggcucgaucccccggaagccgaaguucagauagaccguuugaucacagg
ccgcuugcaaucccugcaaaccuacguuacacaacaacugauucgcgccgccgaaauucgggcauccgccaaucuggccg
caaccaaaauguccgaguguguucucggucaauccaaacgcguggauuucugcggaaaaggauaccauuugaugucauu
uccacaaucagcuccacacgguguuguauuccugcacgugaccuacgugccagcccaggagaagaauuuuacuacugcgc
ccgccauuugucaugacgggaaggcucauuuuccucgggaagggguuuucgucucaaacgguacccauugguucgugac
ucagaggaacuuuuaugaaccucaaaucauaacgaccgauaacacguuuguaaguggcaauugcgacguggucaucggg
auuguaaacaauacugucuaugacccucuccaaccagagcuugacagcuuuaaagaagagcuugauaaauacuuuaagaa
ccauaccucaccagacgucgauuugggagauaucaguggcauuaaugccucugucgucaauauccagaaagagauugacc
gcuugaacgaaguugccaagaaucuuaaugagucucugauugacuugcaagaauugggaaaauaugaacaaggauauau
uccagaagccccucgcgaugggcaagcauauguucgaaaggauggggaaugggugcugcucagcaccuuucucgguuga
uaa (SEQ ID NO: 102)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFRVYSSAN

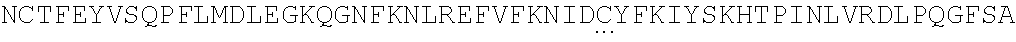

LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYL
YRLFRKSNLKPFCRDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRENGIGVTQNVLY
ENQKLIANQFNSAIGKIQDSLSSTASALGKLQDVVNQNAQALNTLVKQLSSNFGAISS
VLNDILSRLDPPEAEVQIDRLITGRLQSLQTYVTQQLIRAAEIRASANLAATKMSECVL
GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQGYIPEAPRDGQAYVRKDGEWVLLSTFLG** (SEQ ID NO: 103)
WuS_IgE_2P_UpGly_pVax
gccaccatggattggacctggatacttttcctcgtggccgcagcaacaagagtccactcctctcagtgcgttaacctgactactagaacc
caattgcccccggcatacacaaactctttcacccggggtgtctactatcccgacaaagtgtttagaagtagcgtgctgcacagcaccca
agatctctttctgccattcttctcaaacgtcacctggtttcacgccatccatgtaagcgggaccaacggcacaaagcgttttgataaccct
gttttgccattcaatgatggcgtgtattttgcttccactgagaaaagcaacatcattagagggtggatatttggcacaacgcttgactccaa
gacgcagagtcttttgatagtaaacaacgcaactaatgtggtcattaaagtctgtgaatttcaattttgcaatgaccccttccttggagtctat
taccacaagaacaacaaaagctggatggaaagcgaatttagggtctacagctctgccaataactgcacattcgaatacgtcagccaac
cattcttgatggacctggaaggcaagcaaggaaactttaagaatctgagggaatttgtgtttaagaatatcgacggatattttaagatctat
tccaagcatactcccattaatctcgttcgtgaccttcctcagggtttctctgcattggaacccctcgtagatttgcccattgggattaatatca
ctagattccagacgctgcttgcactccatcgatcttatctgacccctggtgactcctcttccgggtggacggcgggtgctgcagcctact
acgttggctatttgcaacctaggacctttctgttgaagtataatgagaatgggactattactgatgccgttgattgcgccctcgatccgctgt
cagaaacaaagtgcaccctgaagagcttcacagtagaaaagggaatctatcaaacctcaaatttccgcgttcaaccaactgaatcaatc
gtgcgttttcctaacatcacaaatctgtgtccgtttggagaagtatttaatgcgacgcgtttcgcaagcgtctacgcgtggaatcgcaaac
gtatctctaattgcgtagcagattattctgtgctgtacaatagcgcatctttctcaacgtttaagtgctacggcgttaatgggaccaagctga
atgatctctgtttcactaatgtgtacgcagacagttttgtaattagaggagacgaggttaggcaaatagcaccgggtcaaactggcaaaa
tcgccgactataactacaagctccctgatgacttcacgggctgcgtaattgcttggaactctaataacctggactctaaagtcggcggga
attataattatctctatcggttgtttcgaaaatccaatctcaaaccctttgagcgggacatcaatactacaatttatcaagctggtagtactcct
tgcaatggggtagaaggcttcaattgttatttcccccttcaatcttacggatttcaacccacgaacggcgtagggtaccagccctatcgag
tggtggtactgtcattcgaacttaatcacgccccagcaacagtctgcgggcctaagaaaagcacgaatcttgtcaagaataagtgtgta
aatttcaacttcaatggtcttacaggcacgggagtgctcactgagtctaataagaaatttcttcctttccaacaattcggtcgtgatattgcc
gatactactgatgcagtccgagatccacaaactctcgaaatcctcgatattactccttgtagttttggcggcgtctccgtgatcaccccag
ggaccaacactagtaaccaagtggcggtgctctaccaagatgttaactgcacagaagtcccggtagcgatccatgccgaccagctca
ctcccacatggcgtgtttacagcacagggtcaaacgttttccagacccgtgccggatgtcttataggagccgaacacgtaaataacagt
tatgaatgcgatatcccaattggtgcaggtatctgtgcgtcatatcaaacccaaactaattctccggggtccgcctcaagcgttgcctcac
aatcaataatcgcctacacaatgtccctcggtgccgaaaattcagtcgcttactctaacaatagcattgctatccctaccaacttcactattt
ctgttaccacggaaattttgcctgtatccatgaccaaaacatctgttgattgcacgatgtacatctgcggggattctaccgaatgttctaac
ctgcttctgcaatacggctccttctgcacccaattgaaccgcgcactgactgggattgctgtggaacaagacaagaatactcaagaagt
atttgcccaggtcaaacagatttacaaaactcccccaattaaagatttcggcggtttcaattttagtcaaattctgccagatccaagtaagc
catccaaacgctcatttattgaggacctgctctttaataaagtcacgctggccgacgccggcttcataaaacagtatggcgattgtcttgg
agacatcgccgcccgcgacctcatttgcgcacaaaagttcaatgggctcaccgtgttgccaccactgctcacagatgagatgatcgca
cagtacacgagcgcccttcttgccggcactatcacgtctggttggacgttcggtgccggagccgctctgcaaattccctttgcaatgcaa
atggcctatagatttaatggaattggcgtaacacagaacgtgttgtacgagaaccagaagctcattgccaaccagttcaattccgctattg
gcaaaatacaagactctctcagctcaactgctagcgcactgggaaaattgcaagacgtagtcaatcaaaatgcccaagccctcaatact
ctcgtcaaacagttgtcttccaactttggggctatcagtagtgtactcaatgacattctttcaagactggacccgcccgaggcggaagtcc
aaattgatcgtctgataactggaaggttgcaaagccttcagacctacgttacgcaacaacttattagggctgccgaaataagggcatcc
gctaatctggcagctacaaagatgtctgaatgtgttttgggacagagcaaacgggttgacttctgcggtaaaggttaccatctcatgtcttt
tccacaaagcgcaccgcacggagtcgtcttcctgcatgtaacatacgtcccagcccaagaaaagaattttaccacagccccagccatc
tgccacgacggcaaggcgcatttcccaagggaaggcgtgtttgtatccaacgggacgcattggtttgtcactcaaaggaacttttacga
accccaaattattaccactgataacaccttcgtttctgggaactgtgatgtcgtgattgggatagtaaacaacacggtatatgatccactgc
aaccagaactggattccttcaaagaagagctggacaaatacttcaagaatcatactagtcctgacgtcgacctgggcgatatcagtgga
atcaacgctagcgtcgtaaacattcaaaaggagatcgatagacttaacgaggtcgccaagaatctcaatgaaagcctcatcgatttgca
agaactcggaaaatatgagcaaagcggatcagggtacattccggaagcccccagggacggacaggcatatgtccgcaaggacgga
gaatgggttcttcttagcacttttctggggtaatga (SEQ ID NO: 104)
gccaccauggauuggaccuggauacuuuuccucguggccgcagcaacaagaguccacuccucucagugcguuaaccugac
uacuagaacccaauugcccccggcauacacaaacucuuucacccggggugucuacuaucccgacaaaguguuuagaagua
gcgugcugcacagcacccaagaucucuuucugccauucuucucaaacgucaccugguuucacgccauccauguaagcggg
accaacggcacaaagcguuuugauaacccuguuuugccauucaaugauggcguguauuuugcuuccacugagaaaagca
acaucauuagaggguggauauuuggcacaacgcuugacuccaagacgcagagucuuuugauaguaaacaacgcaacuaau
guggucauuaaagucugugaauuucaauuuugcaaugaccccuuccuuggagucuauuaccacaagaacaacaaaagcu
ggauggaaagcgaauuuagggucuacagcucugccaauaacugcacauucgaauacgucagccaaccauucuugauggac
cuggaaggcaagcaaggaaacuuuaagaaucugagggaauuuguguuuaagaauaucgacggauauuuuaagaucuauu
ccaagcauacucccauuaaucucguucgugaccuuccucaggguuucucugcauuggaaccccucguagauuugcccau
ugggauuaauaucacuagauuccagacgcugcuugcacuccaucgaucuuaucugaccccuggugacuccucuuccggg
uggacggcgggugcugcagccuacuacguuggcuauuugcaaccuaggaccuuucuguugaaguauaaugagaauggg
acuauuacugaugccguugauugcgcccucgauccgcugucagaaacaaagugcacccugaagagcuucacaguagaaaa
gggaaucuaucaaaccucaaauuuccgcguucaaccaacugaaucaaucgugcguuuuccuaacaucacaaaucuguguc
cguuuggagaaguauuuaaugcgacgcguuucgcaagcgucuacgcguggaaucgcaaacguaucucuaauugcguagc
agauuauucugugcuguacaauagcgcaucuuucucaacguuuaagugcuacggcguuaaugggaccaagcugaaugau
cucuguuucacuaauguguacgcagacaguuuuguaauuagaggagacgagguuaggcaaauagcaccgggucaaacug
gcaaaaucgccgacuauaacuacaagcucccugaugacuucacgggcugcguaauugcuuggaacucuaauaaccuggac
ucuaaagucggcgggaauuauaauuaucucuaucgguuguuucgaaaauccaaucucaaacccuuugagcgggacauca
auacuacaauuuaucaagcugguaguacuccuugcaaugggguagaaggcuucaauuguuauuucccccuucaaucuua
cggauuucaacccacgaacggcguaggguaccagcccuaucgaguggugguacugucauucgaacuuaaucacgccccag
caacagucugcgggccuaagaaaagcacgaaucuugucaagaauaaguguguaaauuucaacuucaauggucuuacaggc
acgggagugcucacugagucuaauaagaaauuucuuccuuuccaacaauucggucgugauauugccgauacuacugaug
caguccgagauccacaaacucucgaaauccucgauauuacuccuuguaguuuuggggcgucuccgugaucaccccagg
gaccaacacuaguaaccaaguggcggugcucuaccaagauguuaacugcacagaagucccgguagcgauccaugccgacc
agcucacucccacauggcguguuuacagcacagggucaaacguuuuccagacccgugccggaugucuuauaggagccga
acacguaaauaacaguuaugaaugcgauaucccaauuggugcagguaucugugcgucauaucaaacccaaacuaauucuc
cgggguccgccucaagcguugccucacaaucaauaaucgccuacacaaugucccucggugccgaaaauucagucgcuuac
ucuaacaauagcauugcuaucccuaccaacuucacuauuucuguuaccacggaaauuuugccuguauccaugaccaaaac
aucuguugauugcacgauguacaucugcggggauucuaccgaauguucuaaccugcuucugcaauacggcuccuucugc
acccaauugaaccgcgcacugacugggauugcuguggaacaagacaagaauacucaagaaguauuugcccaggucaaaca
gauuuacaaaacucccccaauuaaagauuucggcgguuucaauuuuagucaaauucugccagauccaaguaagccaucca
aacgcucauuuauugaggaccugcucuuuaauaaagucacgcuggccgacgccggcuucauaaaacaguauggcgauug
ucuuggagacaucgccgcccgcgaccucauuugcgcacaaaaguucaaugggcucaccguguugccaccacugcucacag
augagaugaucgcacaguacacgagcgcccuucuugccggcacuaucacgucugguuggacguucggugccggagccgc
ucugcaaauucccuuugcaaugcaaauggccuauagauuuaauggaauuggcguaacacagaacguguuguacgagaac
cagaagcucauugccaaccaguucaauuccgcuauuggcaaaauacaagacucucucagcucaacugcuagcgcacuggg
aaaauugcaagacguagucaaucaaaaugcccaagcccucaauacucucgucaaacaguugucuuccaacuuuggggcua
ucaguaguguacucaaugacauucuuucaagacuggaccccccgaggcggaaguccaaauugaucgucugauaacugg
aagguugcaaagccuucagaccuacguuacgcaacaacuuauuagggcugccgaaauaagggcauccgcuaaucuggcag
cuacaaagaugucugaauguguuuugggacagagcaaacggguugacuucugcgguaaagguuaccaucucaugucuuu
uccacaaagcgcaccgcacggagucgucuuccugcauguaacauacgucccagcccaagaaaagaauuuuaccacagcccc
agccaucugccacgacggcaaggcgcauuucccaagggaaggcguguuuguauccaacgggacgcauugguuugucacu
caaaggaacuuuuacgaaccccaaauuauuaccacugauaacaccuucguuucugggaacugugaugucgugauuggga
uaguaaacaacacgguauaugauccacugcaaccagaacuggauuccuucaaagaagagcuggacaaauacuucaagaau
cauacuaguccugacgucgaccugggcgauaucaguggaaucaacgcuagcgucguaaacauucaaaaggagaucgauag
acuuaacgaggucgccaagaaucucaaugaaagccucaucgauuugcaagaacucggaaaauaugagcaaagcggaucag
gguacauuccggaagcccccagggacggacaggcauauguccgcaaggacggagaauggguucuucuuagcacuuuucu
gggguaauga (SEQ ID NO: 105)
MDWTWILFLVAAATRVHSSQCVNLTTRTQLPPAYTNSFTRGVYYPDKVFRSSVLHS
TQDLFLPFFSNVTWFHAIHVSGTNGTKRFDNPVLPFNDGVYFASTEKSNIIRGWIFGT
TLDSKTQSLLIVNNATNVVIKVCEFQFCNDPFLGVYYHKNNKSWMESEFRVYSSAN
NCTFEYVSQPFLMDLEGKQGNFKNLREFVFKNIDGYFKIYSKHTPINLVRDLPQGFSA
LEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGE

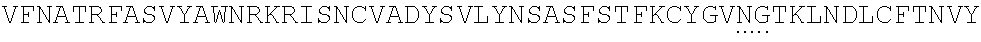

ADSFVIRGDEVRQIAPGQTGKIADYNYKLPDDFTGCVIAWNSNNLDSKVGGNYNYL

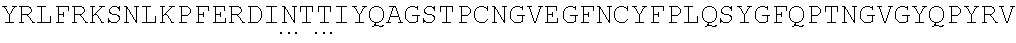

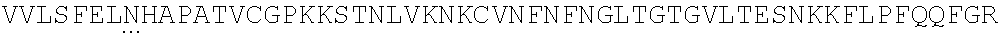

DIADTTDAVRDPQTLEILDITPCSFGGVSVITPGTNTSNQVAVLYQDVNCTEVPVAIH
ADQLTPTWRVYSTGSNVFQTRAGCLIGAEHVNNSYECDIPIGAGICASYQTQTNSPGS
ASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISS

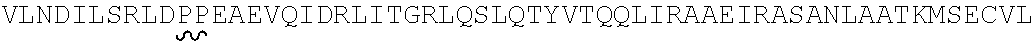

GQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSF
KEELDKYFKNHTSPDVDLGDISGINASVVNIQKEIDRLNEVAKNLNESLIDLQELGKY
EQSGSGYIPEAPRDGQAYVRKDGEWVLLSTFLG** (SEQ ID NO: 106)
WuS_DownDS3_D2P_Furin_pVax
gccaccatggactggacctggatactctttctcgtagcagcagccacacgagtgcattcaatgttcgttttcctcgtgctcttgcctttggtt
tcttctcagtgcgtaaacctcacgactcgaacccaactgcccccagcttatacaaattcctttacgcggggcgtctattacccggataag
gttttcagatccagcgtgctgcatagtacacaagatctctttcttcctttcttctcaaatgtaacctggtttcacgctattcatgtatccggcac
caatggaactaaaagatttgataacccggtgttgcccttcaatgatggtgtgtatttcgcttccacggaaaagtcaaacatcatcagaggg
tggatattcggcacaacattggattccaagtgccagtcactcctcatagtgaacaatgctactaacgtggttataaaggtctgcgaatttca
attttgtaatgatcctttcctcggtgtttactatcacaagaacaataagtcctggatggaatcagaattccgtgtatacagttctgcgaacaat
tgcacattcgaatatgtgtcccaaccctttctcatggatctggaagggaagcagggtaactttaagaatctgagagaattcgtgttcaaga
acattgactgctattttaaaatctatagcaaacacacccctataaacttggtacgggatttgcctcaaggattctcagcactcgaacccttg
gtcgatttgccaatcggcatcaatatcacccggtttcagacactcctggctcttcaccgctcctacttgacacctggtgattcctcatctggt
tggaccgccggcgcagcggcatactatgtcggctatcttcaaccaagaaccttcttgctgaaatataatgagaacggaactataactgat
gccgttgattgtgcccttgatccacttagcgaaacaaagtgcactctgaagtccttcacagttgaaaaggggatctaccaaacatccaac
ttccgggtacaacctactgagtccatagtgcgatttcctaacattaccaatctgtgcccatttggagaagtattcaacgcaactaggttcgc
gtccgtttacgcgtggaacaggaaaaggatttccaattgcgtcgccgactatagcgttctctataacagcgcctcatttagcacgtttaag
tgttacggggttagtccgaccaaactcaatgacttgtgttttaccaatgtctatgcagactcctttgttattagaggcgacgaggtcagaca
aattgcccccggacagacaggtaagattgcagattataattataaactgccggacgacttcacggggtgtgttattgcatggaactccaa
taacctggactctaaagtaggcgggaactataactatctgtatcgcctgtttcgcaaatctaacctgaaacccttctgcagggacatatgt
actgaaatatatcaagctggcagcacaccttgtaatggcgtcgagggattcaattgttacttcccacttcaatcttacggttttcagcctact
aacggcgtagggtatcaaccctatagagttgtagtgctctctttcgaattgctccatgcccccgcgactgtttgtggacctaagaagtcca
cgaacctggtaaagaacaagtgtgttaattttaattttaatggactgaccgggactggagtgctgactgaaagtaacaagaaatttctgcc
tttccaacaatttggccgcgatatcgctgataccaccgacgccgtcagagatccgcagactctcgaaatcctggacatcacgccctgct
cattcggcggggttagcgttattactccaggcactaacactagcaatcaagttgcagttctgtaccaggatgtgaactgtaccgaagtcc
ccgtcgccattcatgccgatcagctgaccccgacttggcgggtatattcaaccggcagcaatgtctttcaaacaagggcgggttgtctc
atcggagcggagcatgtaaataatagttatgaatgcgacatccccattggcgcggggatctgtgcttcatatcaaactcaaaccaattcc
ccacggcggagacgatcagtagccagtcaatcaataattgcgtatacgatgagtcttggggcagaaaatagcgtggcttattctaataat
agcatcgctatacctacaaattttacaatcagtgtaactaccgaaatccttcctgtcagcatgaccaaaactagcgtagattgcacgatgt
atatttgcggagactcaactgagtgcagtaacctgttgttgcaatacggaagtttctgtacccagctgaaccgcgctcttacgggcattgc
agtagaacaagataagaatacccaagaagtgtttgcccaggtgaaacaaatctacaagactcccccgattaaagactttggcgggttca
acttcagccagatattgcccgacccgtctcgtcgtagacggtcctttattgaagacctgctcttcaacaaggtcacactggctgatgcag
gttttattaagcaatacggcgactgtcttggcgacatcgccgctagggaccttatatgtgctcagaaattcaatggtctgacagttctgcca
cccttgctcactgacgaaatgatcgctcaatatacaagcgccttgctggctgggactattacttccggatggacattcggggcgggtgc
cgccttgcaaattccttttgcaatgcaaatggcataccgtttcaacggaatcggcgtaacccagaatgtgctctatgaaaaccagaaattg
atagcaaatcaatttaactcagccataggaaagattcaagactctctcagctcaaccgcgagtgctctcggcaagctccaagacgtagt
aaatcaaaatgcacaagctttgaacactttggtaaagcaattgtcttccaacttcggggcgatctcatctggccctaacgacatcctgtcc
cggttgcccaaagtggaagccgaggtgcagatcgaccgcctcatcaccggccgacttcaatcactccaaacctacgtgactcaacaa
ctgatccgggcagccgagataagggcgagtgcaaacttggcagctacgaaaatgtcagaatgtgttctcggccagagtaaacgggta
gacttttgtgggaaaggttatcacttgatgtctttccctcaaagcgctcctcacggcgtcgtcttcttgcatgtgacttacgtgccagctcaa
gaaaagaacttcaccaccgcccctgctatatgccatgacggtaaagctcacttcccccgagagggcgtgttcgttagtaatggaaccc
attggtttgtgactcaacgaaacttttatgaacctcaaataattaccacggataacacttttgttagtggtaattgtgacgtggtgatcggcat
tgtgaataacacagtctacgatcctctgcaaccagaactggacagctttaaagaggaacttgacaaatatttcaagaaccatacaagcc
ccgacgtcgacctgggcgacatcagtggaatcaatgcgtccgtagtcaatatccagaaggagattgatcggcttaatgaagtcgctaa
gaatttgaatgaaagtcttatagatctgcaagaactcgggaagtacgagcaatatattaaatggccttggtccggacgtagaaggcgca
ggcggggctcaggcggttcagggtcagggtatattcccgaggcgccacgcgatgggcaagcgtacgtgcgtaaagatggcgaatg
ggtgttgctttccacattcttggggtgataa (SEQ ID NO: 107)
gccaccauggacuggaccuggauacucuuucucguagcagcagccacacgagugcauucaauguucguuuuccucgugc
ucuugccuuugguuucuucucagugcguaaaccucacgacucgaacccaacugcccccagcuuauacaaauuccuuuacg
cggggcgucuauuacccggauaagguuuucagauccagcgugcugcauaguacacaagaucucuuucuuccuuucuucu
caaauguaaccugguuucacgcuauucauguauccggcaccaauggaacuaaaagauuugauaacccgguguugcccuu
caaugaugguguguauuucgcuuccacggaaaagucaaacaucaucagaggguggauauucggcacaacauuggauucc
aagugccagucacuccucauagugaacaaugcuacuaacgugguuauaaaggucugcgaauuucaauuuuguaaugauc
cuuuccucgguguuuacuaucacaagaacaauaaguccuggauggaaucagaauuccguguauacaguucugcgaacaa
uugcacauucgaauaugugucccaacccuuucucauggaucuggaagggaagcaggguaacuuuaagaaucugagagaa
uucguguucaagaacauugacugcuauuuuaaaaucuauagcaaacacaccccuauaaacuugguacgggauuugccuca
aggauucucagcacucgaacccuuggucgauuugccaaucggcaucaauaucacccgguuucagacacuccuggcucuuc
accgcuccuacuugacaccuggugauuccucaucugguuggaccgccggcgcagcggcauacuaugucggcuaucuuca
accaagaaccuucuugcugaaauauaaugagaacggaacuauaacugaugccguugauugugcccuugauccacuuagc
gaaacaaagugcacucugaaguccuucacaguugaaaaggggaucuaccaaacauccaacuuccggguacaaccuacuga
guccauagugcgauuuccuaacauuaccaaucugugcccauuuggagaaguauucaacgcaacuagguucgcguccguu
uacgcguggaacaggaaaaggauuuccaauugcgucgccgacuauagcguucucuauaacagcgccucauuuagcacgu
uuaaguguuacgggguuaguccgaccaaacucaaugacuuguguuuuaccaaugucuaugcagacuccuuuguuauuag
aggcgacgaggucagacaaauugcccccggacagacagguaagauugcagauuauaauuauaaacugccggacgacuuca
cgggguguguuauugcauggaacuccaauaaccuggacucuaaaguagggggaacuauaacuaucuguaucgccuguu
ucgcaaaucuaaccugaaacccuucugcagggacauauguacugaaauauaucaagcuggcagcacaccuuguaauggcg
ucgagggauucaauuguuacuucccacuucaaucuuacgguuuucagccuacuaacggcguaggguaucaacccuauag
aguuguagugcucucuuucgaauugcuccaugcccccgcgacuguuuguggaccuaagaaguccacgaaccugguaaag
aacaaguguguuaauuuuaauuuuaauggacugaccgggacuggagugcugacugaaaguaacaagaaauuucugccuu
uccaacaauuuggccgcgauaucgcugauaccaccgacgccgucagagauccgcagacucucgaaauccuggacaucacg
cccugcucauucggcgggguuagcguuauuacuccaggcacuaacacuagcaaucaaguugcaguucuguaccaggaug
ugaacuguaccgaaguccccgucgccauucaugccgaucagcugaccccgacuuggcggguauauucaaccggcagcaau
gucuuucaaacaagggggguugucucaucggagcggagcauguaaauaauaguuaugaaugcgacauccccauuggcg
cggggaucugugcuucauaucaaacucaaaccaauuccccacggcggagacgaucaguagccagucaaucaauaauugcg
uauacgaugagucuuggggcagaaaauagcguggcuuauucuaauaauagcaucgcuauaccuacaaauuuuacaauca
guguaacuaccgaaauccuuccugucagcaugaccaaaacuagcguagauugcacgauguauauuugcggagacucaacu
gagugcaguaaccuguuguugcaauacggaaguuucuguacccagcugaaccgcgcucuuacgggcauugcaguagaac
aagauaagaauacccaagaaguguuugcccaggugaaacaaaucuacaagacucccccgauuaaagacuuuggcggguuc
aacuucagccagauauugcccgacccgucucgucguagacgguccuuuauugaagaccugcucuucaacaaggucacacu
ggcugaugcagguuuuauuaagcaauacggcgacugucuuggcgacaucgccgcuagggaccuuauaugugcucagaaa
uucaauggucugacaguucugccacccuugcucacugacgaaaugaucgcucaauauacaagcgccuugcuggcuggga
cuauuacuuccggauggacauucgggggggugccgccuugcaaauuccuuuugcaaugcaaauggcauaccguuucaa
cggaaucggcguaacccagaaugugcucuaugaaaaccagaaauugauagcaaaucaauuuaacucagccauaggaaaga
uucaagacucucucagcucaaccgcgagugcucucggcaagcuccaagacguaguaaaucaaaaugcacaagcuuugaac
acuuugguaaagcaauugucuuccaacuucggggcgaucucaucuggcccuaacgacauccugucccgguugcccaaag
uggaagccgaggugcagaucgaccgccucaucaccggccgacuucaaucacuccaaaccuacgugacucaacaacugauc
cgggcagccgagauaagggcgagugcaaacuuggcagcuacgaaaaugucagaauguguucucggccagaguaaacggg
uagacuuuugugggaaagguuaucacuugaugucuuucccucaaagcgcuccucacggcgucgucuucuugcaugugac
uuacgugccagcucaagaaaagaacuucaccaccgccccugcuauaugccaugacgguaaagcucacuucccccgagagg
gcguguucguuaguaauggaacccauugguuugugacucaacgaaacuuuuaugaaccucaaauaauuaccacggauaa
cacuuuuuuagugguaauugugacguggugaucggcauugugaauaacacagucuacgauccucugcaaccagaacug
gacagcuuuaaagaggaacuugacaaauauuucaagaaccauacaagccccgacgucgaccugggcgacaucaguggaau
caaugcguccguagucaauauccagaaggagauugaucggcuuaaugaagucgcuaagaauuugaaugaaagucuuaua
gaucugcaagaacucgggaaguacgagcaauauauuaaauggccuugguccggacguagaaggcgcaggcggggcucag
gcgguucagggucaggguauauucccgaggcgccacgcgaugggcaagcguacgugcguaaagauggcgaaugggugu
ugcuuuccacauucuuggggugau (SEQ ID NO: 108)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE

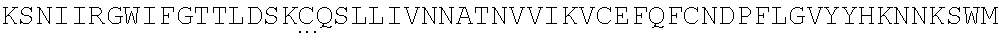

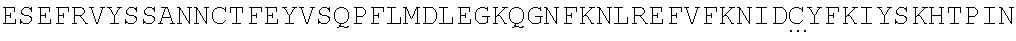

LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS

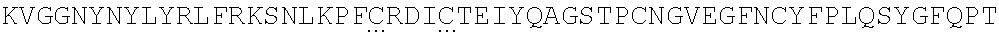

NGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA

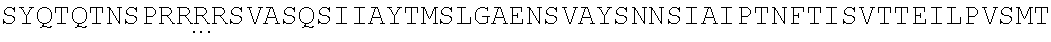

KTSVDCTMYICGDSTECSNLLLQYGSFCTQLNRALTGIAVEQDKNTQEVFAQVKQIY

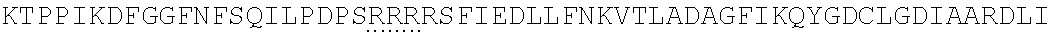

CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK

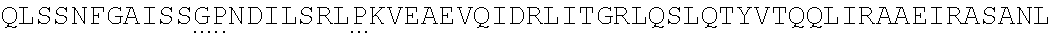

AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 109)
WuS_DownDS3_D2P_F_NoTriCle_pVax
gccaccatggactggacctggatactctttctcgtagcagcagccacacgagtgcattcaatgttcgtgttcctcgtgctcctgcctctcg
ttagcagccaatgtgttaatctcaccaccagaacacagctcccacccgcgtatactaactcttttacgaggggagtttattatcccgataa
ggttttccggtctagcgtactccactccacccaagatctgttcctgcctttctttagcaacgtgacgtggtttcatgcaatccacgtgagtg
gcaccaatggaaccaagcggttcgataatcctgtgttgccgtttaacgatggcgtgtattttgcctcaactgaaaagtctaacataatacg
cggctggatcttcgggaccacattggatagtaagtgtcaatctctgcttatcgtgaacaacgctactaacgtcgttataaaggtctgtgag
ttccaattctgcaacgacccattcctgggtgtgtattaccacaagaataataaatcttggatggagtctgagtttcgcgtatactcttctgcta
acaactgcacctttgaatatgtaagtcaaccattcctcatggatctggaaggaaaacaaggcaactttaagaacttgcgggaatttgtctt
caagaacatcgactgttattttaaaatttactcaaaacacaccccgattaatctggtccgcgatttgccccaagggttctctgcattggaac
cccttgtggacttgcctataggaattaatatcacccgctttcaaactcttctggcgctgcaccgtagctacctgacaccaggagatagctc
tagtggctggactgctggagccgcggcatattatgtggggtatctgcagccacgtacatttctcctcaaatataatgaaaatggtacaata
acggatgcagtcgactgcgcattggaccctctgagtgaaacaaaatgcactctcaagagcttcactgttgaaaagggcatataccaaa
catctaattttagagtccaacccactgaatccattgtccgatttcctaatattacaaacctctgcccatttggagaagtgttcaacgccacta
ggtttgcatccgtgtacgcatggaacagaaaacgaatttctaattgtgtggcagactatagcgtgctgtataactcagcaagctttagcac
atttaagtgttatggagttagcccaaccaaattgaatgatctttgtttcacgaacgtgtacgccgatagcttcgttattcgaggggacgagg
tgaggcaaatcgctccaggtcaaaccggtaaaatcgccgattacaattataaacttcctgatgacttcactggctgtgtcatagcatgga
actctaataatctcgacagcaaggtcggtgggaactataactatctttatcgactctttagaaagagtaatctcaaaccattttgcagagac
atttgtacagagatttatcaggcagggagcacaccatgtaatggggtcgagggcttcaactgttacttccccctgcaatcttatgggttcc
agccgaccaatggagtgggctaccaaccttatcgcgtggtggtcctgtcttttgaactgcttcatgctccagccaccgtatgcggcccta
agaagtctacaaatttggtcaagaacaagtgcgtcaattttaacttcaatggtctgactggaaccggtgtcctcacagaatctaacaagaa
atttctgccatttcaacaatttggaagagatatcgcggatactacggatgctgttagggacccccaaacacttgaaattctcgacattaca
ccctgttcctttggcggggtcagtgtcattaccccgggtacaaatactagtaaccaagtcgcagtactgtatcaagatgttaattgtaccg
aagtgccggtagcaatacacgctgatcaacttacaccaacatggcgagtgtattctacggggagtaatgtcttccaaacgcgggccgg
gtgtctgattggcgcggaacacgtaaacaactcctacgaatgtgatattccaataggcgcaggcatatgtgcgagctatcaaacacaaa
ctaactcccctagacggcgtcggagtgtggctagtcaatcaatcattgcctatacaatgtctctgggagcagaaaacagcgtggcatatt
ccaataattccatcgctatacctaccaactttaccatcagcgtcactactgagattcttcccgtctccatgacgaaaacttccgttgattgta
ctatgtacatctgcggagacagcaccgaatgcagtaaccttctcttgcaatatggcagcttttgtactcagctcaacagagctctcacag
gtattgccgtcgaacaagataagaacacccaagaggtgttcgcccaggtgaaacagatatataagaccccacccatcaaggatttcgg
cgggtttaattttagtcaaatcctgcccgatccctcacggcgtcgcaggtcctttattgaagatcttctgttcaataaggtcacactcgctga
cgcaggctttatcaagcagtatggagattgtctgggcgatatagctgcgagggacttgatctgcgcacaaaagttcaacggccttacag
tgctgcccccgttgctgacagatgagatgattgcgcaatacacttccgcgcttctcgcagggaccatcacgagcggctggacgttcgg
cgctggcgccgctctgcaaatcccgtttgcaatgcaaatggcctataggtttaatggtatcggtgtaacgcaaaacgtactttatgaaaac
cagaaactgatcgctaaccaattcaattccgctattggcaaaattcaagacagcctcagcagcacggctagtgcactgggtaaactcca
agacgtggtgaaccaaaatgcccaagcattgaatacacttgtcaagcaacttagttccaacttcggtgcaatttcaagtggtccaaatga
catacttagcaggctgcctaaagtagaagccgaagtgcaaatcgatagacttatcaccggccgcctgcaatcccttcaaacatacgtga
ctcagcagcttatcagggctgctgagattcgagcaagtgcgaacctggccgccaccaaaatgagtgagtgcgtccttgggcaatcca
agcgcgttgacttttgtggtaaggggtatcatctcatgagcttcccccaatccgcccctcacggagtagtgtttctccatgtgacgtatgtt
cctgcacaagagaagaacttcacaacggctccggctatatgtcatgacggaaaagcgcactttcctcgcgaaggagtgtttgtgtcaaa
tggaacgcactggttcgtgacgcaaaggaatttctacgagcctcaaatcatcactacagataatacttttgtctctgggaattgcgacgtg
gtcattggaatcgtcaacaatacggtttacgatcccctgcaaccagaactggattcattcaaagaagaactcgacaagtacttcaagaat
cataccagtcctgatgtggatctgggcgatatcagtgggatcaatgcaagcgttgtcaacattcaaaaggaaatagaccgcctcaacga
agtcgcaaagaatctcaatgaaagccttattgatcttcaagagctcggaaaatatgagcaatatattaagtggccttggtccggcggctc
aggcggaagtggctcaggatatattcctgaggctccccgagatggacaagcatacgtgagaaaagatggggagtgggtgttgctga
gtacgttccttggatgataa (SEQ ID NO: 110)
gccaccauggacuggaccuggauacucuuucucguagcagcagccacacgagugcauucaauguucguguuccucgug
uccugccucucguuagcagccaauguguuaaucucaccaccagaacacagcucccacccgcguauacuaacucuuuuacg
aggggaguuuauuaucccgauaagguuuuccggucuagcguacuccacuccacccaagaucuguuccugccuuucuuua
gcaacgugacgugguuucaugcaauccacgugaguggcaccaauggaaccaagcgguucgauaauccuguguugccguu
uaacgauggcguguauuuugccucaacugaaaagucuaacauaauacgcggcuggaucuucgggaccacauuggauagu
aagugucaaucucugcuuaucgugaacaacgcuacuaacgucguuauaaaggucugugaguuccaauucugcaacgacc
cauuccuggguguguauuaccacaagaauaauaaaucuuggauggagucugaguuucgcguauacucuucugcuaacaa
cugcaccuuugaauauguaagucaaccauuccucauggaucuggaaggaaaacaaggcaacuuuaagaacuugcgggaau
uugucuucaagaacaucgacuguuauuuuaaaauuuacucaaaacacaccccgauuaaucugguccgcgauuugccccaa
ggguucucugcauuggaaccccuuguggacuugccuauaggaauuaauaucacccgcuuucaaacucuucuggcgcugc
accguagcuaccugacaccaggagauagcucuaguggcuggacugcuggagccgcggcauauuaugugggguaucugca
gccacguacauuucuccucaaauauaaugaaaaugguacaauaacggaugcagucgacugcgcauuggacccucugagug
aaacaaaaugcacucucaagagcuucacuguugaaaagggcauauaccaaacaucuaauuuuagaguccaacccacugaa
uccauuguccgauuuccuaauauuacaaaccucugcccauuuggagaaguguucaacgccacuagguuugcauccgugu
acgcauggaacagaaaacgaauuucuaauuguguggcagacuauagcgugcuguauaacucagcaagcuuuagcacauu
uaaguguuauggaguuagcccaaccaaauugaaugaucuuuguuucacgaacguguacgccgauagcuucguuauucga
ggggacgaggugaggcaaaucgcuccaggucaaaccgguaaaaucgccgauuacaauuauaaacuuccugaugacuucac
uggcugugucauagcauggaacucuaauaaucucgacagcaaggucggugggaacuauaacuaucuuuaucgacucuuu
agaaagaguaaucucaaaccauuuugcagagacauuuguacagagauuuaucaggcagggagcacaccauguaaugggg
ucgagggcuucaacuguuacuucccccugcaaucuuauggguuccagccgaccaauggagugggcuaccaaccuuaucg
cguggugguccugucuuuugaacugcuucaugcuccagccaccguaugcggcccuaagaagucuacaaauuuggucaag
aacaagugcgucaauuuuaacuucaauggucugacuggaaccgguguccucacagaaucuaacaagaaauuucugccauu
ucaacaauuuggaagagauaucgcggauacuacggaugcuguuagggacccccaaacacuugaaauucucgacauuacac
ccuguuccuuuggcggggucagugucauuaccccggguacaaauacuaguaaccaagucgcaguacuguaucaagaugu
uaauuguaccgaagugccgguagcaauacacgcugaucaacuuacaccaacauggcgaguguauucuacggggaguaau
gucuuccaaacgcgggccgggugucugauuggcgcggaacacguaaacaacuccuacgaaugugauauuccaauaggcg
caggcauaugugcgagcuaucaaacacaaacuaacuccccuagacggcgucggaguguggcuagucaaucaaucauugcc
uauacaaugucucugggagcagaaaacagcguggcauauuccaauaauuccaucgcuauaccuaccaacuuuaccaucag
cgucacuacugagauucuucccgucuccaugacgaaaacuuccguugauuguacuauguacaucugcggagacagcacc
gaaugcaguaaccuucucuugcaauauggcagcuuuuguacucagcucaacagagcucucacagguauugccgucgaac
aagauaagaacacccaagagguguucgcccaggugaaacagauauauaagaccccacccaucaaggauuucggcggguuu
aauuuuuagucaaauccugcccgaucccucacggcgucgcagguccuuuauugaagaucuucuguucaauaaggucacac
ucgcugacgcaggcuuuaucaagcaguauggagauugucugggcgauauagcugcgagggacuugaucugcgcacaaaa
guucaacggccuuacagugcugcccccguugcugacagaugagaugauugcgcaauacacuuccgcgcuucucgcaggg
accaucacgagcggcuggacguucggcgcuggcgccgcucugcaaaucccguuugcaaugcaaauggccuauagguuua
augguaucgguguaacgcaaaacguacuuuaugaaaaccagaaacugaucgcuaaccaauucaauuccgcuauuggcaaa
auucaagacagccucagcagcacggcuagugcacuggguaaacuccaagacguggugaaccaaaaugcccaagcauugaa
uacacuugucaagcaacquaguuccaacuucggugcaauuucaagugguccaaaugacauacuuagcaggcugccuaaag
uagaagccgaagugcaaaucgauagacuuaucaccggccgccugcaaucccuucaaacauacgugacucagcagcuuauc
agggcugcugagauucgagcaagugcgaaccuggccgccaccaaaaugagugagugcguccuugggcaauccaagcgcg
uugacuuuugugguaagggguaucaucucaugagcuucccccaauccgccccucacggaguaguguuucuccaugugac
guauguuccugcacaagagaagaacuucacaacggcuccggcuauaugucaugacggaaaagcgcacuuuccucgcgaag
gaguguuugugucaaauggaacgcacugguucgugacgcaaaggaauuucuacgagccucaaaucaucacuacagauaa
uacuuuugucucugggaauugcgacguggucauuggaaucgucaacaauacgguuuacgauccccugcaaccagaacug
gauucauucaaagaagaacucgacaaguacuucaagaaucauaccaguccugauguggaucugggcgauaucaguggga
ucaaugcaagcguugucaacauucaaaaggaaauagaccgccucaacgaagucgcaaagaaucucaaugaaagccuuauu
gaucuucaagagcucggaaaauaugagcaauauauuaaguggccuugguccggcggcucaggcggaaguggcucaggau
auauuccugaggcuccccgagauggacaagcauacgugagaaaagauggggaguggguguugcugaguacguuccuug
gaugauaa (SEQ ID NO: 111)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE

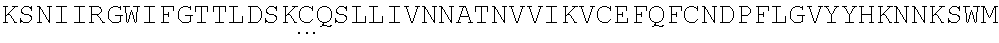

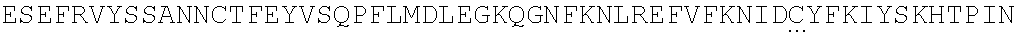

LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS

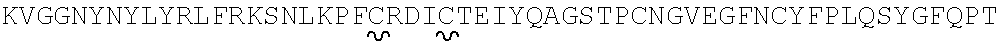

NGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA

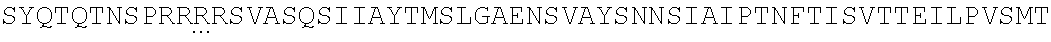

KTSVDCTMYICGDSTECSNLLLQYGSFCTQLNRALTGIAVEQDKNTQEVFAQVKQIY

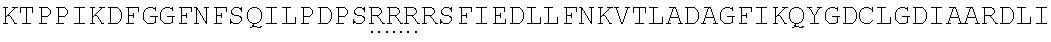

CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK

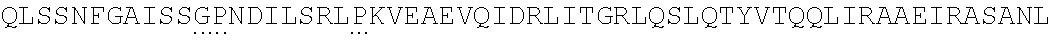

AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGGSGGSGSGYIPEAPRDGQAYVRKDGEWVLLSTFLG**
(SEQ ID NO: 112)
WuS_DownDS2_2P_pVax
gccaccatggactggacctggatactctttctcgtagcagcagccacacgagtgcattcaatgttcgtgttcttggtgctgctgcctcttgt
ctcatcacagtgcgttaatctgaccacccgtacacaactcccacccgcatacacaaatagctttacacgcggagtgtattaccccgataa
agtctttcggagctcagtgctccattctactcaagatcttttcctgccgttctttagtaacgttacttggtttcatgcaatacatgtgtctggca
caaacggaaccaaacgttttgataatccggtgttgccatttaatgatggtgtatattttgcttccacggaaaagtcaaacatcatccgtggg
tggatctttggcaccactcttgatagcaaatgtcaaagccttctgattgttaataacgctacaaacgtcgtaattaaagtgtgtgaattccag
ttctgtaatgaccccttcctcggagtatattaccacaagaataacaaatcttggatggagagcgaatttagagtttacagttcagccaataa
ctgtacatttgaatatgtcagtcagcctttcctcatggacctcgaaggtaaacaaggtaattttaagaacttgagagagttcgtgtttaagaa
catcgatggctatttcaaaatttactctaagcacacaccaatcaacctggttcgagacctgccccagggtttctcagctttggaaccattg
gtggacctgccaatcggcattaacattaccagatttcaaactttgttggcactccaccggtcatatcttacccccggagacagttcctcag
gctggacggcaggcgccgccgcgtactatgttgggtatctccaaccccgaaccttccttctcaaatacaatgaaaacgggacgattac
agatgcagtcgattgcgccctggaccccttgtccgaaactaaatgcactctgaagagtttcacggtagagaagggaatctatcaaacga
gcaattttcgagtccaaccaacggaatctattgtgcggtttcccaatatcacaaacctctgtccattcggagaagtctttaatgctaccagg
tttgcgtctgtatatgcatggaaccgaaagaggatttccaattgcgtagcggactacagtgtcctttataacagcgcttcattttccacgttt
aagtgttatggtgtttctccaacgaaactcaacgacctctgttttactaacgtttacgctgacagctttgttatacgtggggacgaagtcag
gcaaattgctcctggacagactggaaagatcgctgattataattataaacttcctgacgatttcaccggctgcgttattgcatggaactcca
acaatctggattcaaaagtgggtggaaattataattatctgtataggttgtttcggaagagcaatcttaagccctttgagcgggacatatgt
accgaaatttaccaagcaggctccaccccatgcaatggagtagaagggttcaattgctattttcctctgcaaagttatggctttcaaccca
ccaacggagttgggtatcaaccttacagggttgtcgtgctgagtttcgaattgctccacgcacccgctacagtatgtggccccaagaag
tccactaatcttgttaagaataaatgcgtgaacttcaacttcaatggacttacaggtactggagtactcacggaatcaaacaagaaatttct
cccatttcaacagtttggccgagatatagctgacaccacagatgctgttcgcgacccccagacgttggaaatacttgatatcactccctg
cagcttcggcggcgtgagcgtgatcactccaggtactaatacgagcaatcaagttgccgttctgtaccaagatgtgaactgcaccgag
gttccagtggcaattcacgccgaccaacttactcccacctgggggtctattccaccggatcaaacgtcttccaaactcgcgctggttgc
cttatcggtgcagagcacgttaataattcctatgaatgtgacattcccataggagcaggcatctgtgcatcttatcaaacccagactaattc
ccctggttccgcttcctctgttgcatcccagtccataattgcctacactatgagtctcggggctgaaaattccgtggcctattctaataattc
aatcgccatcccaaccaattttaccatatccgtaacgactgaaatacttcctgtcagtatgaccaagacctcagtggactgcaccatgtac
atctgcggcgattctactgaatgttccaatctgcttttgcaatatggttcattctgcacccaactcaacagggctcttacagggatcgccgt
cgaacaggataagaatacccaggaagtgttcgcccaagttaagcaaatttacaagacaccacccatcaaggacttcggcgggttcaa
cttcagccaaattctgcccgacccgtctaagccttctaagcgctctttcattgaggatcttttgttcaataaggttacgcttgccgatgcagg
gtttatcaaacagtatggcgactgtcttggggatatcgcagctagggatcttatttgtgcacagaaatttaatggcctgactgttcttccccc
tttgctcactgacgagatgattgcccagtacacttcagctctcctggccgggactataacttctggttggaccttcggagctggcgccgc
cctgcaaattccatttgcaatgcagatggcttatcgcttcaacggaattggggtgacccaaaatgttctctacgagaaccagaaactcatt
gcaaaccagttcaattctgcgatcgggaagatccaggattccctgtctagtacggctagtgccctcggtaagctccaagacgtcgtcaa
ccaaaacgcccaggccttgaacacccttgtcaaacaactgagctccaattttggggctattagcagtgtgctgaatgatatcctgtcccg
ccttgacccaccggaagcggaagtccaaattgatcgactgatcactgggcgtctccaatcccttcaaacttacgtgacccaacaactca
tccgagcagctgagattagggctagcgctaaccttgctgctactaagatgtcagagtgtgtcctcggccagtctaagagagtggactttt
gtgggaaagggtaccacttgatgtcattcccacaaagcgccccacacggcgtggtgtttctccacgtcacttacgttccagctcaggaa
aagaactttaccaccgcccccgctatatgtcatgatgggaaggcccactttcctcgtgaaggtgtctttgtcagcaatggcacacactgg
tttgtgacccaacggaatttctatgagcctcagattattaccacggataacactttcgtatcagggaattgtgatgtggttatcggcatcgtt
aataatacagtgtatgacccactgcagccagagcttgacagcttcaaagaagagctcgataagtactttaagaatcatacaagtcctgac
gttgatcttggggatattagtgggattaacgccagcgtcgtcaatattcagaaagagattgacaggttgaacgaagtagctaagaatctt
aatgaaagcctgatagatttgcaagaacttggtaagtatgagcaggggtacatacccgaggctcctcgggatgggcaggcctatgtac
gcaaagacggtgaatgggtattgctcagcacttttctcggctgataa (SEQ ID NO: 113)
gccaccauggacuggaccuggauacucuuucucguagcagcagccacacgagugcauucaauguucguguucuuggugc
ugcugccucuugucucaucacagugcguuaaucugaccacccguacacaacucccacccgcauacacaaauagcuuuaca
cgcggaguguauuaccccgauaaagucuuucggagcucagugcuccauucuacucaagaucuuuuccugccguucuuua
guaacguuacuugguuucaugcaauacaugugucuggcacaaacggaaccaaacguuuugauaauccgguguugccauu
uaaugaugguguauauuuugcuuccacggaaaagucaaacaucauccguggguggaucuuuggcaccacucuugauagc
aaaugucaaagccuucugauuguuaauaacgcuacaaacgucguaauuaaagugugugaauuccaguucuguaaugacc
ccuuccucggaguauauuaccacaagaauaacaaaucuuggauggagagcgaauuuagaguuuacaguucagccaauaac
uguacauuugaauaugucagucagccuuuccucauggaccucgaagguaaacaagguaauuuuaagaacuugagagagu
ucguuuuaagaacaucgauggcuauuucaaaauuuacucuaagcacacaccaaucaaccugguucgagaccugccccag
gguuucucagcuuuggaaccauugguggaccugccaaucggcauuaacauuaccagauuucaaacuuuguuggcacucc
accggucauaucuuacccccggagacaguuccucaggcuggacggcaggcgccgccgcguacuauguuggguaucucca
accccgaaccuuccuucucaaauacaaugaaaacgggacgauuacagaugcagucgauugcgcccuggaccccuuguccg
aaacuaaaugcacucugaagaguuucacgguagagaagggaaucuaucaaacgagcaauuuucgaguccaaccaacggaa
ucuauugugcgguuucccaauaucacaaaccucuguccauucggagaagucuuuaaugcuaccagguuugcgucuguau
augcauggaaccgaaagaggauuuccaauugcguagcggacuacaguguccuuuauaacagcgcuucauuuuccacguu
uaaguguuaugguguuucuccaacgaaacucaacgaccucuguuuuacuaacguuuacgcugacagcuuuguuauacgu
ggggacgaagucaggcaaauugcuccuggacagacuggaaagaucgcugauuauaauuauaaacuuccugacgauuuca
ccggcugcguuauugcauggaacuccaacaaucuggauucaaaaguggguggaaauuauaauuaucuguauagguuguu
ucggaagagcaaucuuaagcccuuugagcgggacauauguaccgaaauuuaccaagcaggcuccaccccaugcaauggag
uagaaggguucaauugcuauuuuccucugcaaaguuauggcuuucaacccaccaacggaguuggguaucaaccuuacag
gguugucgugcugaguuucgaauugcuccacgcacccgcuacaguauguggccccaagaaguccacuaaucuuguuaag
aauaaaugcgugaacuucaacuucaauggacuuacagguacuggaguacucacggaaucaaacaagaaauuucucccauu
ucaacaguuuggccgagauauagcugacaccacagaugcuguucgcgacccccagacguuggaaauacuugauaucacuc
ccugcagcuucggcggcgugagcgugaucacuccagguacuaauacgagcaaucaaguugccguucuguaccaagaugu
gaacugcaccgagguuccaguggcaauucacgccgaccaacuuacucccaccuggcgggucuauuccaccggaucaaacg
ucuuccaaacucgcgcugguugccuuaucggugcagagcacguuaauaauuccuaugaaugugacauucccauaggagc
aggcaucugugcaucuuaucaaacccagacuaauuccccugguuccgcuuccucuguugcaucccaguccauaauugccu
acacuaugagucucggggcugaaaauuccguggccuauucuaauaauucaaucgccaucccaaccaauuuuaccauaucc
guaacgacugaaauacuuccugucaguaugaccaagaccucaguggacugcaccauguacaucugcggcgauucuacuga
auguuccaaucugcuuuugcaauaugguucauucugcacccaacucaacagggcucuuacagggaucgccgucgaacag
gauaagaauacccaggaaguguucgcccaaguuaagcaaauuuacaagacaccacccaucaaggacuucggcggguucaa
cuucagccaaauucugcccgacccgucuaagccuucuaagcgcucuuucauugaggaucuuuuguucaauaagguuacg
cuugccgaugcaggguuuaucaaacaguauggcgacugucuuggggauaucgcagcuagggaucuuauuugugcacag
aaauuuaauggccugacuguucuucccccuuugcucacugacgagaugauugcccaguacacuucagcucuccuggccg
ggacuauaacuucugguuggaccuucggagcuggcgccgcccugcaaauuccauuugcaaugcagauggcuuaucgcuu
caacggaauuggggugacccaaaauguucucuacgagaaccagaaacucauugcaaaccaguucaauucugcgaucggga
agauccaggauucccugucuaguacggcuagugcccucgguaagcuccaagacgucgucaaccaaaacgcccaggccuug
aacacccuugucaaacaacugagcuccaauuuuggggcuauuagcagugugcugaaugauauccugucccgccuugacc
caccggaagcggaaguccaaauugaucgacugaucacugggcgucuccaaucccuucaaacuuacgugacccaacaacuc
auccgagcagcugagauuagggcuagcgcuaaccuugcugcuacuaagaugucagaguguguccucggccagucuaaga
gaguggacuuuugugggaaaggguaccacuugaugucauucccacaaagcgccccacacggcgugguguuucuccacgu
cacuuacguuccagcucaggaaaagaacuuuaccaccgcccccgcuauaugucaugaugggaaggcccacuuuccucgug
aaggugucuuugucagcaauggcacacacugguuugugacccaacggaauuucuaugagccucagauuauuaccacgga
uaacacuuucguaucagggaauugugaugugguuaucggcaucguuaauaauacaguguaugacccacugcagccagag
cuugacagcuucaaagaagagcucgauaaguacuuuaagaaucauacaaguccugacguugaucuuggggauauuagug
ggauuaacgccagcgucgucaauauucagaaagagauugacagguugaacgaaguagcuaagaaucuuaaugaaagccu
gauagauuugcaagaacuugguaaguaugagcagggguacauacccgaggcuccucgggaugggcaggccuauguacgc
aaagacggugaauggguauugcucagcacuuuucucggcugauaa (SEQ ID NO: 114)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE

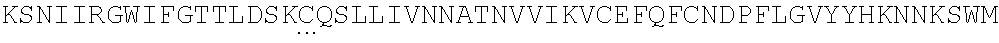

ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS

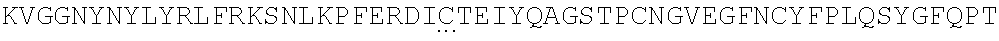

NGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA

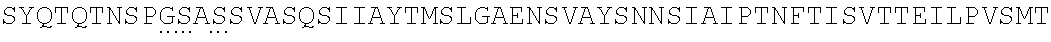

KTSVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAARD
LICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTL

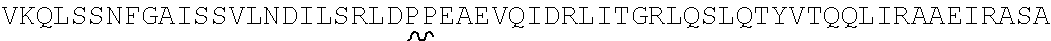

NLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNL
NESLIDLQELGKYEQGYIPEAPRDGQAYVRKDGEWVLLSTFLG** (SEQ ID NO: 115)
WuS_DownDS1_2P_pVax
gccaccatggactggacctggatactctttctcgtagcagcagccacacgagtgcattcaatgtttgttttccttgttctgctcccgcttgtg
tcttcccagtgcgtgaacctgaccacccgaactcaactcccaccagcatacaccaactcatttacaagaggagtttattacccggacaa
ggtatttcgaagttcagttcttcacagcacccaagacctgtttctgccattcttcagtaatgtcacttggtttcacgcgatacatgtcagcgg
tacaaacgggacaaagcgattcgataacccagtactcccattcaacgacggagtgtattttgcatctacagagaaatccaacattatacg
cgggtggatctttggaactactctggactccaagacacagagcctgctcattgtgaacaatgcaacgaatgtcgtcataaaagtctgtga
atttcaattttgcaacgatcctttcctcggagtctattaccataagaacaataagagttggatggagagtgagtttcgcgtctattcttccgc
gaacaattgtacatttgaatatgtatcacaaccctttcttatggatttggaaggcaaacaaggtaacttcaagaacttgcgcgagttcgtgtt
caagaacatagactgttattttaagatctatagtaagcatacgccaatcaatctggtgcgagatttgcctcagggcttttctgctcttgaacc
cttggttgatctgcccatcgggatcaacataaccagatttcaaacgttgctcgcactccaccgcagctatctcactcctggcgattcctcat
ctgggtggaccgccggagctgctgcttattacgtcggctatctccagccgcgtactttcctgctcaagtataatgagaatggcaccatta
ccgatgctgtggattgtgctcttgatccactctctgaaaccaaatgcactctcaagtcttttaccgtggaaaagggtatttatcagacatcta
attttcgggtgcaacctactgagtcaattgtacggtttcctaacataactaacctttgtccatttggggaagtcttcaatgccacgcggttcg
catcagtctatgcatggaacagaaaacgtatctccaactgcgtcgccgattattccgtcctttacaatagcgctagcttttccacattcaaat
gttatggcgtatcaccaaccaaacttaacgatctctgctttactaatgtctacgctgactctttcgttattcgaggtgacgaggtgcgccaa
attgcgcctggtcaaaccggaaagattgccgattataactacaagctccccgacgactttacgggttgtgtgatcgcctggaatagcaat
aacctcgattctaaagttggcggtaattataactatctgtacagactctttaggaaaagtaatctcaagcccttttgcagggatatctcaacc
gaaatctaccaagccggcagcactccttgcaatggtgtcgaggggtttaattgttatttcccactgcaatcttacggctttcaaccgactaa
tggagtcggttatcaaccctatagggtggtggtactctcctttgaacttttgcacgctccggcaacagtttgtggaccaaagaaaagtacg
aaccttgttaagaataagtgtgttaatttcaattttaacggcctcactggaacaggtgtcctcacagaaagcaacaagaagtttctccctttc
caacagtttggacgggatatcgccgacactactgacgccgtcagagatcctcaaactctcgaaatcttggatatcacaccatgttctttcg
gtggtgtctccgtcataacaccaggaactaacacctctaatcaagtggccgtgctctatcaggacgtcaattgcacagaagtgcctgtc
gcaatccatgctgatcagctcactcccacctggcgtgtgtattccactggctctaatgtctttcagacacgggcaggttgccttattgggg
cagagcatgtgaacaattcctacgaatgcgatatacccattggggcaggcatttgcgccagctaccaaacccaaactaacagccccg
ggagtgccagcagcgtggcatctcagtccattattgcctatacgatgagcctgggtgctgaaaatagcgtggcttatagtaataactctat
cgccatacccacaaacttcaccatttcagtgaccaccgaaatccttcctgtttctatgaccaaaacgtccgtcgattgtacaatgtacattt
gcggcgatagcactgaatgttcaaacctgctcctgcaatacggctctttctgtactcagctcaaccgggcactcaccggcatagccgtc
gaacaagacaagaatacccaggaagtctttgcgcaggtgaaacaaatctataagaccccaccaataaaagatttcggcggttttaatttc
agccaaatcttgcctgatcccagcaagccatctaaacggtctttcattgaagatctcctgttcaacaaggttacgctggctgacgccggg
tttattaagcaatatggcgattgccttggggacattgccgcacgagacctcatttgtgcccagaaattcaacgggctcaccgtattgcccc
cgctcctcacagacgaaatgatcgcccaatatacaagcgccctgcttgcgggaaccattacaagcggttggacctttggtgccggcgc
agctctgcaaatacccttcgcaatgcaaatggcatatcggtttaatggaattggcgtaacccaaaacgtgctgtatgaaaaccagaaact
gatcgcaaatcaattcaatagtgctataggaaagatccaagacagtctgtcttccactgctagcgcgctggggaagctccaagacgttg
tgaaccaaaacgcgcaggccctgaataccctggtgaagcaactttcaagcaatttcggtgctatatcttctgtcctcaatgacattctctct
cggctcgatcccccggaagccgaagttcagatagaccgtttgatcacaggccgcttgcaatccctgcaaacctacgttacacaacaac
tgattcgcgccgccgaaattcgggcatccgccaatctggccgcaaccaaaatgtccgagtgtgttctcggtcaatccaaacgcgtgga
tttctgcggaaaaggataccatttgatgtcatttccacaatcagctccacacggtgttgtattcctgcacgtgacctacgtgccagcccag
gagaagaattttactactgcgcccgccatttgtcatgacgggaaggctcattttcctcgggaaggggttttcgtctcaaacggtacccatt
ggttcgtgactcagaggaacttttatgaacctcaaatcataacgaccgataacacgtttgtaagtggcaattgcgacgtggtcatcggga
ttgtaaacaatactgtctatgaccctctccaaccagagcttgacagctttaaagaagagcttgataaatactttaagaaccatacctcacca
gacgtcgatttgggagatatcagtggcattaatgcctctgtcgtcaatatccagaaagagattgaccgcttgaacgaagttgccaagaat
cttaatgagtctctgattgacttgcaagaattgggaaaatatgaacaaggatatattccagaagcccctcgcgatgggcaagcatatgttc
gaaaggatggggaatgggtgctgctcagcacctttctcggttgataa (SEQ ID NO: 116)
gccaccauggacuggaccuggauacucuuucucguagcagcagccacacgagugcauucaauguuuguuuuccuuguuc
ugcucccgcuugugucuucccagugcgugaaccugaccacccgaacucaacucccaccagcauacaccaacucauuuacaa
gaggaguuuuuacccggacaagguauuucgaaguucaguucuucacagcacccaagaccuguuucugccauucuucag
uaaugucacuugguuucacgcgauacaugucagcgguacaaacgggacaaagcgauucgauaacccaguacucccauuca
acgacggaguguauuuugcaucuacagagaaauccaacauuauacgcggguggaucuuuggaacuacucuggacuccaa
gacacagagccugcucauugugaacaaugcaacgaaugucgucauaaaagucugugaauuucaauuuugcaacgauccu
uuccucggagucuauuaccauaagaacaauaagaguuggauggagagugaguuucgcgucuauucuuccgcgaacaauu
guacauuugaauauguaucacaacccuuucuuauggauuuggaaggcaaacaagguaacuucaagaacuugcgcgaguu
cguguucaagaacauagacuguuauuuuaagaucuauaguaagcauacgccaaucaaucuggugcgagauuugccucag
ggcuuuucugcucuugaacccuugguugaucugcccaucgggaucaacauaaccagauuucaaacguugcucgcacucc
accgcagcuaucucacuccuggcgauuccucaucuggguggaccgccggagcugcugcuuauuacgucggcuaucucca
gccgcguacuuuccugcucaaguauaaugagaauggcaccauuaccgaugcuguggauugugcucuugauccacucucu
gaaaccaaaugcacucucaagucuuuuaccguggaaaaggguauuuaucagacaucuaauuuucgggugcaaccuacug
agucaauuguacgguuuccuaacauaacuaaccuuuguccauuuggggaagucuucaaugccacgcgguucgcaucagu
cuaugcauggaacagaaaacguaucuccaacugcgucgccgauuauuccguccuuuacaauagcgcuagcuuuuccacau
ucaaauguuauggcguaucaccaaccaaacuuaacgaucucugcuuuacuaaugucuacgcugacucuuucguuauucg
aggugacgaggugcgccaaauugcgccuggucaaaccggaaagauugccgauuauaacuacaagcuccccgacgacuuua
cggguugugugaucgccuggaauagcaauaaccucgauucuaaaguuggcgguaauuauaacuaucuguacagacucuu
uaggaaaaguaaucucaagcccuuuugcagggauaucucaaccgaaaucuaccaagccggcagcacuccuugcaauggug
ucgagggguuuaauuguuauuucccacugcaaucuuacggcuuucaaccgacuaauggagucgguuaucaacccuauag
gguggugguacucuccuuugaacuuuugcacgcuccggcaacaguuuguggaccaaagaaaaguacgaaccuuguuaag
aauaaguguguuaauuucaauuuuaacggccucacuggaacagguguccucacagaaagcaacaagaaguuucucccuu
uccaacaguuuggacgggauaucgccgacacuacugacgccgucagagauccucaaacucucgaaaucuuggauaucaca
ccauguucuuucgguggugucuccgucauaacaccaggaacuaacaccucuaaucaaguggccgugcucuaucaggacg
ucaauugcacagaagugccugucgcaauccaugcugaucagcucacucccaccuggcguguguauuccacuggcucuaa
ugucuuucagacacgggcagguugccuuauuggggcagagcaugugaacaauuccuacgaaugcgauauacccauuggg
gcaggcauuugcgccagcuaccaaacccaaacuaacagccccgggagugccagcagcguggcaucucaguccauuauugc
cuauacgaugagccugggugcugaaaauagcguggcuuauaguaauaacucuaucgccauacccacaaacuucaccauuu
cagugaccaccgaaauccuuccuguuucuaugaccaaaacguccgucgauuguacaauguacauuugcggcgauagcacu
gaauguucaaaccugcuccugcaauacggcucuuucuguacucagcucaaccgggcacucaccggcauagccgucgaaca
agacaagaauacccaggaagucuuugcgcaggugaaacaaaucuauaagaccccaccaauaaaagauuucggcgguuuua
auuucagccaaaucuugccugaucccagcaagccaucuaaacggucuuucauugaagaucuccuguucaacaagguuacg
cuggcugacgccggguuuauuaagcaauauggcgauugccuuggggacauugccgcacgagaccucauuugugcccaga
aauucaacgggcucaccguauugcccccgcuccucacagacgaaaugaucgcccaauauacaagcgcccugcuugcggga
accauuacaagcgguuggaccuuuggugccggcgcagcucugcaaauacccuucgcaaugcaaauggcauaucgguuua
auggaauuggcguaacccaaaacgugcuguaugaaaaccagaaacugaucgcaaaucaauucaauagugcuauaggaaag
auccaagacagucugucuuccacugcuagcgcgcuggggaagcuccaagacguugugaaccaaaacgcgcaggcccugaa
uacccuggugaagcaacuuucaagcaauuucggugcuauaucuucuguccucaaugacauucucucucggcucgauccc
ccggaagccgaaguucagauagaccguuugaucacaggccgcuugcaaucccugcaaaccuacguuacacaacaacugau
ucgcgccgccgaaauucgggcauccgccaaucuggccgcaaccaaaauguccgaguguguucucggucaauccaaacgcg
uggauuucugccgaaaaggauaccauuugaugucauuuccacaaucagcuccacacgguguuguauuccugcacgugac
cuacgugccagcccaggagaagaauuuuacuacugcgcccgccauuugucaugacgggaaggcucauuuuccucgggaa
gggguuuucgucucaaacgguacccauugguucgugacucagaggaacuuuuaugaaccucaaaucauaacgaccgaua
acacguuuguaaguggcaauugcgacguggucaucgggauuguaaacaauacugucuaugacccucuccaaccagagcu
ugacagcuuuaaagaagagcuugauaaauacuuuaagaaccauaccucaccagacgucgauuugggagauaucaguggca
uuaaugccucugucgucaauauccagaaagagauugaccgcuugaacgaaguugccaagaaucuuaaugagucucugau
ugacuugcaagaauugggaaaauaugaacaaggauauauuccagaagccccucgcgaugggcaagcauauguucgaaag
gauggggaaugggugcugcucagcaccuuucucgguugauaa (SEQ ID NO: 117)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM

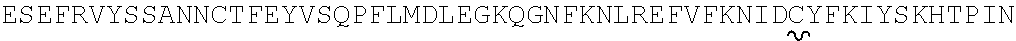

LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS

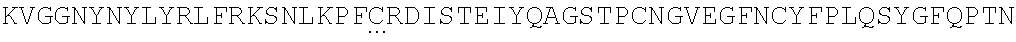

GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS

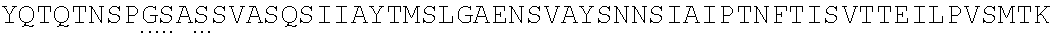

TSVDCTMYICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK

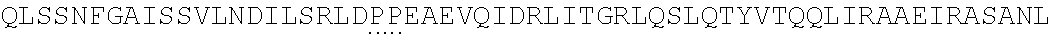

AATKMSECVLGQSKRVDFCGKGYHLMSFPQSAPHGVVFLHVTYVPAQEKNFTTAPA
DPLQPELDSFKEELDKYFKNHTSPDVDLGDISGINASVVNIQKEIDRLNEVAKNLNES
LIDLQELGKYEQGYIPEAPRDGQAYVRKDGEWVLLSTFLG** (SEQ ID NO: 118)
WuS_2P_pVax
gccaccatggactggacctggatactctttctcgtagcagcagccacacgagtgcattcaatgtttgtgtttcttgtcctgttgccattggtg
agctcccaatgtgtcaatctgaccacccggacacaattgccccctgcatatacaaattcattcacgagaggagtatactatcccgacaaa
gttttccggtcctcagtccttcattccactcaagatcttttccttccattcttttctaacgtaacctggttccatgcaattcatgtcagtgggacc
aacggcacgaaacggtttgataatccagttcttccattcaatgacggagtatattttgcatcaactgagaaatctaatatcattagagggtg
gattttcggaacaactcttgactccaagacccaatccttgctcatcgttaacaatgctacaaatgtggttattaaggtctgtgagtttcaattc
tgtaacgacccctttctcggcgtatactaccataagaataataagtcttggatggagtctgaatttcgtgtctactcatcagcgaacaattgt
acatttgaatatgtgtcccaaccattcctgatggatctcgaaggaaagcagggcaattttaagaaccttcgggagttcgtctttaagaatat
cgatggatactttaaaatatatagtaaacacacaccaatcaatctggtccgagatctcccccagggttttagtgctctggagccgctggtg
gatttgcccatcggtatcaatattacgcgcttccaaacattgctcgccctgcatcggagttaccttacgcctggcgacagtagcagcgga
tggaccgctggagccgccgcctactatgtcgggtaccttcaaccacgcacttttctcctgaaatacaacgaaaatgggacaattacaga
cgctgttgattgcgcactcgatcccctgtcagaaacaaaatgtacacttaaatcttttacggtcgagaaagggatttaccagacatctaac
ttccgagtacaaccaaccgaatctatagtgcggttccccaacattacgaacctgtgcccgttcggcgaagtgttcaacgcaacacgattt
gcttctgtttacgcttggaaccggaaacgcatctccaattgcgtcgccgattacagcgttctttataattctgcatctttctccaccttcaaat
gctatggtgtctctcccacaaaactcaatgacctctgttttaccaatgtgtatgcggactccttcgtcatacgcggcgacgaggtgagaca
aatcgcaccagggcagactggcaagatcgctgattataattacaaactgcctgatgattttaccggatgcgttattgcttggaattctaata
acctcgattccaaagttggcgggaactacaattacctctaccgattgtttcgcaaatctaaccttaagccgtttgagagagatatcagcac
agagatttatcaagctggctctaccccttgcaatggagtagaaggctttaactgctattttcctcttcagtcttatggatttcaacctaccaac
ggggtagggtaccaaccctatagagtcgtcgtgctctcatttgaactccttcacgcccccgctaccgtgtgtgggcctaagaaatccact
aatctcgttaagaataagtgtgtgaattttaatttcaatggcctgacagggaccggggttctgactgaatctaacaagaaatttctgccgtt
ccaacaattcgggcgcgatattgcagacacgaccgacgcggtgcgcgatcctcaaacactcgaaatccttgatatcactccttgttcttt
cggcggtgtaagcgtcattactcctggcaccaatacctctaaccaagtggcagtactctatcaagatgtgaactgcactgaggtcccgg
ttgcaatacatgcggatcaactcaccccaacatggcgagtgtattccacagggagcaatgtgtttcaaacgagggccggctgtctcatt
ggggccgaacacgttaataatagttatgagtgcgatattcccattggagcgggcatttgtgccagctatcagacccaaactaactcccc
cgggtccgcctcatcagtcgctagccaatctattattgcgtacacaatgtccctgggagctgaaaacagcgtggcctactcaaataaca
gcattgcaatacccacaaattttacgatttcagtaaccactgaaatcctgcccgtctccatgaccaaaacctctgtcgactgcactatgtac
atatgcggcgactccaccgagtgttccaatctccttctccaatatggaagtttctgcacgcagttgaacagggcacttacagggattgca
gtcgagcaagacaagaacacccaagaagtattcgcacaagtaaaacagatctacaagacacccccaatcaaagattttggtggcttca
acttctcccaaatacttccagatccgtcaaagccatccaaacgctcattcatcgaagaccttctgttcaataaggtcacattggcggatgc
tggatttatcaagcaatatggggattgtttgggagatattgcagcgcgggacctgatatgcgcgcaaaagttcaatgggttgacggtgct
gccccctctcctcactgacgagatgatagctcagtatacgagcgctctcctcgcgggcactatcacctcaggttggaccttcggggctg
gcgcggcacttcaaataccatttgctatgcaaatggcctatcgttttaatggcatcggggtgacccaaaacgtgctctatgaaaaccaga
aactgatagctaatcaattcaatagtgccatcggcaaaatccaggattcattgtccagcaccgcctcagctctcgggaaattgcaagac
gtcgtcaaccaaaatgctcaagcgctcaacaccctcgttaaacaactctcaagtaatttcggcgcgattagtagcgtgctgaacgatatc
ttgagtcgtcttgatccacctgaagcagaagtccaaatcgacaggcttattaccggacgtctgcaaagcctgcaaacctacgttacaca
acaacttataagggcagccgaaataagggcttctgcaaatctggctgccacgaagatgagcgagtgtgtcctcggacaaagcaaaag
agttgacttttgcggcaaagggtaccaccttatgagtttccctcagtctgcgccccatggagtagtgtttctccacgtgacttatgtaccgg
cacaagaaaagaactttaccacagccccagcaatatgtcacgatggaaaagcacactttccacgggaaggggttttcgtgtccaacgg
gacccattggtttgttactcaacgcaacttttatgaaccccaaatcataaccactgataatacatttgtctctgggaactgtgatgtcgtgat
cggaatagtcaacaacacagtgtatgatccgttgcaaccagagctggattccttcaaagaagaactcgacaagtattttaagaatcacac
atcaccggacgtggatcttggagacatatcaggcataaacgctagtgtggtgaatatccaaaaggagatcgacaggcttaacgaagtt
gcaaagaacctcaatgaatctcttatcgatttgcaagaattgggcaaatacgagcaaggctacattcctgaagcaccacgggacgggc
aagcttacgtgcggaaagatggcgaatgggtgctcttgagtacctttctgggttgataa (SEQ ID NO: 119)
gccaccauggacuggaccuggauacucuuucucguagcagcagccacacgagugcauucaauguuuguguuucuugucc
uguugccauuggugagcucccaaugugucaaucugaccacccggacacaauugcccccugcauauacaaauucauucacg
agaggaguauacuaucccgacaaaguuuuccgguccucaguccuucauuccacucaagaucuuuuccuuccauucuuuu
cuaacguaaccugguuccaugcaauucaugucagugggaccaacggcacgaaacgguuugauaauccaguucuuccauu
caaugacggaguauauuuugcaucaacugagaaaucuaauaucauuagaggguggauuuucggaacaacucuugacucc
aagacccaauccuugcucaucguuaacaaugcuacaaaugugguuauuaaggucugugaguuucaauucuguaacgacc
ccuuucucggcguauacuaccauaagaauaauaagucuuggauggagucugaauuucgugucuacucaucagcgaacaa
uuguacauuugaauaugugucccaaccauuccugauggaucucgaaggaaagcagggcaauuuuaagaaccuucgggag
uucgucuuuaagaauaucgauggauacuuuaaaauauauaguaaacacacaccaaucaaucugguccgagaucuccccca
ggguuuuagugcucuggagccgcugguggauuugcccaucgguaucaauauuacgcgcuuccaaacauugcucgcccug
caucggaguuaccuuacgccuggcgacaguagcagcggauggaccgcuggagccgccgccuacuaugucggguaccuuc
aaccacgcacuuuucuccugaaauacaacgaaaaugggacaauuacagacgcuguugauugcgcacucgauccccuguca
gaaacaaaauguacacuuaaaucuuuuacggucgagaaagggauuuaccagacaucuaacuuccgaguacaaccaaccga
aucuauagugcgguuccccaacauuacgaaccugugcccguucggcgaaguguucaacgcaacacgauuugcuucuguu
uacgcuuggaaccggaaacgcaucuccaauugcgucgccgauuacagcguucuuuauaauucugcaucuuucuccaccu
ucaaaugcuauggugucucucccacaaaacucaaugaccucuguuuuaccaauguguaugcggacuccuucgucauacg
cggcgacgaggugagacaaaucgcaccagggcagacuggcaagaucgcugauuauaauuacaaacugccugaugauuuu
accggaugcguuauugcuuggaauucuaauaaccucgauuccaaaguuggcgggaacuacaauuaccucuaccgauugu
uucgcaaaucuaaccuuaagccguuugagagagauaucagcacagagauuuaucaagcuggcucuaccccuugcaaugga
guagaaggcuuuaacugcuauuuuccucuucagucuuauggauuucaaccuaccaacgggguaggguaccaacccuaua
gagucgucgugcucucauuugaacuccuucacgcccccgcuaccgugugugggccuaagaaauccacuaaucucguuaa
gaauaagugugugaauuuuaauuucaauggccugacagggaccgggguucugacugaaucuaacaagaaauuucugccg
uuccaacaauucgggcgcgauauugcagacacgaccgacgcggugcgcgauccucaaacacucgaaauccuugauaucac
uccuuguucuuucggcgguguaagcgucauuacuccuggcaccaauaccucuaaccaaguggcaguacucuaucaagau
gugaacugcacugaggucccgguugcaauacaugcggaucaacucaccccaacauggcgaguguauuccacagggagcaa
uguguuucaaacgagggccggcugucucauuggggccgaacacguuaauaauaguuaugagugcgauauucccauugga
gcgggcauuugugccagcuaucagacccaaacuaacucccccggguccgccucaucagucgcuagccaaucuauuauugc
guacacaaugucccugggagcugaaaacagcguggccuacucaaauaacagcauugcaauacccacaaauuuuacgauuu
caguaaccacugaaauccugcccgucuccaugaccaaaaccucugucgacugcacuauguacauaugcggcgacuccacc
gaguguuccaaucuccuucuccaauauggaaguuucugcacgcaguugaacagggcacuuacagggauugcagucgagc
aagacaagaacacccaagaaguauucgcacaaguaaaacagaucuacaagacacccccaaucaaagauuuugguggcuuca
acuucucccaaauacuuccagauccgucaaagccauccaaacgcucauucaucgaagaccuucuguucaauaaggucaca
uuggcggaugcuggauuuaucaagcaauauggggauuguuugggagauauugcagcgcgggaccugauaugcgcgcaa
aaguucaauggguugacggugcugcccccucuccucacugacgagaugauagcucaguauacgagcgcucuccucgcgg
gcacuaucaccucagguuggaccuucggggcuggcgcggcacuucaaauaccauuugcuaugcaaauggccuaucguuu
uaauggcaucggggugacccaaaacgugcucuaugaaaaccagaaacugauagcuaaucaauucaauagugccaucggca
aaauccaggauucauuguccagcaccgccucagcucucgggaaauugcaagacgucgucaaccaaaaugcucaagcgcuc
aacacccucguuaaacaacucucaaguaauuucggcgcgauuaguagcgugcugaacgauaucuugagucgucuugauc
caccugaagcagaaguccaaaucgacaggcuuauuaccggacgucugcaaagccugcaaaccuacguuacacaacaacuua
uaagggcagccgaaauaagggcuucugcaaaucuggcugccacgaagaugagcgaguguguccucggacaaagcaaaaga
guugacuuuugcggcaaaggguaccaccuuaugaguuucccucagucugcgccccauggaguaguguuucuccacguga
cuuauguaccggcacaagaaaagaacuuuaccacagccccagcaauaugucacgauggaaaagcacacuuuccacgggaa
gggguuuuuguccaacgggacccauugguuuguuacucaacgcaacuuuuaugaaccccaaaucauaaccacugaua
auacauuugucucugggaacugugaugucgugaucggaauagucaacaacacaguguaugauccguugcaaccagagcu
ggauuccuucaaagaagaacucgacaaguauuuuaagaaucacacaucaccggacguggaucuuggagacauaucaggca
uaaacgcuaguguggugaauauccaaaaggagaucgacaggcuuaacgaaguugcaaagaaccucaaugaaucucuuauc
gauuugcaagaauugggcaaauacgagcaaggcuacauuccugaagcaccacgggacgggcaagcuuacgugcggaaaga
uggcgaaugggugcucuugaguaccuuucuggguugauaa (SEQ ID NO: 120)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS

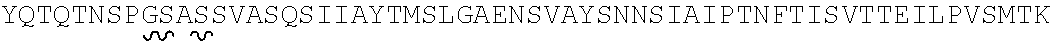

TSVDCTMYICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK

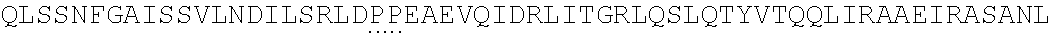

AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQGYIPEAPRDGQAYVRKDGEWVLLSTFLG** (SEQ ID NO: 121)
WuDivS_3F_D2P_Gly_pVax
ggatccgccaccatggattggacatggatattgttcttggttgcagcagctacccgggtacattccatgttcgtcttcctcgtactgctccc
acttgtcagtagtcaatgtgtgaacttgactacccggacgcagttgcccccggcctacactaatagcataacgcgtggagtctattaccc
cgacaaggtgttcaggtcatccgtcctgtatagcactcaagatctcttcttgcccttctttagtaacgtcacttggttccatgcaatccacgt
aagtggcactaatggcaccaagcgattcgacaatcccgtactcccttttaacgatggggtgtatttcgcgagcacagagaagtccaaca
tcatccgtggttggatcttcggcaccacactggattctaaaacccaaagcctgcttatagtaaataatgcaacaaacgtggtcattaaagt
ttgcgaatttcagttttgtaacgaccccttccttggcgtatattaccacaagaacaataaatcctggatggagagcgaatttagggtttaca
gttcagccaataattgtacattcgaatacgtaagccaacccttcttgatggacctggaaggaaagcaaggaaatttcaagaatctccgtg
aattcgtgttcaagaacatagacggctattttaaaatatattcaaaacacacaccgattaacctggtacgagatcttccgcaaggattctct
gcactggaaccgctggtcgatcttcctatcggcattaatatcactcggtttcaaacattgcttgctttgcatcggcgatatcttacacccgg
ggatagctcaagtggatggactgccggggctgctgcctattacgtaggctatctccaaccacggacattcctgctgaaatataacgaga
atgggacaatcacagatgctgttgactgcgctttggaccctttgagcgaaacaaagtgcacactcaaatccttcaccgtggaaaaggga
atctaccaaacgtctaattttcgcgtccaaccaaccgagagcatcgtcagattcccaaacattactaatctttgcccctttggcgaagtctt
caatgctacgcgatttgcgtccgtctacgcgtggaatcggaagcgcattagcaattgcgtcgcagactattcctttctctataactctgcat
ccttttctacctttaaatgttatggagtcaacgggacaaagctcaatgacctttgctttacaaatgtctatgcagactcttttgtcatccgtggt
gatgaggtacgacagatcgcgccaggacaaaccgggaagatcgccgattacaactacaaactgcccgacgatttcaccgggtgcgt
tattgcttggaactccaataatcttgatagtaaagttggcggcaactacaactacctgtatcgacttttccgtaaaagtaatctcaagccattt
gaaagagacatcaacacaacaatttatcaggctggatctaccccatgcaacggcgttgaaggatttaactgctacttccctctccaaagt
tacggtttccaaccaacaaacggcgttggctatcaaccttatagagtcgttgtcctctcttttgagcttaaccatgccccagcgacagtgtg
tgggccgaagaaaagcactaatttggttaagaataaatgtgttaactttaattttaatggattgacggggacaggggttctgacagagtct
aacaagaaatttctgccgttccaacagtttgggcgagatattgcagataccacggacgccgttcgagacccccaaacacttgaaattct
cgatataactccctgcagctttggcggtgtatccgttatcacgcccgggacaaataccagtaaccaagtcgcagtcctgtatcaaggcg
taaattgtacggaagtgcccgttgctatacacgctgaccaactgactcccacatggagagtctatagtactggttctaatgtgttccaaac
acgagccggttgcctgatcggagccgaacatgttaacaactcatacgaatgtgacataccgattggcgccggcatttgcgccagctat
caaacgcagaccaactcaccaagaaggcgtcgcagtgtagcaagtcaatctattatagcgtataccatgtctttgggagcagaaaactc
cgttgcttactctaataattctattgctatcccaaccaattttacaatctcagttactaccgaaatactgccggtaagcatgactaagacatcc
gtggattgcactatgtacatctgtggggactcaacagagtgtagtaatttgctgcttcaatatggctccttctgcactcaactgaatcgtgct
ctcacgggaattgctgttgagcaagataagaatacccaggaagtgtttgcccaagtcaaacaaatttataagacaccaccaattaaagat
tttggtggatttaatttcagccaaatacttccagatccctcacgcagacgacggtctttcatcgaggaccttctgttcaacaaagttactctg
gctgatgcaggcttcattaagcagtacggtgattgtcttggagacatcgctgcgcgcgacctcatatgcgcccagaaatttaatgggctg
accgtacttccccctttgctgactgatgagatgattgcacaatacacttccgcactccttgcgggtactatcacatccgggtggacttttgg
agctggcgccgctcttcaaattcccttcgccatgcaaatggcgtacaggtttaatggcatcggtgtgacacagaatgtgctctatgagaa
ccagaaacttatcgcaaaccagttcaattcagccatcgggaaaatccaagatagtctcagtagtactgcctcagctctcggcaagctcc
aggatgtagtgaatcagaatgcacaagccttgaacactctcgttaaacaactttcttccaactttggtgccatcagcagtgggcctaacg
atatattgagccgcttgcccaaagtggaagcggaagtccaaatagatagacttattaccggccggctgcaatctctgcaaacctatgtg
actcaacaattgatccgagctgccgaaatccgtgccagtgcaaatctcgccgcgaccaagatgagcgaatgtgtcttgggacagagc
aaaagagtcgatttctgcggaaaaggctaccacctgatgtctttccctcaatctgccccgcacggagtggtctttctccatgtgacttatgt
gccagcccaagaaaagaactttacaaccgcaccggcaatttgccatgacggaaaggcgcatttcccccgtgagggagtctttgtgag
caacgggacccattggttcgtgacacaacgcaatttctatgagcctcagatcattaccacggacaatactttcgtgtctggcaactgtga
cgtgctgataggcatcgtgaataataccgtctacgatcccttgcaacttgaactggactcattcaaagaagagctggataagtattttaag
aaccatacaagccctgatgtcgatcttggggatatatcaggcataaacgcatctgttgtgaatatccaaaaggaaattgatagattgaac
gaagttgccaagaacctcaatgaaagtcttatcgacctgcaagaactgggaaaatatgagcaatatataaaatggccatggagcgggc
gccggagacggagaaggggtagcggcggtagtggtagcgggtacatcccagaggcacccagagatggacaagcttacgtaagga
aggacggggaatgggtgctgctcagtacatttcttggatgataa (SEQ ID NO: 122)
ggauccgccaccauggauuggacauggauauuguucuugguugcagcagcuacccggguacauuccauguucgucuucc
ucguacugcucccacuugucaguagucaaugugugaacuugacuacccggacgcaguugcccccggccuacacuaauagc
auaacgcguggagucuauuaccccgacaagguguucaggucauccguccuguauagcacucaagaucucuucuugcccu
ucuuuaguaacgucacuugguuccaugcaauccacguaaguggcacuaauggcaccaagcgauucgacaaucccguacuc
ccuuuuaacgaugggguguauuucgcgagcacagagaaguccaacaucauccgugguuggaucuucggcaccacacugg
auucuaaaacccaaagccugcuuauaguaaauaaugcaacaaacguggucauuaaaguuugcgaauuucaguuuuguaac
gaccccuuccuuggcguauauuaccacaagaacaauaaauccuggauggagagcgaauuuaggguuuacaguucagccaa
uaauuguacauucgaauacguaagccaacccuucuugauggaccuggaaggaaagcaaggaaauuucaagaaucuccgug
aauucguguucaagaacauagacggcuauuuuaaaauauauucaaaacacacaccgauuaaccugguacgagaucuuccg
caaggauucucugcacuggaaccgcuggucgaucuuccuaucggcauuaauaucacucgguuucaaacauugcuugcuu
ugcaucggcgauaucuuacacccggggauagcucaaguggauggacugccggggcugcugccuauuacguaggcuaucu
ccaaccacggacauuccugcugaaauauaacgagaaugggacaaucacagaugcuguugacugcgcuuuggacccuuuga
gcgaaacaaagugcacacucaaauccuucaccguggaaaagggaaucuaccaaacgucuaauuuucgcguccaaccaaccg
agagcaucgucagauucccaaacauuacuaaucuuugccccuuuggcgaagucuucaaugcuacgcgauuugcguccgu
cuacgcguggaaucggaagcgcauuagcaauugcgucgcagacuauuccuuucucuauaacucugcauccuuuucuacc
uuuaaauguuauggagucaacgggacaaagcucaaugaccuuugcuuuacaaaugucuaugcagacucuuuugucaucc
guggugaugagguacgacagaucgcgccaggacaaaccgggaagaucgccgauuacaacuacaaacugcccgacgauuuc
accgggugcguuuuuuggaacuccaauaaucuugauaguaaaguuggcggcaacuacaacuaccuguaucgacuuu
uccguaaaaguaaucucaagccauuugaaagagacaucaacacaacaauuuaucaggcuggaucuaccccaugcaacggc
guugaaggauuuaacugcuacuucccucuccaaaguuacgguuuccaaccaacaaacggcguuggcuaucaaccuuauag
agucuuguccucucuuuugagcuuaaccaugccccagcgacagugugugggccgaagaaaagcacuaauuugguuaag
aauaaauguguuaacuuuaauuuuaauggauugacggggacagggguucugacagagucuaacaagaaauuucugccgu
uccaacaguuugggcgagauauugcagauaccacggacgccguucgagacccccaaacacuugaaauucucgauauaacu
cccugcagcuuuggcgguguauccguuaucacgcccgggacaaauaccaguaaccaagucgcaguccuguaucaaggcg
uaaauuguacggaagugcccguugcuauacacgcugaccaacugacucccacauggagagucuauaguacugguucuaa
uguguuccaaacacgagccgguugccugaucggagccgaacauguuaacaacucauacgaaugugacauaccgauuggc
gccggcauuugcgccagcuaucaaacgcagaccaacucaccaagaaggcgucgcaguguagcaagucaaucuauuauagc
guauaccaugucuuugggagcagaaaacuccguugcuuacucuaauaauucuauugcuaucccaaccaauuuuacaauc
ucaguuacuaccgaaauacugccgguaagcaugacuaagacauccguggauugcacuauguacaucuguggggacucaa
cagaguguaguaauuugcugcuucaauauggcuccuucugcacucaacugaaucgugcucucacgggaauugcuguuga
gcaagauaagaauacccaggaaguguuugcccaagucaaacaaauuuauaagacaccaccaauuaaagauuuugguggau
uuaauuucagccaaauacuuccagaucccucacgcagacgacggucuuucaucgaggaccuucuguucaacaaaguuacu
cuggcugaugcaggcuucauuaagcaguacggugauugucuuggagacaucgcugcgcgcgaccucauaugcgcccaga
aauuuaaugggcugaccguacuucccccuuugcugacugaugagaugauugcacaauacacuuccgcacuccuugcggg
uacuaucacauccggguggacuuuuggagcuggcgccgcucuucaaauucccuucgccaugcaaauggcguacagguuu
aauggcaucggugugacacagaaugugcucuaugagaaccagaaacuuaucgcaaaccaguucaauucagccaucgggaa
aauccaagauagucucaguaguacugccucagcucucggcaagcuccaggauguagugaaucagaaugcacaagccuuga
acacucucguuaaacaacuuucuuccaacuuuggugccaucagcagugggccuaacgauauauugagccgcuugcccaaa
guggaagcggaaguccaaauagauagacuuauuaccggccggcugcaaucucugcaaaccuaugugacucaacaauugau
ccgagcugccgaaauccgugccagugcaaaucucgccgcgaccaagaugagcgaaugugucuugggacagagcaaaagag
ucgauuucugcggaaaaggcuaccaccugaugucuuucccucaaucugccccgcacggaguggucuuucuccaugugac
uuaugugccagcccaagaaaagaacuuuacaaccgcaccggcaauuugccaugacggaaaggcgcauuucccccgugagg
gagucuuugugagcaacgggacccauugguucgugacacaacgcaauuucuaugagccucagaucauuaccacggacaa
uacuuucgugucuggcaacugugacgugcugauaggcaucgugaauaauaccgucuacgaucccuugcaacuugaacug
gacucauucaaagaagagcuggauaaguauuuuaagaaccauacaagcccugaugucgaucuuggggauauaucaggca
uaaacgcaucuguugugaauauccaaaaggaaauugauagauugaacgaaguugccaagaaccucaaugaaagucuuauc
gaccugcaagaacugggaaaauaugagcaauauauaaaauggccauggagcgggcgccggagacggagaagggguagcg
gcgguagugguagcggguacaucccagaggcacccagagauggacaagcuuacguaaggaaggacggggaaugggugcu
gcucaguacauuucuuggaugauaa (SEQ ID NO: 123)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR

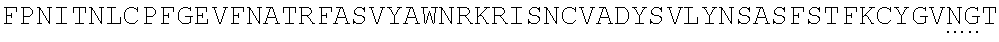

KLNDLCFTNVYADSFVIRGDEVRQIAPGQTGKIADYNYKLPDDFTGCVIAWNSNNLD

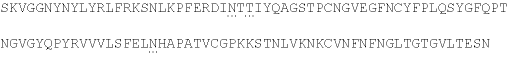

KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA

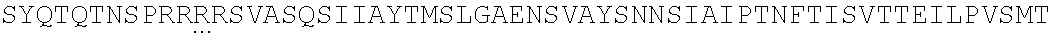

KTSVDCTMYICGDSTECSNLLLQYGSFCTQLNRALTGIAVEQDKNTQEVFAQVKQIY

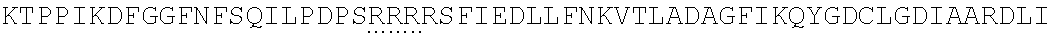

CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK

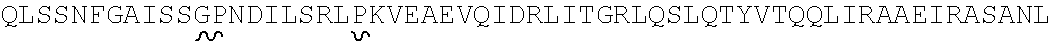

AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 124)
WuS_3F_2P_NoTri_pVax
ggatccgccaccatggactggacgtggattttgtttcttgtcgctgcagctacccgggttcactccatgtttgttttcctggtgctccttccc
cttgtaagctcacaatgcgtcaatttgaccacgcgtacacaactgcccccagcatatactaattctttcacacgcggggtctactatcccg
ataaagtctttagaagtagcgtacttcatagtacccaagatttgtttctgcccttcttcagcaatgtcacgtggtttcatgcgatccatgtatc
cggcacgaacgggacaaaacgatttgataaccccgtgctccccttcaacgacggggtttatttcgccagcaccgagaaatcaaatatta
tcaggggctggattttcgggacaacacttgattccaagacacaatctcttcttatcgtgaataatgcaactaatgtggtgatcaaggtttgc
gagttccaattttgtaatgacccttttcttggcgtgtactatcataagaataacaagagttggatggaatcagagttccgggtctacagcag
tgctaacaattgtacgtttgaatacgtttctcagccttttctgatggaccttgaaggtaagcaaggcaatttcaagaacttgcgggaatttgt
cttcaagaacatagatggctattttaagatatatagcaaacacactcccataaatctcgtcagagatcttccacagggctttagcgccctg
gaaccattggttgatttgccaattggaataaacataactcgattccaaaccttgctcgcactccatcggagctacctgacgcctggagatt
cctcttccggctggactgccggagcagcagcttattatgtaggctacttgcaaccccgcacgttcctgctcaaatataatgaaaatggca
ctataaccgatgcggtagactgcgctcttgatcccctgagtgaaactaaatgtacgttgaaaagctttactgtagagaaaggcatctatca
gactagtaactttagggtgcaacccacggagtccattgtacggttcccaaacattaccaacctctgtccattcggagaagtgtttaatgcc
acaagattcgcttcagtgtatgcctggaaccggaaacgcatctcaaattgcgttgccgattattcagtactttacaactcagccagtttctct
acttttaagtgctatggcgtttccccgacgaagctcaatgatctgtgctttactaacgtttacgcagactctttcgtcatcagaggcgatgaa
gtcaggcaaatagctcctggtcaaaccggcaagatcgccgactacaactataaactgcccgatgatttcactgggtgtgtgatcgcgtg
gaattccaataatttggactctaaggtaggtggcaactataactacctctatcgactcttccgaaaatccaaccttaagccgtttgaacgcg
atattagtaccgaaatataccaagccgggtctacaccctgtaacggcgttgaaggtttcaattgttactttccactgcagagttatggctttc
aacccaccaacggggttggctatcagccctatagggttgtggtcctcagttttgagcttctgcatgcaccagcaaccgtgtgcggacct
aagaagtcaacaaatctcgtgaagaacaagtgtgttaatttcaatttcaatggccttacagggaccggagtgcttacagaaagcaataag
aagttcttgccctttcaacagttcggcagggacatagcggacacgacagatgcagttcgagacccgcaaactctcgaaattctggatat
cacaccttgcagttttggtggcgtgtctgttatcacaccaggcaccaacacttccaaccaggtggcagttttgtaccaggatgttaattgta
cagaggtcccagtggcaatacacgctgaccaactgactccaacttggagagtctactctacaggctcaaacgtcttccaaacacgggc
ggggtgtctgatcggagcagaacacgttaataacagttacgagtgtgatatcccgataggagctggtatttgcgcttcataccagacgc
aaacgaactcaccacgaagacgccggtcagttgcatcacaatccattattgcatacaccatgtcactcggagcggagaattctgtagca
tacagtaacaatagtatcgcaatacctacgaactttaccatttccgtcacaactgaaatcttgcccgtctcaatgacaaagacaagcgtag
attgtacaatgtatatttgcggagattcaacagagtgctccaacctgctgctccagtacggtagtttctgtacccagctcaatagggccct
caccggaattgcagttgaacaagacaagaacacccaagaagtgtttgcacaagtcaaacaaatctataaaacacccccaatcaaagat
ttcggtggcttcaacttttcacaaattctccctgatcctagccgccgccgcagatcattcatcgaagacttgctcttcaataaggttaccctg
gcagacgccggttttattaaacaatacggagattgcctcggtgacatcgccgctagagaccttatctgtgcccaaaagttcaacggact
caccgtgctgcccccattgctgaccgatgaaatgattgctcaatatacatctgcgctcctcgcagggaccattacttcagggtggactttt
ggggctggcgccgcattgcagattcccttcgccatgcagatggcatataggtttaacggcattggagttacccaaaatgtactctacga
gaaccaaaagctgattgcaaatcagttcaacagtgcaataggcaaaatacaagactctctgtcttcaaccgccagcgctcttggaaagc
tccaagatgttgttaatcaaaatgcccaagcgttgaataccctcgtgaagcaactctccagcaattttggtgccatctctagcgtgctgaa
cgacattctgtcacggctcgatcccccggaagccgaggtacaaattgaccgattgataaccgggcgactccaaagccttcagacctac
gttacacaacagctcattcgcgctgcagaaattagagcctctgcaaatcttgcagctacaaagatgtcagagtgcgttctcggtcaaagc
aaaagagtggatttctgcggaaaggggtaccacctcatgagtttcccacagagtgcccctcatggcgtagtctttcttcatgttacttatgt
accagcccaagaaaagaatttcactacagcacccgcgatttgtcatgatggcaaagcgcacttccctcgggaaggcgtgttcgtgtcta
atggaacacattggttcgtgacgcaacggaatttctacgagccccaaattatcactactgataacaccttcgtctccggaaactgcgatgt
tgttattggcattgtcaacaataccgtttacgacccgctccaacctgagctggattcatttaaagaggaattggacaaatattttaagaatca
tacctctccagacgtggatttgggtgacattagcggaataaatgcatctgtggtcaatatccaaaaggaaattgataggctgaacgaggt
cgccaagaatttgaacgaatctttgattgatcttcaagaacttggcaagtatgaacaatacataaaatggccctggtgatag (SEQ ID
NO: 125)
ggauccgccaccauggacuggacguggauuuuguuucuugucgcugcagcuacccggguucacuccauguuuguuuuc
cuggugcuccuuccccuuguaagcucacaaugcgucaauuugaccacgcguacacaacugcccccagcauauacuaauuc
uuucacacgcggggucuacuaucccgauaaagucuuuagaaguagcguacuucauaguacccaagauuuguuucugccc
uucuucagcaaugucacgugguuucaugcgauccauguauccggcacgaacgggacaaaacgauuugauaaccccgugc
uccccuucaacgacgggguuuauuucgccagcaccgagaaaucaaauauuaucaggggcuggauuuucgggacaacacu
ugauuccaagacacaaucucuucuuaucgugaauaaugcaacuaauguggugaucaagguuugcgaguuccaauuuugu
aaugacccuuuucuuggcguguacuaucauaagaauaacaagaguuggauggaaucagaguuccgggucuacagcagug
cuaacaauuguacguuugaauacguuucucagccuuuuucugauggaccuugaagguaagcaaggcaauuucaagaacuu
gcgggaauuugucuucaagaacauagauggcuauuuuaagauauauagcaaacacacucccauaaaucucgucagagauc
uuccacagggcuuuagcgcccuggaaccauugguugauuugccaauuggaauaaacauaacucgauuccaaaccuugcu
cgcacuccaucggagcuaccugacgccuggagauuccucuuccggcuggacugccggagcagcagcuuauuauguaggc
uacuugcaaccccgcacguuccugcucaaauauaaugaaaauggcacuauaaccgaugcgguagacugcgcucuugaucc
ccugagugaaacuaaauguacguugaaaagcuuuacuguagagaaaggcaucuaucagacuaguaacuuuagggugcaa
cccacggaguccauuguacgguucccaaacauuaccaaccucuguccauucggagaaguguuuaaugccacaagauucgc
uucaguguaugccuggaaccggaaacgcaucucaaauugcguugccgauuauucaguacuuuacaacucagccaguuuc
ucuacuuuuaagugcuauggcguuuccccgacgaagcucaaugaucugugcuuuacuaacguuuacgcagacucuuucg
ucaucagaggcgaugaagucaggcaaauagcuccuggucaaaccggcaagaucgccgacuacaacuauaaacugcccgau
gauuucacugggugugugaucgcguggaauuccaauaauuuggacucuaagguagguggcaacuauaacuaccucuauc
gacucuuccgaaaauccaaccuuaagccguuugaacgcgauauuaguaccgaaauauaccaagccgggucuacacccugu
aacggcguugaagguuucaauuguuacuuuccacugcagaguuauggcuuucaacccaccaacgggguuggcuaucagc
ccuauaggguugugguccucaguuuugagcuucugcaugcaccagcaaccgugugcggaccuaagaagucaacaaaucu
cgugaagaacaaguguguuaauuucaauuucaauggccuuacagggaccggagugcuuacagaaagcaauaagaaguuc
uugcccuuucaacaguucggcagggacauagcggacacgacagaugcaguucgagacccgcaaacucucgaaauucugga
uaucacaccuugcaguuuugguggcgugucuguuaucacaccaggcaccaacacuuccaaccagguggcaguuuuguac
caggauguuaauuguacagaggucccaguggcaauacacgcugaccaacugacuccaacuuggagagucuacucuacagg
cucaaacgucuuccaaacacgggggggugucugaucggagcagaacacguuaauaacaguuacgagugugauaucccg
auaggagcugguauuugcgcuucauaccagacgcaaacgaacucaccacgaagacgccggucaguugcaucacaauccau
uauugcauacaccaugucacucggagcggagaauucuguagcauacaguaacaauaguaucgcaauaccuacgaacuuua
ccauuuccgucacaacugaaaucuugcccgucucaaugacaaagacaagcguagauuguacaauguauauuugcggagau
ucaacagagugcuccaaccugcugcuccaguacgguaguuucuguacccagcucaauagggcccucaccggaauugcagu
ugaacaagacaagaacacccaagaaguguuugcacaagucaaacaaaucuauaaaacacccccaaucaaagauuucggugg
cuucaacuuuucacaaauucucccugauccuagccgccgccgcagaucauucaucgaagacuugcucuucaauaagguua
cccuggcagacgccgguuuuauuaaacaauacggagauugccucggugacaucgccgcuagagaccuuaucugugccca
aaaguucaacggacucaccgugcugcccccauugcugaccgaugaaaugauugcucaauauacaucugcgcuccucgcag
ggaccauuacuucaggguggacuuuuggggcuggcgccgcauugcagauucccuucgccaugcagauggcauauaggu
uuaacggcauuggaguuacccaaaauguacucuacgagaaccaaaagcugauugcaaaucaguucaacagugcaauaggc
aaaauacaagacucucugucuucaaccgccagcgcucuuggaaagcuccaagauguuguuaaucaaaaugcccaagcguu
gaauacccucgugaagcaacucuccagcaauuuuggugccaucucuagcgugcugaacgacauucugucacggcucgau
cccccggaagccgagguacaaauugaccgauugauaaccgggcgacuccaaagccuucagaccuacguuacacaacagcu
cauucgcgcugcagaaauuagagccucugcaaaucuugcagcuacaaagaugucagagugcguucucggucaaagcaaaa
gaguggauuucugcggaaagggguaccaccucaugaguuucccacagagugccccucauggcguagucuuucuucaugu
uacuuauguaccagcccaagaaaagaauuucacuacagcacccgcgauuugucaugauggcaaagcgcacuucccucggg
aaggcguguucgugucuaauggaacacauugguucgugacgcaacggaauuucuacgagccccaaauuaucacuacuga
uaacaccuucgucuccggaaacugcgauguuguuauuggcauugucaacaauaccguuuacgacccgcuccaaccugagc
uggauucauuuaaagaggaauuggacaaauauuuuaagaaucauaccucuccagacguggauuugggugacauuagcgg
aauaaaugcaucuguggucaauauccaaaaggaaauugauaggcugaacgaggucgccaagaauuugaacgaaucuuug
auugaucuucaagaacuuggcaaguaugaacaauacauaaaauggcccuggugauag (SEQ ID NO: 126)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS

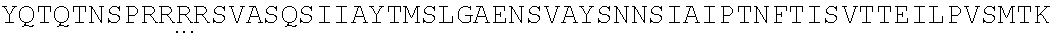

TSVDCTMYICGDSTECSNLLLQYGSFCTQLNRALTGIAVEQDKNTQEVFAQVKQIYK

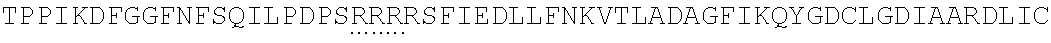

AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ

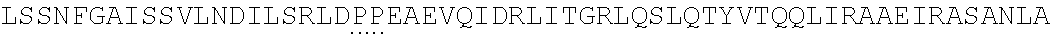

ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPW** (SEQ ID NO: 127)
WuS_3F_D2P_Gly_pVax
ggatccgccaccatggattggacatggatattgttcttggttgcagcagctacccgggtacattccatgttcgtcttcctcgtactgctccc
acttgtcagtagtcaatgtgtgaacttgactacccggacgcagttgcccccggcctacactaatagctttacgcgtggagtctattacccc
gacaaggtgttcaggtcatccgtcctgcatagcactcaagatctcttcttgcccttctttagtaacgtcacttggttccatgcaatccacgta
agtggcactaatggcaccaagcgattcgacaatcccgtactcccttttaacgatggggtgtatttcgcgagcacagagaagtccaacat
catccgtggttggatcttcggcaccacactggattctaaaacccaaagcctgcttatagtaaataatgcaacaaacgtggtcattaaagtt
tgcgaatttcagttttgtaacgaccccttccttggcgtatattaccacaagaacaataaatcctggatggagagcgaatttagggtttacag
ttcagccaataattgtacattcgaatacgtaagccaacccttcttgatggacctggaaggaaagcaaggaaatttcaagaatctccgtga
attcgtgttcaagaacatagacggctattttaaaatatattcaaaacacacaccgattaacctggtacgagatcttccgcaaggattctctg
cactggaaccgctggtcgatcttcctatcggcattaatatcactcggtttcaaacattgcttgctttgcatcggagttatcttacacccgggg
atagctcaagtggatggactgccggggctgctgcctattacgtaggctatctccaaccacggacattcctgctgaaatataacgagaat
gggacaatcacagatgctgttgactgcgctttggaccctttgagcgaaacaaagtgcacactcaaatccttcaccgtggaaaagggaat
ctaccaaacgtctaattttcgcgtccaaccaaccgagagcatcgtcagattcccaaacattactaatctttgcccctttggcgaagtcttca
atgctacgcgatttgcgtccgtctacgcgtggaatcggaagcgcattagcaattgcgtcgcagactattccgtgctctataactctgcatc
cttttctacctttaaatgttatggagtcaacgggacaaagctcaatgacctttgctttacaaatgtctatgcagactcttttgtcatccgtggtg
atgaggtacgacagatcgcgccaggacaaaccgggaagatcgccgattacaactacaaactgcccgacgatttcaccgggtgcgtta
ttgcttggaactccaataatcttgatagtaaagttggcggcaactacaactacctgtatcgacttttccgtaaaagtaatctcaagccatttg
aaagagacatcaacacaacaatttatcaggctggatctaccccatgcaacggcgttgaaggatttaactgctacttccctctccaaagtta
cggtttccaaccaacaaacggcgttggctatcaaccttatagagtcgttgtcctctcttttgagcttaaccatgccccagcgacagtgtgt
gggccgaagaaaagcactaatttggttaagaataaatgtgttaactttaattttaatggattgacggggacaggggttctgacagagtcta
acaagaaatttctgccgttccaacagtttgggcgagatattgcagataccacggacgccgttcgagacccccaaacacttgaaattctc
gatataactccctgcagctttggcggtgtatccgttatcacgcccgggacaaataccagtaaccaagtcgcagtcctgtatcaagacgta
aattgtacggaagtgcccgttgctatacacgctgaccaactgactcccacatggagagtctatagtactggttctaatgtgttccaaacac
gagccggttgcctgatcggagccgaacatgttaacaactcatacgaatgtgacataccgattggcgccggcatttgcgccagctatca
aacgcagaccaactcaccaagaaggcgtcgcagtgtagcaagtcaatctattatagcgtataccatgtctttgggagcagaaaactcc
gttgcttactctaataattctattgctatcccaaccaattttacaatctcagttactaccgaaatactgccggtaagcatgactaagacatccg
tggattgcactatgtacatctgtggggactcaacagagtgtagtaatttgctgcttcaatatggctccttctgcactcaactgaatcgtgctc
tcacgggaattgctgttgagcaagataagaatacccaggaagtgtttgcccaagtcaaacaaatttataagacaccaccaattaaagatt
ttggtggatttaatttcagccaaatacttccagatccctcacgcagacgacggtctttcatcgaggaccttctgttcaacaaagttactctg
gctgatgcaggcttcattaagcagtacggtgattgtcttggagacatcgctgcgcgcgacctcatatgcgcccagaaatttaatgggctg
accgtacttccccctttgctgactgatgagatgattgcacaatacacttccgcactccttgcgggtactatcacatccgggtggacttttgg
agctggcgccgctcttcaaattcccttcgccatgcaaatggcgtacaggtttaatggcatcggtgtgacacagaatgtgctctatgagaa
ccagaaacttatcgcaaaccagttcaattcagccatcgggaaaatccaagatagtctcagtagtactgcctcagctctcggcaagctcc
aggatgtagtgaatcagaatgcacaagccttgaacactctcgttaaacaactttcttccaactttggtgccatcagcagtgggcctaacg
atatattgagccgcttgcccaaagtggaagcggaagtccaaatagatagacttattaccggccggctgcaatctctgcaaacctatgtg
actcaacaattgatccgagctgccgaaatccgtgccagtgcaaatctcgccgcgaccaagatgagcgaatgtgtcttgggacagagc
aaaagagtcgatttctgcggaaaaggctaccacctgatgtctttccctcaatctgccccgcacggagtggtctttctccatgtgacttatgt
gccagcccaagaaaagaactttacaaccgcaccggcaatttgccatgacggaaaggcgcatttcccccgtgagggagtctttgtgag
caacgggacccattggttcgtgacacaacgcaatttctatgagcctcagatcattaccacggacaatactttcgtgtctggcaactgtga
cgtggtcataggcatcgtgaataataccgtctacgatcccttgcaacccgaactggactcattcaaagaagagctggataagtattttaa
gaaccatacaagccctgatgtcgatcttggggatatatcaggcataaacgcatctgttgtgaatatccaaaaggaaattgatagattgaa
cgaagttgccaagaacctcaatgaaagtcttatcgacctgcaagaactgggaaaatatgagcaatatataaaatggccatggagcggg
cgccggagacggagaaggggtagcggcggtagtggtagcgggtacatcccagaggcacccagagatggacaagcttacgtaagg
aaggacggggaatgggtgctgctcagtacatttcttggatgataa (SEQ ID NO: 128)
ggauccgccaccauggauuggacauggauauuguucuugguugcagcagcuacccggguacauuccauguucgucuucc
ucguacugcucccacuugucaguagucaaugugugaacuugacuacccggacgcaguugcccccggccuacacuaauagc
uuuacgcguggagucuauuaccccgacaagguguucaggucauccguccugcauagcacucaagaucucuucuugcccu
ucuuuaguaacgucacuugguuccaugcaauccacguaaguggcacuaauggcaccaagcgauucgacaaucccguacuc
ccuuuuaacgaugggguguauuucgcgagcacagagaaguccaacaucauccgugguuggaucuucggcaccacacugg
auucuaaaacccaaagccugcuuauaguaaauaaugcaacaaacguggucauuaaaguuugcgaauuucaguuuuguaac
gaccccuuccuuggcguauauuaccacaagaacaauaaauccuggauggagagcgaauuuaggguuuacaguucagccaa
uaauuguacauucgaauacguaagccaacccuucuugauggaccuggaaggaaagcaaggaaauuucaagaaucuccgug
aauucguguucaagaacauagacggcuauuuuaaaauauauucaaaacacacaccgauuaaccugguacgagaucuuccg
caaggauucucugcacuggaaccgcuggucgaucuuccuaucggcauuaauaucacucgguuucaaacauugcuugcuu
ugcaucggaguuaucuuacacccggggauagcucaaguggauggacugccggggcugcugccuauuacguaggcuaucu
ccaaccacggacauuccugcugaaauauaacgagaaugggacaaucacagaugcuguugacugcgcuuuggacccuuuga
gcgaaacaaagugcacacucaaauccuucaccguggaaaagggaaucuaccaaacgucuaauuuucgcguccaaccaaccg
agagcaucgucagauucccaaacauuacuaaucuuugccccuuuggcgaagucuucaaugcuacgcgauuugcguccgu
cuacgcguggaaucggaagcgcauuagcaauugcgucgcagacuauuccgugcucuauaacucugcauccuuuucuacc
uuuaaauguuauggagucaacgggacaaagcucaaugaccuuugcuuuacaaaugucuaugcagacucuuuugucaucc
guggugaugagguacgacagaucgcgccaggacaaaccgggaagaucgccgauuacaacuacaaacugcccgacgauuuc
accgggugcguuauugcuuggaacuccaauaaucuugauaguaaaguuggcggcaacuacaacuaccuguaucgacuuu
uccguaaaaguaaucucaagccauuugaaagagacaucaacacaacaauuuaucaggcuggaucuaccccaugcaacggc
guugaaggauuuaacugcuacuucccucuccaaaguuacgguuuccaaccaacaaacggcguuggcuaucaaccuuauag
agucuuguccucucuuuugagcuuaaccaugccccagcgacagugugugggccgaagaaaagcacuaauuugguuaag
aauaaauguguuaacuuuaauuuuaauggauugacggggacagggguucugacagagucuaacaagaaauuucugccgu
uccaacaguuugggcgagauauugcagauaccacggacgccguucgagacccccaaacacuugaaauucucgauauaacu
cccugcagcuuuggcgguguauccguuaucacgcccgggacaaauaccaguaaccaagucgcaguccuguaucaagacgu
aaauuguacggaagugcccguugcuauacacgcugaccaacugacucccacauggagagucuauaguacugguucuaau
guguuccaaacacgagccgguugccugaucggagccgaacauguuaacaacucauacgaaugugacauaccgauuggcgc
cggcauuugcgccagcuaucaaacgcagaccaacucaccaagaaggcgucgcaguguagcaagucaaucuauuauagcgu
auaccaugucuuugggagcagaaaacuccguugcuuacucuaauaauucuauugcuaucccaaccaauuuuacaaucuca
guuacuaccgaaauacugccgguaagcaugacuaagacauccguggauugcacuauguacaucuguggggacucaacag
aguguaguaauuugcugcuucaauauggcuccuucugcacucaacugaaucgugcucucacgggaauugcuguugagca
agauaagaauacccaggaaguguuugcccaagucaaacaaauuuauaagacaccaccaauuaaagauuuugguggauuua
auuucagccaaauacuuccagaucccucacgcagacgacggucuuucaucgaggaccuucuguucaacaaaguuacucug
gcugaugcaggcuucauuaagcaguacggugauugucuuggagacaucgcugcgcgcgaccucauaugcgcccagaaau
uuaaugggcugaccguacuucccccuuugcugacugaugagaugauugcacaauacacuuccgcacuccuugcggguac
uaucacauccggguggacuuuuggagcuggcgccgcucuucaaauucccuucgccaugcaaauggcguacagguuuaau
ggcaucggugugacacagaaugugcucuaugagaaccagaaacuuaucgcaaaccaguucaauucagccaucgggaaaau
ccaagauagucucaguaguacugccucagcucucggcaagcuccaggauguagugaaucagaaugcacaagccuugaaca
cucucguuaaacaacuuucuuccaacuuuggugccaucagcagugggccuaacgauauauugagccgcuugcccaaagu
ggaagcggaaguccaaauagauagacuuauuaccggccggcugcaaucucugcaaaccuaugugacucaacaauugaucc
gagcugccgaaauccgugccagugcaaaucucgccgcgaccaagaugagcgaaugugucuugggacagagcaaaagaguc
gauuucugcggaaaaggcuaccaccugaugucuuucccucaaucugccccgcacggaguggucuuucuccaugugacuu
augugccagcccaagaaaagaacuuuacaaccgcaccggcaauuugccaugacggaaaggcgcauuucccccgugaggga
gucuuugugagcaacgggacccauugguucgugacacaacgcaauuucuaugagccucagaucauuaccacggacaauac
uuucgugucuggcaacugugacguggucauaggcaucgugaauaauaccgucuacgaucccuugcaacccgaacuggac
ucauucasagaagagcuggauaaguauuuuaagaaccauacaagcccugaugucgaucuuggggauauaucaggcauaa
acgcaucuguugugaauauccaaaaggaaauugauagauugaacgaaguugccaagaaccucaaugaaagucuuaucgac
cugcaagaacugggaaaauaugagcaauauauaaaauggccauggagcgggcgccggagacggagaagggguagcggcg
guagugguagcggguacaucccagaggcacccagagauggacaagcuuacguaaggaaggacggggaaugggugcugcu
caguacauuucuuggaugauaa (SEQ ID NO: 129)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR

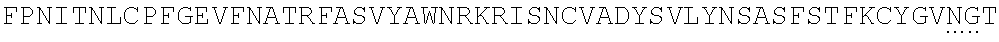

KLNDLCFTNVYADSFVIRGDEVRQIAPGQTGKIADYNYKLPDDFTGCVIAWNSNNLD

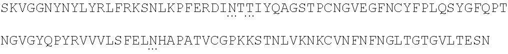

KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA

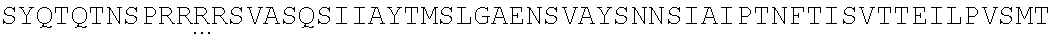

KTSVDCTMYICGDSTECSNLLLQYGSFCTQLNRALTGIAVEQDKNTQEVFAQVKQIY

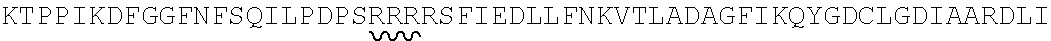

CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK

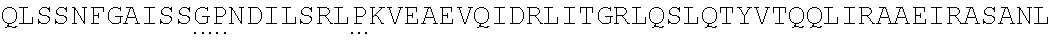

AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 130)
WuS_3F_D2P_rB_pVax
ggatccgccaccatggattggacatggattctgtttctcgttgccgccgctacgcgcgtgcacagcatgttcgttttcctcgtactgttgcc
tctggtatcatctcaatgtgtgaatcttacgacgaggacgcaactgccaccagcttacaccaactcttttactagaggggtctattacccc
gacaaggttttcagatcctcagtgctgcatagtacacaagatttgtttcttcccttcttctccaatgtcacttggtttcacgctatccacgtgtc
cggcactaatggaacgaagcggtttgataacccggtacttccatttaatgacggggtatactttgcaagcaccgagaaaagtaatatcat
tcgtgggtggatctttggcactacactcgactccaagacacaatcccttttgatagtaaataatgctacaaatgtggttataaaggtgtgcg
agtttcaattctgcaatgaccccttcctcggtgtctattatcacaagaacaataaatcttggatggaaagtgagttcagggtatacagctct
gcaaacaactgcacatttgagtatgtgagccaaccgtttcttatggatctggaaggtaagcagggtaactttaagaaccttcgggagttc
gtctttaagaatatagatggctattttaaaatctatagtaaacacactcctattaaccttgtgcgcgatctccctcaagggttctctgcattgg
aaccgcttgttgatttgcctataggaatcaatattacacgatttcaaacactcctcgctctccataggagctaccttaccccaggcgactca
agctctggttggacggcaggagctgcagcatactatgtgggttatcttcagccgcggacattcttgcttaagtataatgagaatggaact
atcactgacgctgttgactgtgccctggaccctctttcagaaacaaaatgtactctcaaatcattcaccgtggagaagggaatatatcaaa
caagtaactttagggtccaacctaccgagagcatcgtgcgattccctaatattaccaatttgtgtcccttcggtgaagtcttcaacgcgac
ccgctttgctagcgtctatgcgtggaacaggaagcgaattagcaactgtgttgcagattacagtgtgctgtacaatagtgcgagcttttcc
acgttcaaatgctatggcgtgaaacctaccaaacttaatgatttgtgcttcactaatgtttatgctgacagcttcgttatccggggtgatgag
gtgaggcagattgcccccggtcaaactggcaaaattgccgactacaattacaagctccctgatgattttactggatgtgtcatagcgtgg
aattccaataatctggactctaaggttggtggtaattataactatctctaccgcctgtttcgtaagagcaatctgaaaccctttgaaagagat
atttggactgagatatatcaagctggctcaactccttgcaacggggtcgaaggtttcaattgttactttccacttcaatcatacgggtttcaa
ccaactaacggtgtaggttatcaaccctatcgggtggttgtcctgagctttgagctgaaccatgccccggctacagtatgcggcccaaa
gaaatccactaacttggtcaagaacaaatgcgtcaactttaactttaacggactcacggggacaggagtccttaccgaatccaacaaga
aattcttgcctttccaacaatttggacgagacattgcggataccacagacgcagtacgcgacccacagactcttgaaatcctcgacataa
caccctgcagtttcggcggtgtaagtgtcattaccccaggcactaatacgagcaaccaagtggcggtgctctaccaagacgttaattgc
actgaggtcccagtggctattcacgctgaccaacttacacccacatggagagtgtatagtacaggctcaaacgtcttccagacacggg
cggggtgccttattggagcagaacatgttaacaattcctatgaatgcgatatcccgattggagccgggatctgtgctagctatcaaaccc
aaacaaatagccccagacgtcgacgttccgtggctagtcaaagcatcatcgcctacactatgagtcttggggccgaaaattccgttgctt
acagtaacaacagtatcgctatccccaccaattttactattagtgtaactacagagattctgccggtttccatgacaaagacttccgtggat
tgtacgatgtatatttgcggcgacagcacagagtgcagcaatctgctgctgcaatacggtagtttctgcacccaattgaaccgtgctctg
acgggaattgcagttgagcaggacaagaatactcaagaagtatttgcacaagtcaaacagatatacaagacgcccccgattaaagattt
cggcgggtttaactttagccaaattcttccggaccccagcagacgccgccgaagctttattgaggacctgctgtttaataaagttaccctt
gctgatgctggttttatcaagcaatacggagattgcctgggagatatcgccgccagggatttgatctgtgcgcaaaagtttaacggcctt
accgttctcccgccccttctgaccgatgaaatgatagcccaatacacttccgcactcctggcaggcacaattacttccggctggacgttt
ggggccggggcagccttgcaaattccgtttgctatgcaaatggcatatcgtttcaatggtatcggcgtaacacaaaatgtcctttatgaga
accagaaactcattgctaatcagtttaattccgctatcggcaagattcaagacagtctcagcagcacggcgagcgcacttggtaaacttc
aagacgttgtcaaccagaatgctcaagccctgaacactctggtaaaacaacttagctctaatttcggtgcaattagctccggtccgaacg
atattctgtcacggctcccgaaagtcgaagccgaagtccagatcgataggctgatcacagggcgcttgcagagtctccaaacctacgt
gacgcaacaactcattcgggggctgaaattcgtgcaagcgctaatctggccgctaccaaaatgagtgagtgtgttctcggtcaatcaa
agagggttgacttttgcggcaaaggatatcatttgatgagttttccgcaatctgcccctcatggggtagtatttctgcacgtaacttatgtac
cagcacaagaaaagaacttcaccacggccccagcaatatgccacgatggcaaagctcatttccctcgcgaaggggtctttgtaagca
atggaacccactggtttgtcacacaacgcaacttttatgagcctcaaatcattacaaccgataacacttttgtctccgggaactgcgacgt
ggtgattggaatcgtcaacaacactgtctatgatcccctgcaacctgaactggattcctttaaagaagagcttgataagtatttcaagaac
cataccagccccgacgtcgatttgggagatattagtgggattaatgctagcgttgttaatatacaaaaggaaatagatcgattgaatgaa
gtggccaagaatctgaatgagtctctgattgacctgcaggagctcggaaagtatgagcaatatataaaatggccctggtcaggccgca
ggcgtcggcggcgcggtagcggcggttcaggatctgggtatatacctgaggccccacgagatgggcaggcttatgtacggaaagat
ggagaatgggtgttgctgagtactttcctcgggtaataa (SEQ ID NO: 131)
ggauccgccaccauggauuggacauggauucuguuucucguugccgccgcuacgcgcgugcacagcauguucguuuucc
ucguacuguugccucugguaucaucucaaugugugaaucuuacgacgaggacgcaacugccaccagcuuacaccaacucu
uuuacuagaggggucuauuaccccgacaagguuuucagauccucagugcugcauaguacacaagauuuguuucuucccu
ucuucuccaaugucacuugguuucacgcuauccacguguccggcacuaauggaacgaagcgguuugauaacccgguacu
uccauuuaaugacgggguauacuuugcaagcaccgagaaaaguaauaucauucguggguggaucuuuggcacuacacuc
gacuccaagacacaucccuuuugauaguaaauaaugcuacaaaugugguuauaaaggugugcgaguuucaauucugca
augaccccuuccucggugucuauuaucacaagaacaauaaaucuuggauggaaagugaguucaggguauacagcucugc
aaacaacugcacauuugaguaugugagccaaccguuucuuauggaucuggaagguaagcaggguaacuuuaagaaccuu
cgggaguucgucuuuaagaauauagauggcuauuuuaaaaucuauaguaaacacacuccuauuaaccuugugcgcgauc
ucccucaaggguucucugcauuggaaccgcuuguugauuugccuauaggaaucaauauuacacgauuucaaacacuccu
cgcucuccauaggagcuaccuuaccccaggcgacucaagcucugguuggacggcaggagcugcagcauacuaugugggu
uaucuucagccgcggacauucuugcuuaaguauaaugagaauggaacuaucacugacgcuguugacugugcccuggacc
cucuuucagaaacaaaauguacucucaaaucauucaccguggagaagggaauauaucaaacaaguaacuuuaggguccaa
ccuaccgagagcaucgugcgauucccuaauauuaccaauuugugucccuucggugaagucuucaacgcgacccgcuuug
cuagcgucuaugcguggaacaggaagcgaauuagcaacuguguugcagauuacagugugcuguacaauagugcgagcuu
uuccacguucaaaugcuauggcgugaaaccuaccaaacuuaaugauuugugcuucacuaauguuuaugcugacagcuuc
guuauccggggugaugaggugaggcagauugcccccggucaaacuggcaaaauugccgacuacaauuacaagcucccug
augauuuuacuggaugugucauagcguggaauuccaauaaucuggacucuaagguuggugguaauuauaacuaucucu
accgccuguuucguaagagcaaucugaaacccuuugaaagagauauuuggacugagauauaucaagcuggcucaacucc
uugcaacggggucgaagguuucaauuguuacuuuccacuucaaucauacggguuucaaccaacuaacgguguagguuau
caacccuaucgggugguuguccugagcuuugagcugaaccaugccccggcuacaguaugcggcccaaagaaauccacuaa
cuuggucaagaacaaaugcgucaacuuuaacuuuaacggacucacggggacaggaguccuuaccgaauccaacaagaaau
ucuugccuuuccaacaauuuggacgagacauugcggauaccacagacgcaguacgcgacccacagacucuugaaauccuc
gacauaacacccugcaguuucggcgguguaagugucauuaccccaggcacuaauacgagcaaccaaguggcggugcucua
ccaagacguuaauugcacugaggucccaguggcuauucacgcugaccaacuuacacccacauggagaguguauaguacag
gcucaaacgucuuccagacacgggggggugccuuauuggagcagaacauguuaacaauuccuaugaaugcgauauccc
gauuggagccgggaucugugcuagcuaucaaacccaaacaaauagccccagacgucgacguuccguggcuagucaaagca
ucaucgccuacacuaugagucuuggggccgaaaauuccguugcuuacaguaacaacaguaucgcuauccccaccaauuuu
acuauuaguguaacuacagagauucugccgguuuccaugacaaagacuuccguggauuguacgauguauauuugcggcg
acagcacagagugcagcaaucugcugcugcaauacgguaguuucugcacccaauugaaccgugcucugacgggaauugc
aguugagcaggacaagaauacucaagaaguauuugcacaagucaaacagauauacaagacgcccccgauuaaagauuucg
gcggguuuaacuuuagccaaauucuuccggaccccagcagacgccgccgaagcuuuauugaggaccugcuguuuaauaa
aguuacccuugcugaugcugguuuuaucaagcaauacggagauugccugggagauaucgccgccagggauuugaucug
ugcgcaaaaguuuaacggccuuaccguucucccgccccuucugaccgaugaaaugauagcccaauacacuuccgcacucc
uggcaggcacaauuacuuccggcuggacguuuggggccggggcagccuugcaaauuccguuugcuaugcaaauggcaua
ucguuucaaugguaucggcguaacacaaaauguccuuuaugagaaccagaaacucauugcuaaucaguuuaauuccgcu
aucggcaagauucaagacagucucagcagcacggcgagcgcacuugguaaacuucaagacguugucaaccagaaugcuca
agcccugaacacucugguaaaacaacuuagcucuaauuucggugcaauuagcuccgguccgaacgauauucugucacggc
ucccgaaagucgaagccgaaguccagaucgauaggcugaucacagggcgcuugcagagucuccaaaccuacgugacgcaa
caacucauucgggcggcugaaauucgugcaagcgcuaaucuggccgcuaccaaaaugagugaguguguucucggucaau
caaagaggguugacuuuugcggcaaaggauaucauuugaugaguuuuccgcaaucugccccucaugggguaguauuuc
ugcacguaacuuauguaccagcacaagaaaagaacuucaccacggccccagcaauaugccacgauggcaaagcucauuucc
cucgcgaaggggucuuuguaagcaauggaacccacugguuugucacacaacgcaacuuuuaugagccucaaaucauuaca
accgauaacacuuuugucuccgggaacugcgacguggugauuggaaucgucaacaacacugucuaugauccccugcaacc
ugaacuggauuccuuuaaagaagagcuugauaaguauuucaagaaccauaccagccccgacgucgauuugggagauauu
agugggauuaaugcuagcguuguuaauauacaaaaggaaauagaucgauugaaugaaguggccaagaaucugaaugagu
cucugauugaccugcaggagcucggaaaguaugagcaauauauaaaauggcccuggucaggccgcaggcgucggcggcg
cgguagcggcgguucaggaucuggguauauaccugaggccccacgagaugggcaggcuuauguacggaaagauggagaa
uggguguugcugaguacuuuccucggguaauaa (SEQ ID NO: 132)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR

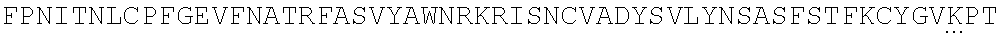

KLNDLCFTNVYADSFVIRGDEVRQIAPGQTGKIADYNYKLPDDFTGCVIAWNSNNLD

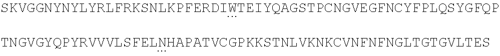

NKKFLPFQQFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECDIPIGAGIC

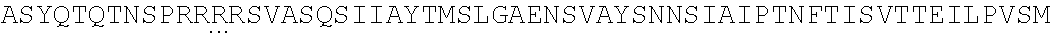

TKTSVDCTMYICGDSTECSNLLLQYGSFCTQLNRALTGIAVEQDKNTQEVFAQVKQI

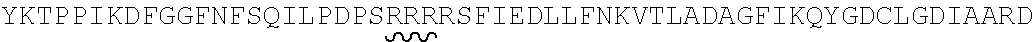

LICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTL

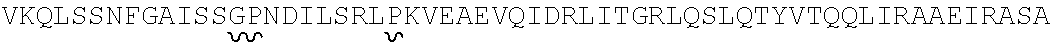

NLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNL
NESLIDLQELGKYEQYIKWPWSGRRRRRRGSGGSGSGYIPEAPRDGQAYVRKDGEW
VLLSTFLG** (SEQ ID NO: 133)
WuS_3F_D2P_pVax
ggatccgccaccatggactggacctggatactctttctcgtagcagcagccacacgagtgcattcaatgtttgtcttcttggtcctcttgcc
actggttagctcccagtgtgtgaatcttactacaaggacacaactgcccccagcttacacaaactcctttactaggggtgtatattaccca
gacaaagtgtttcgcagttctgtcctgcatagcacccaagaccttttccttccgttcttcagcaacgtcacctggttccatgctatccatgtc
agtggaacgaatggcacaaagcggttcgataaccctgtcctgccctttaacgacggcgtctatttcgcttcaacagagaagagtaacat
tatcagaggatggatatttggtacaactcttgatagcaagacacaaagcctgctgattgtaaacaacgcgacaaatgtcgtcatcaaggt
ttgcgagtttcaattttgcaacgatcccttcttgggcgtgtactatcataagaacaataaaagctggatggagagcgaatttagggtgtata
gctcagctaataactgtacatttgaatatgtctctcaacccttcctcatggaccttgagggaaagcaaggaaatttcaagaatctcagaga
atttgtcttcaagaacatcgacgggtatttcaagatctactccaagcatacacccatcaacttggttagggaccttccgcaaggtttctcag
cactggagcctctggtagatctccctattgggattaatattacaagatttcaaacactcctggccttgcatagatcctatcttacccctggg
gattccagctcaggttggaccgcgggtgccgcggcgtactatgtcggatatctccaacctcggacattcctgctgaaatacaatgaaaa
tgggaccatcactgatgccgttgattgtgctctcgatcctctgagtgagaccaaatgcactcttaagagttttacagtggagaaaggtatc
tatcaaactagtaatttcagagttcaaccaaccgagtcaatagtgcgttttccaaatatcactaatctgtgtccatttggggaagtcttcaat
gctacccgattcgcaagtgtgtacgcctggaaccggaaacggatttctaactgcgttgccgattatagtgtcctctataattctgcttctttc
tctacttttaagtgctatggggtgtcccccaccaagctgaacgatctgtgtttcactaacgtctacgccgatagttttgtcattagagggga
cgaggtacggcaaatcgcgcccggccaaacggggaaaattgccgattacaactacaagcttccagacgacttcacaggttgcgtgat
tgcatggaattctaataatctggacagtaaagtgggcggcaactataactatctttaccggctgtttcggaagagcaacttgaagcccttc
gaacgcgacatatccaccgagatctatcaagccggaagtaccccgtgcaacggggtagaaggatttaattgttattttccattgcagtctt
atggatttcagcccaccaatggtgtgggataccaaccttatagggttgttgttctctccttcgaactcctgcacgctccagctactgtatgt
gggcctaagaaaagtactaatctcgttaagaataaatgcgtcaatttcaatttcaacggcttgaccgggactggagtgctcaccgaaag
caacaagaagtttctcccgtttcagcaattcggtagggatattgccgatacgacagatgcagtacgagatccccaaacactcgaaatcc
tggacattacgccatgtagctttggcggagtaagtgtcatcaccccagggactaacaccagtaaccaagttgcggtactctatcaggat
gtgaactgcactgaggtacctgtagcaattcacgcagaccaattgacgccgacgtggcgcgtctatagtacaggaagtaacgtctttca
gacaagagcgggttgtttgattggcgctgaacacgttaacaattcttacgagtgtgatatccccatcggtgcggggatctgcgccagct
atcagacacaaaccaattccccacgaaggagacgttccgtggccagccagtcaataatcgcgtatactatgtctctgggtgcggagaa
ttcagtggcctattccaataattctatagccattccaaccaattttactataagcgtcactacagagatcttgccagttagcatgacgaaaac
cagcgtcgattgtaccatgtatatatgcggcgacagtaccgaatgctcaaatctgctgctccaatatggctcattttgcactcaacttaata
gagctctgacagggatcgctgtcgaacaagataagaacactcaggaagttttcgcccaagttaagcagatatacaagaccccgcccat
caaggattttggcggatttaatttctctcagatcctgccggaccctagccgccgacgccggagctttatcgaagacttgctgtttaataag
gttactctcgcagatgcaggcttcatcaagcaatacggtgactgccttggggatatcgctgctcgggacctgatctgtgctcagaaattc
aacggtctcacggtgctgcccccactcctgaccgacgaaatgattgcccagtatacgtccgcattgctcgctggcaccatcactagcg
gctggacctttggggccggagccgcgctccaaataccttttgctatgcaaatggcttatcgcttcaatggtattggggttacgcaaaatgt
cctctacgaaaatcaaaagctcatagctaaccaattcaatagcgctatagggaaaattcaagacagcctgagttccacagcaagcgcc
ctcggcaaacttcaagatgtagtgaaccaaaatgctcaagcactcaatacactggtcaaacaactctcaagcaatttcggggcaatctc
atctggtcctaatgacatattgagcaggctccccaaagtggaagcagaagtacaaatcgacaggctgattaccggacgactccaaagc
ttgcaaacttatgtaacccaacaacttatcagggctgcagaaatccgtgcaagcgctaacctcgccgctacgaagatgtcagaatgtgt
acttgggcagtctaagagggttgatttctgtggaaaagggtaccatctgatgagttttccacagagcgctccacatggggtggtgtttctg
catgtaacctatgttcccgctcaagaaaagaattttactactgcccccgcaatttgccatgacgggaaagcccatttcccccgagaggg
agttttcgtgagtaacggaacgcactggtttgtcactcagagaaatttctacgagccccaaatcattacgaccgataatacattcgtaagc
ggtaactgcgatgtcgtcattggcatcgttaacaacactgtttatgatccccttcaacccgagcttgactcatttaaagaggaactggataa
gtactttaagaatcacacctctcccgatgtcgacctgggcgacatctctggaattaatgcctctgtcgtaaacatccaaaaggaaattgac
cgactgaatgaggtggcaaagaatcttaatgaatccctgatcgatctgcaggagcttgggaagtatgagcaatacatcaaatggccatg
gtctggcagacggcgccggagaaggggctctggcggctctggaagcgggtatattccagaggcgcccagggatgggcaagcatat
gttcggaaggatggggagtgggtgttgttgtccacgttccttggctagtga (SEQ ID NO: 134)
ggauccgccaccauggacuggaccuggauacucuuucucguagcagcagccacacgagugcauucaauguuugucuucu
ugguccucuugccacugguuagcucccagugugugaaucuuacuacaaggacacaacugcccccagcuuacacaaacucc
uuuacuagggguguauauuacccagacaaguguuucgcaguucuguccugcauagcacccaagaccuuuuccuuccgu
ucuucagcaacgucaccugguuccaugcuauccaugucaguggaacgaauggcacaaagcgguucgauaacccuguccu
gcccuuuaacgacggcgucuauuucgcuucaacagagaagaguaacauuaucagaggauggauauuugguacaacucuu
gauagcaagacacaaagccugcugauuguaaacaacgcgacaaaugucgucaucaagguuugcgaguuucaauuuugcaa
cgaucccuucuugggcguguacuaucauaagaacaauaaaagcuggauggagagcgaauuuaggguguauagcucagcu
aauaacuguacauuugaauaugucucucaacccuuccucauggaccuugagggaaagcaaggaaauuucaagaaucucag
agaauuugucuucaagaacaucgacggguauuucaagaucuacuccaagcauacacccaucaacuugguuagggaccuuc
cgcaagguuucucagcacuggagccucugguagaucucccuauugggauuaauauuacaagauuucaaacacuccuggc
cuugcauagauccuaucuuaccccuggggauuccagcucagguuggaccgcgggugccgcggcguacuaugucggauau
cuccaaccucggacauuccugcugaaauacaaugaaaaugggaccaucacugaugccguugauugugcucucgauccucu
gagugagaccaaaugcacucuuaagaguuuuacaguggagaaagguaucuaucaaacuaguaauuucagaguucaaccaa
ccgagucaauagugcguuuuccaaauaucacuaaucuguguccauuuggggaagucuucaaugcuacccgauucgcaag
uguguacgccuggaaccggaaacggauuucuaacugcguugccgauuauaguguccucuauaauucugcuucuuucucu
acuuuuaagugcuauggggugucccccaccaagcugaacgaucuguguuucacuaacgucuacgccgauaguuuuguca
uuagaggggacgagguacggcaaaucgcgcccggccaaacggggaaaauugccgauuacaacuacaagcuuccagacgac
uucacagguugcgugauugcauggaauucuaauaaucuggacaguaaagugggggcaacuauaacuaucuuuaccggc
uguuucggaagagcaacuugaagcccuucgaacgcgacauauccaccgagaucuaucaagccggaaguaccccgugcaac
gggguagaaggauuuaauuguuauuuuccauugcagucuuauggauuucagcccaccaauggugugggauaccaaccu
uauaggguuguuguucucuccuucgaacuccugcacgcuccagcuacuguaugugggccuaagaaaaguacuaaucucg
uuaagaauaaaugcgucaauuucaauuucaacggcuugaccgggacuggagugcucaccgaaagcaacaagaaguuucuc
ccguuucagcaauucgguagggauauugccgauacgacagaugcaguacgagauccccaaacacucgaaauccuggacau
uacgccauguagcuuuggcggaguaagugucaucaccccagggacuaacaccaguaaccaaguugcgguacucuaucag
gaugugaacugcacugagguaccuguagcaauucacgcagaccaauugacgccgacguggcgcgucuauaguacaggaa
guaacgucuuucagacaagagcggguuguuugauuggcgcugaacacguuaacaauucuuacgagugugauauccccau
cggugcggggaucugcgccagcuaucagacacaaaccaauuccccacgaaggagacguuccguggccagccagucaauaa
ucgcguauacuaugucucugggugcggagaauucaguggccuauuccaauaauucuauagccauuccaaccaauuuuac
uauaagcgucacuacagagaucuugccaguuagcaugacgaaaaccagcgucgauuguaccauguauauaugcggcgaca
guaccgaaugcucaaaucugcugcuccaauauggcucauuuugcacucaacuuaauagagcucugacagggaucgcugu
cgaacaagauaagaacacucaggaaguuuucgcccaaguuaagcagauauacaagaccccgcccaucaaggauuuuggcg
gauuuaauuucucucagauccugccggacccuagccgccgacgccggagcuuuaucgaagacuugcuguuuaauaaggu
uacucucgcagaugcaggcuucaucaagcaauacggugacugccuuggggauaucgcugcucgggaccugaucugugcu
cagaaauucaacggucucacggugcugcccccacuccugaccgacgaaaugauugcccaguauacguccgcauugcucgc
uggcaccaucacuagcggcuggaccuuuggggccggagccgcgcuccaaauaccuuuugcuaugcaaauggcuuaucgc
uucaaugguauugggguuacgcaaaauguccucuacgaaaaucaaaagcucauagcuaaccaauucaauagcgcuauagg
gaaaauucaagacagccugaguuccacagcaagcgcccucggcaaacuucaagauguagugaaccaaaaugcucaagcac
ucaauacacuggucaaacaacucucaagcaauuucggggcaaucucaucugguccuaaugacauauugagcaggcucccc
aaaguggaagcagaaguacaaaucgacaggcugauuaccggacgacuccaaagcuugcaaacuuauguaacccaacaacu
uaucagggcugcagaaauccgugcaagcgcuaaccucgccgcuacgaagaugucagaauguguacuugggcagucuaag
aggguugauuucuguggaaaaggguaccaucugaugaguuuuccacagagcgcuccacauggggugguguuucugcau
guaaccuauguucccgcucaagaaaagaauuuuacuacugcccccgcaauuugccaugacgggaaagcccauuucccccg
agagggaguuuucgugaguaacggaacgcacugguuugucacucagagaaauuucuacgagccccaaaucauuacgacc
gaucgaucugcaggagcuugggaaguaugagcaauacaucaaauggccauggucuggcagacggcgccggagaaggggc
agcuugacucauuuaaagaggaacuggauaaguacuuuaagaaucacaccucucccgaugucgaccugggcgacaucucu
ggaauuaaugccucugucguaaacauccaaaaggaaauugaccgacugaaugagguggcaaagaaucuuaaugaaucccu
gaucgaucugcaggagcuugggaaguaugagcaauacaucaaauggccauggucuggcagacggcgccggagaaggggc
ucuggcggcucuggaagcggguauauuccagaggcgcccagggaugggcaagcauauguucggaaggauggggagugg
guguuguuguccacguuccuuggcuaguga (SEQ ID NO: 135)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRRRRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSRRRRSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSGPNDILSRLPKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 136)
WuS_3F_2P_GlyDSol_pVax
ggatccgccaccatggactggacatggattttgtttcttgtggcggctgcaacgcgagttcattccatgtttgttttcctggttctcttgccg
ctcgtctctagtcaatgcgtcaatctgacgacaagaacgcaacttccccctgcctacaccaatagttttacccgtggcgtctattatccag
ataaagtgtttcgaagttcctgccttcattccacgcaagatctcttccttccattcttctctaatgtcacctggtttcatgcgatccacgtgtct
ggaaccaacgggactaaacgattcgacaatcccgtcctgccatttaacgatggagtatacttcgcatccaccgagaaatctggcattat
aagagggtggatattcgggactacactcgacagcaagacacaaagtctcctgattgttaacaacgcgacaaacgtcgtaattaaagttt
gcgaatttcaattttgtaatgacccgttcttgggcgtgtattatcacaagaataacaaaagttggatggaatccgaattccgggtttattcca
gcgcgaataattgcacatttgaatacgttagccaacctttcctgatggatctcgaaggcaaacaaggaaactttaagaatcttcgggaatt
cgttttcaagaacatcgacgggtactttaagatatactcaaaacacaccccaattaatttggtccgagatctgccgcaaggctttagtgcg
cttgagcccctcgtagatctcccaattggtattaatatcacacgctttcaaaccctgctggcattgcatcggagctatctgactcccggcg
atagttcttcagggtggacggcgggtgccgctgcttactatgtaggctatctgcagcctcgtacatttctcctcaaatacaatgaaaatgg
cactattaccgacgccgttgattgcgctctggacccactgagcgagactaaatgcaccctcaaatcattcactgttgagaagggaattta
ccaaacatcaaacttcagggtccaacctacggaaagcatcgtgcggttccccaacatcactaacctctgcccctttggagaagtatttaa
cgctacaaggttcgcttccgtctacgcctggaacaggaaaagaatcagtaattgcgtggctgattactccgtgctgtacaattccgcctc
attttctacatttaagtgttatggcgttaacgggactaagcttaacgacctctgcttcacaaacgtctatgccgacagctttgtcattcgcgg
ggatgaagtaagacagatagcacccggtcaaactggcaaaattgctgattacaattacaagttgccagatgatttcactggatgcgttat
agcatggaactctaacaaccttgactcaaaggttggtggcaactataattatttgtatcgcctgtttcgcaaatctaatctcaagcctttcga
gcgcgacataaatacgaccatataccaagcggggtccaccccttgtaatggagtcgaggggtttaattgctattttccgttgcaatcctac
gggttccaaccaacaaacggcgtcggctatcaaccctatcgggttgtcgtactctcattcgagctcaaccatgcaccagcaacagtttgt
ggccccaagaagagcacaaatttggtcaagaataaatgcgttaattttaatttcaatggtctgactggcacaggggttcttaccgaatcaa
ataagaagtttctgccatttcagcagttcggaagggactgtgcagggaccacagatgccgttagagacccccaaacactcgaaattctg
gacatcacgccatgcagtttcggtggtgttagcgtgattactccgggtactaatacgtccaaccaagtggctgtgttgtatcaagacgtta
actgtaccgaagttcctgtagcaatccatgccggtcaactgacccccacgtggcgagtttatagcaccggttccaacgtctttcaaacaa
gagccggatgtctcataggcgctgaacatgtgaataattcatacgaatgtgacattccaatcggcgcagggatttgcgcctcatatcaga
cacaaactaactccccgagaagacgtcgctcagtggcgtcacaaagcatcatcgcttatacgatgagcctctgcgccgagaactctgt
cgcatattctaacaactctattgcaattcctacaaattttacaatttcttgcactactgagatcctgcccgtaagcatgacgaaaacatcctg
cgactgcacaatgtatatctgtggcgactcaactgagtgctccaatctcctcttgcaatacggatctttctgtactcaactcaacagagcac
ttacaggaatagccgtcgaacaagacaagaacacacaagaggtcttcgcccaagtaaagcaatgttacaaaaccccacctattaaaga
ctttggtgggtttaatttctcacagattcttccagatccttcccgtagaaggagaagctttattgaagacctcttgtttaataaagtcactcttg
cagacgctgggtttattaaacaatatggagactgcttgggagacatagcggcaagagacctgatctgcgctcaaaagtttaatgggtgc
actgtgttgccaccccttctgaccgacgagatgatcgctcagtataccagtgccttgctggcagggaccataactagcggatggactttc
ggtgcaggagctgctctgcaaatcccttttgcgatgcaaatggcctacaggtttaatggtataggagttactcagaatgtcctgtacgaaa
atcaaaagctgatcgccaatcaattcaacagtgctattgggaaaatacaggacagtttgagttcaacagcgagcgctctcggcaaactg
caggatgttgtgaatcaaaacgcgcaagctttgaacactcttgtgaagcagctttcatccaacttcggagcgatctcatccgtcctgaac
gacatattgtcaagacttgacccacctgaagcggaagttcagatagaccgactcataacgggccgacttcagtccttgcagacatacgt
gacccaacaacttatccgcgcagccgaaataagggcttcagctaaccttgcagcaaccaaaatgtcagagtgcgtgctcggtcaaag
caagcgggtagacttttgtggcaaggggtatcatcttatgtcctttcctcaatccgcccctcacggggtggtcttcttgcactgcacttatgt
acctgctcaagagaagaattttacgaccgcccctgcgatctgtcacgacgggaaagcacatttcccccgcgagggagtctttgtgtcta
atggtactcattggtttgttacgcagcggaacttttacgaacctcaaataattacaacggataatacagatgttagtgggaattgcgacgtg
gtgatcggtatagtcaacaatacggtgtatgatccacttcaaccagaacttgattcctttaaggaagagctggacaaatatttcaagaacc
atacatcccctgacgtggaccttggcgatataagcggcattaatgcttcagtggtcaatatacaaaaggaaatcgatcgcctgaatgag
gtcgcaaagaatttgaatgagtccctgatcgacctgcaagagctcgggaaatatgagcagtacatcaagtggccctggtcaggtagac
gtaggcggcgccggggcagtggcggctcagggagcggttatatacccgaagcccctagagatgggcaagcttatgtccgaaagga
cggcgaatgggtgctcctttccactttcttgggataatag (SEQ ID NO: 137)
ggauccgccaccauggacuggacauggauuuuguuucuuguggcggcugcaacgcgaguucauuccauguuuguuuuc
cugguucucuugccgcucgucucuagucaaugcgucaaucugacgacaagaacgcaacuucccccugccuacaccaauag
uuuuacccguggcgucuauuauccagauaaaguguuucgaaguuccugccuucauuccacgcaagaucucuuccuucca
uucuucucuaaugucaccugguuucaugcgauccacgugucuggaaccaacgggacuaaacgauucgacaaucccgucc
ugccauuuaacgauggaguauacuucgcauccaccgagaaaucuggcauuauaagaggguggauauucgggacuacacu
cgacagcaagacacaaagucuccugauuguuaacaacgcgacaaacgucguaauuaaaguuugcgaauuucaauuuugua
augacccguucuugggcguguauuaucacaagaauaacaaaaguuggauggaauccgaauuccggguuuauuccagcgc
gaauaauugcacauuugaauacuuagccaaccuuuccugauggaucucgaaggcaaacaaggaaacuuuaagaaucuuc
gggaauucguuuucaagaacaucgacggguacuuuaagauauacucaaaacacaccccaauuaauuugguccgagaucug
ccgcaaggcuuuagugcgcuugagccccucguagaucucccaauugguauuaauaucacacgcuuucaaacccugcugg
cauugcaucggagcuaucugacucccggcgauaguucuucaggguggacgggggugccgcugcuuacuauguaggcu
aucugcagccucguacauuucuccucaaauacaaugaaaauggcacuauuaccgacgccguugauugcgcucuggaccca
cugagcgagacuaaaugcacccucaaaucauucacuguugagaagggaauuuaccaaacaucaaacuucaggguccaacc
uacauuuaaguguuauggcguuaacgggacuaagcuuaacgaccucugcuucacaaacgucuaugccgacagcuuuguc
ccgucuacgccuggaacaggaaaagaaucaguaauugcguggcugauuacuccgugcuguacaauuccgccucauuuuc
uacauuuaaguguuauggcguuaacgggacuaagcuuaacgaccucugcuucacaaacgucuaugccgacagcuuuguc
auucgcggggaugaaguaagacagauagcacccggucaaacuggcaaaauugcugauuacaauuacaaguugccagauga
uuucacuggaugcguuauagcauggaacucuaacaaccuugacucaaagguugguggcaacuauaauuauuuguaucgc
cuguuucgcaaaucuaaucucaagccuuucgagcgcgacauaaauacgaccauauaccaagcgggguccaccccuuguaa
uggagucgagggguuuaauugcuauuuuccguugcaauccuacggguuccaaccaacaaacggcgucggcuaucaaccc
uaucggguugucguacucucauucgagcucaaccaugcaccagcaacaguuuguggccccaagaagagcacaaauuugg
ucaagaauaaaugcguuaauuuuaauuucaauggucugacuggcacagggguucuuaccgaaucaaauaagaaguuucu
gccauuucagcaguucggaagggacugugcagggaccacagaugccguuagagacccccaaacacucgaaauucuggaca
ucacgccaugcaguuucggugguguuagcgugauuacuccggguacuaauacguccaaccaaguggcuguguuguauca
agacguuaacuguaccgaaguuccuguagcaauccaugccggucaacugacccccacguggcgaguuuauagcaccggu
uccaacgucuuucaaacaagagccggaugucucauaggcgcugaacaugugaauaauucauacgaaugugacauuccaau
cggcgcagggauuugcgccucauaucagacacaaacuaacuccccgagaagacgucgcucaguggcgucacaaagcauca
ucgcuuauacgaugagccucugcgccgagaacucugucgcauauucuaacaacucuauugcaauuccuacaaauuuuaca
auuucuugcacuacugagauccugcccguaagcaugacgaaaacauccugcgacugcacaauguauaucuguggcgacuc
aacugagugcuccaaucuccucuugcaauacggaucuuucuguacucaacucaacagagcacuuacaggaauagccgucg
aacaagacaagaacacacaagaggucuucgcccaaguaaagcaauguuacaaaaccccaccuauuaaagacuuugguggg
uuuaauuucucacagauucuuccagauccuucccguagaaggagaagcuuuauugaagaccucuuguuuaauaaaguca
cucuugcagacgcuggguuuauuaaacaauauggagacugcuugggagacauagcggcaagagaccugaucugcgcuca
aaaguuuaaugggugcacuguguugccaccccuucugaccgacgagaugaucgcucaguauaccagugccuugcuggca
gggaccauaacuagcggauggacuuucggugcaggagcugcucugcaaaucccuuuugcgaugcaaauggccuacaggu
uuaaugguauaggaguuacucagaauguccuguacgaaaaucaaaagcugaucgccaaucaauucaacagugcuauugg
gaaaauacaggacaguuugaguucaacagcgagcgcucucggcaaacugcaggauguugugaaucaaaacgcgcaagcuu
ugaacacucuugugaagcagcuuucauccaacuucggagcgaucucauccguccugaacgacauauugucaagacuugac
ccaccugaagcggaaguucagauagaccgacucauaacgggccgacuucaguccuugcagacauacgugacccaacaacu
uauccgcgcagccgaaauaagggcuucagcuaaccuugcagcaaccaaaaugucagagugcgugcucggucaaagcaagc
ggguagacuuuuguggcaagggguaucaucuuauguccuuuccucaauccgccccucacgggguggucuucuugcacu
gcacuuauguaccugcucaagagaagaauuuuacgaccgccccugcgaucugucacgacgggaaagcacauuucccccgc
gagggaguuuugugucuaaugguacucauugguuuguuacgcagcggaacuuuuacgaaccucaaauaauuacaacgg
auaauacagauguuagugggaauugcgacguggugaucgguauagucaacaauacgguguaugauccacuucaaccaga
acuugauuccuuuaaggaagagcuggacaaauauuucaagaaccauacauccccugacguggaccuuggcgauauaagcg
gcauuaaugcuucaguggucaauauacaaaaggaaaucgaucgccugaaugaggucgcaaagaauuugaaugagucccu
gaucgaccugcaagagcucgggaaauaugagcaguacaucaaguggcccuggucagguagacguaggcggcgccggggc
aguggcggcucagggagcgguuauauacccgaagccccuagagaugggcaagcuuauguccgaaaggacggcgaauggg
ugcuccuuuccacuuucuugggauaauag (SEQ ID NO: 138)
MDWTWILFLVAAATRVHSMFVFLVLLPLVSSQCVNLTTRTQLPPAYTNSFTRGVYY
PDKVFRSSCLHSTQDLFLPFFSNVTWFHAIHVSGTNGTKRFDNPVLPFNDGVYFASTE
KSGIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDCAGTTDAVRDPQTLEILDITPCSFGGVSVITPGTNTSNQVAVLYQD
VNCTEVPVAIHAGQLTPTWRVYSTGSNVFQTRAGCLIGAEHVNNSYECDIPIGAGICA
SYQTQTNSPRRRRSVASQSIIAYTMSLCAENSVAYSNNSIAIPTNFTISCTTEILPVSMT
KTSCDCTMYICGDSTECSNLLLQYGSFCTQLNRALTGIAVEQDKNTQEVFAQVKQCY
KTPPIKDFGGFNFSQILPDPSRRRRSFIEDLLFNKVTLADAGFIKQYGDCLGDIAARDLI
CAQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTDVSGNCDVVIGIVNNTV
YDPLQPELDSFKEELDKYFKNHTSPDVDLGDISGINASVVNIQKEIDRLNEVAKNLNE
SLIDLQELGKYEQYIKWPWSGRRRRRRGSGGSGSGYIPEAPRDGQAYVRKDGEWVL
LSTFLG** (SEQ ID NO: 139)
WuS_3F_2P_Gly_pVax
ggatccgccaccatggattggacctggatacttttcctcgtggccgcagcaacaagagtccactccatgttcgtcttcttggtacttttgcc
actcgtcagttctcagtgcgttaacctgactactagaacccaattgcccccggcatacacaaactctttcacccggggtgtctactatccc
gacaaagtgtttagaagtagcgtgctgcacagcacccaagatctctttctgccattcttctcaaacgtcacctggtttcacgccatccatgt
aagcgggaccaacggcacaaagcgttttgataaccctgttttgccattcaatgatggcgtgtattttgcttccactgagaaaagcaacatc
attagagggtggatatttggcacaacgcttgactccaagacgcagagtcttttgatagtaaacaacgcaactaatgtggtcattaaagtct
gtgaatttcaattttgcaatgaccccttccttggagtctattaccacaagaacaacaaaagctggatggaaagcgaatttagggtctacag
ctctgccaataactgcacattcgaatacgtcagccaaccattcttgatggacctggaaggcaagcaaggaaactttaagaatctgaggg
aatttgtgtttaagaatatcgacggatattttaagatctattccaagcatactcccattaatctcgttcgtgaccttcctcagggtttctctgcat
tggaacccctcgtagatttgcccattgggattaatatcactagattccagacgctgcttgcactccatcgatcttatctgacccctggtgac
tcctcttccgggtggacggcgggtgctgcagcctactacgttggctatttgcaacctaggacctttctgttgaagtataatgagaatggga
ctattactgatgccgttgattgcgccctcgatccgctgtcagaaacaaagtgcaccctgaagagcttcacagtagaaaagggaatctatc
aaacctcaaatttccgcgttcaaccaactgaatcaatcgtgcgttttcctaacatcacaaatctgtgtccgtttggagaagtatttaatgcga
cgcgtttcgcaagcgtctacgcgtggaatcgcaaacgtatctctaattgcgtagcagattattctgtgctgtacaatagcgcatctttctca
acgtttaagtgctacggcgttaatgggaccaagctgaatgatctctgtttcactaatgtgtacgcagacagttttgtaattagaggagacg
aggttaggcaaatagcaccgggtcaaactggcaaaatcgccgactataactacaagctccctgatgacttcacgggctgcgtaattgct
tggaactctaataacctggactctaaagtcggcgggaattataattatctctatcggttgtttcgaaaatccaatctcaaaccctttgagcg
ggacatcaatactacaatttatcaagctggtagtactccttgcaatggggtagaaggcttcaattgttatttcccccttcaatcttacggattt
caacccacgaacggcgtagggtaccagccctatcgagtggtggtactgtcattcgaacttaatcacgccccagcaacagtctgcggg
cctaagaaaagcacgaatcttgtcaagaataagtgtgtaaatttcaacttcaatggtcttacaggcacgggagtgctcactgagtctaata
agaaatttcttcctttccaacaattcggtcgtgatattgccgatactactgatgcagtccgagatccacaaactctcgaaatcctcgatatta
ctccttgtagttttggcggcgtctccgtgatcaccccagggaccaacactagtaaccaagtggcggtgctctaccaagatgttaactgca
cagaagtcccggtagcgatccatgccgaccagctcactcccacatggcgtgtttacagcacagggtcaaacgttttccagacccgtgc
cggatgtcttataggagccgaacacgtaaataacagttatgaatgcgatatcccaattggtgcaggtatctgtgcgtcatatcaaaccca
aactaattctccgagacgacgacggagcgttgcctcacaatcaataatcgcctacacaatgtccctcggtgccgaaaattcagtcgctta
ctctaacaatagcattgctatccctaccaacttcactatttctgttaccacggaaattttgcctgtatccatgaccaaaacatctgttgattgc
acgatgtacatctgcggggattctaccgaatgttctaacctgcttctgcaatacggctccttctgcacccaattgaaccgcgcactgactg
ggattgctgtggaacaagacaagaatactcaagaagtatttgcccaggtcaaacagatttacaaaactcccccaattaaagatttcggc
ggtttcaattttagtcaaattctgccagatccaagtcgacgccgcaggagctttattgaggacctgctctttaataaagtcacgctggccg
acgccggcttcataaaacagtatggcgattgtcttggagacatcgccgcccgcgacctcatttgcgcacaaaagttcaatgggctcacc
gtgttgccaccactgctcacagatgagatgatcgcacagtacacgagcgcccttcttgccggcactatcacgtctggttggacgttcgg
tgccggagccgctctgcaaattccctttgcaatgcaaatggcctatagatttaatggaattggcgtaacacagaacgtgttgtacgagaa
ccagaagctcattgccaaccagttcaattccgctattggcaaaatacaagactctctcagctcaactgctagcgcactgggaaaattgca
agacgtagtcaatcaaaatgcccaagccctcaatactctcgtcaaacagttgtcttccaactttggggctatcagtagtgtactcaatgac
attctttcaagactggacccgcccgaggcggaagtccaaattgatcgtctgataactggaaggttgcaaagccttcagacctacgttac
gcaacaacttattagggctgccgaaataagggcatccgctaatctggcagctacaaagatgtctgaatgtgttttgggacagagcaaac
gggttgacttctgcggtaaaggttaccatctcatgtcttttccacaaagcgcaccgcacggagtcgtcttcctgcatgtaacatacgtccc
agcccaagaaaagaattttaccacagccccagccatctgccacgacggcaaggcgcatttcccaagggaaggcgtgtttgtatccaa
cgggacgcattggtttgtcactcaaaggaacttttacgaaccccaaattattaccactgataacaccttcgtttctgggaactgtgatgtcg
tgattgggatagtaaacaacacggtatatgatccactgcaaccagaactggattccttcaaagaagagctggacaaatacttcaagaat
catactagtcctgacgtcgacctgggcgatatcagtggaatcaacgctagcgtcgtaaacattcaaaaggagatcgatagacttaacga
ggtcgccaagaatctcaatgaaagcctcatcgatttgcaagaactcggaaaatatgagcaatacataaaatggccatggtctggcagg
agaagacgcaggagaggtagcggcggcagcggatcagggtacattccggaagcccccagggacggacaggcatatgtccgcaa
ggacggagaatgggttcttcttagcacttttctggggtaataa (SEQ ID NO: 140)
ggauccgccaccauggauuggaccuggauacuuuuccucguggccgcagcaacaagaguccacuccauguucgucuucu
ugguacuuuugccacucgucaguucucagugcguuaaccugacuacuagaacccaauugcccccggcauacacaaacucu
uucacccggggugucuacuaucccgacaaaguguuuagaaguagcgugcugcacagcacccaagaucucuuucugccau
ucuucucaaacgucaccugguuucacgccauccauguaagcgggaccaacggcacaaagcguuuugauaacccuguuuu
gccauucaaugauggcguguauuuugcuuccacugagaaaagcaacaucauuagaggguggauauuuggcacaacgcuu
gacuccaagacgcagagucuuuugauaguaaacaacgcaacuaauguggucauuaaagucugugaauuucaauuuugca
augaccccuuccuuggagucuauuaccacaagaacaacaaaagcuggauggaaagcgaauuuagggucuacagcucugcc
aauaacugcacauucgaauacgucagccaaccauucuugauggaccuggaaggcaagcaaggaaacuuuaagaaucugag
ggaauuuguguuuaagaauaucgacggauauuuuaagaucuauuccaagcauacucccauuaaucucguucgugaccuu
ccucaggguuucucugcauuggaaccccucguagauuugcccauugggauuaauaucacuagauuccagacgcugcuug
cacuccaucgaucuuaucugaccccuggugacuccucuuccggguggacgggggugcugcagccuacuacguuggcua
uuugcaaccuaggaccuuucuguugaaguauaaugagaaugggacuauuacugaugccguugauugcgcccucgauccg
cugucagaaacaaagugcacccugaagagcuucacaguagaaaagggaaucuaucaaaccucaaauuuccgcguucaacc
aacugaaucaaucgugcguuuuccuaacaucacaaaucuguguccguuuggagaaguauuuaaugcgacgcguuucgca
agcgucuacgcguggaaucgcaaacguaucucuaauugcguagcagauuauucugugcuguacaauagcgcaucuuucu
caacguuuaagugcuacggcguuaaugggaccaagcugaaugaucucuguuucacuaauguguacgcagacaguuuugu
aauuagaggagacgagguuaggcaaauagcaccgggucaaacuggcaaaaucgccgacuauaacuacaagcucccugaug
acuucacgggcugcguaauugcuuggaacucuaauaaccuggacucuaaagucggcgggaauuauaauuaucucuaucg
guuguuucgaaaauccaaucucaaacccuuugagcgggacaucaauacuacaauuuaucaagcugguaguacuccuugca
augggguagaaggcuucaauuguuauuucccccuucaaucuuacggauuucaacccacgaacggcguaggguaccagcc
cuaucgaguggugguacugucauucgaacuuaaucacgccccagcaacagucugcgggccuaagaaaagcacgaaucuug
ucaagaauaaguguguaaauuucaacuucaauggucuuacaggcacgggagugcucacugagucuaauaagaaauuucu
uccuuuccaacaauucggucgugauauugccgauacuacugaugcaguccgagauccacaaacucucgaaauccucgaua
uuacuccuuguaguuuuggcggcgucuccgugaucaccccagggaccaacacuaguaaccaaguggcggugcucuacca
agauguuaacugcacagaagucccgguagcgauccaugccgaccagcucacucccacauggcguguuuacagcacagggu
caaacguuuuccagacccgugccggaugucuuauaggagccgaacacguaaauaacaguuaugaaugcgauaucccaauu
ggugcagguaucugugcgucauaucaaacccaaacuaauucuccgagacgacgacggagcguugccucacaaucaauaau
cgccuacacaaugucccucggugccgaaaauucagucgcuuacucuaacaauagcauugcuaucccuaccaacuucacua
uuucuguuaccacggaaauuuugccuguauccaugaccaaaacaucuguugauugcacgauguacaucugcggggauuc
uaccgaauguucuaaccugcuucugcaauacggcuccuucugcacccaauugaaccgcgcacugacugggauugcugug
gaacaagacaagaauacucaagaaguauuugcccaggucaaacagauuuacaaaacucccccaauuaaagauuucggcgg
uuucaauuuuagucaaauucugccagauccaagucgacgccgcaggagcuuuauugaggaccugcucuuuaauaaaguc
acgcuggccgacgccggcuucauaaaacaguauggcgauugucuuggagacaucgccgcccgcgaccucauuugcgcaca
aaaguucaaugggcucaccguguugccaccacugcucacagaugagaugaucgcacaguacacgagcgcccuucuugccg
gcacuaucacgucugguuggacguucggugccggagccgcucugcaaauucccuuugcaaugcaaauggccuauagauu
uaauggaauuggcguaacacagaacguguuguacgagaaccagaagcucauugccaaccaguucaauuccgcuauuggca
aaauacaagacucucucagcucaacugcuagcgcacugggaaaauugcaagacguagucaaucaaaaugcccaagcccuca
auacucucgucaaacaguugucuuccaacuuuggggcuaucaguaguguacucaaugacauucuuucaagacuggaccc
gcccgaggcggaaguccaaauugaucgucugauaacuggaagguugcaaagccuucagaccuacguuacgcaacaacuua
uuagggcugccgaaauaagggcauccgcuaaucuggcagcuacaaagaugucugaauguguuuugggacagagcaaacg
gguugacuucugcgguaaagguuaccaucucaugucuuuuccacaaagcgcaccgcacggagucgucuuccugcaugua
acauacgucccagcccaagaaaagaauuuuaccacagccccagccaucugccacgacggcaaggcgcauuucccaagggaa
ggcguguuuguauccaacgggacgcauugguuugucacucaaaggaacuuuuacgaaccccaaauuauuaccacugaua
acaccuucguuucugggaacugugaugucgugauugggauaguaaacaacacgguauaugauccacugcaaccagaacu
ggauuccuucaaagaagagcuggacaaauacuucaagaaucauacuaguccugacgucgaccugggcgauaucaguggaa
ucaacgcuagcgucguaaacauucaaaaggagaucgauagacuuaacgaggucgccaagaaucucaaugaaagccucauc
gauuugcaagaacucggaaaauaugagcaauacauaaaauggccauggucuggcaggagaagacgcaggagagguagcg
gcggcagcggaucaggguacauuccggaagcccccagggacggacaggcauauguccgcaaggacggagaauggguucu
ucuuagcacuuuucugggguaau (SEQ ID NO: 141)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFAQVKQIY
KTPPIKDFGGFNFSQILPDPSRRRRSFIEDLLFNKVTLADAGFIKQYGDCLGDIAARDLI
CAQKFNGLTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANL
AATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 142)
WuS_3F_2P_NoClev_pVax
ggatccgccaccatggattggacgtggattctgtttctggtggccgcagcgacaagggtgcattcaatgtttgtgttcctggtcctgctgc
cactggtctcatcacagtgtgtaaacctgactacaagaacgcagcttccgcctgcctacacgaacagcttcaccaggggagtgtattat
cctgacaaagtctttaggagctctgttctccactccactcaagacctgtttctgcccttcttcagtaacgtgacttggtttcacgcaatacat
gtctccggcacaaatggaaccaaaagattcgataaccctgttctcccattcaatgatggagtatattttgctagcactgaaaagtctaacat
tattagaggctggatatttggcacgacattggactccaagacgcaaagtctcttgattgtgaacaacgcaacaaacgtggtgataaaagt
ttgcgaattccaattttgcaatgacccattcctgggagtttactaccacaagaataacaaaagctggatggaatccgagttccgggtttact
cctctgctaacaactgtacctttgagtatgtgagtcaaccattccttatggatctcgaaggaaaacaaggtaacttcaagaacctgaggg
agtttgtgtttaagaatatcgatggctattttaagatttatagcaaacacactccgattaatctggtgagagatctcccgcaaggattttctgc
tttggagccattggttgacttgcctattggaatcaacatcacccgttttcaaactctgcttgcgctgcatagatcctaccttacgcctggcga
ttcaagcagtggctggaccgcgggagcggccgcctattatgtaggctacttgcagcctcgcacctttctcctcaagtacaatgaaaacg
gcacaattacagacgcagtggattgtgctctggaccccctcagtgaaactaaatgtaccctgaaaagcttcactgttgagaaaggcatat
atcaaacctcaaactttagagtgcaacctactgaaagcattgtaagattccctaacattacaaacctgtgcccctttggcgaagtctttaat
gcaacccggtttgctagcgtgtatgcttggaaccgcaagaggatatccaattgcgtcgcagattattccgtcctgtataactctgccagct
ttagtaccttcaaatgttatggggtatctcccacaaaactcaatgatctttgtttcacaaatgtctatgctgactcctttgttatcagagggga
cgaagttcgccaaattgctccaggtcaaacaggaaagattgcagattataactacaagcttcccgacgattttacaggttgtgtgatagct
tggaactccaataatctggattccaaggtaggcgggaactacaattatctctacaggctcttccggaaatccaatctcaagccgttcgaa
agggatataagcactgagatctatcaagcaggcagtacaccctgtaacggagtagagggcttcaactgctactttccactgcaatcctat
gggtttcaaccgactaacggtgtcgggtaccaaccctatcgtgtcgtggtcctgtcctttgagcttctgcacgctcctgctaccgtttgcg
gccccaagaaaagcacgaatttggtcaagaacaaatgtgtcaactttaacttcaacggattgacagggaccggagtattgaccgaatct
aataagaaatttctgcccttccaacaattcggacgggacatagcagacacaaccgatgctgtcagggacccacagacacttgaaatac
tcgatatcaccccatgcagctttggcggagtctcagtcattacgcctggcaccaatacttccaatcaagttgcagtgctctatcaggatgt
caattgtactgaggtccccgtcgccatccacgcggatcaacttacccccacatggcgagtatatagtaccgggagcaacgtctttcaaa
cccgagcaggatgtctgataggtgccgaacacgtaaacaacagctacgaatgtgatatcccgatcggcgcagggatttgcgctagct
accaaacccaaactaattctccgcgccgccgcaggtccgtagcaagtcaatcaataatagcatacaccatgtcattgggagctgaaaa
cagcgtggcatatagcaacaattccatagctatccctacaaatttcacgatttctgttaccaccgaaattctgccagtgagcatgaccaaa
acctcagtggattgtacgatgtacatatgcggcgattccacggaatgttccaatctccttttgcaatacggcagcttttgtacccaactgaa
tagagctctgacgggtatagcagtagagcaggataagaacactcaagaggtgtttgcccaggtcaaacaaatttacaagactccccca
ataaaagactttggcggcttcaatttcagccaaatcttgccagacccttccaggcggcggcgctcatttatcgaagatttgcttttcaataa
agtcaccctggccgacgccggatttattaaacaatacggcgattgtctgggcgacatcgccgcaagggacctcatctgtgcgcaaaag
ttcaatggcctgacggtgcttccaccactcctgactgatgagatgattgcccaatacacatctgccctgctggctggtacaataacgagt
gggtggacctttggggctggagcagcattgcaaattccattcgccatgcaaatggcatatcgttttaacggcattggagtgactcaaaat
gtgctgtatgaaaaccaaaagcttattgcaaatcagtttaattccgccattggcaaaatccaggatagcctcagtagtacagcaagcgcc
ttggggaaactgcaagatgtggttaatcaaaatgcacaagctctcaataccctggtcaagcaacttagtagtaactttggtgccatcagc
agcgttctcaacgacatcctgagtcgtcttgatcccccagaggcagaggttcaaattgaccggcttatcactggaaggcttcaatccctg
caaacttacgtgactcagcaactgatacgcgctgcagaaattcgggcctcagcaaaccttgccgcgacaaagatgagcgaatgcgtg
ctgggacaatccaagcgggtcgacttttgtggtaaaggctatcatctgatgagcttcccacagtccgctccacacggcgtcgttttcctg
cacgtgacctatgtgccagcacaggagaagaactttacaacagccccggctatctgccacgatggcaaagctcactttcctagagagg
gagtgtttgtaagcaatggaacccattggttcgttacacaaagaaacttttatgagccgcaaattatcacaacagataatacattcgtctcc
gggaactgtgacgttgtgatagggattgtcaacaacacagtgtacgaccccctgcaacccgagctggattcatttaaagaagaactcg
acaagtacttcaagaatcatactagtccagatgtggatctgggcgatatatcaggaatcaatgccagcgtggtcaatattcaaaaggag
attgatagactgaacgaggttgccaagaatctgaatgaaagcctgatcgatctgcaagaattgggcaagtatgagcagtacattaaatg
gccctggtctggcgggagcggcggatctgggtctggatatattcccgaagctcctagagatggacaagcttacgtccgtaaagacgg
cgagtgggttcttctctccacattcctcggctgatga (SEQ ID NO: 143)
ggauccgccaccauggauuggacguggauucuguuucugguggccgcagcgacaagggugcauucaauguuuguguuc
cugguccugcugccacuggucucaucacaguguguaaaccugacuacaagaacgcagcuuccgccugccuacacgaacag
cuucaccaggggaguguauuauccugacaaagucuuuaggagcucuguucuccacuccacucaagaccuguuucugccc
uucuucaguaacgugacuugguuucacgcaauacaugucuccggcacaaauggaaccaaaagauucgauaacccuguucu
cccauucaaugauggaguauauuuugcuagcacugaaaagucuaacauuauuagaggcuggauauuuggcacgacauug
gacuccaagacgcaaagucucuugauugugaacaacgcaacaaacguggugauaaaaguuugcgaauuccaauuuugcaa
ugacccauuccugggaguuuacuaccacaagaauaacaaaagcuggauggaauccgaguuccggguuuacuccucugcu
aacaacuguaccuuugaguaugugagucaaccauuccuuauggaucucgaaggaaaacaagguaacuucaagaaccugag
ggaguuuguguuuaagaauaucgauggcuauuuuaagauuuauagcaaacacacuccgauuaaucuggugagagaucuc
ccgcaaggauuuucugcuuuggagccauugguugacuugccuauuggaaucaacaucacccguuuucaaacucugcuug
cgcugcauagauccuaccuuacgccuggcgauucaagcaguggcuggaccgcgggagcggccgccuauuauguaggcua
cuugcagccucgcaccuuucuccucaaguacaaugaaaacggcacaauuacagacgcaguggauugugcucuggaccccc
ucagugaaacuaaauguacccugaaaagcuucacuguugagaaaggcauauaucaaaccucaaacuuuagagugcaaccu
acugaaagcauuguaagauucccuaacauuacaaaccugugccccuuuggcgaagucuuuaaugcaacccgguuugcua
gcguguaugcuuggaaccgcaagaggauauccaauugcgucgcagauuauuccguccuguauaacucugccagcuuuag
uaccuucaaauguuaugggguaucucccacaaaacucaaugaucuuuguuucacaaaugucuaugcugacuccuuuguu
aucagaggggacgaaguucgccaaauugcuccaggucaaacaggaaagauugcagauuauaacuacaagcuucccgacga
uuuuacagguugugugauagcuuggaacuccaauaaucuggauuccaagguaggcgggaacuacaauuaucucuacagg
cucuuccggaaauccaaucucaagccguucgaaagggauauaagcacugagaucuaucaagcaggcaguacacccuguaa
cggaguagagggcuucaacugcuacuuuccacugcaauccuauggguuucaaccgacuaacggugucggguaccaaccc
uaucgugucgugguccuguccuuugagcuucugcacgcuccugcuaccguuugcggccccaagaaaagcacgaauuugg
ucaagaacaaaugugucaacuuuaacuucaacggauugacagggaccggaguauugaccgaaucuaauaagaaauuucug
cccuuccaacaauucggacgggacauagcagacacaaccgaugcugucagggacccacagacacuugaaauacucgauau
caccccaugcagcuuuggcggagucucagucauuacgccuggcaccaauacuuccaaucaaguugcagugcucuaucagg
augucaauuguacugagguccccgucgccauccacgcggaucaacuuacccccacauggcgaguauauaguaccgggagc
aacgucuuucaaacccgagcaggaugucugauaggugccgaacacguaaacaacagcuacgaaugugauaucccgaucgg
cgcagggauuugcgcuagcuaccaaacccaaacuaauucuccgcgccgccgcagguccguagcaagucaaucaauaauag
cauacaccaugucauugggagcugaaaacagcguggcauauagcaacaauuccauagcuaucccuacaaauuucacgauu
ucuguuaccaccgaaauucugccagugagcaugaccaaaaccucaguggauuguacgauguacauaugcggcgauuccac
ggaauguuccaaucuccuuuugcaauacggcagcuuuuguacccaacugaauagagcucugacggguauagcaguagag
caggauaagaacacucaagagguguuugcccaggucaaacaaauuuacaagacucccccaauaaaagacuuuggcggcuu
caauuucagccaaaucuugccagacccuuccaggcggcggcgcucauuuaucgaagauuugcuuuucaauaaagucaccc
uggccgacgccggauuuauuaaacaauacggcgauugucugggcgacaucgccgcaagggaccucaucugugcgcaaaa
guucaauggccugacggugcuuccaccacuccugacugaugagaugauugcccaauacacaucugcccugcuggcuggu
acaauaacgaguggguggaccuuuggggcuggagcagcauugcaaauuccauucgccaugcaaauggcauaucguuuua
acggcauuggagugacucaaaaugugcuguaugaaaaccaaaagcuuauugcaaaucaguuuaauuccgccauuggcaaa
auccaggauagccucaguaguacagcaagcgccuuggggaaacugcaagaugugguuaaucaaaaugcacaagcucucaa
uacccuggucaagcaacuuaguaguaacuuuggugccaucagcagcguucucaacgacauccugagucgucuugauccc
ccagaggcagagguucaaauugaccggcuuaucacuggaaggcuucaaucccugcaaacuuacgugacucagcaacugau
acgcgcugcagaaauucgggccucagcaaaccuugccgcgacaaagaugagcgaaugcgugcugggacaauccaagcggg
ucgacuuuugugguaaaggcuaucaucugaugagcuucccacaguccgcuccacacggcgucguuuuccugcacgugac
cuaugugccagcacaggagaagaacuuuacaacagccccggcuaucugccacgauggcaaagcucacuuuccuagagagg
gaguguuuguaagcaauggaacccauugguucguuacacaaagaaacuuuuaugagccgcaaauuaucacaacagauaau
acauucgucuccgggaacugugacguugugauagggauugucaacaacacaguguacgacccccugcaacccgagcugg
auucauuuaaagaagaacucgacaaguacuucaagaaucauacuaguccagauguggaucuggggauauaucaggaauc
aaugccagcguggucaauauucaaaaggagauugauagacugaacgagguugccaagaaucugaaugaaagccugaucg
aucugcaagaauugggcaaguaugagcaguacauuaaauggcccuggucuggcgggagcggcggaucugggucuggau
auauucccgaagcuccuagagauggacaagcuuacguccguaaagacggcgaguggguucuucucuccacauuccucgg
cugauga (SEQ ID NO: 144)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRRRRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSRRRRSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGGSGGSGSGYIPEAPRDGQAYVRKDGEWVLLSTFLG**
(SEQ ID NO: 145)
WuS_3F_2P_pVax
ggatccgccaccatggattggacctggattctctttcttgtggcggcggcaacacgcgtccacagcatgttcgtctttctggtattgctgc
cgcttgtgagtagccagtgtgtgaacttgactaccaggacccagctcccaccggcttataccaattccttcacaagaggtgtctactatc
cagataaagttttccgcagctcagtgttgcatagcacacaggatctctttctgccattcttcagcaacgtcacgtggtttcatgcgatacac
gttagtggaacaaacggaacaaaacgcttcgacaaccctgttctgccattcaatgacggagtttactttgcgagtaccgagaaatctaac
atcattagagggtggatctttgggactacattggattctaaaacccagtcactcctcatagtcaataacgctacaaatgtggtgattaaggt
atgcgaatttcagttttgcaacgacccatttctcggtgtatattatcacaagaataataaaagttggatggagtccgagttccgcgtctattc
atcagccaataattgtactttcgaatatgtttctcaaccgtttctcatggatctcgaaggaaagcaagggaattttaagaatctccgggagtt
tgtcttcaagaacatagatggctattttaaaatttactcaaagcatactcctattaacctcgttcgggatctcccccagggttttagcgccct
ggagccactggttgatctgccaattgggattaatatcacacgctttcaaactctcctggcccttcacagatcttacttgaccccaggtgatt
caagtagtggttggacagctggagctgctgcatattatgtaggttatctccaaccccgcacctttctcctcaaatataacgagaacggca
ccattacagatgcggtggactgtgccttggaccctctttctgagaccaagtgcacactcaaaagcttcactgtcgaaaaggggatttacc
agacatcaaattttagagttcaacccaccgaaagcattgtgagatttcctaacattacaaacttgtgcccatttggggaagtctttaacgct
acacgctttgctagcgtctatgcttggaaccgaaaacggattagtaactgcgtagctgattattccgtcttgtacaacagcgcatcttttag
cactttcaagtgttatggagtaagcccaacaaagctcaacgacctttgttttactaacgtctatgctgattcattcgtgattcgtggggatga
ggttcgtcagatcgccccaggccaaaccgggaaaatcgctgattataattataaattgcctgacgattttaccggctgtgtaatcgcctg
gaattccaacaatcttgattccaaggttggcggcaactacaactatctctaccgtctgtttcgcaaatccaatctcaagcccttcgaacgc
gatatttcaactgaaatctatcaggcagggtccactccgtgtaacggcgtagaaggatttaattgttacttcccattgcaaagttatggcttt
caacccaccaacggagtcgggtaccaaccatacagagtcgtcgtgctctcatttgagctccttcatgcacctgccacggtgtgcggcc
caaagaaatcaacgaaccttgtgaagaataaatgtgtcaattttaactttaatggcctgacagggactggcgtcctcacagaatctaataa
gaagtttctccctttccagcaatttggtcgcgatatagctgataccacagatgcagttagagacccacagacacttgagattctcgatatta
ccccgtgctcctttggcggcgtgtccgtcattactcccggtaccaatacgtctaaccaggtagcagtgctctaccaagatgtaaattgtac
tgaggtacccgtggcaatccatgccgaccaactgactccaacgtgggggtttattcaaccggaagcaacgtgtttcaaacacgggct
ggctgccttataggcgctgagcacgtgaataatagttacgagtgtgatatcccgatcggagccggcatctgtgcatcttatcaaacacaa
acaaactccccgcgccggcggagaagcgtggctagccaaagtataatcgcttatacaatgtccttgggcgcggaaaattcagtggctt
attccaataattcaattgccattcctaccaactttacaattagcgtgaccacagaaatcttgcctgtgtctatgaccaagaccagcgtcgatt
gcaccatgtatatctgtggagatagcaccgagtgttcaaatttgctcctgcaatacggttccttttgtacacagcttaaccgcgccctcaca
ggtattgctgttgaacaagacaagaatactcaagaggtatttgctcaggtaaaacaaatttataagaccccaccgataaaagattttggc
ggtttcaatttctcccaaatattgccagatcctagtaggcgtcgtagatcatttatcgaggatctcctgttcaataaagtaaccctcgccgac
gctggtttcatcaaacaatatggcgactgcctgggagatattgcagctagggatttgatttgtgcacagaagttcaatggactcaccgttc
tcccgcctctcctgacagatgagatgattgcacaatacacctctgctcttttggccgggaccattacgagcggttggacttttggcgcgg
gtgcggctctccaaattcctttcgcgatgcaaatggcgtatagatttaatggaattggcgttactcaaaacgtcttgtacgagaatcagaa
actgatcgccaaccaatttaacagtgcaattggcaaaatccaagatagccttagttctactgcttcagcattgggtaagttgcaagatgtg
gtcaaccaaaacgcacaagcactcaataccctcgtgaagcaattgtccagcaattttggagctatctcaagtgtgctcaacgacatccttt
ctaggcttgatccacccgaggcagaggttcaaatcgacagactgataactggcaggctccaatctctgcaaacgtacgtgacacagca
actgattagggctgctgagatcagggcgtccgcgaatttggcagcaaccaaaatgagcgaatgcgtgctgggacaatcaaagagagt
tgatttctgtggaaagggttaccatctcatgtccttccctcaatcagctccccatggagttgtgtttctgcacgttacttacgtgccggcaca
agaaaagaatttcaccactgcaccggctatatgtcatgatgggaaagcccacttcccgcgggaaggcgttttcgtgtccaacgggact
cattggttcgtcacacaaaggaacttctatgagccacaaataattacaacagacaacacctttgtctctgggaactgcgatgtcgtgattg
gaatcgtgaacaacactgtctacgatccgctgcaacccgaactcgactcattcaaagaggaactggataagtatttcaagaaccatacc
agccccgatgtcgatctgggcgatatctccgggataaatgcttcagtagtaaacattcaaaaggaaatcgaccggctgaacgaggttg
cgaagaatcttaatgagtcattgatcgacctgcaagaacttggtaagtatgagcagtacatcaagtggccttggtcaggccgcaggcgt
cggcgtcgtgggagcggcggcagtgggagcggatatattccagaagcgccccgagacggacaagcttacgtacgaaaagacgga
gaatgggtactgctttccacttttcttggctaatga (SEQ ID NO: 146)
ggauccgccaccauggauuggaccuggauucucuuucuuguggcggcggcaacacgcguccacagcauguucgucuuuc
ugguauugcugccgcuugugaguagccagugugugaacuugacuaccaggacccagcucccaccggcuuauaccaauuc
cuucacaagaggugucuacuauccagauaaaguuuuccgcagcucaguguugcauagcacacaggaucucuuucugcca
uucuucagaacgucacgugguuucaugcgauacacguuaguggaacaaacggaacaaaacgcuucgacaacccuguucu
gccauucaaugacggaguuuacuuugcgaguaccgagaaaucuaacaucauuagaggguggaucuuugggacuacauug
gauucuaaaacccagucacuccucauagucaauaacgcuacaaauguggugauuaagguaugcgaauuucaguuuugca
acgacccauuucucgguguauauuaucacaagaauaauaaaaguuggauggaguccgaguuccgcgucuauucaucagc
caauaauuguacuuucgaauauguuucucaaccguuucucauggaucucgaaggaaagcaagggaauuuuaagaaucuc
cgggaguuugucuucaagaacauagauggcuauuuuaaaauuuacucaaagcauacuccuauuaaccucguucgggauc
ucccccaggguuuuagcgcccuggagccacugguugaucugccaauugggauuaauaucacacgcuuucaaacucuccu
ggcccuucacagaucuuacuugaccccaggugauucaaguagugguuggacagcuggagcugcugcauauuauguaggu
uaucuccaaccccgcaccuuucuccucaaauauaacgagaacggcaccauuacagaugcgguggacugugccuuggaccc
ucuuucugagaccaagugcacacucaaaagcuucacugucgaaaaggggauuuaccagacaucaaauuuuagaguucaac
ccaccgaaagcauugugagauuuccuaacauuacaaacuugugcccauuuggggaagucuuuaacgcuacacgcuuugc
uagcgucuaugcuuggaaccgaaaacggauuaguaacugcguagcugauuauuccgucuuguacaacagcgcaucuuuu
agcacuuucaaguguuauggaguaagcccaacaaagcucaacgaccuuuguuuuacuaacgucuaugcugauucauucg
ugauucguggggaugagguucgucagaucgccccaggccaaaccgggaaaaucgcugauuauaauuauaaauugccuga
cgauuuuaccggcuguguaaucgccuggaauuccaacaaucuugauuccaagguuggcggcaacuacaacuaucucuac
cgucuguuucgcaaauccaaucucaagcccuucgaacgcgauauuucaacugaaaucuaucaggcaggguccacuccgug
uaacggcguagaaggauuuaauuguuacuucccauugcaaaguuauggcuuucaacccaccaacggagucggguaccaa
ccauacagagucgucgugcucucauuugagcuccuucaugcaccugccacggugugcggcccaaagaaaucaacgaaccu
ugugaagaauaaaugugucaauuuuaacuuuaauggccugacagggacuggcguccucacagaaucuaauaagaaguuu
cucccuuuccagcaauuuggucgcgauauagcugauaccacagaugcaguuagagacccacagacacuugagauucucga
uauuaccccgugcuccuuuggcggcguguccgucauuacucccgguaccaauacgucuaaccagguagcagugcucuac
caagauguaaauuguacugagguacccguggcaauccaugccgaccaacugacuccaacguggcggguuuauucaaccg
gaagcaacguguuucaaacacgggcuggcugccuuauaggcgcugagcacgugaauaauaguuacgagugugauauccc
gaucggagccggcaucugugcaucuuaucaaacacaaacaaacuccccgcgccggcggagaagcguggcuagccaaagua
uaaucgcuuauacaauguccuugggcgcggaaaauucaguggcuuauuccaauaauucaauugccauuccuaccaacuu
uacaauuagcgugaccacagaaaucuugccugugucuaugaccaagaccagcgucgauugcaccauguauaucugugga
gauagcaccgaguguucaaauuugcuccugcaauacgguuccuuuuguacacagcuuaaccgcgcccucacagguauug
cuguugaacaagacaagaauacucaagagguauuugcucagguaaaacaaauuuauaagaccccaccgauaaaagauuuu
ggcgguuucaauuucucccaaauauugccagauccuaguaggcgucguagaucauuuaucgaggaucuccuguucaaua
aaguaacccucgccgacgcugguuucaucaaacaauauggcgacugccugggagauauugcagcuagggauuugauuug
ugcacagaaguucaauggacucaccguucucccgccucuccugacagaugagaugauugcacaauacaccucugcucuuu
uggccgggaccauuacgagcgguuggacuuuuggcgcgggugcggcucuccaaauuccuuucgcgaugcaaauggcgu
auagauuuaauggaauuggcguuacucaaaacgucuuguacgagaaucagaaacugaucgccaaccaauuuaacagugca
auuggcaaaauccaagauagccuuaguucuacugcuucagcauuggguaaguugcaagauguggucaaccaaaacgcaca
agcacucaauacccucgugaagcaauuguccagcaauuuuggagcuaucucaagugugcucaacgacauccuuucuaggc
uugauccacccgaggcagagguucaaaucgacagacugauaacuggcaggcuccaaucucugcaaacguacgugacacag
caacugauuagggcugcugagaucagggcguccgcgaauuuggcagcaaccaaaaugagcgaaugcgugcugggacaau
caaagagaguugauuucuguggaaaggguuaccaucucauguccuucccucaaucagcuccccauggaguuguguuucu
gcacguuacuuacgugccggcacaagaaaagaauuucaccacugcaccggcuauaugucaugaugggaaagcccacuucc
cgcgggaaggcguuuucguguccaacgggacucauugguucgucacacaaaggaacuucuaugagccacaaauaauuac
aacagacaacaccuuugucucugggaacugcgaugucgugauuggaaucgugaacaacacugucuacgauccgcugcaac
ccgaacucgacucauucaaagaggaacuggauaaguauuucaagaaccauaccagccccgaugucgaucugggcgauauc
uccgggauaaaugcuucaguaguaaacauucaaaaggaaaucgaccggcugaacgagguugcgaagaaucuuaaugagu
cauugaucgaccugcaagaacuugguaaguaugagcaguacaucaaguggccuuggucaggccgcaggcgucggcgucg
ugggagcggcggcagugggagcggauauauuccagaagcgccccgagacggacaagcuuacguacgaaaagacggagaa
uggguacugcuuuccacuuuucuuggcuaaug (SEQ ID NO: 147)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQPTN
GVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQDV
NCTEVPVAIHADQLTPTWRVYSTGSNVFQTRAGCLIGAEHVNNSYECDIPIGAGICAS
YQTQTNSPRRRRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQVKQIYK
TPPIKDFGGFNFSQILPDPSRRRRSFIEDLLFNKVTLADAGFIKQYGDCLGDIAARDLIC
AQKFNGLTVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 148)
WuS_3F_D2P_GlyDSol_pVax
ggatccgccaccatggattggacctggatcttgtttctcgtcgcagctgccacaagagtccacagtatgtttgtatttctcgttctcctgcct
cttgtgtcctctcagtgtgtgaatctgacgaccagaactcagctcccacccgcatacactaacagtatcacgaggggcgtttattaccca
gacaaggttttccgaagcagttgtctgtatagtacacaggatttgttcctgccattctttagtaacgtgacttggtttcatgcgatccacgttt
ctggcacaaacggtaccaaaagattcgataaccccgtgctgcctttcaatgacggcgtgtatttcgcttctactgaaaagtccggcatca
tccggggatggatcttcggcactactttggatagcaaaacccaatccctgctgattgtgaataatgcaaccaacgtggtgatcaaagtct
gtgagtttcaattctgtaatgacccatttctgggcgtttactaccacaagaacaacaaaagttggatggagtccgaatttcgcgtctactca
tccgcgaacaattgcacatttgagtatgtgagtcaaccattcttgatggatcttgaagggaagcaaggcaatttcaagaacctgcggga
gtttgtatttaagaacattgacggctatttcaagatatattctaaacatactcccattaacctcgtgcgcgacttgccccagggattcagtgc
tctggaaccactggtcgatctccccatcggcattaatattacacgctttcaaactctcctcgctttgcatcggtcctatcttactccgggaga
tagctcaagcggatggacggcaggcgcggcggcatattatgttggatatctccaaccacgcacgttccttctgaaatataatgaaaatg
gcactattactgatgcagtcgactgcgccctggaccctctgtctgagaccaagtgtaccctgaagtcattcaccgttgagaagggaatct
accaaacctctaactttagggtacaacctactgagagcattgtccgcttcccgaatatcaccaatctttgtccgtttggtgaggtgtttaac
gcgacacgatttgcttccgtatacgcctggaatcgcaaacgcatcagcaactgtgtggctgattactcatttctctataattccgcttccttc
tctactttcaagtgttatggggtgaatgggactaaacttaatgacttgtgctttactaacgtgtatgctgatagcttcgtcattcgtggagatg
aggtcaggcaaatagctcccggacaaacagggaagatagcggactataactacaaactgcctgatgatttcaccgggtgcgtcatcg
cgtggaacagcaataacctcgatagcaaggttggcggaaactataattatctctatcgtttgtttaggaaaagcaatctcaagcccttcga
gcgggatattaatacaacgatatatcaagctggctctaccccgtgcaacggagtagagggcttcaattgctactttcctttgcagtcctac
ggattccaacccaccaacggagtgggctaccaaccataccgtgtcgtggttttgagtttcgaactgaaccacgcaccagcaacagtct
gcggaccgaagaagagtacaaaccttgtgaagaataagtgcgtgaactttaatttcaatggcctgactggaaccggagttctgacgga
atccaataagaaatttctgccgtttcagcaatttggacgggattgtgctggaacgactgatgccgtacgtgatcctcaaacactggaaatc
ctggacataaccccttgttcctttggtggcgtaagcgttattactccaggcacaaacacatcaaatcaagtcgccgtactgtatcaaggtg
tcaactgtactgaagtacctgtagccattcatgcaggacaactgacccctacatggcgagtgtattcaacgggaagcaacgtatttcaaa
ccagggccggctgtctcatcggagcagagcatgtcaataatagttatgaatgcgacatcccaataggtgctgggatctgcgcgagcta
ccaaacccaaactaatagcccacgaagacggagatctgtcgcgtcccaaagcattattgcgtacacgatgagcctctgtgcagaaaat
tcagttgcctacagcaacaatagcatcgctattccaaccaatttcactatcagctgtacaacagaaattctcccagtctccatgacgaaga
catcctgcgattgtacaatgtatatatgcggcgactcaacagaatgttcaaatttgttgctgcaatacgggtccttctgcacccaactcaat
cgagctcttacaggcatagcggtcgaacaagacaagaacacacaagaagtgtttgcccaagttaaacagtgttacaagacaccaccta
tcaaagatttcggcggttttaacttttctcagatcttgccagacccatctaggcggcggcgatcctttatcgaggaccttctcttcaataagg
taactcttgcagacgctggatttattaagcaatacggcgactgtctcggggatatcgccgctagggatctgatctgtgcccagaaatttaa
cggctgcacggtgctgccccctctgctgactgatgaaatgatagcacaatatacttctgcattgctggccggtaccattacatcaggatg
gacatttggtgccggggggcgctccaaattcccttcgccatgcaaatggcctataggtttaacggcatcggggtgacccaaaacgtc
ctctatgagaatcaaaagctgattgctaaccagtttaactcagcaataggaaagattcaagactctctgtcaagtaccgcatccgcccttg
gaaagctccaagacgttgttaaccagaatgcacaagctctcaacacgctcgtgaaacaactctcttcaaattttggtgcgatctcttctgg
cccaaatgacattttgagccggcttcccaaggtagaagctgaagtacaaattgatcgcctgatcaccggacggctccaaagtctgcag
acgtacgtcacccagcaactgatacgggcagcggagatccgggcttctgccaacctggccgccacgaagatgagcgaatgcgtgct
cggacagtccaaaagagtagatttctgtggcaagggctatcatctcatgtcctttccccaatccgcccctcacggagttgtcttccttcatt
gcacttacgtccccgctcaagaaaagaattttactacggcacctgctatctgtcacgacgggaaagcccattttcctagagaaggtgtgt
ttgtatctaacggcacgcactggttcgtcacgcaacgtaacttttacgagccccagatcatcaccacagacaatacggatgtatcaggta
attgtgatgtcctgattggtatcgtcaataacactgtatacgatcctttgcaaccggaactggactcctttaaagaggaacttgataagtatt
tcaagaatcacacttccccagatgtcgatctcggggacatctcaggaattaatgcatcagtggtcaatattcaaaaggaaattgatcgctt
gaatgaggttgcaaagaatttgaatgaaagccttatcgaccttcaagagctgggcaaatatgagcagtacattaaatggccttggagcg
gtcgccggcgccgaaggcggggttccggcggtagcggtagcggttatattccagaagctcctcgcgatgggcaggcttatgtgagg
aaagatggtgaatgggtccttttgtccacgttcctcgggtagtaa (SEQ ID NO: 149)
ggauccgccaccauggauuggaccuggaucuuguuucucgucgcagcugccacaagaguccacaguauguuuguauuuc
ucguucuccugccucuuguguccucucagugugugaaucugacgaccagaacucagcucccacccgcauacacuaacagu
aucacgaggggcguuuauuacccagacaagguuuuccgaagcaguugucuguauaguacacaggauuuguuccugccau
ucuuuaguaacgugacuugguuucaugcgauccacguuucuggcacaaacgguaccaaaagauucgauaaccccgugcu
gccuuucaaugacggcguguauuucgcuucuacugaaaaguccggcaucauccggggauggaucuucggcacuacuuug
gauagcaaaacccaaucccugcugauugugaauaaugcaaccaacguggugaucaaagucugugaguuucaauucugua
augacccauuucugggcguuuacuaccacaagaacaacaaaaguuggauggaguccgaauuucgcgucuacucauccgcg
aacaauugcacauuugaguaugugagucaaccauucuugauggaucuugaagggaagcaaggcaauuucaagaaccugc
gggaguuuguauuuaagaacauugacggcuauuucaagauauauucuaaacauacucccauuaaccucgugcgcgacuu
gccccagggauucagugcucuggaaccacuggucgaucuccccaucggcauuaauauuacacgcuuucaaacucuccucg
cuuugcaucgguccuaucuuacuccgggagauagcucaagcggauggacggcaggcgcggcggcauauuauguuggaua
ucuccaaccacgcacguuccuucugaaauauaaugaaaauggcacuauuacugaugcagucgacugcgcccuggacccuc
ugucugagaccaaguguacccugaagucauucaccguugagaagggaaucuaccaaaccucuaacuuuaggguacaaccu
acugagagcauuguccgcuucccgaauaucaccaaucuuuguccguuuggugagguguuuaacgcgacacgauuugcuu
ccguauacgccuggaaucgcaaacgcaucagcaacuguguggcugauuacucauuucucuauaauuccgcuuccuucuc
uacuuucaaguguuauggggugaaugggacuaaacuuaaugacuugugcuuuacuaacguguaugcugauagcuucgu
cauucguggagaugaggucaggcaaauagcucccggacaaacagggaagauagcggacuauaacuacaaacugccugaug
auuucaccgggugcgucaucgcguggaacagcaauaaccucgauagcaagguuggcggaaacuauaauuaucucuaucg
uuuguuuaggaaaagcaaucucaagcccuucgagcgggauauuaauacaacgauauaucaagcuggcucuaccccgugca
acggaguagagggcuucaauugcuacuuuccuuugcaguccuacggauuccaacccaccaacggagugggcuaccaacca
uaccgugucgugguuuugaguuucgaacugaaccacgcaccagcaacagucugcggaccgaagaagaguacaaaccuug
ugaagaauaagugcgugaacuuuaauuucaauggccugacuggaaccggaguucugacggaauccaauaagaaauuucu
gccguuucagcaauuuggacgggauugugcuggaacgacugaugccguacgugauccucaaacacuggaaauccuggac
auaaccccuuguuccuuugguggcguaagcguuauuacuccaggcacaaacacaucaaaucaagucgccguacuguauca
aggugucaacuguacugaaguaccuguagccauucaugcaggacaacugaccccuacauggcgaguguauucaacggga
agcaacguauuucaaaccagggccggcugucucaucggagcagagcaugucaauaauaguuaugaaugcgacaucccaau
aggugcugggaucugcgcgagcuaccaaacccaaacuaauagcccacgaagacggagaucugucgcgucccaaagcauua
uugcguacacgaugagccucugugcagaaaauucaguugccuacagcaacaauagcaucgcuauuccaaccaauuucacu
aucagcuguacaacagaaauucucccagucuccaugacgaagacauccugcgauuguacaauguauauaugcggcgacuc
aacagaauguucaaauuuguugcugcaauacggguccuucugcacccaacucaaucgagcucuuacaggcauagcgguc
gaacaagacaagaacacacaagaaguguuugcccaaguuaaacaguguuacaagacaccaccuaucaaagauuucggcgg
uuuuaacuuuucucagaucuugccagacccaucuaggcggcggcgauccuuuaucgaggaccuucucuucaauaaggua
acucuugcagacgcuggauuuauuaagcaauacggcgacugucucggggauaucgccgcuagggaucugaucugugccc
agaaauuuaacggcugcacggugcugcccccucugcugacugaugaaaugauagcacaauauacuucugcauugcuggc
cgguaccauuacaucaggauggacauuuggugccggggcggcgcuccaaauucccuucgccaugcaaauggccuauagg
uuuaacggcaucggggugacccaaaacguccucuaugagaaucaaaagcugauugcuaaccaguuuaacucagcaauagg
aaagauucaagacucucugucaaguaccgcauccgcccuuggaaagcuccaagacguuguuaaccagaaugcacaagcuc
ucaacacgcucgugaaacaacucucuucaaauuuuggugcgaucucuucuggcccaaaugacauuuugagccggcuucc
caagguagaagcugaaguacaaauugaucgccugaucaccggacggcuccaaagucugcagacguacgucacccagcaac
ugauacgggcagcggagauccgggcuucugccaaccuggccgccacgaagaugagcgaaugcgugcucggacaguccaa
aagaguagauuucuguggcaagggcuaucaucucauguccuuuccccaauccgccccucacggaguugucuuccuucau
ugcacuuacguccccgcucaagaaaagaauuuuacuacggcaccugcuaucugucacgacgggaaagcccauuuuccuag
agaagguguguuuguaucuaacggcacgcacugguucgucacgcaacguaacuuuuacgagccccagaucaucaccacag
acaauacggauguaucagguaauugugauguccugauugguaucgucaauaacacuguauacgauccuuugcaaccgga
acuggacuccuuuaaagaggaacuugauaaguauuucaagaaucacacuuccccagaugucgaucucggggacaucucag
gaauuaaugcaucaguggucaauauucaaaaggaaauugaucgcuugaaugagguugcaaagaauuugaaugaaagccu
uaucgaccuucaagagcugggcaaauaugagcaguacauuaaauggccuuggagcggucgccggcgccgaaggcggggu
uccggcgguagcgguagcgguuauauuccagaagcuccucgcgaugggcaggcuuaugugaggaaagauggugaaugg
guccuuuuguccacguuccucggguaguaa (SEQ ID NO: 150)
MDWTWILFLVAAATRVHSMFVFLVLLPLVSSQCVNLTTRTQLPPAYTNSITRGVYYP
DKVFRSSCLYSTQDLFLPFFSNVTWFHAIHVSGTNGTKRFDNPVLPFNDGVYFASTEK
SGIIRGWIFGTTLDSKTQSLLIVNNATNVVIKVCEFQFCNDPFLGVYYHKNNKSWMES
EFRVYSSANNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAAAYYVGYLQ
PRTFLLKYNENGTITDAVDCALDPLSETKCTLKSFTVEKGIYQTSNFRVQPTESIVRFP
NITNLCPFGEVFNATRFASVYAWNRKRISNCVADYSFLYNSASFSTFKCYGVNGTKL
NDLCFTNVYADSFVIRGDEVRQIAPGQTGKIADYNYKLPDDFTGCVIAWNSNNLDSK
VGGNYNYLYRLFRKSNLKPFERDINTTIYQAGSTPCNGVEGFNCYFPLQSYGFQPTN
GVGYQPYRVVVLSFELNHAPATVCGPKKSTNLVKNKCVNFNFNGLTGTGVLTESNK
KFLPFQQFGRDCAGTTDAVRDPQTLEILDITPCSFGGVSVITPGTNTSNQVAVLYQGV
NCTEVPVAIHAGQLTPTWRVYSTGSNVFQTRAGCLIGAEHVNNSYECDIPIGAGICAS
YQTQTNSPRRRRSVASQSIIAYTMSLCAENSVAYSNNSIAIPTNFTISCTTEILPVSMTK
TSCDCTMYICGDSTECSNLLLQYGSFCTQLNRALTGIAVEQDKNTQEVFAQVKQCYK
TPPIKDFGGFNFSQILPDPSRRRRSFIEDLLFNKVTLADAGFIKQYGDCLGDIAARDLIC
AQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTDVSGNCDVLIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 151)
WuS_3F_D2P_GlyD3_pVax
ggatccgccaccatggactggacatggatacttttcttggtagcggcggcgacacgcgtgcactccatgtttgttttcctcgtcctgctcc
cacttgtctcaagtcaatgcgttaacctgactacgaggacgcagctcccgcccgcctacacaaactcttttacccggggtgtgtactacc
ccgacaaagttttccgcagttcatgtctccactcaacacaggacctctttctgccattcttctcaaatgtcacatggtttcacgccatccacg
tttccggcactaacggtaccaaacggttcgacaaccctgttctgccattcaatgatggggtgtattttgcgagcacagagaagtccaatat
aatcagaggttggatcttcggtacaacgctggacagtaaaactcaatctctgctgatagtgaataacgctacgaacgtcgtcattaaggt
gtgcgagtttcaattttgcaacgatccattcttgggagtgtactatcataagaacaacaaatcatggatggagagcgagtttagggtgtatt
cctctgcaaacaactgtacatttgaatacgtgagccagccttttcttatggacctcgaaggtaagcaaggtaacttcaagaacttgcggg
aatttgttttcaagaacattgatggatacttcaaaatttactccaaacatacccctatcaatctggtccgcgaccttccacaaggattttccgc
acttgaacccttggtcgacctgcctattggaatcaatatcacgcggtttcagacgcttctcgctctccatagatcctacctcacgcccggc
gacagttcaagtgggtggaccgcaggcgcggcggcctattatgtgggatacttgcaaccccgcacttttctcctgaaatataatgagaa
tgggaccataaccgatgcagttgattgtgccttggaccccctgtccgagaccaaatgcacgctgaagtctttcacagtagagaaggga
atttaccaaacttccaacttcagagttcaacccacagaatctatcgttcgctttcccaatattacaaatttgtgtccgtttggagaggtgttca
atgctacaaggtttgcttccgtatatgcctggaatcgtaaacgcatctctaattgcgtagcggactactcagttttgtataacagtgctagct
tctccactttcaagtgttacggcgttaatgggaccaagctgaatgacctgtgttttaccaacgtgtatgctgactccttcgtaataagaggg
gatgaggttaggcaaatcgcccctggccagacagggaaaatcgctgattacaattacaagttgccagatgactttaccgggtgtgtcat
cgcttggaactccaataatctggattccaaagttggtgggaactataattacctctatcggctgttcagaaaatccaaccttaagcccttcg
aaagagatatcaacactacaatttatcaggctggttcaactccgtgtaatggggtcgagggtttcaactgctacttcccgttgcagagttat
gggttccagccgacgaatggggtcgggtaccaaccgtacagagtagtagttctgtcctttgagttgaatcatgccccagcaacagtgtg
cggcccaaagaaatcaacaaaccttgttaagaataaatgcgtgaacttcaactttaacgggcttactgggactggggtgctcacagaat
ccaacaagaaattcttgccattccaacaatttggccgcgattgtgcagatacaaccgacgccgtgagagatccccaaacattggagata
cttgatatcactccctgctcttttggtggcgtcagcgtcatcaccccaggaaccaatacaagcaatcaagtggctgtcctttatcaagatgt
caattgtaccgaagtcccagtcgcaatacatgcggatcaactgaccccaacatggagagtttactcaacgggatctaacgtgtttcaaa
ctcgtgctggctgcctgataggagcggagcatgtgaataattcctatgaatgcgacattcccattggggctggaatctgtgcatcctatc
aaacacaaactaactctccccgccggcggcggagcgtcgccagccaaagcattattgcatatacgatgtccctgtgcgcagaaaattc
tgttgcatacagcaataactccatcgctatccctacaaactttaccatcagctgtacaaccgaaatcttgcccgtttctatgactaaaacaa
gttgtgactgcactatgtacatctgtggcgactcaacagagtgttctaaccttctgcttcaatatggatctttctgtacacaacttaatcgcgc
tctcaccggtatagctgttgagcaagataagaacactcaggaagttttcgcccaagtcaaacaatgttataaaacaccacccataaaag
acttcggcggatttaatttctctcaaatactgccggacccatccaggagacgaagaagcttcatagaagatcttctcttcaacaaggtgac
cctggccgatgcggggtttatcaagcaatatggcgactgtctcggcgatattgctgcacgcgatctgatatgtgcacagaaattcaatgg
gtgtaccgtgctcccacctctgctgacagatgaaatgatcgctcaatataccagtgcgctcttggctggaacaattactagtggttggact
tttggggctggagccgcactccaaatcccttttgccatgcaaatggcctatcgctttaatgggataggggtcactcagaatgtcttgtatg
aaaaccagaagttgattgctaaccaatttaattcagctatagggaaaattcaagacagcctcagtagtactgccagtgccctgggcaaa
ctgcaagatgtcgtgaaccaaaatgctcaagccctgaataccctcgttaagcaacttagctcaaactttggtgcgatttcctcaggcccta
atgacatcctctcaaggctgcctaaagtggaagctgaggtccaaatcgatcgcctgattacgggtcgcctgcaatcactccaaacatat
gtcacccagcagttgatcagagcggccgagatacgggcatcagcaaatttggcggccacgaaaatgtcagagtgcgtacttggtcaa
agtaaaagagttgatttctgcggaaaaggttaccaccttatgtctttcccccagtccgctccacatggagtggtctttctgcattgtacttat
gtgccagcccaagaaaagaattttactaccgcccccgctatttgtcatgatggtaaggcgcacttccccagagaaggagtgtttgtgtcc
aacgggactcactggtttgtgactcaaaggaacttttatgaacctcaaattatcaccacagataacacatttgtgtccgggaattgcgatgt
ggttatcggcattgttaataataccgtttacgatcccttgcaacctgagttggatagtttcaaggaagaacttgacaaatactttaagaatca
cacttccccggatgtagacctcggggacatttccggaattaatgcgagtgttgtgaatatacagaaagagatagaccgactcaacgag
gttgctaagaacctcaacgagagccttatcgatcttcaagaactcggcaaatacgagcaatacattaaatggccttggtccggcagaag
gagacggcgaaggggaagtggcggcagcggctctggatacatcccggaagctccacgggatgggcaagcatatgttcgcaaggat
ggagaatgggtccttcttagcaccttcttgggataatga (SEQ ID NO: 152)
ggauccgccaccauggacuggacauggauacuuuucuugguagcggcggcgacacgcgugcacuccauguuuguuuucc
ucguccugcucccacuugucucaagucaaugcguuaaccugacuacgaggacgcagcucccgcccgccuacacaaacucu
uuuacccgggguguguacuaccccgacaaaguuuuccgcaguucaugucuccacucaacacaggaccucuuucugccau
ucuucucaaaugucacaugguuucacgccauccacguuuccggcacuaacgguaccaaacgguucgacaacccuguucug
ccauucaaugaugggguguauuuugcgagcacagagaaguccaauauaaucagagguuggaucuucgguacaacgcugg
acaguaaaacucaaucucugcugauagugaauaacgcuacgaacgucgucauuaaggugugcgaguuucaauuuugcaa
cgauccauucuugggaguguacuaucauaagaacaacaaaucauggauggagagcgaguuuaggguguauuccucugca
aacaacuguacauuugaauacgugagccagccuuuucuuauggaccucgaagguaagcaagguaacuucaagaacuugc
gggaauuuguuuucaagaacauugauggauacuucaaaauuuacuccaaacauaccccuaucaaucugguccgcgaccuu
ccacaaggauuuuccgcacuugaacccuuggucgaccugccuauuggaaucaauaucacgcgguuucagacgcuucucg
cucuccauagauccuaccucacgcccggcgacaguucaaguggguggaccgcaggcgcggcggccuauuaugugggaua
cuugcaaccccgcacuuuucuccugaaauauaaugagaaugggaccauaaccgaugcaguugauugugccuuggaccccc
uguccgagaccaaaugcacgcugaagucuuucacaguagagaagggaauuuaccaaacuuccaacuucagaguucaaccc
acagaaucuaucguucgcuuucccaauauuacaaauuuguguccguuuggagagguguucaaugcuacaagguuugcuu
ccguauaugccuggaaucguaaacgcaucucuaauugcguagcggacuacucaguuuuguauaacagugcuagcuucuc
cacuuucaaguguuacggcguuaaugggaccaagcugaaugaccuguguuuuaccaacguguaugcugacuccuucgua
auaagaggggaugagguuaggcaaaucgccccuggccagacagggaaaaucgcugauuacaauuacaaguugccagaug
acuuuaccgggugugucaucgcuuggaacuccaauaaucuggauuccaaaguuggugggaacuauaauuaccucuaucg
gcuguucagaaaauccaaccuuaagcccuucgaaagagauaucaacacuacaauuuaucaggcugguucaacuccgugua
auggggucgaggguuucaacugcuacuucccguugcagaguuauggguuccagccgacgaauggggucggguaccaacc
guacagaguaguaguucuguccuuugaguugaaucaugccccagcaacagugugcggcccaaagaaaucaacaaaccuug
uuaagaauaaaugcgugaacuucaacuuuaacgggcuuacugggacuggggugcucacagaauccaacaagaaauucuu
gccauuccaacaauuuggccgcgauugugcagauacaaccgacgccgugagagauccccaaacauuggagauacuugaua
ucacucccugcucuuuugguggcgucagcgucaucaccccaggaaccaauacaagcaaucaaguggcuguccuuuaucaa
gaugucaauuguaccgaagucccagucgcaauacaugcggaucaacugaccccaacauggagaguuuacucaacgggauc
uaacguguuucaaacucgugcuggcugccugauaggagcggagcaugugaauaauuccuaugaaugcgacauucccauu
ggggcuggaaucugugcauccuaucaaacacaaacuaacucuccccgccggcggcggagcgucgccagccaaagcauuau
ugcauauacgaugucccugugcgcagaaaauucuguugcauacagcaauaacuccaucgcuaucccuacaaacuuuacca
ucagcuguacaaccgaaaucuugcccguuucuaugacuaaaacaaguugugacugcacuauguacaucuguggcgacuc
aacagaguguucuaaccuucugcuucaauauggaucuuucuguacacaacuuaaucgcgcucucaccgguauagcuguu
gagcaagauaagaacacucaggaaguuuucgcccaagucaaacaauguuauaaaacaccacccauaaaagacuucggcgga
uuuaauuucucucaaauacugccggacccauccaggagacgaagaagcuucauagaagaucuucucuucaacaaggugac
ccuggccgaugcgggguuuaucaagcaauauggcgacugucucggcgauauugcugcacgcgaucugauaugugcacag
aaauucaauggguguaccgugcucccaccucugcugacagaugaaaugaucgcucaauauaccagugcgcucuuggcug
gaacaauuacuagugguuggacuuuuggggcuggagccgcacuccaaaucccuuuugccaugcaaauggccuaucgcuu
uaaugggauaggggucacucagaaugucuuguaugaaaaccagaaguugauugcuaaccaauuuaauucagcuauaggg
aaaauucaagacagccucaguaguacugccagugcccugggcaaacugcaagaugucgugaaccaaaaugcucaagcccu
gaauacccucguuaagcaacuuagcucaaacuuuggugcgauuuccucaggcccuaaugacauccucucaaggcugccua
aaguggaagcugagguccaaaucgaucgccugauuacgggucgccugcaaucacuccaaacauaugucacccagcaguug
aucagagcggccgagauacgggcaucagcaaauuuggcggccacgaaaaugucagagugcguacuuggucaaaguaaaa
gaguugauuucugcggaaaagguuaccaccuuaugucuuucccccaguccgcuccacauggaguggucuuucugcauug
uacuuaugugccagcccaagaaaagaauuuuacuaccgcccccgcuauuugucaugaugguaaggcgcacuuccccagag
aaggaguguuuguguccaacgggacucacugguuugugacucaaaggaacuuuuaugaaccucaaauuaucaccacaga
uaacacauuuguguccgggaauugcgaugugguuaucggcauuguuaauaauaccguuuacgaucccuugcaaccugag
uuggauaguuucaaggaagaacuugacaaauacuuuaagaaucacacuuccccggauguagaccucggggacauuuccg
gaauuaaugcgaguguugugaauauacagaaagagauagaccgacucaacgagguugcuaagaaccucaacgagagccuu
aucgaucuucaagaacucggcaaauacgagcaauacauuaaauggccuugguccggcagaaggagacggcgaaggggaag
uggcggcagcggcucuggauacaucccggaagcuccacgggaugggcaagcauauguucgcaaggauggagaauggguc
cuucuuagcaccuucuugggauaauga (SEQ ID NO: 153)
MDWTWILFLVAAATRVHSMFVFLVLLPLVSSQCVNLTTRTQLPPAYTNSFTRGVYY
PDKVFRSSCLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDCADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLCAENSVAYSNNSIAIPTNFTISCTTEILPVSMT
KTSCDCTMYICGDSTECSNLLLQYGSFCTQLNRALTGIAVEQDKNTQEVFAQVKQCY
KTPPIKDFGGFNFSQILPDPSRRRRSFIEDLLFNKVTLADAGFIKQYGDCLGDIAARDLI
CAQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 154)
WuS_3F_D2P_GlyD2_pVax
ggatccgccaccatggattggacatggatactgtttctggtcgctgctgccacacgtgtccacagcatgtttgtcttcttggtgctcttgcc
tcttgtgagctcccaatgtgtgaatctgactacacgtacgcaacttccgcctgcctacaccaactctttcaccagaggcgtgtattatccg
gataaggtgttcaggagctcctgccttcattcaacacaggatttgtttctgcctttcttttcaaacgttacttggttccatgccatccacgtgtc
aggaacaaatggtaccaagagattcgataacccagttctcccttttaatgatggagtctattttgcaagcactgagaaaagtaatattatac
gaggttggattttcggaacgacactcgacagtaaaacacaatccctgttgatagtcaacaatgccacgaacgtagttataaaagtttgcg
aatttcaattttgcaacgatcctttcctgggtgtgtactatcacaagaacaacaaatcttggatggaaagcgagtttcgagtgtattcttcag
caaacaactgtactttcgaatatgtttctcaaccattcctgatggatctcgaaggtaaacagggcaactttaagaatctgagagagtttgtg
tttaagaacattgacggctattttaagatttacagcaaacatacgcctataaaccttgtgagagacctgcctcaagggtttagcgccctgg
aaccactcgtggacctgcctatcggcatcaatattaccagatttcaaacgctccttgccctgcataggagctatttgacacctggggactc
ttctagcggctggactgcaggcgctgccgcttattacgtgggatatctccagcctagaactttcctcttgaaatacaacgagaatggaac
cataacagacgcagttgattgtgctctcgaccccttgtccgagaccaaatgcacactgaaaagttttaccgtggagaaagggatctatca
aactagtaatttccgcgttcaacccactgagagcatagtgaggtttcctaacattacaaatctttgcccgtttggggaagtgtttaatgcca
ctcgttttgctagtgtatacgcctggaatcgaaagcggatttccaattgcgttgctgactacagtgtactctataatagcgcttcatttagca
ccttcaagtgctacggggttaacgggaccaaactcaatgacctctgcttcacgaacgtttacgccgactcctttgtcattcgaggtgacg
aagtaagacaaatcgccccaggccagactggaaagatcgcggactacaactataagctgccagacgacttcactggatgtgtgatcg
cctggaatagtaacaacctcgactccaaggtgggtggcaattacaattatctctataggctgttcaggaagagtaatttgaaaccattcga
gcgcgacataaatacaacaatctaccaagcgggttctaccccttgcaacggcgtggaaggttttaattgttacttccctctccaaagctac
gggtttcaaccaacaaacggcgtgggataccaaccatacagggtggttgtgttgagcttcgaattgaatcatgcacctgcaacagtgtg
tgggcccaagaagtccaccaatctcgttaagaataaatgcgtgaacttcaactttaacgggttgacagggaccggcgtgcttacggaa
agtaataagaaattccttcccttccagcaatttggtcgcgactgtgcggatacaacggacgcagtgcgagacccacagacattggagat
cctggacataacaccttgctcttttggggggtctccgtaataacacctggaacaaataccagcaatcaagtagcggtcttgtatcaaga
tgtaaactgtactgaagtcccagttgctatacatgcagaccaacttacaccgacgtggcgcgtgtattctacgggctccaacgtattcca
aaccagagcagggtgcttgataggggcagagcacgtcaacaatagctatgagtgtgatatcccgataggtgctggaatctgcgcaag
ttaccagacccaaaccaatagcccccgccggagacgatcagtggcaagccagtctataatagcctacacgatgtcactgtgtgccga
aaatagcgttgcctatagtaacaatagcatcgccattccaaccaatttcacaatatcagtcactactgagattctgcctgtgtcaatgacta
aaactagtgtggactgcacaatgtatatttgcggcgattccacagaatgtagcaatcttctgctgcaatatgggagtttctgtacacaattg
aatcgggcccttactggaatcgccgtagagcaggacaagaacacccaagaagtctttgcgcaagtcaaacaatgttataagactcccc
caattaaagattttggcggctttaattttagccaaatacttcccgaccccagccgccgacgacgctcctttatcgaagatctgttgtttaata
aagtcacattggctgatgctggctttatcaaacaatacggtgattgtctgggtgatattgcagcccgagatctgatctgcgcccaaaagtt
taacggcttgaccgttctcccgccactcctgacagatgagatgatcgcgcaatatacctctgcactcctggcgggaacaatcactagtg
gttggactttcggcgccggcgctgcactgcaaattcccttcgccatgcaaatggcctatcggtttaacggaattggtgtgactcagaatg
tgctttacgaaaatcagaaactcatagctaatcagtttaacagcgcaatcgggaaaattcaggattccctcagcagcaccgctagcgcct
tgggcaagctgcaggacgttgtaaaccagaacgctcaggccctcaacactctcgttaaacaattgagctctaactttggggccataagc
agtggtcctaacgacatcctgagtcgtctgccaaaggtagaggccgaagtgcaaatcgaccggctcatcactggaagactgcaaagc
ctgcaaacctatgtcacacagcaacttatacgggccgccgaaatcagggcctcagcaaacctcgcagcaacaaagatgagcgagtgt
gtgctgggccaatccaagcgcgtggacttctgtggtaagggataccatctgatgtcctttccccaatccgcgcctcatggagtagttttcc
tgcacgttacgtatgtgcctgcccaagagaagaactttacaacagcaccagccatttgtcatgacggaaaagcccattttcctagagaa
ggagtctttgtttccaatgggacacattggtttgttacccagcgtaacttttatgagccacaaatcatcaccacggacaatactttcgtgag
cggtaattgtgatgtggtcattggcatagtgaataacactgtttacgaccccctgcaaccggaattggacagcttcaaagaagaactgga
caagtacttcaagaaccacacatccccagacgtagacctcggagatatttccggaattaacgcatcagtagttaacatccagaaagaaa
tagatcgactgaatgaggtcgctaagaacttgaacgaatcacttatagatctccaggaactcggcaaatatgagcaatatattaaatggc
cctggtcaggtcgcagaagacgccgccggggttccggcggatctggatctggatatattcccgaagctccacgggatgggcaagcc
tacgtaagaaaggatggagaatgggtacttttgtccacgttcttgggctagtag (SEQ ID NO: 155)
ggauccgccaccauggauuggacauggauacuguuucuggucgcugcugccacacguguccacagcauguuugucuucu
uggugcucuugccucuugugagcucccaaugugugaaucugacuacacguacgcaacuuccgccugccuacaccaacuc
uuucaccagaggcguguauuauccggauaagguguucaggagcuccugccuucauucaacacaggauuuguuucugccu
uucuuuucaacguuacuugguuccaugccauccacgugucaggaacaaaugguaccaagagauucgauaacccaguuc
ucccuuuuaaugauggagucuauuuugcaagcacugagaaaaguaauauuauacgagguuggauuuucggaacgacacu
cgacaguaaaacacaaucccuguugauagucaacaaugccacgaacguaguuauaaaaguuugcgaauuucaauuuugca
acgauccuuuccuggguguguacuaucacaagaacaacaaaucuuggauggaaagcgaguuucgaguguauucuucagc
aaacaacuguacuuucgaauauguuucucaaccauuccugauggaucucgaagguaaacagggcaacuuuaagaaucuga
gagaguuuguguuuaagaacauugacggcuauuuuaagauuuacagcaaacauacgccuauaaaccuugugagagaccu
gccucaaggguuuagcgcccuggaaccacucguggaccugccuaucggcaucaauauuaccagauuucaaacgcuccuug
cccugcauaggagcuauuugacaccuggggacucuucuagcggcuggacugcaggcgcugccgcuuauuacgugggaua
ucuccagccuagaacuuuccucuugaaauacaacgagaauggaaccauaacagacgcaguugauugugcucucgaccccu
uguccgagaccaaaugcacacugaaaaguuuuaccguggagaaagggaucuaucaaacuaguaauuuccgcguucaaccc
acugagagcauagugagguuuccuaacauuacaaaucuuugcccguuuggggaaguguuuaaugccacucguuuugcua
guguauacgccuggaaucgaaagcggauuuccaauugcguugcugacuacaguguacucuauaauagcgcuucauuuag
caccuucaagugcuacgggguuaacgggaccaaacucaaugaccucugcuucacgaacguuuacgccgacuccuuuguca
uucgaggugacgaaguaagacaaaucgccccaggccagacuggaaagaucgcggacuacaacuauaagcugccagacgac
uucacuggaugugugaucgccuggaauaguaacaaccucgacuccaagguggguggcaauuacaauuaucucuauaggc
uguucaggaagaguaauuugaaaccauucgagcgcgacauaaauacaacaaucuaccaagcggguucuaccccuugcaac
ggcguggaagguuuuaauuguuacuucccucuccaaagcuacggguuucaaccaacaaacggcgugggauaccaaccau
acagggugguuguguugagcuucgaauugaaucaugcaccugcaacagugugugggcccaagaaguccaccaaucucgu
uaagaauaaaugcgugaacuucaacuuuaacggguugacagggaccggcgugcuuacggaaaguaauaagaaauuccuu
cccuuccagcaauuuggucgcgacugugcggauacaacggacgcagugcgagacccacagacauuggagauccuggacau
aacaccuugcucuuuuggcggggucuccguaauaacaccuggaacaaauaccagcaaucaaguagcggucuuguaucaag
auguaaacuguacugaagucccaguugcuauacaugcagaccaacuuacaccgacguggcgcguguauucuacgggcuc
caacguauuccaaaccagagcagggugcuugauaggggcagagcacgucaacaauagcuaugagugugauaucccgaua
ggugcuggaaucugcgcaaguuaccagacccaaaccaauagcccccgccggagacgaucaguggcaagccagucuauaau
agccuacacgaugucacugugugccgaaaauagcguugccuauaguaacaauagcaucgccauuccaaccaauuucacaa
uaucagucacuacugagauucugccugugucaaugacuaaaacuaguguggacugcacaauguauauuugcggcgauuc
cacagaauguagcaaucuucugcugcaauaugggaguuucuguacacaauugaaucgggcccuuacuggaaucgccgua
gagcaggacaagaacacccaagaagucuuugcgcaagucaaacaauguuauaagacucccccaauuaaagauuuuggcgg
cuuuaauuuuagccaaauacuucccgaccccagccgccgacgacgcuccuuuaucgaagaucuguuguuuaauaaaguca
cauuggcugaugcuggcuuuaucaaacaauacggugauugucugggugauauugcagcccgagaucugaucugcgccca
aaaguuuaacggcuugaccguucucccgccacuccugacagaugagaugaucgcgcaauauaccucugcacuccuggcgg
gaacaaucacuagugguuggacuuucggcgccggcgcugcacugcaaauucccuucgccaugcaaauggccuaucgguu
uaacggaauuggugugacucagaaugugcuuuacgaaaaucagaaacucauagcuaaucaguuuaacagcgcaaucggg
aaaauucaggauucccucagcagcaccgcuagcgccuugggcaagcugcaggacguuguaaaccagaacgcucaggcccu
caacacucucguuaaacaauugagcucuaacuuuggggccauaagcagugguccuaacgacauccugagucgucugccaa
agguagaggccgaagugcaaaucgaccggcucaucacuggaagacugcaaagccugcaaaccuaugucacacagcaacuu
auacgggccgccgaaaucagggccucagcaaaccucgcagcaacaaagaugagcgagugugugcugggccaauccaagcg
cguggacuucugugguaagggauaccaucugauguccuuuccccaauccgcgccucauggaguaguuuuccugcacguu
acguaugugccugcccaagagaagaacuuuacaacagcaccagccauuugucaugacggaaaagcccauuuuccuagaga
aggagucuuuguuuccaaugggacacauugguuuguuacccagcguaacuuuuaugagccacaaaucaucaccacggac
aaucuuucugagcgguaauugugauguggucauuggcauagugaauaacacuguuuacgacccccugcaaccggaau
uggacagcuucaaagaagaacuggacaaguacuucaagaaccacacauccccagacguagaccucggagauauuuccgga
auuaacgcaucaguaguuaacauccagaaagaaauagaucgacugaaugaggucgcuaagaacuugaacgaaucacuuau
agaucuccaggaacucggcaaauaugagcaauauauuaaauggcccuggucaggucgcagaagacgccgccgggguucc
ggcggaucuggaucuggauauauucccgaagcuccacgggaugggcaagccuacguaagaaaggauggagaauggguac
uuuuguccacguucuugggcuaguag (SEQ ID NO: 156)
MDWTWILFLVAAATRVHSMFVFLVLLPLVSSQCVNLTTRTQLPPAYTNSFTRGVYY
PDKVFRSSCLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDCADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLCAENSVAYSNNSIAIPTNFTISVTTEILPVSMT
KTSVDCTMYICGDSTECSNLLLQYGSFCTQLNRALTGIAVEQDKNTQEVFAQVKQC
YKTPPIKDFGGFNFSQILPDPSRRRRSFIEDLLFNKVTLADAGFIKQYGDCLGDIAARD
LICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTL
VKQLSSNFGAISSGPNDILSRLPKVEAEVQIDRLITGRLQSLQTYVTQQLIRAAEIRASA
NLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNL
NESLIDLQELGKYEQYIKWPWSGRRRRRRGSGGSGSGYIPEAPRDGQAYVRKDGEW
VLLSTFLG** (SEQ ID NO: 157)
WuS_3F_D2P_GlyD1_pVax
ggatccgccaccatggactggacatggatacttttcttggtggcagctgctacacgcgtccactcaatgttcgtctttctggtgctcttgcc
actggtgagcagccaatgcgttaacctcaccacacgcacgcagcttccacccgcatacactaactcctttacgcgcggcgtgtactatc
cagataaagtgttccgaagtagcgtcttgcatagcacccaggatctgtttctcccattctttagcaatgtcacatggttccacgctatccac
gtgtctgggacgaatggaactaaacgttttgacaatcctgttcttccttttaacgacggcgtatactttgctagtactgagaagtctaacatt
atccgcggctggattttcgggacaaccctggactccaaaacccagtctctgctgatagtaaacaatgccaccaacgtcgtcattaaagt
gtgcgagtttcaattctgcaacgacccctttctgggtgtctattaccacaagaacaataagtcttggatggagtcagaatttcgtgtctattc
ttctgccaataattgtacatttgagtatgtttctcaaccctttctcatggacctcgaaggcaagcaggggaattttaagaacctgcgggaatt
cgtctttaagaatatcgacggctatttcaaaatttacagcaaacacacgcctataaacctcgtgcgagatctcccccaaggcttctcagca
ttggagccattggtcgacttgccaatcggaattaatatcacaaggtttcagactctgctggccctgcatcgctcctatcttacccctggcg
attcctcaagtggctggacggccggcgcagcagcctattacgtcggctatctccagccaaggacgtttcttttgaagtataatgaaaatg
ggactattactgacgccgtcgactgcgctttggaccccctgagcgagacaaagtgcacattgaaaagcttcacggtggagaagggtat
ttatcaaacttccaactttagggtgcaaccaacagagagcatcgtgaggttccctaatatcactaatctctgtccatttggcgaggtgttta
acgcgaccagatttgcaagcgtatatgcctggaataggaagagaataagcaattgtgttgccgattactctgtcttgtataacagcgcat
ctttcagcacttttaagtgctatggtgtcaacgggacaaaacttaacgatctttgcttcaccaacgtttacgcagactcttttgtcatacgcg
gagatgaggtccgacaaatagctcccggccagactgggaaaatcgctgattataactataagcttccagatgacttcacaggatgcgta
attgcatggaactctaacaacctggactcaaaagttggtggcaactataactatctctatcgtttgttccgaaaatcaaaccttaaacccttt
gaacgggatattaatacgacaatttaccaagcagggagcactccttgtaacggtgtagaaggtttcaattgttattttcctctgcaatcata
cggattccaaccaacaaacggtgtgggttatcaaccttatcgggttgtagttttgagcttcgagcttaaccatgcacccgccacagtatgc
ggaccgaagaagagtacaaacctggttaagaataaatgtgtaaacttcaactttaatggactgacggggacgggagtactcactgaaa
gcaataagaaattcttgccttttcagcaattcggggggacatagcggacactacagacgccgtgcgcgacccccagactctcgaaat
cctggacataaccccgtgctcatttggcggagtttcagtcatcactccagggaccaatacctcaaaccaagtagctgtgctgtatcaaga
tgtgaattgcaccgaagtaccagtggccattcacgccgatcagctgaccccgacatgggggtgtactcaaccggttcaaatgtgtttc
aaacaagagcaggttgtcttattggcgctgaacacgtgaataactcctatgaatgcgacatcccaattggtgccggaatctgtgcctctta
tcaaacacaaactaattcaccaaggcgtaggcgcagcgtcgcctctcaatcaattatagcctacaccatgtcactgggtgccgaaaact
ccgtcgcgtacagcaacaatagcattgccatccctaccaacttcaccatcagctgtacaactgagatcctgcctgtatccatgacaaaga
catcctgcgattgcactatgtacatctgtggagactctactgagtgtagcaacctcttgctccaatacgggagtttctgtacgcaactcaac
cgtgccctcaccggcatagccgtagagcaagataagaatacccaggaagtatttgcccaagtaaagcaaatttataagacgccaccca
ttaaagactttggcggtttcaacttcagtcaaatactgccagacccgtctcgcaggagaaggagttttattgaagacctgctctttaacaa
ggtgactcttgccgatgctggatttattaaacaatatggggattgtctcggagatatcgctgctcgggatcttatctgcgcgcagaaattca
acgggtgtaccgtgctcccacccttgctcactgacgaaatgatcgcgcaatatacctcagcacttctggcgggaactattacatctggtt
ggacattcggcgcaggggcagctctccaaattcccttcgcaatgcaaatggcttacaggttcaatggcataggtgtcacacaaaacgt
gctgtacgagaatcaaaagcttatagccaatcagtttaatagcgccataggcaagatccaagattccctgagctccacggcaagcgctc
tgggaaaattgcaagacgtagtcaatcaaaacgctcaagcgctgaatacccttgtgaaacaactttcttcaaactttggagctatctcatct
gggcccaacgatattctgagtcgactgccaaaggttgaagctgaagtccaaattgatcggttgatcacaggaaggctgcaatccctgc
agacttacgtgacccagcaactgatcagggcagccgaaataagggcttccgccaatctggcagccacaaagatgtctgaatgtgtctt
gggtcaaagcaaacgcgtcgatttctgtggcaaggggtaccatctgatgtcattccctcaatctgcccctcacggtgtggtatttctccatt
gcacttatgttcccgcacaggagaagaacttcacaacagctcccgccatttgccacgacggaaaggcgcattttccccgcgaaggtgt
cttcgtgtccaatgggactcattggtttgtgactcagaggaatttctatgagccgcagattatcaccaccgacaacactttcgtctccggta
actgcgacgtcgttatcggaatcgtcaataacacagtgtatgatcctctgcagccggagctggactcattcaaagaggagttggataaat
attttaagaatcatacaagccccgacgtcgatctgggcgatattagtggtatcaatgcgtccgtggttaacattcagaaagagattgaca
gactcaatgaggtcgccaagaacttgaacgaatccttgattgatctccaggagttgggcaagtatgagcaatatatcaagtggccatgg
tctgggcgaaggcgccgtcgcagagggtccggcggtagtggttccgggtacataccagaagctccacgagatggtcaagcttatgta
aggaaagacggagagtgggtcctgcttagcacattcttgggttgataa (SEQ ID NO: 158)
ggauccgccaccauggacuggacauggauacuuuucuugguggcagcugcuacacgcguccacucaauguucgucuuuc
uggugcucuugccacuggugagcagccaaugcguuaaccucaccacacgcacgcagcuuccacccgcauacacuaacucc
uuuacgcgcggcguguacuauccagauaaaguguuccgaaguagcgucuugcauagcacccaggaucuguuucucccau
ucuuuagcaaugucacaugguuccacgcuauccacgugucugggacgaauggaacuaaacguuuugacaauccuguucu
uccuuuuaacgacggcguauacuuugcuaguacugagaagucuaacauuauccgcggcuggauuuucgggacaacccug
gacuccaaaacccagucucugcugauaguaaacaaugccaccaacgucgucauuaaagugugcgaguuucaauucugcaa
cgaccccuuucugggugucuauuaccacaagaacaauaagucuuggauggagucagaauuucgugucuauucuucugcc
aauaauuguacauuugaguauguuucucaacccuuucucauggaccucgaaggcaagcaggggaauuuuaagaaccugc
gggaauucgucuuuaagaauaucgacggcuauuucaaaauuuacagcaaacacacgccuauaaaccucgugcgagaucuc
ccccaaggcuucucagcauuggagccauuggucgacuugccaaucggaauuaauaucacaagguuucagacucugcugg
cccugcaucgcuccuaucuuaccccuggcgauuccucaaguggcuggacggccggcgcagcagccuauuacgucggcua
ucuccagccaaggacguuucuuuugaaguauaaugaaaaugggacuauuacugacgccgucgacugcgcuuuggacccc
cugagcgagacaaagugcacauugaaaagcuucacgguggagaaggguauuuaucaaacuuccaacuuuagggugcaac
caacagagagcaucgugagguucccuaauaucacuaaucucuguccauuuggcgagguguuuaacgcgaccagauuugc
aagcguauaugccuggaauaggaagagaauaagcaauuguguugccgauuacucugucuuguauaacagcgcaucuuuc
agcacuuuuaagugcuauggugucaacgggacaaaacuuaacgaucuuugcuucaccaacguuuacgcagacucuuuug
ucauacgcggagaugagguccgacaaauagcucccggccagacugggaaaaucgcugauuauaacuauaagcuuccagau
gacuucacaggaugcguaauugcauggaacucuaacaaccuggacucaaaaguugguggcaacuauaacuaucucuaucg
uuuguuccgaaaaucaaaccuuaaacccuuugaacgggauauuaauacgacaauuuaccaagcagggagcacuccuugua
acgguguagaagguuucaauuguuauuuuccucugcaaucauacggauuccaaccaacaaacgguguggguuaucaacc
uuaucgguuguaguuuugagcuucgagcuuaaccaugcacccgccacaguaugcggaccgaagaagaguacaaaccug
guuaaaaaaauguguaaacuucaacuuuaauggacugacggggacgggaguacucacugaaagcaauaagaaauucu
ugccuuuucagcaauucggggggacauagcggacacuacagacgccgugcgcgacccccagacucucgaaauccuggac
auaaccccgugcucauuuggcggaguuucagucaucacuccagggaccaauaccucaaaccaaguagcugugcuguauca
agaugugaauugcaccgaaguaccaguggccauucacgccgaucagcugaccccgacauggcggguguacucaaccggu
ucaaauguguuucaaacaagagcagguugucuuauuggcgcugaacacgugaauaacuccuaugaaugcgacaucccaa
uuggugccggaaucugugccucuuaucaaacacaaacuaauucaccaaggcguaggcgcagcgucgccucucaaucaauu
auagccuacaccaugucacugggugccgaaaacuccgucgcguacagcaacaauagcauugccaucccuaccaacuucacc
aucagcuguacaacugagauccugccuguauccaugacaaagacauccugcgauugcacuauguacaucuguggagacuc
uacugaguguagcaaccucuugcuccaauacgggaguuucuguacgcaacucaaccgugcccucaccggcauagccguag
agcaagauaagaauacccaggaaguauuugcccaaguaaagcaaauuuauaagacgccacccauuaaagacuuuggcggu
uucaacuucagucaaauacugccagacccgucucgcaggagaaggaguuuuauugaagaccugcucuuuaacaagguga
cucuugccgaugcuggauuuauuaaacaauauggggauugucucggagauaucgcugcucgggaucuuaucugcgcgc
agaaauucaacggguguaccgugcucccacccuugcucacugacgaaaugaucgcgcaauauaccucagcacuucuggcg
ggaacuauuacaucugguuggacauucggcgcaggggcagcucuccaaauucccuucgcaaugcaaauggcuuacaggu
ucaauggcauaggugucacacaaaacgugcuguacgagaaucaaaagcuuauagccaaucaguuuaauagcgccauaggc
aagauccaagauucccugagcuccacggcaagcgcucugggaaaauugcaagacguagucaaucaaaacgcucaagcgcu
gaauacccuugugaaacaacuuucuucaaacuuuggagcuaucucaucugggcccaacgauauucugagucgacugccaa
agguugaagcugaaguccaaauugaucgguugaucacaggaaggcugcaaucccugcagacuuacgugacccagcaacu
gaucagggcagccgaaauaagggcuuccgccaaucuggcagccacaaagaugucugaaugugucuugggucaaagcaaac
gcgucgauuucuguggcaagggguaccaucugaugucauucccucaaucugccccucacggugugguauuucuccauug
cacuuauguucccgcacaggagaagaacuucacaacagcucccgccauuugccacgacggaaaggcgcauuuuccccgcg
aaggugucuucguguccaaugggacucauugguuugugacucagaggaauuucuaugagccgcagauuaucaccaccga
caacacuuucgucuccgguaacugcgacgucguuaucggaaucgucaauaacacaguguaugauccucugcagccggag
cuggacucauucaaagaggaguuggauaaauauuuuaagaaucauacaagccccgacgucgaucugggcgauauuagug
guaucaaugcguccgugguuaacauucagaaagagauugacagacucaaugaggucgccaagaacuugaacgaauccuu
gauugaucuccaggaguugggcaaguaugagcaauauaucaaguggccauggucugggcgaaggcgccgucgcagaggg
uccggcgguagugguuccggguacauaccagaagcuccacgagauggucaagcuuauguaaggaaagacggagaguggg
uccugcuuagcacauucuuggguugauaa (SEQ ID NO: 159)
MDWTWILFLVAAATRVHSMFVFLVLLPLVSSQCVNLTTRTQLPPAYTNSFTRGVYY
PDKVFRSSVLHSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRVQPTESIVR
FPNITNLCPFGEVFNATRFASVYAWNRKRISNCVADYSVLYNSASFSTFKCYGVNGT
KLNDLCFTNVYADSFVIRGDEVRQIAPGQTGKIADYNYKLPDDFTGCVIAWNSNNLD
SKVGGNYNYLYRLFRKSNLKPFERDINTTIYQAGSTPCNGVEGFNCYFPLQSYGFQPT
NGVGYQPYRVVVLSFELNHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQD
VNCTEVPVAIHADQLTPTWRVYSTGSNVFQTRAGCLIGAEHVNNSYECDIPIGAGICA
SYQTQTNSPRRRRSVASQSIIAYTMSLGAENSVAYSNNSIAIPTNFTISCTTEILPVSMT
KTSCDCTMYICGDSTECSNLLLQYGSFCTQLNRALTGIAVEQDKNTQEVFAQVKQIY
KTPPIKDFGGFNFSQILPDPSRRRRSFIEDLLFNKVTLADAGFIKQYGDCLGDIAARDLI
CAQKFNGCTVLPPLLTDEMIAQYTSALLAGTITSGWTFGAGAALQIPFAMQMAYRFN
GIGVTQNVLYENQKLIANQFNSAIGKIQDSLSSTASALGKLQDVVNQNAQALNTLVK
QLSSNFGAISSGPNDILSRLPKVEAEVQIDRLITGRLQSLQTYVTQQLIRAAEIRASANL
AATKMSECVLGQSKRVDFCGKGYHLMSFPQSAPHGVVFLHCTYVPAQEKNFTTAPA
ICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNES
LIDLQELGKYEQYIKWPWSGRRRRRRGSGGSGSGYIPEAPRDGQAYVRKDGEWVLL
STFLG** (SEQ ID NO: 160)

In some embodiments therefore, the expressible nucleic acid sequence comprised in the composition of the disclosure comprises at least about 70%, 75%, 80%, 85%, 90%, 91%, 92%, 93%, 94%, 95%, 96%, 97%, 98% or 99% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the expressible nucleic acid sequence comprises the nucleic acid sequence of SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the expressible nucleic acid sequence comprised in the composition of the disclosure encodes a polypeptide comprising at least about 70%, 75%, 80%, 85%, 90%, 91%, 92%, 93%, 94%, 95%, 96%, 97%, 98% or 99% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the expressible nucleic acid sequence encodes a polypeptide comprising the amino acid sequence of SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

### B. Nucleic Acid Molecule

In some embodiments, the present disclosure also relates to a nucleic acid molecule that comprises any of the disclosed expressible nucleic acid sequences. For example, the expressible nucleic acid sequence disclosed herein can be part of a plasmid and thus the nucleic acid molecule is a plasmid comprising such an expressible nucleic acid sequence. In some embodiments, provided herein is a vector or plasmid that is capable of expressing at least a monomer of a self-assembling nanoparticle and a viral antigen construct or constructs in the cell of a mammal in a quantity effective to elicit an immune response in the mammal. The vector or plasmid may comprise heterologous nucleic acid encoding the one or more viral antigens (such as SARS-CoV-2 antigens). In some embodiments, provided herein is a vector or plasmid that is capable of expressing at least one soluble trimer of a coronavirus or SARS-CoV-2 envelope polypeptide or constructs in the cell of a mammal in a quantity effective to elicit an immune response in the mammal. In some embodiments, the nucleic acid expresses a trimer of the spike protein of SARS-CoV-2 or a functional fragment or variant thereof. The vector may be a plasmid. The plasmid may be useful for transfecting cells with nucleic acid encoding a viral antigen, which the transformed host cell is cultured and maintained under conditions wherein expression of the viral antigen takes place and wherein the structure of the nanoparticle with the antigen or trimer elicits an immune response of a magnitude greater than and/or more therapeutically effective than the immune response elicited by the antigen alone. The plasmid may further comprise an initiation codon, which may be upstream of the expressible sequence, and a stop codon, which may be downstream of the coding sequence. The initiation and termination codon may be in frame with the expressible sequence.

The plasmid may also comprise a promoter that is operably linked to the coding sequence. The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication No. US20040175727, the contents of which are incorporated herein in its entirety. The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhancers are described in U.S. Pat. Nos. 5,593,972, 5,962,428, and WO94/016737, the contents of each are fully incorporated by reference. The plasmid may also comprise a mammalian origin of replication in order to maintain the plasmid extrachromosomally and produce multiple copies of the plasmid in a cell. The plasmid may be pVAX1, pCEP4 or pREP4 from ThermoFisher Scientific (San Diego, Calif.), which may comprise the Epstein Barr virus origin of replication and nuclear antigen EBNA-1 coding region, which may produce high copy episomal replication without integration.

In some embodiments, the vector can be pVAX1 or a pVax1 variant with changes such as the variant plasmid described herein. The variant pVax1 plasmid is a 2998 basepair variant of the backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993. The vaccine may comprise the consensus antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some embodiments, pharmaceutical compositions according to the present disclosure comprise from about 1 nanogram to about 1000 micrograms of DNA. The nucleic acid sequence for the pVAX1 backbone sequence is as follows:

(SEQ ID NO: 161)
gactcttcgcgatgtacgggccagatatacgcgttgacattgattattga
ctagttattaatagtaatcaattacggggtcattagttcatagcccatat
atggagttccgcgttacataacttacggtaaatggcccgcctggctgacc
gcccaacgacccccgcccattgacgtcaataatgacgtatgttcccatag
taacgccaatagggactttccattgacgtcaatgggtggactatttacgg
taaactgcccacttggcagtacatcaagtgtatcatatgccaagtacgcc
ccctattgacgtcaatgacggtaaatggcccgcctggcattatgcccagt
acatgaccttatgggactttcctacttggcagtacatctacgtattagtc
atcgctattaccatggtgatgcggttttggcagtacatcaatgggcgtgg
atagcggtttgactcacggggatttccaagtctccaccccattgacgtca
atgggagtttgttttggcaccaaaatcaacgggactttccaaaatgtcgt
aacaactccgccccattgacgcaaatgggcggtaggcgtgtacggtggga
ggtctatataagcagagctctctggctaactagagaacccactgcttact
ggcttatcgaaattaatacgactcactatagggagacccaagctggctag
cgtttaaacttaagcttggtaccgagctcggatccactagtccagtgtgg
tggaattctgcagatatccagcacagtggcggccgctcgagtctagaggg
cccgtttaaacccgctgatcagcctcgactgtgccttctagttgccagcc
atctgttgtttgcccctcccccgtgccttccttgaccctggaaggtgcca
ctcccactgtcctttcctaataaaatgaggaaattgcatcgcattgtctg
agtaggtgtcattctattctggggggtggggtggggcaggacagcaaggg
ggaggattgggaagacaatagcaggcatgctggggatgcggtgggctcta
tggcttctactgggcggttttatggacagcaagcgaaccggaattgccag
ctggggcgccctctggtaaggttgggaagccctgcaaagtaaactggatg
gctttctcgccgccaaggatctgatggcgcaggggatcaagctctgatca
agagacaggatgaggatcgtttcgcatgattgaacaagatggattgcacg
caggttctccggccgcttgggtggagaggctattcggctatgactgggca
caacagacaatcggctgctctgatgccgccgtgttccggctgtcagcgca
ggggcgcccggttctttttgtcaagaccgacctgtccggtgccctgaatg
aactgcaagacgaggcagcgcggctatcgtggctggccacgacgggcgtt
ccttgcgcagctgtgctcgacgttgtcactgaagcgggaagggactggct
gctattgggcgaagtgccggggcaggatctcctgtcatctcaccttgctc
ctgccgagaaagtatccatcatggctgatgcaatgcggcggctgcatacg
cttgatccggctacctgcccattcgaccaccaagcgaaacatcgcatcga
gcgagcacgtactcggatggaagccggtcttgtcgatcaggatgatctgg
acgaagagcatcaggggctcgcgccagccgaactgttcgccaggctcaag
gcgagcatgcccgacggcgaggatctcgtcgtgacccatggcgatgcctg
cttgccgaatatcatggtggaaaatggccgcttttctggattcatcgact
gtggccggctgggtgtggcggaccgctatcaggacatagcgttggctacc
cgtgatattgctgaagagcttggcggcgaatgggctgaccgcttcctcgt
gctttacggtatcgccgctcccgattcgcagcgcatcgccttctatcgcc
ttcttgacgagttcttctgaattattaacgcttacaatttcctgatgcgg
tattttctccttacgcatctgtgcggtatttcacaccgcatacaggtggc
acttttcggggaaatgtgcgcggaacccctatttgtttatttttctaaat
acattcaaatatgtatccgctcatgagacaataaccctgataaatgcttc
aataatagcacgtgctaaaacttcatttttaatttaaaaggatctaggtg
aagatcctttttgataatctcatgaccaaaatcccttaacgtgagttttc
gttccactgagcgtcagaccccgtagaaaagatcaaaggatcttcttgag
atcctttttttctgcgcgtaatctgctgcttgcaaacaaaaaaaccaccg
ctaccagcggtggtttgtttgccggatcaagagctaccaactctttttcc
gaaggtaactggcttcagcagagcgcagataccaaatactgtccttctag
tgtagccgtagttaggccaccacttcaagaactctgtagcaccgcctaca
tacctcgctctgctaatcctgttaccagtggctgctgccagtggcgataa
gtcgtgtcttaccgggttggactcaagacgatagttaccggataaggcgc
agcggtcgggctgaacggggggttcgtgcacacagcccagcttggagcga
acgacctacaccgaactgagatacctacagcgtgagctatgagaaagcgc
cacgcttcccgaagggagaaaggcggacaggtatccggtaagcggcaggg
tcggaacaggagagcgcacgagggagcttccagggggaaacgcctggtat
ctttatagtcctgtcgggtttcgccacctctgacttgagcgtcgattttt
gtgatgctcgtcaggggggcggagcctatggaaaaacgccagcaacgcgg
cctttttacggttcctgggcttttgctggccttttgctcacatgttctt

Other vectors or plasmids that can be used herein to produce the vaccine of the present disclosure include, but not limited to, pcDNA3.1(+), pCI mammalian expression vector, pSI vector, pZeoSV2(+), phCMV1, pTCP and pIRES with their respective backbone sequence as follows.

The pcDNA3.1(+) backbone sequence (SEQ ID NO: 162):
gacggatcgggagatctcccgatcccctatggtgcactctcagtacaatctgctctgatgccgcatagttaagccagtatctgctccctg
cttgtgtgttggaggtcgctgagtagtgcgcgagcaaaatttaagctacaacaaggcaaggcttgaccgacaattgcatgaagaatctg
cttagggttaggcgttttgcgctgcttcgcgatgtacgggccagatatacgcgttgacattgattattgactagttattaatagtaatcaatta
cggggtcattagttcatagcccatatatggagttccgcgttacataacttacggtaaatggcccgcctggctgaccgcccaacgacccc
cgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatg
cccagtacatgaccttatgggactttcctacttggcagtacatctacgtattagtcatcgctattaccatggtgatgcggttttggcagtaca
tcaatgggcgtggatagcggtttgactcacggggatttccaagtctccaccccattgacgtcaatgggagtttgttttggcaccaaaatca
acgggactttccaaaatgtcgtaacaactccgccccattgacgcaaatgggcggtaggcgtgtacggtgggaggtctatataagcaga
gctctctggctaactagagaacccactgcttactggcttatcgaaattaatacgactcactatagggagacccaagctggctagcgttta
aacttaagcttggtaccgagctcggatccactagtccagtgtggtggaattctgcagatatccagcacagtggcggccgctcgagtcta
gagggcccgtttaaacccgctgatcagcctcgactgtgccttctagttgccagccatctgttgtttgcccctcccccgtgccttccttgac
cctggaaggtgccactcccactgtcctttcctaataaaatgaggaaattgcatcgcattgtctgagtaggtgtcattctattctggggggtg
gggtggggcaggacagcaagggggaggattgggaagacaatagcaggcatgctggggatgcggtgggctctatggcttctgagg
cggaaagaaccagctggggctctagggggtatccccacgcgccctgtagcggcgcattaagcgcggcgggtgtggtggttacgcg
cagcgtgaccgctacacttgccagcgccctagcgcccgctcctttcgctttcttcccttcctttctcgccacgttcgccggctttccccgtc
aagctctaaatcgggggctccctttagggttccgatttagtgctttacggcacctcgaccccaaaaaacttgattagggtgatggttcacg
tagtgggccatcgccctgatagacggtttttcgccctttgacgttggagtccacgttctttaatagtggactcttgttccaaactggaacaa
cactcaaccctatctcggtctattcttttgatttataagggattttgccgatttcggcctattggttaaaaaatgagctgatttaacaaaaattta
acgcgaattaattctgtggaatgtgtgtcagttagggtgtggaaagtccccaggctccccagcaggcagaagtatgcaaagcatgcat
ctcaattagtcagcaaccaggtgtggaaagtccccaggctccccagcaggcagaagtatgcaaagcatgcatctcaattagtcagcaa
ccatagtcccgcccctaactccgcccatcccgcccctaactccgcccagttccgcccattctccgccccatggctgactaattttttttattt
atgcagaggccgaggccgcctctgcctctgagctattccagaagtagtgaggaggcttttttggaggcctaggcttttgcaaaaagctc
ccgggagcttgtatatccattttcggatctgatcaagagacaggatgaggatcgtttcgcatgattgaacaagatggattgcacgcaggt
tctccggccgcttgggtggagaggctattcggctatgactgggcacaacagacaatcggctgctctgatgccgccgtgttccggctgt
cagcgcaggggcgcccggttctttttgtcaagaccgacctgtccggtgccctgaatgaactgcaggacgaggcagcgcggctatcgt
ggctggccacgacgggcgttccttgcgcagctgtgctcgacgttgtcactgaagcgggaagggactggctgctattgggcgaagtgc
cggggcaggatctcctgtcatctcaccttgctcctgccgagaaagtatccatcatggctgatgcaatgcggcggctgcatacgcttgat
ccggctacctgcccattcgaccaccaagcgaaacatcgcatcgagcgagcacgtactcggatggaagccggtcttgtcgatcaggat
gatctggacgaagagcatcaggggctcgcgccagccgaactgttcgccaggctcaaggcgcgcatgcccgacggcgaggatctcg
tcgtgacccatggcgatgcctgcttgccgaatatcatggtggaaaatggccgcttttctggattcatcgactgtggccggctgggtgtgg
cggaccgctatcaggacatagcgttggctacccgtgatattgctgaagagcttggcggcgaatgggctgaccgcttcctcgtgctttac
ggtatcgccgctcccgattcgcagcgcatcgccttctatcgccttcttgacgagttcttctgagcgggactctggggttcgaaatgaccg
accaagcgacgcccaacctgccatcacgagatttcgattccaccgccgccttctatgaaaggttgggcttcggaatcgttttccgggac
gccggctggatgatcctccagcgcggggatctcatgctggagttcttcgcccaccccaacttgtttattgcagcttataatggttacaaat
aaagcaatagcatcacaaatttcacaaataaagcatttttttcactgcattctagttgtggtttgtccaaactcatcaatgtatcttatcatgtct
gtataccgtcgacctctagctagagcttggcgtaatcatggtcatagctgtttcctgtgtgaaattgttatccgctcacaattccacacaac
atacgagccggaagcataaagtgtaaagcctggggtgcctaatgagtgagctaactcacattaattgcgttgcgctcactgcccgctttc
cagtcgggaaacctgtcgtgccagctgcattaatgaatcggccaacgcgcggggagaggcggtttgcgtattgggcgctcttccgctt
cctcgctcactgactcgctgcgctcggtcgttcggctgcggcgagcggtatcagctcactcaaaggcggtaatacggttatccacaga
atcaggggataacgcaggaaagaacatgtgagcaaaaggccagcaaaaggccaggaaccgtaaaaaggccgcgttgctggcgttt
ttccataggctccgcccccctgacgagcatcacaaaaatcgacgctcaagtcagaggtggcgaaacccgacaggactataaagatac
caggcgtttccccctggaagctccctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctgggctgtgtgcacgaaccccccg
ttcagcccgaccgctgcgccttatccggtaactatcgtcttgagtccaacccggtaagacacgacttatcgccactggcagcagccact
ggtaacaggattagcagagcgaggtatgtaggcggtgctacagagttcttgaagtggtggcctaactacggctacactagaagaaca
gtatttggtatctgcgctctgctgaagccagttaccttcggaaaaagagttggtagctcttgatccggcaaacaaaccaccgctggtagc
ggtttttttgtttgcaagcagcagattacgcgcagaaaaaaaggatctcaagaagatcctttgatcttttctacggggtctgacgctcagtg
gaacgaaaactcacgttaagggattttggtcatgagattatcaaaaaggatcttcacctagatccttttaaattaaaaatgaagttttaaatc
aatctaaagtatatatgagtaaacttggtctgacagttaccaatgcttaatcagtgaggcacctatctcagcgatctgtctatttcgttcatcc
atagttgcctgactccccgtcgtgtagataactacgatacgggagggcttaccatctggccccagtgctgcaatgataccgcgagacc
cacgctcaccggctccagatttatcagcaataaaccagccagccggaagggccgagcgcagaagtggtcctgcaactttatccgcct
ccatccagtctattaattgttgccgggaagctagagtaagtagttcgccagttaatagtttgcgcaacgttgttgccattgctacaggcatc
gtggtgtcacgctcgtcgtttggtatggcttcattcagctccggttcccaacgatcaaggcgagttacatgatcccccatgttgtgcaaaa
aagcggttagctccttcggtcctccgatcgttgtcagaagtaagttggccgcagtgttatcactcatggttatggcagcactgcataattct
cttactgtcatgccatccgtaagatgcttttctgtgactggtgagtactcaaccaagtcattctgagaatagtgtatgcggcgaccgagttg
ctcttgcccggcgtcaatacgggataataccgcgccacatagcagaactttaaaagtgctcatcattggaaaacgttcttcggggcgaa
aactctcaaggatcttaccgctgttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgttgaatactcatactcttc
ctttttcaatattattgaagcatttatcagggttattgtctcatgagcggatacatatttgaatgtatttagaaaaataaacaaataggggttcc
gcgcacatttccccgaaaagtgccacctgacgtc
The pCI mammalian expression vector backbone sequence (SEQ ID NO: 163):
tcaatattggccattagccatattattcattggttatatagcataaatcaatattggctattggccattgcatacgttgtatctatatcataatatg
tacatttatattggctcatgtccaatatgaccgccatgttggcattgattattgactagttattaatagtaatcaattacggggtcattagttcat
agcccatatatggagttccgcgttacataacttacggtaaatggcccgcctggctgaccgcccaacgacccccgcccattgacgtcaat
aatgacgtatgttcccatagtaacgccaatagggactttccattgacgtcaatgggtggagtatttacggtaaactgcccacttggcagta
catcaagtgtatcatatgccaagtccgccccctattgacgtcaatgacggtaaatggcccgcctggcattatgcccagtacatgacctta
cgggactttcctacttggcagtacatctacgtattagtcatcgctattaccatggtgatgcggttttggcagtacaccaatgggcgtggata
gcggtttgactcacggggatttccaagtctccaccccattgacgtcaatgggagtttgttttggcaccaaaatcaacgggactttccaaaa
tgtcgtaataaccccgccccgttgacgcaaatgggcggtaggcgtgtacggtgggaggtctatataagcagagctcgtttagtgaacc
gtcagatcactagaagctttattgcggtagtttatcacagttaaattgctaacgcagtcagtgcttctgacacaacagtctcgaacttaagc
tgcagaagttggtcgtgaggcactgggcaggtaagtatcaaggttacaagacaggtttaaggagaccaatagaaactgggcttgtcga
gacagagaagactcttgcgtttctgataggcacctattggtcttactgacatccactttgcctttctctccacaggtgtccactcccagttca
attacagctcttaaggctagagtacttaatacgactcactataggctagcctcgagaattcacgcgtggtacctctagagtcgacccggg
cggccgcttcgagcagacatgataagatacattgatgagtttggacaaaccacaactagaatgcagtgaaaaaaatgctttatttgtgaa
atttgtgatgctattgctttatttgtaaccattataagctgcaataaacaagttaacaacaacaattgcattcattttatgtttcaggttcagggg
gagatgtgggaggttttttaaagcaagtaaaacctctacaaatgtggtaaaatcgataaggatccgggctggcgtaatagcgaagagg
cccgcaccgatcgcccttcccaacagttgcgcagcctgaatggcgaatggacgcgccctgtagcggcgcattaagcgcggcgggtg
tggtggttacgcgcagcgtgaccgctacacttgccagcgccctagcgcccgctcctttcgctttcttcccttcctttctcgccacgttcgc
cggctttccccgtcaagctctaaatcgggggctccctttagggttccgatttagtgctttacggcacctcgaccccaaaaaacttgattag
ggtgatggttcacgtagtgggccatcgccctgatagacggtttttcgccctttgacgttggagtccacgttctttaatagtggactcttgttc
caaactggaacaacactcaaccctatctcggtctattcttttgatttataagggattttgccgatttcggcctattggttaaaaaatgagctga
tttaacaaaaatttaacgcgaattttaacaaaatattaacgcttacaatttcctgatgcggtattttctccttacgcatctgtgcggtatttcaca
ccgcatatggtgcactctcagtacaatctgctctgatgccgcatagttaagccagccccgacacccgccaacacccgctgacgcgccc
tgacgggcttgtctgctcccggcatccgcttacagacaagctgtgaccgtctccgggagctgcatgtgtcagaggttttcaccgtcatca
ccgaaacgcgcgagacgaaagggcctcgtgatacgcctatttttataggttaatgtcatgataataatggtttcttagacgtcaggtggca
cttttcggggaaatgtgcgcggaacccctatttgtttatttttctaaatacattcaaatatgtatccgctcatgagacaataaccctgataaat
cacccagaaacgctggtgaaagtaaaagatgctgaagatcagttgggtgcacgagtgggttacatcgaactggatctcaacagcggt
aagatccttgagagttttcgccccgaagaacgttttccaatgatgagcacttttaaagttctgctatgtggcgcggtattatcccgtattgac
gccgggcaagagcaactcggtcgccgcatacactattctcagaatgacttggttgagtactcaccagtcacagaaaagcatcttacgg
atggcatgacagtaagagaattatgcagtgctgccataaccatgagtgataacactgcggccaacttacttctgacaacgatcggagga
ccgaaggagctaaccgcttttttgcacaacatgggggatcatgtaactcgccttgatcgttgggaaccggagctgaatgaagccatacc
aaacgacgagcgtgacaccacgatgcctgtagcaatggcaacaacgttgcgcaaactattaactggcgaactacttactctagcttccc
ggcaacaattaatagactggatggaggcggataaagttgcaggaccacttctgcgctcggcccttccggctggctggtttattgctgat
aaatctggagccggtgagcgtgggtctcgcggtatcattgcagcactggggccagatggtaagccctcccgtatcgtagttatctacac
gacggggagtcaggcaactatggatgaacgaaatagacagatcgctgagataggtgcctcactgattaagcattggtaactgtcagac
caagtttactcatatatactttagattgatttaaaacttcatttttaatttaaaaggatctaggtgaagatcctttttgataatctcatgaccaaaa
tcccttaacgtgagttttcgttccactgagcgtcagaccccgtagaaaagatcaaaggatcttcttgagatcctttttttctgcgcgtaatct
gctgcttgcaaacaaaaaaaccaccgctaccagcggtggtttgtttgccggatcaagagctaccaactctttttccgaaggtaactggct
tcagcagagcgcagataccaaatactgttcttctagtgtagccgtagttaggccaccacttcaagaactctgtagcaccgcctacatacc
tcgctctgctaatcctgttaccagtggctgctgccagtggcgataagtcgtgtcttaccgggttggactcaagacgatagttaccggata
aggcgcagcggtcgggctgaacggggggttcgtgcacacagcccagcttggagcgaacgacctacaccgaactgagatacctaca
gcgtgagctatgagaaagcgccacgcttcccgaagggagaaaggcggacaggtatccggtaagcggcagggtcggaacaggag
agcgcacgagggagcttccagggggaaacgcctggtatctttatagtcctgtcgggtttcgccacctctgacttgagcgtcgatttttgt
gatgctcgtcaggggggcggagcctatggaaaaacgccagcaacgcggcctttttacggttcctggccttttgctggccttttgctcac
atggctcgacagatct
The pSI vector backbone sequence (SEQ ID NO: 164):
gcgcagcaccatggcctgaaataacctctgaaagaggaacttggttaggtaccttctgaggcggaaagaaccagctgtggaatgtgtg
tcagttagggtgtggaaagtccccaggctccccagcaggcagaagtatgcaaagcatgcatctcaattagtcagcaaccaggtgtgg
aaagtccccaggctccccagcaggcagaagtatgcaaagcatgcatctcaattagtcagcaaccatagtcccgcccctaactccgcc
catcccgcccctaactccgcccagttccgcccattctccgccccatggctgactaattttttttatttatgcagaggccgaggccgcctcg
gcctctgagctattccagaagtagtgaggaggcttttttggaggcctaggcttttgcaaaaagcttgattcttctgacacaacagtctcga
acttaagctgcagaagttggtcgtgaggcactgggcaggtaagtatcaaggttacaagacaggtttaaggagaccaatagaaactgg
gcttgtcgagacagagaagactcttgcgtttctgataggcacctattggtcttactgacatccactttgcctttctctccacaggtgtccact
cccagttcaattacagctcttaaggctagagtacttaatacgactcactataggctagcctcgagaattcacgcgtggtacctctagagtc
gacccgggcggccgcttcgagcagacatgataagatacattgatgagtttggacaaaccacaactagaatgcagtgaaaaaaatgctt
tatttgtgaaatttgtgatgctattgctttatttgtaaccattataagctgcaataaacaagttaacaacaacaattgcattcattttatgtttcag
gttcagggggaggtgtgggaggttttttaaagcaagtaaaacctctacaaatgtggtaaaatcgataaggatccgggctggcgtaatag
cgaagaggcccgcaccgatcgcccttcccaacagttgcgcagcctgaatggcgaatggacgcgccctgtagcggcgcattaagcg
cggcgggtgtggtggttacgcgcagcgtgaccgctacacttgccagcgccctagcgcccgctcctttcgctttcttcccttcctttctcgc
cacgttcgccggctttccccgtcaagctctaaatcgggggctccctttagggttccgatttagtgctttacggcacctcgaccccaaaaa
acttgattagggtgatggttcacgtagtgggccatcgccctgatagacggtttttcgccctttgacgttggagtccacgttctttaatagtg
gactcttgttccaaactggaacaacactcaaccctatctcggtctattcttttgatttataagggattttgccgatttcggcctattggttaaaa
aatgagctgatttaacaaaaatttaacgcgaattttaacaaaatattaacgcttacaatttcctgatgcggtattttctccttacgcatctgtgc
ggtatttcacaccgcatatggtgcactctcagtacaatctgctctgatgccgcatagttaagccagccccgacacccgccaacacccgc
tgacgcgccctgacgggcttgtctgctcccggcatccgcttacagacaagctgtgaccgtctccgggagctgcatgtgtcagaggtttt
caccgtcatcaccgaaacgcgcgagacgaaagggcctcgtgatacgcctatttttataggttaatgtcatgataataatggtttcttagac
gtcaggtggcacttttcggggaaatgtgcgcggaacccctatttgtttatttttctaaatacattcaaatatgtatccgctcatgagacaata
accctgataaatgcttcaataatattgaaaaaggaagagtatgagtattcaacatttccgtgtcgcccttattcccttttttgcggcattttgcc
ttcctgtttttgctcacccagaaacgctggtgaaagtaaaagatgctgaagatcagttgggtgcacgagtgggttacatcgaactggatc
tcaacagcggtaagatccttgagagttttcgccccgaagaacgttttccaatgatgagcacttttaaagttctgctatgtggcgcggtatta
tcccgtattgacgccgggcaagagcaactcggtcgccgcatacactattctcagaatgacttggttgagtactcaccagtcacagaaaa
gcatcttacggatggcatgacagtaagagaattatgcagtgctgccataaccatgagtgataacactgcggccaacttacttctgacaac
gatcggaggaccgaaggagctaaccgcttttttgcacaacatgggggatcatgtaactcgccttgatcgttgggaaccggagctgaat
gaagccataccaaacgacgagcgtgacaccacgatgcctgtagcaatggcaacaacgttgcgcaaactattaactggcgaactactt
actctagcttcccggcaacaattaatagactggatggaggcggataaagttgcaggaccacttctgcgctcggcccttccggctggctg
gtttattgctgataaatctggagccggtgagcgtgggtctcgcggtatcattgcagcactggggccagatggtaagccctcccgtatcgt
agttatctacacgacggggagtcaggcaactatggatgaacgaaatagacagatcgctgagataggtgcctcactgattaagcattggt
aactgtcagaccaagtttactcatatatactttagattgatttaaaacttcatttttaatttaaaaggatctaggtgaagatcctttttgataatct
catgaccaaaatcccttaacgtgagttttcgttccactgagcgtcagaccccgtagaaaagatcaaaggatcttcttgagatcctttttttct
gcgcgtaatctgctgcttgcaaacaaaaaaaccaccgctaccagcggtggtttgtttgccggatcaagagctaccaactctttttccgaa
ggtaactggcttcagcagagcgcagataccaaatactgttcttctagtgtagccgtagttaggccaccacttcaagaactctgtagcacc
gcctacatacctcgctctgctaatcctgttaccagtggctgctgccagtggcgataagtcgtgtcttaccgggttggactcaagacgata
gttaccggataaggcgcagcggtcgggctgaacggggggttcgtgcacacagcccagcttggagcgaacgacctacaccgaactg
agatacctacagcgtgagctatgagaaagcgccacgcttcccgaagggagaaaggcggacaggtatccggtaagcggcagggtcg
gaacaggagagcgcacgagggagcttccagggggaaacgcctggtatctttatagtcctgtcgggtttcgccacctctgacttgagcg
tcgatttttgtgatgctcgtcaggggggcggagcctatggaaaaacgccagcaacgcggcctttttacggttcctggccttttgctggcc
ttttgctcacatggctcgacagatct
The pZeoSV2(+) backbone sequence (SEQ ID NO: 165):
ggatcgatccggctgtggaatgtgtgtcagttagggtgtggaaagtccccaggctccccagcaggcagaagtatgcaaagcatgcat
ctcaattagtcagcaaccaggtgtggaaagtccccaggctccccagcaggcagaagtatgcaaagcatgcatctcaattagtcagcaa
ccatagtcccgcccctaactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttttgcaaaaagctc
tctggctaactagagaacccactgcttactggcttatcgaaattaatacgactcactatagggagacccaagctggctagcgtttaaactt
aagcttggtaccgagctcggatccactagtccagtgtggtggaattctgcagatatccagcacagtggcggccgctcgagtctagagg
gcccgtttaaacccgctgatcagcctcgactgtgccttctagttgccagccatctgttgtttgcccctcccccgtgccttccttgaccctgg
aaggtgccactcccactgtcctttcctaataaaatgaggaaattgcatcgcattgtctgagtaggtgtcattctattctggggggtggggt
ggggcaggacagcaagggggaggattgggaagacaatagcaggcatgctggggatgcggtgggctctatggcttctgaggcgga
aagaaccagcatgtgagcaaaaggccagcaaaaggccaggaaccgtaaaaaggccgcgttgctggcgtttttccataggctccgcc
cccctgacgagcatcacaaaaatcgacgctcaagtcagaggtggcgaaacccgacaggactataaagataccaggcgtttccccctg
gaagctccctcgtgcgctctcctgttccgaccctgccgcttaccggatacctgtccgcctttctcccttcgggaagcgtggcgctttctca
tagctcacgctgtaggtatctcagttcggtgtaggtcgttcgctccaagctgggctgtgtgcacgaaccccccgttcagcccgaccgct
gcgccttatccggtaactatcgtcttgagtccaacccggtaagacacgacttatcgccactggcagcagccactggtaacaggattagc
agagcgaggtatgtaggcggtgctacagagttcttgaagtggtggcctaactacggctacactagaagaacagtatttggtatctgcgc
tctgctgaagccagttaccttcggaaaaagagttggtagctcttgatccggcaaacaaaccaccgctggtagcggtggtttttttgtttgc
aagcagcagattacgcgcagaaaaaaaggatctcaagaagatcctttgatcttttctacggggtctgacgctcagtggaacgaaaactc
acgttaagggattttggtcatgacattaacctataaaaataggcgtatcacgaggccctttcgtctcgcgcgtttcggtgatgacggtgaa
aacctctgacacatgcagctcccggagacggtcacagcttgtctgtaagcggatgccgggagcagacaagcccgtcagggcgcgtc
agcgggtgttggcgggtgtcggggctggcttaactatgcggcatcagagcagattgtactgagagtgcaccatatgcggtgtgaaata
ccgcacagatgcgtaaggagaaaataccgcatcaggacgcgccctgtagcggcgcattaagcgcggcgggtgtggtggttacgcg
cagcgtgaccgctacacttgccagcgccctagcgcccgctcctttcgctttcttcccttcctttctcgccacgttcgccggctttccccgtc
aagctctaaatcgggggctccctttagggttccgatttagtgctttacggcacctcgaccccaaaaaacttgattagggtgatggttcacg
tagtgggccatcgccctgatagacggtttttcgccctttgacgttggagtccacgttctttaatagtggactcttgttccaaactggaacaa
cactcaaccctatctcggtctattcttttgatttataagggattttgccgatttcggcctattggttaaaaaatgagctgatttaacaaaaattta
acgcgaattttaacaaaatattaacgcttacaatttccattcgccattcaggctgaactagatctagagtccgttacataacttacggtaaat
ggcccgcctggctgaccgcccaacgacccccgcccattgacgtcaataatgacgtatgttcccatagtaacgccaatagggactttcc
attgacgtcaatgggtggagtatttacggtaaactgcccacttggcagtacatcaagtgtatcatatgccaagtacgccccctattgacgt
caatgacggtaaatggcccgcctggcattatgcccagtacatgaccttatgggactttcctacttggcagtacatctacgtattagtcatc
gctattaccatggtgatgcggttttggcagtacatcaatgggcgtggatagcggtttgactcacggggatttccaagtctccaccccattg
acgtcaatgggagtttgttttggcaccaaaatcaacgggactttccaaaatgtcgtaacaactccgccccattgacgcaaatgggcggt
aggcgtgtacggtgggaggtctatataagcagagctcgtttagtgaaccgtcagatcgcctggagacgccatccacgctgttttgacct
ccatagaagacaccgggaccgatccagcctccgcggccgggaacggtgcattggaacggaccgtgttgacaattaatcatcggcat
agtatatcggcatagtataatacgacaaggtgaggaactaaaccatggccaagttgaccagtgccgttccggtgctcaccgcgcgcga
cgtcgccggagcggtcgagttctggaccgaccggctcgggttctcccgggacttcgtggaggacgacttcgccggtgtggtccggg
acgacgtgaccctgttcatcagcgcggtccaggaccaggtggtgccggacaacaccctggcctgggtgtgggtgcgcggcctgga
cgagctgtacgccgagtggtcggaggtcgtgtccacgaacttccgggacgcctccgggccggccatgaccgagatcggcgagcag
ccgtggggggggagttcgccctgcgcgacccggccggcaactgcgtgcacttcgtggccgaggagcaggactgacactcgacct
cgaaacttgtttattgcagcttataatggttacaaataaagcaatagcatcacaaatttcacaaataaagcatttttttcactgcattctagttg
tggtttgtccaaactcatcaatgtatcttatcatgtct
The phCMV1 backbone sequence (SEQ ID NO: 166):
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctcgtttagtgaaccgtcagatcgcctggagacgccatccacgctgttttgacctccatagaagacac
cgggaccgatccagcctccgcggccgggaacggtgcattggaacgcggattccccgtgccaagagtgacgtaagtaccgcctatag
actctataggcacacccctttggctcttatgcatgaattaatacgactcactatagggagacagactgttcctttcctgggtcttttctgcag
gcaccgtcgtcgacttaacagatctcgagctcaagcttcgaattctgcagtcgacggtaccgcgggcccgggatccaccgggtacaa
gtaaagcggccgcgactctagatcataatcagccataccacatttgtagaggttttacttgctttaaaaaacctcccacacctccccctga
acctgaaacataaaatgaatgcaattgttgttgttaacttgtttattgcagcttataatggttacaaataaagcaatagcatcacaaatttcac
aaataaagcatttttttcactgcattctagttgtggtttgtccaaactcatcaatgtatcttaaggcgtaaattgtaagcgttaatattttgttaaa
attcgcgttaaatttttgttaaatcagctcattttttaaccaataggccgaaatcggcaaaatcccttataaatcaaaagaatagaccgagat
agggttgagtgttgttccagtttggaacaagagtccactattaaagaacgtggactccaacgtcaaagggcgaaaaaccgtctatcagg
gcgatggcccactacgtgaaccatcaccctaatcaagttttttggggtcgaggtgccgtaaagcactaaatcggaaccctaaagggag
cccccgatttagagcttgacggggaaagccggcgaacgtggcgagaaaggaagggaagaaagcgaaaggagcgggcgctagg
gcgctggcaagtgtagcggtcacgctgcgcgtaaccaccacacccgccgcgcttaatgcgccgctacagggcgcgtcaggtggca
cttttcggggaaatgtgcgcggaacccctatttgtttatttttctaaatacattcaaatatgtatccgctcatgagacaataaccctgataaat
gcttcaataatattgaaaaaggaagagtcctgaggcggaaagaaccagctgtggaatgtgtgtcagttagggtgtggaaagtccccag
gctccccagcaggcagaagtatgcaaagcatgcatctcaattagtcagcaaccaggtgtggaaagtccccaggctccccagcaggc
agaagtatgcaaagcatgcatctcaattagtcagcaaccatagtcccgcccctaactccgcccatcccgcccctaactccgcccagttc
cgcccattctccgccccatggctgactaattttttttatttatgcagaggccgaggccgcctcggcctctgagctattccagaagtagtga
ggaggcttttttggaggcctaggcttttgcaaagatcgatcaagagacaggatgaggatcgtttcgcatgattgaacaagatggattgca
cgcaggttctccggccgcttgggtggagaggctattcggctatgactgggcacaacagacaatcggctgctctgatgccgccgtgttc
cggctgtcagcgcaggggcgcccggttctttttgtcaagaccgacctgtccggtgccctgaatgaactgcaagacgaggcagcgcg
gctatcgtggctggccacgacgggcgttccttgcgcagctgtgctcgacgttgtcactgaagcgggaagggactggctgctattgggc
gaagtgccggggcaggatctcctgtcatctcaccttgctcctgccgagaaagtatccatcatggctgatgcaatgcggcggctgcatac
caggatgatctggacgaagagcatcaggggctcgcgccagccgaactgttcgccaggctcaaggcgagcatgcccgacggcgag
gatctcgtcgtgacccatggcgatgcctgcttgccgaatatcatggtggaaaatggccgcttttctggattcatcgactgtggccggctg
ggtgtggcggaccgctatcaggacatagcgttggctacccgtgatattgctgaagagcttggcggcgaatgggctgaccgcttcctcg
tgctttacggtatcgccgctcccgattcgcagcgcatcgccttctatcgccttcttgacgagttcttctgagcgggactctggggttcgaa
atgaccgaccaagcgacgcccaacctgccatcacgagatttcgattccaccgccgccttctatgaaaggttgggcttcggaatcgttttc
cgggacgccggctggatgatcctccagcgcggggatctcatgctggagttcttcgcccaccctagggggaggctaactgaaacacg
gaaggagacaataccggaaggaacccgcgctatgacggcaataaaaagacagaataaaacgcacggtgttgggtcgtttgttcataa
acgcggggttcggtcccagggctggcactctgtcgataccccaccgagaccccattggggccaatacgcccgcgtttcttccttttccc
caccccaccccccaagttcgggtgaaggcccagggctcgcagccaacgtcggggcggcaggccctgccatagcctcaggttactc
atatatactttagattgatttaaaacttcatttttaatttaaaaggatctaggtgaagatcctttttgataatctcatgaccaaaatcccttaacgt
gagttttcgttccactgagcgtcagaccccgtagaaaagatcaaaggatcttcttgagatcctttttttctgcgcgtaatctgctgcttgcaa
acaaaaaaaccaccgctaccagcggtggtttgtttgccggatcaagagctaccaactctttttccgaaggtaactggcttcagcagagc
gcagataccaaatactgtccttctagtgtagccgtagttaggccaccacttcaagaactctgtagcaccgcctacatacctcgctctgcta
atcctgttaccagtggctgctgccagtggcgataagtcgtgtcttaccgggttggactcaagacgatagttaccggataaggcgcagcg
gtcgggctgaacggggggttcgtgcacacagcccagcttggagcgaacgacctacaccgaactgagatacctacagcgtgagctat
gagaaagcgccacgcttcccgaagggagaaaggcggacaggtatccggtaagcggcagggtcggaacaggagagcgcacgag
ggagcttccagggggaaacgcctggtatctttatagtcctgtcgggtttcgccacctctgacttgagcgtcgatttttgtgatgctcgtcag
gggggcggagcctatggaaaaacgccagcaacgcggcctttttacggttcctggccttttgctggccttttgctcacatgttctttcctgc
gttatcccctgattctgtggataaccgtattaccgccatgcat
The pTCP backbone sequence (SEQ ID NO: 167):
tagttattaatagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacgtcaatg
ggtggagtatttacggtaaactgcccacttggcagtacatcaagtgtatcatatgccaagtacgccccctattgacgtcaatgacggtaa
atggcccgcctggcattatgcccagtacatgaccttatgggactttcctacttggcagtacatctacgtattagtcatcgctattaccatggt
gatgcggttttggcagtacatcaatgggcgtggatagcggtttgactcacggggatttccaagtctccaccccattgacgtcaatggga
gtttgttttggcaccaaaatcaacgggactttccaaaatgtcgtaacaactccgccccattgacgcaaatgggcggtaggcgtgtacggt
gggaggtctatataagcagagctggtttagtgaaccgtggatcccgtcgcttaccgattcagaatggttgatatccgccattctgaatcg
gtaagcgacgaagcttaataaaggatcttttattttcattggatctgtgtgttggttttttgtgtgcggccgccctcgactgtgccttctagaa
gacaatagcaggcatgctggggatgcggtgggctctatggcttctgaggcggaaagaaccagctggggctctagggggtatcccca
cgcgccctgtagcggcgcattaagcgcggcgggtgtggtggttacgcgcagcgtgaccgctacacttgccagcgccctagcgcccg
ctcctttcgctttcttcccttcctttctcgccacgttcgccggctttccccgtcaagctctaaatcgggggctccctttagggttccgatttagt
gctttacggcacctcgaccccaaaaaacttgattagggtgatggttcacgtagtgggccatcgccctgatagacggtttttcgccctttga
cgttggagtccacgttctttaatagtggactcttgttccaaactggaacaacactcaaccctatctcggtctattcttttgatttataagggatt
ttgccgatttcggcctattggttaaaaaatgagctgatttaacaaaaatttaacgcgaattaattctgtggaatgtgtgtcagttagggtgtg
gaaagtccccaggctccccagcaggcagaagtatgcaaagcatgcatctcaattagtcagcaaccaggtgtggaaagtccccaggct
ccccagcaggcagaagtatgcaaagcatgcatctcaattagtcagcaaccatagtcccgcccctaactccgcccatcccgcccctaac
tccgcccagttccgcccattctccgccccatggctgactaattttttttatttatgcagaggccgaggccgcctctgcctctgagctattcc
agaagtagtgaggaggcttttttggaggcctaggcttttgcaaaaagctcccgggatgaccgagtacaagcccacggtgcgcctcgc
cacccgcgacgacgtcccgcgggccgtacgcaccctcgccgccgcgttcgccgactaccccgccacgcgccacaccgtcgaccc
ggaccgccacatcgagcgggtcaccgagctgcaagaactcttcctcacgcgcgtcgggctcgacatcggcaaggtgtgggtcgcg
gacgacggcgccgcggtggcggtctggaccacgccggagagcgtcgaagcgggggcggtgttcgccgagatcggcccgcgcat
ggccgagttgagcggttcccggctggccgcgcagcaacagatggaaggcctcctggcgccgcaccggcccaaggagcccgcgtg
gttcctggccaccgtcggcgtctcgcccgaccaccagggcaagggtctgggcagcgccgtcgtgctccccggagtggaggcggcc
gagcgcgccggggtgcccgccttcctggagacctccgcgccccgcaacctccccttctacgagcggctcggcttcaccgtcaccgc
cgacgtcgaggtgcccgaaggaccgcgcacctggtgcatgacccgcaagcccggtgcctgattcgaatgaccgaccaagcgacgc
ccaacctgccatcacgagatttcgattccaccgccgccttctatgaaaggttgggcttcggaatcgttttccgggacgccggctggatg
atcctccagcgcggggatctcatgctggagttcttcgcccaccccaacttgtttattgcagcttataatggttacaaataaagcaatagcat
cacaaatttcacaaataaagcatttttttcactgcattctagttgtggtttgtccaaactcatcaatgtatcttatcatgtctgtataccgtcgac
ctctagctagagcttggcgtaatcatggtcatagctgtttcctgtgtgaaattgttatccgctcacaattccacacaacatacgagccggaa
gcataaagtgtaaagcctggggtgcctaatgagtgagctaactcacattaattgcgttgcgctcactgcccgctttccagtcgggaaacc
tgtcgtgccagctgcattaatgaatcggccaacgcgcggggagaggcggtttgcgtattgggcgctcttccgcttcctcgctcactgac
tcgctgcgctcggtcgttcggctgcggcgagcggtatcagctcactcaaaggcggtaatacggttatccacagaatcaggggataac
gcaggaaagaacatgtgagcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaagataccaggcgtttcccc
ctggaagctccctcgtgcgctctcctgttccgaccctgccgcttaccggatacctgtccgcctttctcccttcgggaagcgtggcgctttc
tcatagctcacgctgtaggtatctcagttcggtgtaggtcgttcgctccaagctgggctgtgtgcacgaaccccccgttcagcccgacc
gctgcgccttatccggtaactatcgtcttgagtccaacccggtaagacacgacttatcgccactggcagcagccactggtaacaggatt
agcagagcgaggtatgtaggcggtgctacagagttcttgaagtggtggcctaactacggctacactagaagaacagtatttggtatctg
cgctctgctgaagccagttaccttcggaaaaagagttggtagctcttgatccggcaaacaaaccaccgctggtagcggtttttttgtttgc
aagcagcagattacgcgcagaaaaaaaggatctcaagaagatcctttgatcttttctacggggtctgacgctcagtggaacgaaaactc
acgttaagggattttggtcatgagattatcaaaaaggatcttcacctagatccttttaaattaaaaatgaagttttaaatcaatctaaagtatat
atgagtaaacttggtctgacagttaccaatgcttaatcagtgaggcacctatctcagcgatctgtctatttcgttcatccatagttgcctgact
ccccgtcgtgtagataactacgatacgggagggcttaccatctggccccagtgctgcaatgataccgcgagacccacgctcaccggc
tccagatttatcagcaataaaccagccagccggaagggccgagcgcagaagtggtcctgcaactttatccgcctccatccagtctatta
attgttgccgggaagctagagtaagtagttcgccagttaatagtttgcgcaacgttgttgccattgctacaggcatcgtggtgtcacgctc
gtcgtttggtatggcttcattcagctccggttcccaacgatcaaggcgagttacatgatcccccatgttgtgcaaaaaagcggttagctcc
ttcggtcctccgatcgttgtcagaagtaagttggccgcagtgttatcactcatggttatggcagcactgcataattctcttactgtcatgcca
tccgtaagatgcttttctgtgactggtgagtactcaaccaagtcattctgagaatagtgtatgcggcgaccgagttgctcttgcccggcgt
caatacgggataataccgcgccacatagcagaactttaaaagtgctcatcattggaaaacgttcttcggggcgaaaactctcaaggatc
ttaccgctgttgagatccagttcgatgtaacccactcgtgcacccaactgatcttcagcatcttttactttcaccagcgtttctgggtgagca
aaaacaggaaggcaaaatgccgcaaaaaagggaataagggcgacacggaaatgttgaatactcatactcttcctttttcaatattattga
agcatttatcagggttattgtctcatgagcggatacatatttgaatgtatttagaaaaataaacaaataggggttccgcgcacatttccccg
aaaagtgccacctgacgtcgacggatcgggagatctcccgatcccctatggtgcactctcagtacaatctgctctgatgccgcatagtt
aagccagtatctgctccctgcttgtgtgttggaggtcgctgagtagtgcgcgagcaaaatttaagctacaacaaggcaaggcttgaccg
acaattgcatgaagaatctgcttagggttaggcgttttgcgctgcttcgcgatgtacgggccagatatacgcgttgacattgattattgac
The pIRES backbone sequence (SEQ ID NO: 168):
tcaatattggccattagccatattattcattggttatatagcataaatcaatattggctattggccattgcatacgttgtatctatatcataatatg
tacatttatattggctcatgtccaatatgaccgccatgttggcattgattattgactagttattaatagtaatcaattacggggtcattagttcat
agcccatatatggagttccgcgttacataacttacggtaaatggcccgcctggctgaccgcccaacgacccccgcccattgacgtcaat
aatgacgtatgttcccatagtaacgccaatagggactttccattgacgtcaatgggtggagtatttacggtaaactgcccacttggcagta
catcaagtgtatcatatgccaagtccgccccctattgacgtcaatgacggtaaatggcccgcctggcattatgcccagtacatgacctta
cgggactttcctacttggcagtacatctacgtattagtcatcgctattaccatggtgatgcggttttggcagtacaccaatgggcgtggata
gcggtttgactcacggggatttccaagtctccaccccattgacgtcaatgggagtttgttttggcaccaaaatcaacgggactttccaaaa
tgtcgtaacaactgcgatcgcccgccccgttgacgcaaatgggcggtaggcgtgtacggtgggaggtctatataagcagagctcgttt
agtgaaccgtcagatcactagaagctttattgcggtagtttatcacagttaaattgctaacgcagtcagtgcttctgacacaacagtctcga
acttaagctgcagtgactctcttaaggtagccttgcagaagttggtcgtgaggcactgggcaggtaagtatcaaggttacaagacaggt
ttaaggagaccaatagaaactgggcttgtcgagacagagaagactcttgcgtttctgataggcacctattggtcttactgacatccacttt
gcctttctctccacaggtgtccactcccagttcaattacagctcttaaggctagagtacttaatacgactcactataggctagcctcgagaa
ttcacgcgtcgagcatgcatctagggggccaattccgcccctctcccccccccccctctccctcccccccccctaacgttactggccg
aagccgcttggaataaggccggtgtgcgtttgtctatatgttattttccaccatattgccgtcttttggcaatgtgagggcccggaaacctg
gccctgtcttcttgacgagcattcctaggggtctttcccctctcgccaaaggaatgcaaggtctgttgaatgtcgtgaaggaagcagttcc
tctggaagcttcttgaagacaaacaacgtctgtagcgaccctttgcaggcagcggaaccccccacctggcgacaggtgcctctgcgg
ccaaaagccacgtgtataagatacacctgcaaaggcggcacaaccccagtgccacgttgtgagttggatagttgtggaaagagtcaa
atggctctcctcaagcgtattcaacaaggggctgaaggatgcccagaaggtaccccattgtatgggatctgatctggggcctcggtgc
acatgctttacatgtgtttagtcgaggttaaaaaaacgtctaggccccccgaaccacggggacgtggttttcctttgaaaaacacgatgat
aagcttgccacaacccgggatcctctagagtcgacccgggcggccgcttccctttagtgagggttaatgcttcgagcagacatgataa
gatacattgatgagtttggacaaaccacaactagaatgcagtgaaaaaaatgctttatttgtgaaatttgtgatgctattgctttatttgtaac
cattataagctgcaataaacaagttaacaacaacaattgcattcattttatgtttcaggttcagggggagatgtgggaggttttttaaagcaa
gtaaaacctctacaaatgtggtaaaatccgataaggatcgatccgggctggcgtaatagcgaagaggcccgcaccgatcgcccttcc
caacagttgcgcagcctgaatggcgaatggacgcgccctgtagcggcgcattaagcgcggcgggtgtggtggttacgcgcagcgtg
accgctacacttgccagcgccctagcgcccgctcctttcgctttcttcccttcctttctcgccacgttcgccggctttccccgtcaagctct
aaatcgggggctccctttagggttccgatttagtgctttacggcacctcgaccccaaaaaacttgattagggtgatggttcacgtagtgg
gccatcgccctgatagacggtttttcgccctttgacgttggagtccacgttctttaatagtggactcttgttccaaactggaacaacactca
accctatctcggtctattcttttgatttataagggattttgccgatttcggcctattggttaaaaaatgagctgatttaacaaaaatttaacgcg
aattttaacaaaatattaacgcttacaatttcctgatgcggtattttctccttacgcatctgtgcggtatttcacaccgcatacgcggatctgc
gcagcaccatggcctgaaataacctctgaaagaggaacttggttaggtaccttctgaggcggaaagaaccagctgtggaatgtgtgtc
agttagggtgtggaaagtccccaggctccccagcaggcagaagtatgcaaagcatgcatctcaattagtcagcaaccaggtgtggaa
agtccccaggctccccagcaggcagaagtatgcaaagcatgcatctcaattagtcagcaaccatagtcccgcccctaactccgcccat
cccgcccctaactccgcccagttccgcccattctccgccccatggctgactaattttttttatttatgcagaggccgaggccgcctcggcc
tctgagctattccagaagtagtgaggaggcttttttggaggcctaggcttttgcaaaaagcttgattcttctgacacaacagtctcgaactt
aaggctagagccaccatgattgaacaagatggattgcacgcaggttctccggccgcttgggtggagaggctattcggctatgactggg
cacaacagacaatcggctgctctgatgccgccgtgttccggctgtcagcgcaggggcgcccggttctttttgtcaagaccgacctgtcc
ggtgccctgaatgaactgcaggacgaggcagcgcggctatcgtggctggccacgacgggcgttccttgcgcagctgtgctcgacgtt
gtcactgaagcgggaagggactggctgctattgggcgaagtgccggggcaggatctcctgtcatctcaccttgctcctgccgagaaa
gtatccatcatggctgatgcaatgcggcggctgcatacgcttgatccggctacctgcccattcgaccaccaagcgaaacatcgcatcg
agcgagcacgtactcggatggaagccggtcttgtcgatcaggatgatctggacgaagagcatcaggggctcgcgccagccgaactg
ttcgccaggctcaaggcgcgcatgcccgacggcgaggatctcgtcgtgacccatggcgatgcctgcttgccgaatatcatggtggaa
aatggccgcttttctggattcatcgactgtggccggctgggtgtggcggaccgctatcaggacatagcgttggctacccgtgatattgct
gaagagcttggcggcgaatgggctgaccgcttcctcgtgctttacggtatcgccgctcccgattcgcagcgcatcgccttctatcgcctt
cttgacgagttcttctgagcgggactctggggttcgaaatgaccgaccaagcgacgcccaacctgccatcacgatggccgcaataaa
atatctttattttcattacatctgtgtgttggttttttgtgtgaatcgatagcgataaggatccgcgtatggtgcactctcagtacaatctgctct
gatgccgcatagttaagccagccccgacacccgccaacacccgctgacgcgccctgacgggcttgtctgctcccggcatccgcttac
agacaagctgtgaccgtctccgggagctgcatgtgtcagaggttttcaccgtcatcaccgaaacgcgcgagacgaaagggcctcgtg
atacgcctatttttataggttaatgtcatgataataatggtttcttagacgtcaggtggcacttttcggggaaatgtgcgcggaacccctattt
gtttatttttctaaatacattcaaatatgtatccgctcatgagacaataaccctgataaatgcttcaataatattgaaaaaggaagagtatgag
tattcaacatttccgtgtcgcccttattcccttttttgcggcattttgccttcctgtttttgctcacccagaaacgctggtgaaagtaaaagatg
ctgaagatcagttgggtgcacgagtgggttacatcgaactggatctcaacagcggtaagatccttgagagttttcgccccgaagaacgt
tttccaatgatgagcacttttaaagttctgctatgtggcgcggtattatcccgtattgacgccgggcaagagcaactcggtcgccgcatac
actattctcagaatgacttggttgagtactcaccagtcacagaaaagcatcttacggatggcatgacagtaagagaattatgcagtgctg
ccataaccatgagtgataacactgcggccaacttacttctgacaacgatcggaggaccgaaggagctaaccgcttttttgcacaacatg
ggggatcatgtaactcgccttgatcgttgggaaccggagctgaatgaagccataccaaacgacgagcgtgacaccacgatgcctgta
gcaatggcaacaacgttgcgcaaactattaactggcgaactacttactctagcttcccggcaacaattaatagactggatggaggcgga
taaagttgcaggaccacttctgcgctcggcccttccggctggctggtttattgctgataaatctggagccggtgagcgtgggtctcgcg
gtatcattgcagcactggggccagatggtaagccctcccgtatcgtagttatctacacgacggggagtcaggcaactatggatgaacg
aaatagacagatcgctgagataggtgcctcactgattaagcattggtaactgtcagaccaagtttactcatatatactttagattgatttaaa
acttcatttttaatttaaaaggatctaggtgaagatcctttttgataatctcatgaccaaaatcccttaacgtgagttttcgttccactgagcgt
cagaccccgtagaaaagatcaaaggatcttcttgagatcctttttttctgcgcgtaatctgctgcttgcaaacaaaaaaaccaccgctacc
agcggtggtttgtttgccggatcaagagctaccaactctttttccgaaggtaactggcttcagcagagcgcagataccaaatactgttctt
ctagtgtagccgtagttaggccaccacttcaagaactctgtagcaccgcctacatacctcgctctgctaatcctgttaccagtggctgctg
ccagtggcgataagtcgtgtcttaccgggttggactcaagacgatagttaccggataaggcgcagcggtcgggctgaacggggggtt
cgtgcacacagcccagcttggagcgaacgacctacaccgaactgagatacctacagcgtgagctatgagaaagcgccacgcttccc
gaagggagaaaggcggacaggtatccggtaagcggcagggtcggaacaggagagcgcacgagggagcttccagggggaaacg
cctggtatctttatagtcctgtcgggtttcgccacctctgacttgagcgtcgatttttgtgatgctcgtcaggggggcggagcctatggaaa
aacgccagcaacgcggcctttttacggttcctggccttttgctggccttttgctcacatggctcgacagatct

In some embodiments therefore, the composition of the disclosure comprises a nucleic acid molecule comprising at least about 70%, 75%, 80%, 85%, 90%, 91%, 92%, 93%, 94%, 95%, 96%, 97%, 98% or 99% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof. In some embodiments, the composition of the disclosure comprises a nucleic acid molecule comprising the nucleotide sequence of SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof. In some embodiments, the composition of the disclosure comprises a nucleic acid molecule that is a pVax variant.

In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising a first nucleic acid sequence encoding a scaffold domain comprising any of the self-assembling polypeptides disclosed herein, or a functional fragment or variant thereof, and a second nucleic acid sequence encoding an antigen domain comprising any of the viral antigens disclosed herein, or a functional fragment or variant thereof. In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising a first nucleic acid sequence encoding a scaffold domain comprising a self-assembling polypeptide comprising at least about 70%, 75%, 80%, 85%, 90%, 91%, 92%, 93%, 94%, 95%, 96%, 97%, 98%, 99% or 100% sequence identity to SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof, and a second nucleic acid sequence encoding an antigen domain comprising a viral antigen comprising at least about 70%, 75%, 80%, 85%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising a first nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment or variant thereof, and a second nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 59, SEQ ID NO: 61, SEQ ID NO: 63 or SEQ ID NO: 65, or a functional fragment or variant thereof. In some embodiments, such nucleic acid molecules or plasmids may further comprise a third nucleic acid sequence encoding a leader sequence comprising at least about 70%, 75%, 80%, 85%, 90%, 91%, 92%, 93%, 94%, 95%, 96%, 97%, 98%, 99% or 100% sequence identity to SEQ ID NO: 1 or SEQ ID NO: 5, or a functional fragment or variant thereof. In such embodiments, the third nucleic acid sequence encoding a leader sequence may comprising at least about 70%, 75%, 80%, 85%, 90%, 91%, 92%, 93%, 94%, 95%, 96%, 97%, 98%, 99% or 100% sequence identity to SEQ ID NO: 2, SEQ ID NO: 3, SEQ ID NO: 4 or SEQ ID NO: 6, or a functional fragment or variant thereof.

In some embodiments, the nucleic acid molecules or plasmids of the disclosure may additionally comprise another nucleic acid sequence encoding a linker comprising at least about 70%, 75%, 80%, 85%, 90%, 91%, 92%, 93%, 94%, 95%, 96%, 97%, 98%, 99% or 100% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the nucleic acid sequence encoding a linker may comprising at least about 70%, 75%, 80%, 85%, 90%, 91%, 92%, 93%, 94%, 95%, 96%, 97%, 98%, 99% or 100% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising a first nucleic acid sequence encoding a leader sequence comprising any of the leader sequences disclosed herein, or a functional fragment or variant thereof, and a second nucleic acid sequence encoding a viral trimer (or three viral monomers) comprising any of the viral antigens disclosed herein, or a functional fragment or variant thereof. In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising a first nucleic acid sequence encoding a leader sequence comprising at least about 70%, 75%, 80%, 85%, 90%, 91%, 92%, 93%, 94%, 95%, 96%, 97%, 98%, 99% or 100% sequence identity to SEQ ID NO: 1 or SEQ ID NO: 5, or a functional fragment or variant thereof, and a second nucleic acid sequence encoding three viral monomers, each viral monomer independently comprising at least about 70%, 75%, 80%, 85%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising a first nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 2, SEQ ID NO: 3, SEQ ID NO: 4 or SEQ ID NO: 6, or a functional fragment or variant thereof, and a second nucleic acid sequence encoding three viral monomers, each viral monomer independently being encoded by a nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 59, SEQ ID NO: 61, SEQ ID NO: 63 or SEQ ID NO: 65, or a functional fragment or variant thereof. In some embodiments, each of the viral monomers is linked by one or more linker peptides comprising at least about 70%, 75%, 80%, 85%, 90%, 91%, 92%, 93%, 94%, 95%, 96%, 97%, 98%, 99% or 100% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, each of the viral monomers is linked by one or more linker peptides encoded by a nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

In some embodiments, any of the nucleic acid molecules or plasmids of the disclosure additionally comprises a nucleic acid sequence encoding a furin cleavage site comprising at least about 60%, 65%, 70%, 75%, 80%, 85%, 90%, 95%, 99% or 100% sequence identity to SEQ ID NO: 67, or a functional fragment or variant thereof.

In some embodiments, the nucleic acid molecule or plasmid may further comprises a nucleic acid encoding a transmembrane domain and a foldon domain. A non-limiting example of the transmembrane domain is the transmembrane domain of a platelet derived growth factor receptor comprising the sequence of AVGQDTQEVIVVPHSLPFKVVVISAILALVVLTIISLIILIMLWQKKPR (SEQ ID NO: 169). A non-limiting example of the foldon domain may comprise the sequence of YIPEAPRDGQAYVRKDGEWVLLSTFL (SEQ ID NO: 170). Thus, in some embodiments, the composition of the disclosure comprises a nucleic acid molecule or a plasmid comprising a nucleic acid sequence encoding a transmembrane domain comprising at least about 70%, 75%, 80%, 85%, 90%, 95%, 96%, 97%, 98% 99% or 100% sequence identity to SEQ ID NO: 169, or a functional fragment or variant thereof. In some embodiments, the composition of the disclosure comprises a nucleic acid molecule or a plasmid comprising a nucleic acid sequence encoding a foldon domain comprising at least about 70%, 75%, 80%, 85%, 90%, 95%, 96%, 97%, 98% 99% or 100% sequence identity to SEQ ID NO: 170, or a functional fragment or variant thereof.

In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising at least about 70%, 75%, 80%, 85%, 90%, 91%, 92%, 93%, 94%, 95%, 96%, 97%, 98%, 99% or 100% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the composition of the disclosure comprises a nucleic acid molecule or a plasmid comprising the nucleotide sequence of SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence comprising the nucleotide sequence of SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof. In some embodiments, the composition of the disclosure comprises a nucleic acid molecule or a plasmid comprising at least about 70%, 75%, 80%, 85%, 90%, 91%, 92%, 93%, 94%, 95%, 96%, 97%, 98%, 99% or 100% sequence identity to SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the composition of the disclosure comprises a nucleic acid molecule or a plasmid comprising the nucleotide sequence of SEQ ID NO: 161, SEQ ID NO: 162, SEQ ID NO: 163, SEQ ID NO: 164, SEQ ID NO: 165, SEQ ID NO: 166, SEQ ID NO: 167 or SEQ ID NO: 168, or a functional fragment or variant thereof, and an expressible nucleic acid sequence encoding a polypeptide comprising the amino acid sequence of SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof.

In some embodiments, the disclosure relates to a vector or a plasmid comprising one or a plurality of regulatory sequences operably linked to one or more of any of the disclosed expressible nucleic acid sequences. In some embodiments, the disclosure relates to a composition comprising a nucleic acid molecule comprising at least about 70%, 75%, 80%, 85%, 90%, 91%, 92%, 93%, 94%, 95%, 96%, 97%, 99% or 100% sequence identity to SEQ ID NO: 161, or a functional fragment or variant thereof, and positioned within the multiple cloning site thereof is one or more expressible nucleic acid sequences according to the present disclosure. In some embodiments, the disclosure relates to a composition comprising one or a plurality of RNA molecules, each individually comprising the RNA sequences disclosed herein, including but not limited to SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156 or SEQ ID NO: 159, or a functional fragment or variant thereof.

### C. Polypeptide Sequences

Disclosed are the polypeptide sequences encoded by the disclosed nucleic acid sequences. In some embodiments, the disclosure relates to compositions comprising polypeptide sequences encoded by the expressible nucleic acid molecules of the present disclosure comprising a scaffold domain comprising a self-assembling polypeptide and an antigen domain comprising a viral antigen, and optionally comprising a leader domain comprising a leader sequence and/or a linker domain comprising a linker peptide. In some embodiments, the disclosure relates to compositions comprising polypeptide sequences encoded by the expressible nucleic acid molecules of the present disclosure comprising a leader domain comprising a leader sequence and an antigen domain comprising three viral monomers (trimer), and optionally comprising one or plurality of linker domains each comprising a linker peptide. The disclosure also relates to cells expressing one or more such polypeptides disclosed herein.

In some embodiments, the polypeptide encoded by the expressible nucleic acid molecule of the present disclosure comprises at least about 70%, 75%, 80%, 85%, 90%, 91%, 92%, 93%, 94%, 95%, 96%, 97%, 98%, 99% or 100% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the polypeptide is encoded by a nucleic acid sequence comprises at least about 70%, 75%, 80%, 85%, 90%, 91%, 92%, 93%, 94%, 95%, 96%, 97%, 98%, 99% or 100% sequence identity to SEQ ID NO: 68, SEQ ID NO: 69, SEQ ID NO: 71, SEQ ID NO: 72, SEQ ID NO: 74, SEQ ID NO: 75, SEQ ID NO: 77, SEQ ID NO: 78, SEQ ID NO: 80, SEQ ID NO: 81, SEQ ID NO: 83, SEQ ID NO: 84, SEQ ID NO: 86, SEQ ID NO: 87, SEQ ID NO: 89, SEQ ID NO: 90, SEQ ID NO: 92, SEQ ID NO: 93, SEQ ID NO: 95, SEQ ID NO: 96, SEQ ID NO: 98, SEQ ID NO: 99, SEQ ID NO: 101, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, SEQ ID NO: 114, SEQ ID NO: 116, SEQ ID NO: 117, SEQ ID NO: 119, SEQ ID NO: 120, SEQ ID NO: 122, SEQ ID NO: 123, SEQ ID NO: 125, SEQ ID NO: 126, SEQ ID NO: 128, SEQ ID NO: 129, SEQ ID NO: 131, SEQ ID NO: 132, SEQ ID NO: 134, SEQ ID NO: 135, SEQ ID NO: 137, SEQ ID NO: 138, SEQ ID NO: 140, SEQ ID NO: 141, SEQ ID NO: 143, SEQ ID NO: 144, SEQ ID NO: 146, SEQ ID NO: 147, SEQ ID NO: 149, SEQ ID NO: 150, SEQ ID NO: 152, SEQ ID NO: 153, SEQ ID NO: 155, SEQ ID NO: 156, SEQ ID NO: 158 or SEQ ID NO: 159, or a functional fragment or variant thereof.

In some embodiments, the leader sequence encoded by the expressible nucleic acid sequence of the disclosure comprises at least about 70%, 75%, 80%, 85%, 90%, 91%, 92%, 93%, 94%, 95%, 96%, 97%, 98%, 99% or 100% sequence identity to SEQ ID NO: 1 or SEQ ID NO: 5, or a functional fragment or variant thereof. In some embodiments, the leader sequence is encoded by a nucleic acid sequence comprises at least about 70%, 75%, 80%, 85%, 90%, 91%, 92%, 93%, 94%, 95%, 96%, 97%, 98%, 99% or 100% sequence identity to SEQ ID NO: 2, SEQ ID NO: 3, SEQ ID NO: 4 or SEQ ID NO: 6, or a functional fragment or variant thereof.

In some embodiments, the self-assembling polypeptide encoded by the expressible nucleic acid sequence of the disclosure comprises at least about 70%, 75%, 80%, 85%, 90%, 91%, 92%, 93%, 94%, 95%, 96%, 97%, 98%, 99% or 100% sequence identity to SEQ ID NO: 8, SEQ ID NO: 10, SEQ ID NO: 12, SEQ ID NO: 14, SEQ ID NO: 16, SEQ ID NO: 18 or SEQ ID NO: 20, or a functional fragment or variant thereof. In some embodiments, the self-assembling polypeptide is encoded by a nucleic acid sequence comprises at least about 70%, 75%, 80%, 85%, 90%, 91%, 92%, 93%, 94%, 95%, 96%, 97%, 98%, 99% or 100% sequence identity to SEQ ID NO: 7, SEQ ID NO: 9, SEQ ID NO: 11, SEQ ID NO: 13, SEQ ID NO: 15, SEQ ID NO: 17 or SEQ ID NO: 19, or a functional fragment or variant thereof.

In some embodiments, the linker peptide encoded by the expressible nucleic acid sequence of the disclosure comprises at least about 70%, 75%, 80%, 85%, 90%, 91%, 92%, 93%, 94%, 95%, 96%, 97%, 98%, 99% or 100% sequence identity to SEQ ID NO: 22, SEQ ID NO: 23, SEQ ID NO: 24, SEQ ID NO: 25, SEQ ID NO: 26, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56 or SEQ ID NO: 58, or a functional fragment or variant thereof. In some embodiments, the linker peptide is encoded by a nucleic acid sequence comprises at least about 70%, 75%, 80%, 85%, 90%, 91%, 92%, 93%, 94%, 95%, 96%, 97%, 98%, 99% or 100% sequence identity to SEQ ID NO: 21, SEQ ID NO: 27, SEQ ID NO: 28, SEQ ID NO: 29, SEQ ID NO: 30, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55 or SEQ ID NO: 57 or a functional fragment or variant thereof.

In some embodiments, the viral antigen or monomer encoded by the expressible nucleic acid sequence of the disclosure comprises at least about 70%, 75%, 80%, 85%, 90%, 91%, 92%, 93%, 94%, 95%, 96%, 97%, 98%, 99% or 100% sequence identity to SEQ ID NO: 60, SEQ ID NO: 62, SEQ ID NO: 64, SEQ ID NO: 66, SEQ ID NO: 171, SEQ ID NO: 172, SEQ ID NO: 173, SEQ ID NO: 174, SEQ ID NO: 175, SEQ ID NO: 176 or SEQ ID NO: 177, or a functional fragment or variant thereof. In some embodiments, the viral antigen or monomer is encoded by a nucleic acid sequence comprises at least about 70%, 75%, 80%, 85%, 90%, 91%, 92%, 93%, 94%, 95%, 96%, 97%, 98%, 99% or 100% sequence identity to SEQ ID NO: 59, SEQ ID NO: 61, SEQ ID NO: 63 or SEQ ID NO: 65, or a functional fragment or variant thereof.

In some embodiments, the polypeptides encoded by the expressible nucleic acid molecule of the present disclosure comprises a furin cleavage site comprising at least about 70%, 75%, 80%, 85%, 90%, 91%, 92%, 93%, 94%, 95%, 96%, 97%, 98%, 99% or 100% sequence identity to SEQ ID NO: 67. In some embodiments, the polypeptides encoded by the expressible nucleic acid molecule of the present disclosure comprises a transmembrane domain comprising at least about 70%, 75%, 80%, 85%, 90%, 91%, 92%, 93%, 94%, 95%, 96%, 97%, 98%, 99% or 100% sequence identity to SEQ ID NO: 169. In some embodiments, the polypeptides encoded by the expressible nucleic acid molecule of the present disclosure comprises a foldon domain comprising at least about 70%, 75%, 80%, 85%, 90%, 91%, 92%, 93%, 94%, 95%, 96%, 97%, 98%, 99% or 100% sequence identity to SEQ ID NO: 170.

### D. Pharmaceutical Compositions

Disclosed are pharmaceutical compositions comprising any one or more of the disclosed compositions and a pharmaceutically acceptable carrier. In some embodiments the pharmaceutical composition comprising a therapeutically effective amount of a nucleic acid sequence that encodes LS-3 or a variant comprising at least 70% sequence identity to the LS-3 sequence.

In some embodiments, any of the disclosed compositions is from about 1 to about 30 micrograms of the disclosed DNA and/or RNA vaccine. For example, any of the disclosed compositions can be from about 1 to about 5 micrograms the disclosed DNA and/or RNA vaccine. In some preferred embodiments, the pharmaceutical compositions contain from about 5 nanograms to about 800 micrograms of the disclosed DNA and/or RNA vaccine. In some preferred embodiments, the pharmaceutical compositions contain about 25 to about 250 micrograms, from about 100 to about 200 micrograms, from about 1 nanogram to 100 milligrams; from about 1 microgram to about 10 milligrams; from about 0.1 microgram to about 10 milligrams; from about 1 milligram to about 2 milligrams, from about 5 nanograms to about 1000 micrograms, from about 10 nanograms to about 800 micrograms, from about 0.1 to about 500 micrograms, from about 1 to about 350 micrograms, from about 25 to about 250 micrograms, from about 100 to about 200 micrograms of the DNA and/or RNA vaccine or plasmid thereof. The pharmaceutical compositions can comprise from about 5 nanograms to about 10 mg of the disclosed DNA and/or RNA vaccine. In some embodiments, pharmaceutical compositions according to the present invention comprise from about 25 nanograms to about 5 mg of the disclosed DNA and/or RNA vaccine. In some embodiments, the pharmaceutical compositions contain from about 50 nanograms to about 1 mg of the disclosed DNA and/or RNA vaccine. In some embodiments, the pharmaceutical compositions contain about from about 0.1 to about 500 micrograms of the disclosed DNA and/or RNA vaccine. In some embodiments, the pharmaceutical compositions contain from about 1 to about 350 micrograms of the disclosed DNA and/or RNA vaccine. In some embodiments, the pharmaceutical compositions contain from about 5 to about 250 micrograms of the disclosed DNA and/or RNA vaccine. In some embodiments, the pharmaceutical compositions contain from about 10 to about 200 micrograms of the disclosed DNA and/or RNA vaccine. In some embodiments, the pharmaceutical compositions contain from about 15 to about 150 micrograms of the disclosed DNA and/or RNA vaccine. In some embodiments, the pharmaceutical compositions contain about 20 to about 100 micrograms of the disclosed DNA and/or RNA vaccine. In some embodiments, the pharmaceutical compositions contain about 25 to about 75 micrograms of the disclosed DNA and/or RNA vaccine. In some embodiments, the pharmaceutical compositions contain about 30 to about 50 micrograms of the disclosed DNA and/or RNA vaccine. In some embodiments, the pharmaceutical compositions contain about 35 to about 40 micrograms of the disclosed DNA and/or RNA vaccine. In some embodiments, the pharmaceutical compositions contain about 100 to about 200 micrograms the disclosed DNA and/or RNA vaccine. In some embodiments, the pharmaceutical compositions comprise about 10 micrograms to about 100 micrograms of the disclosed DNA and/or RNA vaccine. In some embodiments, the pharmaceutical compositions comprise about 20 micrograms to about 80 micrograms of the disclosed DNA and/or RNA vaccine. In some embodiments, the pharmaceutical compositions comprise about 25 micrograms to about 60 micrograms of the disclosed DNA and/or RNA vaccine. In some embodiments, the pharmaceutical compositions comprise about 30 nanograms to about 50 micrograms of the disclosed DNA and/or RNA vaccine. In some embodiments, the pharmaceutical compositions comprise about 35 nanograms to about 45 micrograms of the disclosed DNA and/or RNA vaccine. In some preferred embodiments, the pharmaceutical compositions contain about 0.1 to about 500 micrograms of the disclosed DNA and/or RNA vaccine. In some preferred embodiments, the pharmaceutical compositions contain about 1 to about 350 micrograms of the disclosed DNA and/or RNA vaccine. In some preferred embodiments, the pharmaceutical compositions contain about 1 to about 250 micrograms of the disclosed DNA and/or RNA vaccine. In some preferred embodiments, the pharmaceutical compositions contain about 2 to about 200 micrograms the disclosed DNA and/or RNA vaccine.

In some embodiments, pharmaceutical compositions according to the present invention comprise at least about 10, 15, 20, 25, 30, 35, 40, 45, 50, 55, 60, 65, 70, 75, 80, 85, 90, 95 or 100 nanograms of the disclosed DNA and/or RNA vaccine. In some embodiments, the pharmaceutical compositions can comprise at least about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms of the disclosed DNA and/or RNA vaccine. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of the disclosed DNA and/or RNA vaccine.

In other embodiments, the pharmaceutical composition can comprise up to and including about 10, 15, 20, 25, 30, 35, 40, 45, 50, 55, 60, 65, 70, 75, 80, 85, 90, 95 or 100 nanograms of the disclosed DNA and/or RNA vaccine. In some embodiments, the pharmaceutical composition can comprise up to and including about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms of the disclosed DNA and/or RNA vaccine. In some embodiments, the pharmaceutical composition can comprise up to and including about 1.5, 2, 2.5, 3, 3.5, 4, 4.5, 5, 5.5, 6, 6.5, 7, 7.5, 8, 8.5, 9, 9.5 or about 10 mg of the disclosed DNA and/or RNA vaccine. The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or other known transfection facilitating agents. In some embodiments, the vaccine is a composition comprising a plasmid DNA molecule, RNA molecule or DNA/RNA hybrid molecule encoding an expressible nucleic acid sequence, the expressible nucleic acid sequence comprising a first nucleic acid encoding a self-assembling nanoparticle comprising a viral antigen, optionally encoding a leader sequence disclosed herein.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are deneurological system as proteins in combination with the plasmid above in the vaccine. The adjuvant can be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof.

In some embodiments adjuvant may be one or more proteins and/or nucleic acid molecules that encode proteins selected from the group consisting of: CCL-20, IL-12, IL-15, IL-28, CTACK, TECK, MEC or RANTES. Examples of IL-12 constructs and sequences are disclosed in PCT application No. PCT/US1997/019502 (published as WO98/017799) and corresponding U.S. application Ser. No. 08/956,865, and U.S. Provisional Application No. 61/569,600 filed Dec. 12, 2011, which are each incorporated herein by reference in their entireties. Examples of IL-15 constructs and sequences are disclosed in PCT application No. PCT/US04/18962 (published as WO2005/000235) and corresponding U.S. application Ser. No. 10/560,650, and in PCT application No. PCT/US07/00886 (published as WO2007/087178) and corresponding U.S. application Ser. No. 12/160,766, and in PCT Application Ser. No. PCT/US10/048,827 (published as WO2011/032179), which are each incorporated herein by reference in their entireties. Examples of IL-28 constructs and sequences are disclosed in PCT application no. PCT/US09/039648 (published as WO2009/124309) and corresponding U.S. application Ser. No. 12/936,192, which are each incorporated herein by reference in their entireties. Examples of RANTES and other constructs and sequences are disclosed in PCT application No. PCT/US 1999/004332 (published as WO99/043839) and corresponding U.S. application Ser. No. 09/622,452, which are each incorporated herein by reference in their entireties. Other examples of RANTES constructs and sequences are disclosed in PCT Application No. PCT/US Ser. No. 11/024,098 (published as WO2011/097640), which is incorporated herein by reference. Examples of RANTES and other constructs and sequences are disclosed in PCT Application No. PCT/US 1999/004332 and corresponding U.S. application Ser. No. 09/622,452, which are each incorporated herein by reference. Other examples of RANTES constructs and sequences are disclosed in PCT application No. PCT/US11/024098 (published as WO2011/097640), which is incorporated herein by reference in its entirety. Examples of chemokines CTACK, TECK and MEC constructs and sequences are disclosed in PCT Application No. PCT/US2005/042231 (published as WO2007/050095) and corresponding U.S. application Ser. No. 11/719,646, which are each incorporated herein by reference in their entireties. Examples of OX40 and other immunomodulators are disclosed in U.S. application Ser. No. 10/560,653, which is incorporated herein by reference in its entirety. Examples of DR5 and other immunomodulators are disclosed in U.S. application Ser. No. 09/622,452, which is incorporated herein by reference in its entirety.

The pharmaceutical composition may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

The vaccine can be a DNA or RNA vaccine. In some embodiments, the vaccine is a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. Examples of attenuated live vaccines, those using recombinant vectors to foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference in their entireties.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

In some embodiments, the disclosure relates to a DNA vector pVAX1 comprising any one or more of the expressible nucleic acid sequences disclosed herein or an RNA transcript thereof. In some embodiments, the disclosure relates to a pharmaceutical composition comprising a nucleic acid sequence that includes one or a plurality of the expressible nucleic acid sequences disclosed herein or an RNA transcript thereof, and a pharmaceutically acceptable carrier.

### E. Methods

Disclosed are methods of vaccinating a subject comprising administering a therapeutically effective amount of any of the disclosed nucleic acid molecules, compositions, cells or pharmaceutical compositions to the subject. In some embodiments, the vaccination is against viral infection. In some embodiments, the viral infection is an infection of a virus from the family of Coronaviridae. In some embodiments, the viral infection is an infection of a coronavirus. In some embodiments, the viral infection is an infection of SARS-CoV. In some embodiments, the viral infection is an infection of HCoV NL63. In some embodiments, the viral infection is an infection of HKU1. In some embodiments, the viral infection is an infection of MERS-CoV. In some embodiments, the viral infection is an infection of SARS-CoV-2.

Disclosed are methods of inducing an immune response in a subject comprising administering to the subject any of the disclosed pharmaceutical compositions. In some embodiments, the methods are for inducing an immune response to a viral antigen in the subject. In some embodiments, the immune response induced by the disclosed methods is against a viral antigen from a virus from the family of Coronaviridae. In some embodiments, the immune response induced by the disclosed methods is against a viral antigen from a coronavirus. In some embodiments, the immune response induced by the disclosed methods is against a viral antigen from SARS-CoV. In some embodiments, the immune response induced by the disclosed methods is against a viral antigen from HCoV NL63. In some embodiments, the immune response induced by the disclosed methods is against a viral antigen from HKU1. In some embodiments, the immune response induced by the disclosed methods is against a viral antigen from MERS-CoV. In some embodiments, the immune response induced by the disclosed methods is against a viral antigen from SARS-CoV-2.

Disclosed are methods of neutralizing one or a plurality of viruses in a subject comprising administering to the subject any of the disclosed pharmaceutical compositions. In some embodiments, the virus being neutralized by the disclosed method is a virus from the family of Coronaviridae. In some embodiments, the virus being neutralized by the disclosed method is a coronavirus. In some embodiments, the virus being neutralized by the disclosed method is SARS-CoV. In some embodiments, the virus being neutralized by the disclosed method is HCoV NL63. In some embodiments, the virus being neutralized by the disclosed method is HKU1. In some embodiments, the virus being neutralized by the disclosed method is MERS-CoV. In some embodiments, the virus being neutralized by the disclosed method is SARS-CoV-2.

Disclosed are methods of neutralizing infection of one or a plurality of viruses in a subject comprising administering to the subject any of the disclosed pharmaceutical compositions. In some embodiments, the viral infection being neutralized by the disclosed method is an infection of a virus from the family of Coronaviridae. In some embodiments, the viral infection being neutralized by the disclosed method is an infection of coronavirus. In some embodiments, the viral infection being neutralized by the disclosed method is an infection of SARS-CoV. In some embodiments, the viral infection being neutralized by the disclosed method is an infection of HCoV NL63. In some embodiments, the viral infection being neutralized by the disclosed method is an infection of HKU1. In some embodiments, the viral infection being neutralized by the disclosed method is an infection of MERS-CoV. In some embodiments, the viral infection being neutralized by the disclosed method is an infection of SARS-CoV-2.

Disclosed are methods of stimulating a therapeutically effective antigen-specific immune response against a virus in a mammal infected with the virus comprising administering any of the disclosed pharmaceutical compositions. In some embodiments, the disclosed method is a method of stimulating a therapeutically effective antigen-specific immune response against a virus from the family of Coronaviridae. In some embodiments, the disclosed method is a method of stimulating a therapeutically effective antigen-specific immune response against a coronavirus. In some embodiments, the disclosed method is a method of stimulating a therapeutically effective antigen-specific immune response against SARS-CoV. In some embodiments, the disclosed method is a method of stimulating a therapeutically effective antigen-specific immune response against HCoV NL63. In some embodiments, the disclosed method is a method of stimulating a therapeutically effective antigen-specific immune response against HKU1. In some embodiments, the disclosed method is a method of stimulating a therapeutically effective antigen-specific immune response against MERS-CoV. In some embodiments, the disclosed method is a method of stimulating a therapeutically effective antigen-specific immune response against SARS-CoV-2.

Disclosed are methods of inducing expression of a self-assembling vaccine in a subject comprising administering any of the disclosed pharmaceutical compositions. Also disclosed are methods of treating a subject having a viral infection or susceptible to becoming infected with a virus comprising administering to the subject a therapeutically effective amount of any of the disclosed pharmaceutical compositions. In some embodiments, the viral infection is an infection of a virus from the family of Coronaviridae. In some embodiments, the viral infection is an infection of coronavirus. In some embodiments, the viral infection is an infection of SARS-CoV. In some embodiments, the viral infection is an infection of HCoV NL63. In some embodiments, the viral infection is an infection of HKU1. In some embodiments, the viral infection is an infection of MERS-CoV. In some embodiments, the viral infection is an infection of SARS-CoV-2.

The disclosed pharmaceutical compositions may be administered by any route of administration. Accordingly, in some embodiments, the administering can be accomplished by oral administration. In some embodiments, the administering can be accomplished by parenteral administration. In some embodiments, the administering can be accomplished by sublingual administration. In some embodiments, the administering can be accomplished by transdermal administration. In some embodiments, the administering can be accomplished by rectal administration. In some embodiments, the administering can be accomplished by transmucosal administration. In some embodiments, the administering can be accomplished by topical administration. In some embodiments, the administering can be accomplished by inhalation. In some embodiments, the administering can be accomplished by buccal administration. In some embodiments, the administering can be accomplished by intrapleural administration. In some embodiments, the administering can be accomplished by intravenous administration. In some embodiments, the administering can be accomplished by intraarterial administration. In some embodiments, the administering can be accomplished by intraperitoneal administration. In some embodiments, the administering can be accomplished by subcutaneous administration. In some embodiments, the administering can be accomplished by intramuscular administration. In some embodiments, the administering can be accomplished by intranasal administration. In some embodiments, the administering can be accomplished by intrathecal administration. In some embodiments, the administering can be accomplished by intraarticular administration. In some embodiments, the administering can be accomplished by intradermal administration. In some embodiments, the above modes of action are accomplished by injection of the pharmaceutical compositions disclosed herein. In some embodiments, the therapeutically effective dose can be from about 1 to about 30 micrograms of expressible nucleic acid sequence. In some embodiments, the therapeutically effective dose can be from about 0.001 micrograms of the composition per kilogram of subject to about 0.050 micrograms per kilogram of subject.

In some embodiments, any of the disclosed methods can be free of activating any mannose-binding lectin or complement process.

In some embodiments, the subject can be a human. In some embodiments, the subject is diagnosed with or suspected of having a viral infection. In some embodiments, the subject is diagnosed with or suspected of having an infection of a virus from the family of Coronaviridae. In some embodiments, the subject is diagnosed with or suspected of having an infection of coronavirus. In some embodiments, the subject is diagnosed with or suspected of having an infection of SARS-CoV. In some embodiments, the subject is diagnosed with or suspected of having an infection of HCoV NL63. In some embodiments, the subject is diagnosed with or suspected of having an infection of HKU1. In some embodiments, the subject is diagnosed with or suspected of having an infection of MERS-CoV. In some embodiments, the subject is diagnosed with or suspected of having an infection of SARS-CoV-2.

In some embodiments of the methods of inducing an immune response, the immune response can be an antigen-specific immune response. In some embodiments, the antigen-specific immune response can be an antigen-specific to SARS-CoV-2 antigen immune response. In some embodiments, the antigen-specific immune response can be a therapeutically effective CD-4+ antigen-specific SARS-CoV-2 immune response. In some embodiments, the antigen-specific immune response can be a therapeutically effective CD-8+ antigen-specific SARS-CoV-2 immune response. In some embodiments, the antigen-specific immune response can be a therapeutically effective CD-4+ and CD-8+ antigen-specific SARS-CoV-2 immune response.

In some embodiments, the methods are free of administering any polypeptide directly to the subject.

In some embodiments, any of the disclosed methods can further comprise administering to the subject a pharmaceutical composition comprising one or more pharmaceutically active agents, such as antiviral drugs, among many others. In some embodiments, the one or more pharmaceutically active agents include other anticoronarival medications used to inhibit coronavirus, for example nucleoside analog reverse transcriptase inhibitors, non-nucleoside reverse transcriptase inhibitors, and protease inhibitors. Among the available drugs that may be used as a pharmaceutically active agent are zidovudine or AZT (or Retrovir®), didanosine or DDI (or Videx®), stavudine or D4T (or Zerit®), lamivudine or 3TC (or Epivir®), zalcitabine or DDC (or Hivid®), abacavir succinate (or Ziagen”), tenofovir disoproxil fumarate salt (or Viread®), emtricitabine (or Emtriva®), Combivir® (contains 3TC and AZT), Trizivir® (contains abacavir, 3TC and AZT); three non-nucleoside reverse transcriptase inhibitors: nevirapine (or Viramune®), delavirdine (or Rescriptor®) and efavirenz (or Sustiva®), eight peptidomimetic protease inhibitors or approved formulations: saquinavir (or Invirase® or Fortovase”), indinavir (or Crixivan®), ritonavir (or Norvir®), nelfinavir (or Viracept”), amprenavir (or Agenerase®), atazanavir (Reyataz), fosamprenavir (or Lexiva), Kaletra® (contains lopinavir and ritonavir), and one fusion inhibitor enfuvirtide (or T-20 or Fuzeon®).

In some embodiments, the methods of inducing an immune response can include inducing a humoral or cellular immune response. A humoral immune response mainly refers to antibody production. A cellular immune response can include activation of CD4+ T-cells and activation CD8+ cells and associated cytotoxic activity. In one aspect, the present disclosure features a method of inducing an immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein. In one aspect, the present disclosure features a method of inducing a CD8+ T cell immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein.

In one aspect, the present disclosure features a method of enhancing an immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein.

In one aspect, the present disclosure features a method of enhancing a CD8+ T cell immune response in a subject, the method comprising administering to the subject in need thereof a pharmaceutically effective amount of any of the nucleic acid molecules comprising any one or a plurality of the disclosed expressible nucleic acid sequences or embodiments herein, or any one of the pharmaceutical compositions disclosed herein.

In some embodiments, the subject has a viral infection and is in need of therapy for the viral infection. In some embodiments, the viral infection is an infection of a virus from the family of Coronaviridae. In some embodiments, the viral infection is an infection of coronavirus. In some embodiments, the viral infection is an infection of SARS-CoV. In some embodiments, the viral infection is an infection of HCoV NL63. In some embodiments, the viral infection is an infection of HKU1. In some embodiments, the viral infection is an infection of MERS-CoV. In some embodiments, the viral infection is an infection of SARS-CoV-2.

In some embodiments, the subject has previously been treated, and not responded to anti-viral therapy. In some embodiments, the nucleic acid molecule and/or the expressible nucleic acid sequence of the disclosure is administered to the subject by electroporation.

The vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns,” or other physical methods such as electroporation (“EP”), “hydrodynamic method,” or ultrasound.

The plasmid of the vaccine may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The antigen may be delivered via DNA injection and along with in vivo electroporation.

The vaccine or pharmaceutical composition can be administered by electroporation. Administration of the vaccine via electroporation of the plasmids of the vaccine may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, Pa.) or Elgen electroporator (Inovio Pharmaceuticals, Inc.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers the same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 ρs, 20 ρs, 10 ρs or 1 ρs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present disclosure, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference in its entirety. The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes. The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to a method of injecting DNA are contemplated herein. The above-patents are incorporated by reference in their entireties.

Methods of preparing the nucleic acid sequences are disclosed. In some embodiments, plasmid sequences with one or more multiple cloning sites my be purchased from commercially available vendors and the expressible nucleic acid sequences disclosed herein may be ligated into the plasmids after a digestion with a known restriction enzyme needed to cute the plasmid DNA. In another alternative embodiment, membrane-based purification methods disclosed herein offer reduced cost, high binding capacity, and high flow rates, resulting in a superior purification process. The purification process is further demonstrated to produce plasmid products substantially free of genomic DNA, RNA, protein, and endotoxin.

In some embodiments, all of the described aspects of the present disclosure are advantageously combined to provide an integrated process for preparing substantially purified cellular components of interest from cells in bioreactors. Again, the cells are most preferably plasmid-containing cells, and the cellular components of interest are most preferably plasmids. The substantially purified plasmids are suitable for various uses, including, but not limited to, gene therapy, plasmid-mediated therapy, as DNA vaccines for human, veterinary, or agricultural use, or for any other application that requires large quantities of purified plasmid. In this aspect, all of the advantages described for individual aspects of the present disclosure accrue to the complete, integrated process, providing a highly advantageous method that is rapid, scalable, and inexpensive. Enzymes and other animal-derived or biologically sourced products are avoided, as are carcinogenic, mutagenic, or otherwise toxic substances. Potentially flammable, explosive, or toxic organic solvents are similarly avoided.

One aspect of the present disclosure is an apparatus for isolating plasmid DNA from a suspension of cells having both plasmid DNA and genomic DNA. An embodiment of the apparatus comprises a first tank and second tank in fluid communication with a mixer. The first tank is used for holding the suspension cells and the second tank is used for holding a lysis solution. The suspension of cells from the first tank and the lysis solution from the second tank are both allowed to flow into the mixer forming a lysate mixture or lysate fluid. The mixer comprises a high shear, low residence-time mixing device with a residence time of equal to or less than about 1 second. In a preferred embodiment, the mixing device comprises a flow through, rotor/stator mixer or emulsifier having linear flow rates from about 0.1 L/min to about 20 L/min. The lysate-mixture flows from the mixer into a holding coil for a period of time sufficient to lyse the cells and forming a cell lysate suspension, wherein the lysate-mixture has resident time in the holding coil in a range of about 2-8 minutes with a continuous linear flow rate.

The cell lysate suspension is then allowed to flow into a bubble-mixer chamber for precipitation of cellular components from the plasmid DNA. In the bubble mixer chamber, the cell lysate suspension and a precipitation solution or a neutralization solution from a third tank are mixed together using gas bubbles, which forms a mixed gas suspension comprising a precipitate and an unclarified lysate or plasmid containing fluid. The precipitate of the mixed gas suspension is less dense than the plasmid containing fluid, which facilitates the separation of the precipitate from the plasmid containing fluid. The precipitate is removed from the mixed gas suspension to give a clarified lysate having the plasmid DNA, and the precipitate having cellular debris and genomic DNA.

In some embodiments, the bubble mixer-chamber comprises a closed vertical column with a top, a bottom, a first, and a second side with a vent proximal to the top of the column. A first inlet port of the bubble mixer-chamber is on the first side proximal to the bottom of the column and in fluid communication with the holding coil. A second inlet port of the bubble mixer-chamber is proximal to the bottom on a second side opposite of the first inlet port and in fluid communication with a third tank, wherein the third tank is used for holding a precipitation or a neutralization solution. A third inlet port of the bubble mixer-chamber is proximal to the bottom of the column and about in the middle of the first and second inlets and is in fluid communication with a gas source the third inlet entering the bubble-mixer-chamber. A preferred embodiment utilizes a sintered sparger inside the closed vertical column of the third inlet port. The outlet port exiting the bubble mixing chamber is proximal to the top of the closed vertical column. The outlet port is in fluid communication with a fourth tank, wherein the mixed gas suspension containing the plasmid DNA is allowed to flow from the bubble-mixer-chamber into the fourth tank. The fourth tank is used for separating the precipitate of the mixed gas suspension having a plasmid containing fluid, and can also include an impeller mixer sufficient to provide uniform mixing of fluid without disturbing the precipitate. A fifth tank is used for a holding the clarified lysate or clarified plasmid containing fluid. The clarified lysate is then filtered at least once. A first filter has a particle size limit of about 5-10 μm and the second filter has a cut of about 0.2 μm. Although gravity, pressure, vacuum, or a mixture thereof can be used for transporting: suspension of cells; lysis solutions; precipitation solutions; neutralization solutions; or mixed gas suspensions from any of the tanks to mixers, holding coils or different tanks, pumps are utilized in a preferred embodiments. In a more preferred embodiment, at least one pump having a linear flow rate from about 0.1 to about 1 ft/second is used.

In another specific embodiment, a Y-connector having a having a first bifurcated branch, a second bifurcated branch and an exit branch is used to contact the cell suspension and the lysis solutions before they enter the high shear, low residence-time mixing device. The first tank holding the cell suspension is in fluid communication with the first bifurcated branch of the Y-connector through the first pump and the second tank holding the lysis solution is in fluid communication with the second bifurcated branch of the Y-connector through the second pump. The high shear, low residence-time mixing device is in fluid communication with an exit branch of the Y-connector, wherein the first and second pumps provide a linear flow rate of about 0.1 to about 2 ft/second for a contacted fluid exiting the Y-connector.

Another specific aspect of the present disclosure is a method of substantially separating plasmid DNA and genomic DNA from a bacterial cell lysate. The method comprises: delivering a cell lysate into a chamber; delivering a precipitation fluid or a neutralization fluid into the chamber; mixing the cell lysate and the precipitation fluid or a neutralization fluid in the chamber with gas bubbles forming a gas mixed suspension, wherein the gas mixed suspension comprises the plasmid DNA in a fluid portion (i.e. an unclarified lysate) and the genomic DNA is in a precipitate that is less dense than the fluid portion; floating the precipitate on top of the fluid portion; removing the fluid portion from the precipitate forming a clarified lysate, whereby the plasmid DNA in the clarified lysate is substantially separated from genomic DNA in the precipitate. In some embodiments, the chamber is the bubble mixing chamber as described above; the lysing solution comprises an alkali, an acid, a detergent, an organic solvent, an enzyme, a chaotrope, or a denaturant; the precipitation fluid or the neutralization fluid comprises potassium acetate, ammonium acetate, or a mixture thereof; and the gas bubbles comprise compressed air or an inert gas. Additionally, the decanted-fluid portion containing the plasmid DNA is preferably further purified with one or more purification steps selected from a group consisting of: ion exchange, hydrophobic interaction, size exclusion, reverse phase purification, endotoxin depletion, affinity purification, adsorption to silica, glass, or polymeric materials, expanded bed chromatography, mixed mode chromatography, displacement chromatography, hydroxyapatite purification, selective precipitation, aqueous two-phase purification, DNA condensation, thiophilic purification, ion-pair purification, metal chelate purification, filtration through nitrocellulose, or ultrafiltration.

In some embodiments, a method for isolating a plasmid DNA from cells comprising: mixing a suspension of cells having the plasmid DNA and genomic DNA with a lysis solution in a high-shear-low-residence-time-mixing-device for a first period of time forming a cell lysate fluid; incubating the cell lysate fluid for a second period of time in a holding coil forming a cell lysate suspension; delivering the cell lysate suspension into a chamber; delivering a precipitation/neutralization fluid into the chamber; mixing the cell lysate suspension and the a precipitation/neutralization fluid in the chamber with gas bubbles forming a gas mixed suspension, wherein the gas mixed suspension comprises an unclarified lysate containing the plasmid DNA and a precipitate containing the genomic DNA, wherein the precipitate is less dense than the unclarified lysate; floating the precipitate on top of the unclarified lysate; removing the precipitate from the unclarified lysate forming a clarified lysate, whereby the plasmid DNA is substantially separated from genomic DNA; precipitating the plasmid DNA from the clarified lysate forming a precipitated plasmid DNA; and resuspending the precipitated plasmid DNA in an aqueous solution.

The disclosure also relates to a method of producing a polypeptide of interest in a mammalian cell, the method comprising contacting the cell with a composition comprising one or a plurality of the RNA molecules disclosed herein. In some embodiments, the therapeutic and/or prophylactic agent is an mRNA, and wherein the mRNA encodes the polypeptide of interest, whereby the mRNA is capable of being translated in the cell to produce the polypeptide of interest (e.g., nanoparticle or trimer of the disclosure). Compositions comprising RNA nucleic acid sequences of the disclosure can be delivered via lipid-containing nanoparticles and/or modification of the RNA nucleic acid sequence encoding the one or more viral polypeptides.

In some embodiments, the composition includes at least one RNA polynucleotide having an open reading frame encoding at least one SARS-CoV-2 antigenic polypeptide having at least one modification, at least one 5; terminal cap, and is formulated within a lipid nanoparticle.

In some embodiments, a 5′ terminal cap is 7mG(5′)ppp(5′)NlmpNp. In some embodiments, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments, a lipid nanoparticle comprises a cationic lipid, a PEG-modified lipid, a sterol, and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)-N,N-dimethyl-2-nonylhenicosa-12,15-dien-1-amine (L608), and N,N-dimethyl-1-[(1S,2R)-2-octylcyclopropyl]heptadecan-8-amine (L530).

In some embodiments, SARS-CoV-2 RNA (e.g. mRNA) vaccines are formulated in a lipid nanoparticle. In some embodiments, SARS-CoV-2 RNA (e.g. mRNA) vaccines are formulated in a lipid-polycation complex, referred to as a cationic lipid nanoparticle. The formation of the lipid nanoparticle may be accomplished by methods known in the art and/or as described in U.S. Publication No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Publication No. WO2012013326 or U.S. Publication No. US20130142818; each of which is herein incorporated by reference in its entirety. In some embodiments, SARS-CoV-2 RNA (e.g. mRNA) vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components, and biophysical parameters such as size. In one example by Semple et al. (Nature Biotech. 2010 28:172-176; herein incorporated by reference in its entirety), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid was shown to more effectively deliver siRNA to various antigen presenting cells (Basha et al. Mol Ther. 2011 19:2186-2200; herein incorporated by reference in its entirety).

In some embodiments, lipid nanoparticle formulations may comprise 35% to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1, and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0%, and/or 3.0% to 6.0% of the lipid molar ratio of PEG-c-DOMG (R-3-[(co-methoxy-poly(ethyleneglycol)2000) carbamoyl)]-1,2-dimyristyloxypropyl-3-amine) (also referred to herein as PEG-DOMG) as compared to the cationic lipid, DSPC, and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200, and DLin-KC2-DMA.

In some embodiments, a SARS-CoV-2 RNA (e.g., mRNA) vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, (12Z,15Z)-N,N-dimethyl-2-nonylhenicosa-12,15-dien-1-amine (L608), N,N-dimethyl-1-[(1S,2R)-2-octylcyclopropyl]heptadecan-8-amine (L530), PEGylated lipids, and amino alcohol lipids.

In some embodiments, a lipid nanoparticle formulation includes 25% to 75% on a molar basis of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35% to 65%, 45% to 65%, 60%, 57.5%, 50% or 40% on a molar basis.

In some embodiments, a lipid nanoparticle formulation includes 0.5% to 15% on a molar basis of the neutral lipid, e.g., 3% to 12%, 5% to 10% or 15%, 10%, or 7.5% on a molar basis. Examples of neutral lipids include, without limitation, DSPC, POPC, DPPC, DOPE, and SM. In some embodiments, the formulation includes 5% to 50% on a molar basis of the sterol (e.g., 15% to 45%, 20% to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5% to 10%, 0.5% to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), and PEG-cDMA (further discussed in Reyes et al. J. Controlled Release, 107, 276-287 (2005) the content of which is herein incorporated by reference in its entirety).

In some embodiments, lipid nanoparticle formulations include 25-75% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.5% of the neutral lipid, 31% of the sterol, and 1.5% of the PEG or PEG-modified lipid on a molar basis.

Some embodiments of the present disclosure provide a SARS-CoV-2 vaccine that includes at least one ribonucleic acid (RNA) polynucleotide having an open reading frame encoding at least one SARS-CoV-2 antigenic polypeptide, wherein at least about 80% of the uracil in the open reading frame have a chemical modification, optionally wherein the SARS-CoV-2 vaccine is formulated in a lipid nanoparticle. In some embodiments, the RNA vaccine pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel). In some embodiments, the RNA vaccines may be formulated in a lyophilized gel-phase liposomal composition as described in U.S. Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Publication No. WO2013033438 or U.S. Publication No. US20130196948, the content of each of which is herein incorporated by reference in its entirety. As a non-limiting example, the phosphate conjugates may include a compound of any one of the formulas described in International Publication No. WO2013033438, herein incorporated by reference in its entirety. In particular, the present invention relates to a pharmaceutical composition comprising nanoparticles which comprise RNA encoding at least one antigen, wherein:
(i) the number of positive charges in the nanoparticles does not exceed the number of negative charges in the nanoparticles and/or
(ii) the nanoparticles have a neutral or net negative charge and/or
(iii) the charge ratio of positive charges to negative charges in the nanoparticles is 1.4:1 or less and/or
(iv) the zeta potential of the nanoparticles is 0 or less.

In some embodiments, the nanoparticles described herein are colloidally stable for at least 2 hours in the sense that no aggregation, precipitation or increase of size and polydispersity index by more than 30% as measured by dynamic light scattering takes place. In some embodiments, the charge ratio of positive charges to negative charges in the nanoparticles is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, the zeta potential of the nanoparticles is −5 or less, −10 or less, −15 or less, −20 or less or −25 or less. In various embodiments, the zeta potential of the nanoparticles is −35 or higher, −30 or higher or −25 or higher. In some embodiments, the nanoparticles have a zeta potential from 0 mV to −50 mV, preferably 0 mV to −40 mV or −10 mV to −30 mV.

In some embodiments pharmaceutical compositions of the disclosure comprise a nanoparticle or a liposome that encapsulates a DNA, RNA or DNA/RNA hybrid comprising at least one expressible nucleic acid sequence. Liposomes are microscopic lipidic vesicles often having one or more bilayers of a vesicle-forming lipid, such as a phospholipid, and are capable of encapsulating a drug. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation and the selection of the type of vesicles to be used will depend on the preferred mode of administration. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. The described phases may be present in the nanoparticulate RNA formulations of the present invention.

For formation of RNA lipoplexes from RNA and liposomes, any suitable method of forming liposomes can be used so long as it provides the envisaged RNA lipoplexes. Liposomes may be formed using standard methods such as the reverse evaporation method (REV), the ethanol injection method, the dehydration-rehydration method (DRV), sonication or other suitable methods.

After liposome formation, the liposomes can be sized to obtain a population of liposomes having a substantially homogeneous size range.

Bilayer-forming lipids have typically two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition of the present invention include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. Most preferred is DOTMA.

In addition, the nanoparticles described herein preferably further include a neutral lipid in view of structural stability and the like. The neutral lipid can be appropriately selected in view of the delivery efficiency of the RNA-lipid complex. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside. Preferred is DOPE and/or DOPC. Most preferred is DOPE. In the case where a cationic liposome includes both a cationic lipid and a neutral lipid, the molar ratio of the cationic lipid to the neutral lipid can be appropriately determined in view of stability of the liposome and the like.

According to one embodiment, the nanoparticles described herein may comprise phospholipids. The phospholipids may be a glycerophospholipid. Examples of glycerophospholipid include, without being limited thereto, three types of lipids: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxy-polyethylene,glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of RNA to the lipid carrier can occur, for example, by the RNA filling interstitial spaces of the carrier, such that the carrier physically entraps the RNA, or by covalent, ionic, or hydrogen bonding, or by means of adsorption by non-specific bonds. Whatever the mode of association, the RNA must retain its therapeutic, i.e. antigen-encoding, properties.

In some embodiments, the nanoparticles comprise at least one lipid. In some embodiments, the nanoparticles comprise at least one cationic lipid. The cationic lipid can be monocationic or polycationic. Any cationic amphiphilic molecule, eg, a molecule which comprises at least one hydrophilic and lipophilic moiety is a cationic lipid within the meaning of the present invention. In some embodiments, the positive charges are contributed by the at least one cationic lipid and the negative charges are contributed by the RNA. In some embodiments, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In some embodiments, the cationic lipid and/or the helper lipid is a bilayer forming lipid.

In some embodiments, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof. In some embodiments, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof. In some embodiments, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In some embodiments, in this ratio, the molar amount of the cationic lipid results from the molar amount of the cationic lipid multiplied by the number of positive charges in the cationic lipid. In various embodiments, the lipids are not functionalized such as functionalized by mannose, histidine and/or imidazole, the nanoparticles do not comprise a targeting ligand such as mannose functionalized lipids and/or the nanoparticles do not comprise one or more of the following: pH dependent compounds, cationic polymers such as polymers containing histidine and/or polylysine, wherein the polymers may optionally be PEGylated and/or histidylated, or divalent ions such as Ca 2+.

In various embodiments, the RNA nanoparticles may comprise peptides, preferentially with a molecular weight of up to 2500 Da.

In the nanoparticles described herein the lipid may form a complex with and/or may encapsulate the RNA. In some embodiments, the nanoparticles comprise a lipoplex or liposome. In some embodiments, the lipid is comprised in a vesicle encapsulating said RNA. The vesicle may be a multilamellar vesicle, an unilamellar vesicle, or a mixture thereof. The vesicle may be a liposome. In some embodiments, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In some embodiments, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In some embodiments, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In some embodiments, the nanoparticles are lipoplexes comprising DOTMA and DOPE in a molar ratio of 2:1 to 1:2, preferably 2:1 to 1:1, and wherein the charge ratio of positive charges in DOTMA to negative charges in the RNA is 1.4:1 or less. In some embodiments, the nanoparticles are lipoplexes comprising DOTMA and cholesterol in a molar ratio of 2:1 to 1:2, preferably 2:1 to 1:1, and wherein the charge ratio of positive charges in DOTMA to negative charges in the RNA is 1.4:1 or less. In some embodiments, the nanoparticles are lipoplexes comprising DOTAP and DOPE in a molar ratio of 2:1 to 1:2, preferably 2:1 to 1:1, and wherein the charge ratio of positive charges in DOTAP to negative charges in the RNA is 1.4:1 or less. In some embodiments, the nanoparticles have an average diameter in the range of from about 50 nm to about 1000 nm, preferably from about 50 nm to about 400 nm, preferably about 100 nm to about 300 nm such as about 150 nm to about 200 nm. In some embodiments, the nanoparticles have a diameter in the range of about 200 to about 700 nm, about 200 to about 600 nm, preferably about 250 to about 550 nm, in particular about 300 to about 500 nm or about 200 to about 400 nm.

In some embodiments, the polydispersity index of the nanoparticles described herein as measured by dynamic light scattering is 0.5 or less, preferably 0.4 or less or even more preferably 0.3 or less. In some embodiments, the nanoparticles described herein are obtainable by one or more of the following: (i) incubation of liposomes in an aqueous phase with the RNA in an aqueous phase, (ii) incubation of the lipid dissolved in an organic, water miscible solvent, such as ethanol, with the RNA in aqueous solution, (iii) reverse phase evaporation technique, (iv) freezing and thawing of the product, (v) dehydration and rehydration of the product, (vi) lyophilization and rehydration of the of the product, or (vii) spray drying and rehydration of the product.

The nanoparticle formulation may comprise a polymer conjugate. The polymer conjugate may be a water-soluble conjugate. The polymer conjugate may have a structure as described in U.S. Publication No. 20130059360, the content of which is herein incorporated by reference in its entirety. In some aspects, polymer conjugates with the polynucleotides of the present invention may be made using the methods and/or segmented polymeric reagents described in U.S. Publication No. 20130072709, herein incorporated by reference in its entirety. In other aspects, the polymer conjugate may have pendant side groups comprising ring moieties such as, but not limited to, the polymer conjugates described in U.S. Publication No. US20130196948, the contents of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In some aspects, the conjugate may be a “self” peptide designed from the human membrane protein CD47 (e.g., the “self” particles described by Rodriguez et al. (Science 2013, 339, 971-975), herein incorporated by reference in its entirety). As shown by Rodriguez et al., the self peptides delayed macrophage-mediated clearance of nanoparticles which enhanced delivery of the nanoparticles. In other aspects, the conjugate may be the membrane protein CD47 (e.g., see Rodriguez et al. Science 2013, 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, efficacy of RNA vaccines RNA (e.g., mRNA) can be significantly enhanced when combined with a flagellin adjuvant, in particular, when one or more antigen-encoding mRNAs is combined with an mRNA encoding flagellin.

RNA (e.g., mRNA) vaccines combined with the flagellin adjuvant (e.g., mRNA-encoded flagellin adjuvant) have superior properties in that they may produce much larger antibody titers and produce responses earlier than commercially available vaccine formulations. While not wishing to be bound by theory, it is believed that the RNA vaccines, for example, as mRNA polynucleotides, are better designed to produce the appropriate protein conformation upon translation, for both the antigen and the adjuvant, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Some embodiments of the present disclosure provide RNA (e.g., mRNA) vaccines that include at least one RNA (e.g., mRNA) polynucleotide having an open reading frame encoding at least one antigenic polypeptide or an immunogenic fragment thereof (e.g., an immunogenic fragment capable of inducing an immune response to the antigenic polypeptide) and at least one RNA (e.g., mRNA polynucleotide) having an open reading frame encoding a flagellin adjuvant.

In some embodiments, at least one flagellin polypeptide (e.g., encoded flagellin polypeptide) is a flagellin protein. In some embodiments, at least one flagellin polypeptide (e.g., encoded flagellin polypeptide) is an immunogenic flagellin fragment. In some embodiments, at least one flagellin polypeptide and at least one antigenic polypeptide are encoded by a single RNA (e.g., mRNA) polynucleotide. In other embodiments, at least one flagellin polypeptide and at least one antigenic polypeptide are each encoded by a different RNA polynucleotide.

Some embodiments of the present disclosure provide methods of inducing an antigen specific immune response in a subject, comprising administering to the subject a SARS-CoV-2 vaccine in an amount effective to produce an antigen specific immune response.

In some aspects, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In exemplary aspects of the invention, antigen-specific antibodies are measured in units of g/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

In some embodiments, the SARS-CoV-2 vaccine includes at least one RNA polynucleotide having an open reading frame encoding at least one SARS-CoV-2 antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source. When transfected into mammalian cells, the modified mRNAs have a stability of from about 12 to about 18 hours or more than about 18 hours, e.g., 24, 36, 48, 60, 72, or greater than about 72 hours.

In some embodiments, a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxyuridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (mlG), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQ0base, preQ1base, and combinations of two or more thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polyribonucleotide (e.g., RNA polyribonucleotide, such as mRNA polyribonucleotide) includes a combination of at least two (e.g., 2, 3, 4 or more) of the aforementioned modified nucleobases. In some embodiments, polynucleotides (e.g., RNA polynucleotides, such as mRNA polynucleotides) include a combination of at least two (e.g., 2, 3, 4 or more) of the aforementioned modified nucleobases.

The expressible nucleic acid sequence of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may be any one of nucleotides A, G, U, C, or any one of the combinations A+G, A+U, A+C, G+U, G+C, U+C, A+G+U, A+G+C, G+U+C, or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from about 1% to about 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acid sequences may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4, or more unique structures). In some embodiments, at least 5%, at least 10%, at least 25%, at least 50%, at least 80%, at least 90%, or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4, or more unique structures).

Thus, in some embodiments, the RNA vaccines and/or RNA nucleic acid sequences comprise a 5′UTR element, an optionally codon optimized open reading frame, and a 3′UTR element, a poly(A) sequence and/or a polyadenylation signal wherein the RNA is not chemically modified.

Viral vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, the at least one RNA polynucleotide has at least one chemical modification. The at least one chemical modification may include, but is expressly not limited to, any modification described herein.

In vitro transcription of RNA is known in the art and is described in WO/2014/152027, which is incorporated by reference herein in its entirety. For example, in some embodiments, the RNA transcript is generated using a non-amplified, linearized DNA template in an in vitro transcription reaction to generate the RNA transcript. In some embodiments, the RNA transcript is capped via enzymatic capping. In some embodiments, the RNA transcript is purified via chromatographic methods, e.g., use of an oligo dT substrate. Some embodiments exclude the use of DNase. In some embodiments, the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present invention. The polymerase may be selected from, but is not limited to, a phage RNA polymerase, e.g., a T7 RNA polymerase, a T3 RNA polymerase, a SP6 RNa polymerase, and/or mutant polymerases such as, but not limited to, polymerases able to incorporate modified nucleic acids and/or modified nucleotides, including chemically modified nucleic acids and/or nucleotides.

In some embodiments, anon-amplified, linearized plasmid DNA is utilized as the template DNA for in vitro transcription. In some embodiments, the template DNA is isolated DNA. In some embodiments, the template DNA is cDNA. In some embodiments, the cDNA is formed by reverse transcription of a RNA polynucleotide, for example, but not limited to SARS-CoV-2 RNA, e.g. SARS-CoV-2 mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

### F. Vaccines

Disclosed are DNA vaccines comprising at least about 70%, 75%, 80%, 85%, 90%, 91%, 92%, 93%, 94%, 95%, 96%, 97%, 98%, 99% or 100% sequence identity to SEQ ID NO: 68, SEQ ID NO: 71, SEQ ID NO: 74, SEQ ID NO: 77, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155 or SEQ ID NO: 158, or a functional fragment or variant thereof. Also disclosed are RNA vaccines comprising at least about 70%, 75%, 80%, 85%, 90%, 91%, 92%, 93%, 94%, 95%, 96%, 97%, 98%, 99% or 100% sequence identity to SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 102, SEQ ID NO: 105, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132, SEQ ID NO: 135, SEQ ID NO: 138, SEQ ID NO: 141, SEQ ID NO: 144, SEQ ID NO: 147, SEQ ID NO: 150, SEQ ID NO: 153, SEQ ID NO: 156 or SEQ ID NO: 159, or a functional fragment or variant thereof. In some embodiment, the DNA or RNA vaccine disclosed herein encodes a polypeptide comprising at least about 70%, 75%, 80%, 85%, 90%, 91%, 92%, 93%, 94%, 95%, 96%, 97%, 98%, 99% or 100% sequence identity to SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94, SEQ ID NO: 97, SEQ ID NO: 100, SEQ ID NO: 103, SEQ ID NO: 106, SEQ ID NO: 109, SEQ ID NO: 112, SEQ ID NO: 115, SEQ ID NO: 118, SEQ ID NO: 121, SEQ ID NO: 124, SEQ ID NO: 127, SEQ ID NO: 130, SEQ ID NO: 133, SEQ ID NO: 136, SEQ ID NO: 139, SEQ ID NO: 142, SEQ ID NO: 145, SEQ ID NO: 148, SEQ ID NO: 151, SEQ ID NO: 154, SEQ ID NO: 157 or SEQ ID NO: 160, or a functional fragment or variant thereof. In some embodiments, the disclosed DNA vaccine further comprises a pharmaceutically acceptable excipient. In some embodiments, the pharmaceutically acceptable excipient is an adjuvant.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the elements of the disclosed nucleic acid compositions. For example, disclosed are kits comprising nucleic acid sequences comprising a leader sequence, a linker sequence, a nucleic acid sequence encoding a self-assembling polypeptide, and/or a nucleic acid sequence encoding a viral antigen. In some embodiments, the kits can further comprise a plasmid backbone.

Vaccine constructs in accordance with the present disclosure are provided below and may comprise contiguously or non-contiguously a nucleic acid that encodes the following protein sequences:

Key:
IgE leader sequence- LS3-Epitope- linker (contiguous)
1. LS3_SARS-COV Spike
MDWTWILFLVAAATRVHS (IgE leader)
LRFGIVASRANHALV (LS-3 epitope)
GGSGGSGGSGGSGGG (linker)
MFIFLLFLTLTSGSDLDRCTTFDDVQAPNYTQHTSSMRGVYYPDEIFRSDTLYLTQDL
FLPFYSNVTGFHTINHTFGNPVIPFKDGIYFAATEKSNVVRGWVFGSTMNNKSQSVIII
NNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAI
LTAFSPAQDIWGTSAAAYFVGYLKPTTFMLKYDENGTITDAVDCSQNPLAELKCSVK
SFEIDKGIYQTSNFRVVPSGDVVRFPNITNLCPFGEVFNATKFPSVYAWERKKISNCV
ADYSVLYNSTFFSTFKCYGVSATKLNDLCFSNVYADSFVVKGDDVRQIAPGQTGVIA
DYNYKLPDDFMGCVLAWNTRNIDATSTGNYNYKYRYLRHGKLRPFERDISNVPFSP
DGKPCTPPALNCYWPLNDYGFYTTTGIGYQPYRVVVLSFELLNAPATVCGPKLSTDL
IKNQCVNFNFNGLTGTGVLTPSSKRFQPFQQFGRDVSDFTDSVRDPKTSEILDISPCSF
GGVSVITPGTNASSEVAVLYQDVNCTDVSTAIHADQLTPAWRIYSTGNNVFQTQAGC
LIGAEHVDTSYECDIPIGAGICASYHTVSLLRSTSQKSIVAYTMSLGADSSIAYSNNTIA
IPTNFSISITTEVMPVSMAKTSVDCNMYICGDSTECANLLLQYGSFCTQLNRALSGIAA
EQDRNTREVFAQVKQMYKTPTLKYFGGFNFSQILPDPLKPTKRSFIEDLLFNKVTLAD
AGFMKQYGECLGDINARDLICAQKFNGLTVLPPLLTDDMIAAYTAALVSGTATAGW
TFGAGAALQIPFAMQMAYRFNGIGVTQNVLYENQKQIANQFNKAISQIQESLTTTST
ALGKLQDVVNQNAQALNTLVKQLSSNFGAISSVLNDILSRLDKVEAEVQIDRLITGRL
QSLQTYVTQQLIRAAEIRASANLAATKMSECVLGQSKRVDFCGKGYHLMSFPQAAP
HGVVFLHVTYVPSQERNFTTAPAICHEGKAYFPREGVFVFNGTSWFITQRNFFSPQIIT
TDNTFVSGNCDVVIGIINNTVYDPLQPELDSFKEELDKYFKNHTSPDVDLGDISGINAS
VVNIQKEIDRLNEVAKNLNESLIDLQELGKYEQYIKWPWYVWLGFIAGLIAIVMVTIL
LCCMTSCCSCLKGACSCGSCCKFDEDDSEPVLKGVKLHYT
DNA sequence (each plus identifies a subpart of th enucleica cid seqeunce;
yet seqeunce below is contiguous)
ATGGACTGGACCTGGATCCTGTTCCTGGTGGCCGCCGCCACCAGGGTGCACAGC +
CTGAGGTTCGGCATCGTGGCCAGCAGGGCCAACCACGCCCTGGTG +
GGCGGCAGCGGCGGCAGCGGCGGCAGCGGCGGCAGCGGCGGCGGC +
ATGTTCATCTTCCTGCTGTTCCTGACCCTGACCAGCGGCAGCGACCTGGACAGGTGCACCACCTTCGACGACGTGCAGG
CCCCCAACTACACCCAGCACACCAGCAGCATGAGGGGCGTGTACTACCCCGACGAGATCTTCAGGAGCGACACCCTGTA
CCTGACCCAGGACCTGTTCCTGCCCTTCTACAGCAACGTGACCGGCTTCCACACCATCAACCACACCTTCGGCAACCCC
GTGATCCCCTTCAAGGACGGCATCTACTTCGCCGCCACCGAGAAGAGCAACGTGGTGAGGGGCTGGGTGTTCGGCAGCA
CCATGAACAACAAGAGCCAGAGCGTGATCATCATCAACAACAGCACCAACGTGGTGATCAGGGCCTGCAACTTCGAGCT
GTGCGACAACCCCTTCTTCGCCGTGAGCAAGCCCATGGGCACCCAGACCCACACCATGATCTTCGACAACGCCTTCAAC
TGCACCTTCGAGTACATCAGCGACGCCTTCAGCCTGGACGTGAGCGAGAAGAGCGGCAACTTCAAGCACCTGAGGGAGT
TCGTGTTCAAGAACAAGGACGGCTTCCTGTACGTGTACAAGGGCTACCAGCCCATCGACGTGGTGAGGGACCTGCCCAG
CGGCTTCAACACCCTGAAGCCCATCTTCAAGCTGCCCCTGGGCATCAACATCACCAACTTCAGGGCCATCCTGACCGCC
TTCAGCCCCGCCCAGGACATCTGGGGCACCAGCGCCGCCGCCTACTTCGTGGGCTACCTGAAGCCCACCACCTTCATGC
TGAAGTACGACGAGAACGGCACCATCACCGACGCCGTGGACTGCAGCCAGAACCCCCTGGCCGAGCTGAAGTGCAGCGT
GAAGAGCTTCGAGATCGACAAGGGCATCTACCAGACCAGCAACTTCAGGGTGGTGCCCAGCGGCGACGTGGTGAGGTTC
CCCAACATCACCAACCTGTGCCCCTTCGGCGAGGTGTTCAACGCCACCAAGTTCCCCAGCGTGTACGCCTGGGAGAGGA
AGAAGATCAGCAACTGCGTGGCCGACTACAGCGTGCTGTACAACAGCACCTTCTTCAGCACCTTCAAGTGCTACGGCGT
GAGCGCCACCAAGCTGAACGACCTGTGCTTCAGCAACGTGTACGCCGACAGCTTCGTGGTGAAGGGCGACGACGTGAGG
CAGATCGCCCCCGGCCAGACCGGCGTGATCGCCGACTACAACTACAAGCTGCCCGACGACTTCATGGGCTGCGTGCTGG
CCTGGAACACCAGGAACATCGACGCCACCAGCACCGGCAACTACAACTACAAGTACAGGTACCTGAGGCACGGCAAGCT
GAGGCCCTTCGAGAGGGACATCAGCAACGTGCCCTTCAGCCCCGACGGCAAGCCCTGCACCCCCCCCGCCCTGAACTGC
TACTGGCCCCTGAACGACTACGGCTTCTACACCACCACCGGCATCGGCTACCAGCCCTACAGGGTGGTGGTGCTGAGCT
TCGAGCTGCTGAACGCCCCCGCCACCGTGTGCGGCCCCAAGCTGAGCACCGACCTGATCAAGAACCAGTGCGTGAACTT
CAACTTCAACGGCCTGACCGGCACCGGCGTGCTGACCCCCAGCAGCAAGAGGTTCCAGCCCTTCCAGCAGTTCGGCAGG
GACGTGAGCGACTTCACCGACAGCGTGAGGGACCCCAAGACCAGCGAGATCCTGGACATCAGCCCCTGCAGCTTCGGCG
GCGTGAGCGTGATCACCCCCGGCACCAACGCCAGCAGCGAGGTGGCCGTGCTGTACCAGGACGTGAACTGCACCGACGT
GAGCACCGCCATCCACGCCGACCAGCTGACCCCCGCCTGGAGGATCTACAGCACCGGCAACAACGTGTTCCAGACCCAG
GCCGGCTGCCTGATCGGCGCCGAGCACGTGGACACCAGCTACGAGTGCGACATCCCCATCGGCGCCGGCATCTGCGCCA
GCTACCACACCGTGAGCCTGCTGAGGAGCACCAGCCAGAAGAGCATCGTGGCCTACACCATGAGCCTGGGCGCCGACAG
CAGCATCGCCTACAGCAACAACACCATCGCCATCCCCACCAACTTCAGCATCAGCATCACCACCGAGGTGATGCCCGTG
AGCATGGCCAAGACCAGCGTGGACTGCAACATGTACATCTGCGGCGACAGCACCGAGTGCGCCAACCTGCTGCTGCAGT
ACGGCAGCTTCTGCACCCAGCTGAACAGGGCCCTGAGCGGCATCGCCGCCGAGCAGGACAGGAACACCAGGGAGGTGTT
CGCCCAGGTGAAGCAGATGTACAAGACCCCCACCCTGAAGTACTTCGGCGGCTTCAACTTCAGCCAGATCCTGCCCGAC
CCCCTGAAGCCCACCAAGAGGAGCTTCATCGAGGACCTGCTGTTCAACAAGGTGACCCTGGCCGACGCCGGCTTCATGA
AGCAGTACGGCGAGTGCCTGGGCGACATCAACGCCAGGGACCTGATCTGCGCCCAGAAGTTCAACGGCCTGACCGTGCT
GCCCCCCCTGCTGACCGACGACATGATCGCCGCCTACACCGCCGCCCTGGTGAGCGGCACCGCCACCGCCGGCTGGACC
TTCGGCGCCGGCGCCGCCCTGCAGATCCCCTTCGCCATGCAGATGGCCTACAGGTTCAACGGCATCGGCGTGACCCAGA
ACGTGCTGTACGAGAACCAGAAGCAGATCGCCAACCAGTTCAACAAGGCCATCAGCCAGATCCAGGAGAGCCTGACCAC
CACCAGCACCGCCCTGGGCAAGCTGCAGGACGTGGTGAACCAGAACGCCCAGGCCCTGAACACCCTGGTGAAGCAGCTG
AGCAGCAACTTCGGCGCCATCAGCAGCGTGCTGAACGACATCCTGAGCAGGCTGGACAAGGTGGAGGCCGAGGTGCAGA
TCGACAGGCTGATCACCGGCAGGCTGCAGAGCCTGCAGACCTACGTGACCCAGCAGCTGATCAGGGCCGCCGAGATCAG
GGCCAGCGCCAACCTGGCCGCCACCAAGATGAGCGAGTGCGTGCTGGGCCAGAGCAAGAGGGTGGACTTCTGCGGCAAG
GGCTACCACCTGATGAGCTTCCCCCAGGCCGCCCCCCACGGCGTGGTGTTCCTGCACGTGACCTACGTGCCCAGCCAGG
AGAGGAACTTCACCACCGCCCCCGCCATCTGCCACGAGGGCAAGGCCTACTTCCCCAGGGAGGGCGTGTTCGTGTTCAA
CGGCACCAGCTGGTTCATCACCCAGAGGAACTTCTTCAGCCCCCAGATCATCACCACCGACAACACCTTCGTGAGCGGC
AACTGCGACGTGGTGATCGGCATCATCAACAACACCGTGTACGACCCCCTGCAGCCCGAGCTGGACAGCTTCAAGGAGG
AGCTGGACAAGTACTTCAAGAACCACACCAGCCCCGACGTGGACCTGGGCGACATCAGCGGCATCAACGCCAGCGTGGT
GAACATCCAGAAGGAGATCGACAGGCTGAACGAGGTGGCCAAGAACCTGAACGAGAGCCTGATCGACCTGCAGGAGCTG
GGCAAGTACGAGCAGTACATCAAGTGGCCCTGGTACGTGTGGCTGGGCTTCATCGCCGGCCTGATCGCCATCGTGATGG
TGACCATCCTGCTGTGCTGCATGACCAGCTGCTGCAGCTGCCTGAAGGGCGCCTGCAGCTGCGGCAGCTGCTGCAAGTT
CGACGAGGACGACAGCGAGCCCGTGCTGAAGGGCGTGAAGCTGCACTACACC
2. LS3_SARS-COV2 Spike
MDWTWILFLVAAATRVHSLRFGIVASRANHALVGGSGGSGGSGGSGGGMFVFLVLLPLV
SSQCVNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATVCGPKKS
TNLVKNKCVNFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYSTGSNVFQT
RAGCLIGAEHVNNSYECDIPIGAGICASYQTQTNSPRRARSVASQSIIAYTMSLGAENS
VAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLL
FNKVTLADAGFIKQYGDCLGDIAARDLICAQKFNGLTVLPPLLTDEMIAQYTSALLA
GTITSGWTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKVEAEVQID
RLITGRLQSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTHWFVTQR
NFYEPQUITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAG
LIAIVMVTIMLCCMTSCCSCLKGCCSCGSCCKFDEDDSEPVLKGVKLHYT
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATGTTCGTG
TTCCTGGTGCTGCTGCCCCTGGTGAGCAGCCAGTGCGTGAACCTGACCACCAGGACCCAGCTGCCCCCC
GCCTACACCAACAGCTTCACCAGGGGCGTGTACTACCCCGACAAGGTGTTCAGGAGCAGCGTGCTGCAC
AGCACCCAGGACCTGTTCCTGCCCTTCTTCAGCAACGTGACCTGGTTCCACGCCATCCACGTGAGCGGCA
CCAACGGCACCAAGAGGTTCGACAACCCCGTGCTGCCCTTCAACGACGGCGTGTACTTCGCCAGCACCG
AGAAGAGCAACATCATCAGGGGCTGGATCTTCGGCACCACCCTGGACAGCAAGACCCAGAGCCTGCTG
ATCGTGAACAACGCCACCAACGTGGTGATCAAGGTGTGCGAGTTCCAGTTCTGCAACGACCCCTTCCTG
GGCGTGTACTACCACAAGAACAACAAGAGCTGGATGGAGAGCGAGTTCAGGGTGTACAGCAGCGCCAA
CAACTGCACCTTCGAGTACGTGAGCCAGCCCTTCCTGATGGACCTGGAGGGCAAGCAGGGCAACTTCAA
GAACCTGAGGGAGTTCGTGTTCAAGAACATCGACGGCTACTTCAAGATCTACAGCAAGCACACCCCCAT
CAACCTGGTGAGGGACCTGCCCCAGGGCTTCAGCGCCCTGGAGCCCCTGGTGGACCTGCCCATCGGCAT
CAACATCACCAGGTTCCAGACCCTGCTGGCCCTGCACAGGAGCTACCTGACCCCCGGCGACAGCAGCAG
CGGCTGGACCGCCGGCGCCGCCGCCTACTACGTGGGCTACCTGCAGCCCAGGACCTTCCTGCTGAAGTA
CAACGAGAACGGCACCATCACCGACGCCGTGGACTGCGCCCTGGACCCCCTGAGCGAGACCAAGTGCA
CCCTGAAGAGCTTCACCGTGGAGAAGGGCATCTACCAGACCAGCAACTTCAGGGTGCAGCCCACCGAG
AGCATCGTGAGGTTCCCCAACATCACCAACCTGTGCCCCTTCGGCGAGGTGTTCAACGCCACCAGGTTC
GCCAGCGTGTACGCCTGGAACAGGAAGAGGATCAGCAACTGCGTGGCCGACTACAGCGTGCTGTACAA
CAGCGCCAGCTTCAGCACCTTCAAGTGCTACGGCGTGAGCCCCACCAAGCTGAACGACCTGTGCTTCAC
CAACGTGTACGCCGACAGCTTCGTGATCAGGGGCGACGAGGTGAGGCAGATCGCCCCCGGCCAGACCG
GCAAGATCGCCGACTACAACTACAAGCTGCCCGACGACTTCACCGGCTGCGTGATCGCCTGGAACAGCA
ACAACCTGGACAGCAAGGTGGGCGGCAACTACAACTACCTGTACAGGCTGTTCAGGAAGAGCAACCTG
AAGCCCTTCGAGAGGGACATCAGCACCGAGATCTACCAGGCCGGCAGCACCCCCTGCAACGGCGTGGA
GGGCTTCAACTGCTACTTCCCCCTGCAGAGCTACGGCTTCCAGCCCACCAACGGCGTGGGCTACCAGCC
CTACAGGGTGGTGGTGCTGAGCTTCGAGCTGCTGCACGCCCCCGCCACCGTGTGCGGCCCCAAGAAGAG
CACCAACCTGGTGAAGAACAAGTGCGTGAACTTCAACTTCAACGGCCTGACCGGCACCGGCGTGCTGAC
CGAGAGCAACAAGAAGTTCCTGCCCTTCCAGCAGTTCGGCAGGGACATCGCCGACACCACCGACGCCGT
GAGGGACCCCCAGACCCTGGAGATCCTGGACATCACCCCCTGCAGCTTCGGCGGCGTGAGCGTGATCAC
CCCCGGCACCAACACCAGCAACCAGGTGGCCGTGCTGTACCAGGACGTGAACTGCACCGAGGTGCCCGT
GGCCATCCACGCCGACCAGCTGACCCCCACCTGGAGGGTGTACAGCACCGGCAGCAACGTGTTCCAGAC
CAGGGCCGGCTGCCTGATCGGCGCCGAGCACGTGAACAACAGCTACGAGTGCGACATCCCCATCGGCG
CCGGCATCTGCGCCAGCTACCAGACCCAGACCAACAGCCCCAGGAGGGCCAGGAGCGTGGCCAGCCAG
AGCATCATCGCCTACACCATGAGCCTGGGCGCCGAGAACAGCGTGGCCTACAGCAACAACAGCATCGC
CATCCCCACCAACTTCACCATCAGCGTGACCACCGAGATCCTGCCCGTGAGCATGACCAAGACCAGCGT
GGACTGCACCATGTACATCTGCGGCGACAGCACCGAGTGCAGCAACCTGCTGCTGCAGTACGGCAGCTT
CTGCACCCAGCTGAACAGGGCCCTGACCGGCATCGCCGTGGAGCAGGACAAGAACACCCAGGAGGTGT
TCGCCCAGGTGAAGCAGATCTACAAGACCCCCCCCATCAAGGACTTCGGCGGCTTCAACTTCAGCCAGA
TCCTGCCCGACCCCAGCAAGCCCAGCAAGAGGAGCTTCATCGAGGACCTGCTGTTCAACAAGGTGACCC
TGGCCGACGCCGGCTTCATCAAGCAGTACGGCGACTGCCTGGGCGACATCGCCGCCAGGGACCTGATCT
GCGCCCAGAAGTTCAACGGCCTGACCGTGCTGCCCCCCCTGCTGACCGACGAGATGATCGCCCAGTACA
CCAGCGCCCTGCTGGCCGGCACCATCACCAGCGGCTGGACCTTCGGCGCCGGCGCCGCCCTGCAGATCC
CCTTCGCCATGCAGATGGCCTACAGGTTCAACGGCATCGGCGTGACCCAGAACGTGCTGTACGAGAACC
AGAAGCTGATCGCCAACCAGTTCAACAGCGCCATCGGCAAGATCCAGGACAGCCTGAGCAGCACCGCC
AGCGCCCTGGGCAAGCTGCAGGACGTGGTGAACCAGAACGCCCAGGCCCTGAACACCCTGGTGAAGCA
GCTGAGCAGCAACTTCGGCGCCATCAGCAGCGTGCTGAACGACATCCTGAGCAGGCTGGACAAGGTGG
AGGCCGAGGTGCAGATCGACAGGCTGATCACCGGCAGGCTGCAGAGCCTGCAGACCTACGTGACCCAG
CAGCTGATCAGGGCCGCCGAGATCAGGGCCAGCGCCAACCTGGCCGCCACCAAGATGAGCGAGTGCGT
GCTGGGCCAGAGCAAGAGGGTGGACTTCTGCGGCAAGGGCTACCACCTGATGAGCTTCCCCCAGAGCG
CCCCCCACGGCGTGGTGTTCCTGCACGTGACCTACGTGCCCGCCCAGGAGAAGAACTTCACCACCGCCC
CCGCCATCTGCCACGACGGCAAGGCCCACTTCCCCAGGGAGGGCGTGTTCGTGAGCAACGGCACCCACT
GGTTCGTGACCCAGAGGAACTTCTACGAGCCCCAGATCATCACCACCGACAACACCTTCGTGAGCGGCA
ACTGCGACGTGGTGATCGGCATCGTGAACAACACCGTGTACGACCCCCTGCAGCCCGAGCTGGACAGCT
TCAAGGAGGAGCTGGACAAGTACTTCAAGAACCACACCAGCCCCGACGTGGACCTGGGCGACATCAGC
GGCATCAACGCCAGCGTGGTGAACATCCAGAAGGAGATCGACAGGCTGAACGAGGTGGCCAAGAACCT
GAACGAGAGCCTGATCGACCTGCAGGAGCTGGGCAAGTACGAGCAGTACATCAAGTGGCCCTGGTACA
TCTGGCTGGGCTTCATCGCCGGCCTGATCGCCATCGTGATGGTGACCATCATGCTGTGCTGCATGACCAG
CTGCTGCAGCTGCCTGAAGGGCTGCTGCAGCTGCGGCAGCTGCTGCAAGTTCGACGAGGACGACAGCGA
GCCCGTGCTGAAGGGCGTGAAGCTGCACTACACC
3. LS3_SARS-COV2 S RBD
MDWTWILFLVAAATRVHSLRFGIVASRANHALVGGSGGSGGSGGSGGGRVQPTESIVRFP
NITNLCPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGFNCYFPLQSYGFQPTNG
VGYQPYRVVVLSFELLHAPATVCGPKKSTNLVKNKCVNF
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCAGGGTGCAGCCCAC
CGAGAGCATCGTGAGGTTCCCCAACATCACCAACCTGTGCCCCTTCGGCGAGGTGTTCAACGCCACCAGGTTCGCCAGC
GTGTACGCCTGGAACAGGAAGAGGATCAGCAACTGCGTGGCCGACTACAGCGTGCTGTACAACAGCGCCAGCTTCAGCA
CCTTCAAGTGCTACGGCGTGAGCCCCACCAAGCTGAACGACCTGTGCTTCACCAACGTGTACGCCGACAGCTTCGTGAT
CAGGGGCGACGAGGTGAGGCAGATCGCCCCCGGCCAGACCGGCAAGATCGCCGACTACAACTACAAGCTGCCCGACGAC
TTCACCGGCTGCGTGATCGCCTGGAACAGCAACAACCTGGACAGCAAGGTGGGCGGCAACTACAACTACCTGTACAGGC
TGTTCAGGAAGAGCAACCTGAAGCCCTTCGAGAGGGACATCAGCACCGAGATCTACCAGGCCGGCAGCACCCCCTGCAA
CGGCGTGGAGGGCTTCAACTGCTACTTCCCCCTGCAGAGCTACGGCTTCCAGCCCACCAACGGCGTGGGCTACCAGCCC
TACAGGGTGGTGGTGCTGAGCTTCGAGCTGCTGCACGCCCCCGCCACCGTGTGCGGCCCCAAGAAGAGCACCAACCTGG
TGAAGAACAAGTGCGTGAACTTC
4. LS3_MERS-COV2 Spike
MDWTWILFLVAAATRVHSLRFGIVASRANHALVGGSGGSGGSGGSGGGMIHSVFLLMFL
LTPTESYVDVGPDSVKSACIEVDIQQTFFDKTWPRPIDVSKADGIIYPQGRTYSNITITY
QGLFPYQGDHGDMYVYSAGHATGTTPQKLFVANYSQDVKQFANGFVVRIGAAANS
TGTVIISPSTSATIRKIYPAFMLGSSVGNFSDGKMGRFFNHTLVLLPDGCGTLLRAFYC
ILEPRSGNHCPAGNSYTSFATYHTPATDCSDGNYNRNASLNSFKEYFNLRNCTFMYT
YNITEDEILEWFGITQTAQGVHLFSSRYVDLYGGNMFQFATLPVYDTIKYYSIIPHSIR
SIQSDRKAWAAFYVYKLQPLTFLLDFSVDGYIRRAIDCGFNDLSQLHCSYESFDVESG
VYSVSSFEAKPSGSVVEQAEGVECDFSPLLSGTPPQVYNFKRLVFTNCNYNLTKLLSL
FSVNDFTCSQISPAAIASNCYSSLILDYFSYPLSMKSDLSVSSAGPISQFNYKQSFSNPT
CLILATVPHNLTTITKPLKYSYINKCSRFLSDDRTEVPQLVNANQYSPCVSIVPSTVWE
DGDYYRKQLSPLEGGGWLVASGSTVAMTEQLQMGFGITVQYGTDTNSVCPKLEFA
NDTKIASQLGNCVEYSLYGVSGRGVFQNCTAVGVRQQRFVYDAYQNLVGYYSDDG
NYYCLRACVSVPVSVIYDKETKTHATLFGSVACEHISSTMSQYSRSTRSMLKRRDST
YGPLQTPVGCVLGLVNSSLFVEDCKLPLGQSLCALPDTPSTLTPRSVRSVPGEMRLAS
IAFNHPIQVDQLNSSYFKLSIPTNFSFGVTQEYIQTTIQKVTVDCKQYVCNGFQKCEQL
LREYGQFCSKINQALHGANLRQDDSVRNLFASVKSSQSSPIIPGFGGDFNLTLLEPVSI
STGSRSARSAIEDLLFDKVTIADPGYMQGYDDCMQQGPASARDLICAQYVAGYKVL
PPLMDVNMEAAYTSSLLGSIAGVGWTAGLSSFAAIPFAQSIFYRLNGVGITQQVLSEN
QKLIANKFNQALGAMQTGFTTTNEAFHKVQDAVNNNAQALSKLASELSNTFGAISA
SIGDIIQRLDVLEQDAQIDRLINGRLTTLNAFVAQQLVRSESAALSAQLAKDKVNECV
KAQSKRSGFCGQGTHIVSFVVNAPNGLYFMHVGYYPSNHIEVVSAYGLCDAANPTN
CIAPVNGYFIKTNNTRIVDEWSYTGSSFYAPEPITSLNTKYVAPQVTYQNISTNLPPPL
LGNSTGIDFQDELDEFFKNVSTSIPNFGSLTQINTTLLDLTYEMLSLQQVVKALNESYI
DLKELGNYTYYNKWPWYIWLGFIAGLVALALCVFFILCCTGCGTNCMGKLKCNRCC
DRYEEYDLEPHKVHVH
DNA sequence
ATGGACTGGACCTGGATCCTGTTCCTGGTGGCCGCCGCCACCAGGGTGCACAGCCTGAGGTTCGGCATC
GTGGCCAGCAGGGCCAACCACGCCCTGGTGGGCGGCAGCGGCGGCAGCGGCGGCAGCGGCGGCAGCGG
CGGCGGCATGATCCACAGCGTGTTCCTGCTGATGTTCCTGCTGACCCCCACCGAGAGCTACGTGGACGT
GGGCCCCGACAGCGTGAAGAGCGCCTGCATCGAGGTGGACATCCAGCAGACCTTCTTCGACAAGACCTG
GCCCAGGCCCATCGACGTGAGCAAGGCCGACGGCATCATCTACCCCCAGGGCAGGACCTACAGCAACA
TCACCATCACCTACCAGGGCCTGTTCCCCTACCAGGGCGACCACGGCGACATGTACGTGTACAGCGCCG
GCCACGCCACCGGCACCACCCCCCAGAAGCTGTTCGTGGCCAACTACAGCCAGGACGTGAAGCAGTTCG
CCAACGGCTTCGTGGTGAGGATCGGCGCCGCCGCCAACAGCACCGGCACCGTGATCATCAGCCCCAGCA
CCAGCGCCACCATCAGGAAGATCTACCCCGCCTTCATGCTGGGCAGCAGCGTGGGCAACTTCAGCGACG
GCAAGATGGGCAGGTTCTTCAACCACACCCTGGTGCTGCTGCCCGACGGCTGCGGCACCCTGCTGAGGG
CCTTCTACTGCATCCTGGAGCCCAGGAGCGGCAACCACTGCCCCGCCGGCAACAGCTACACCAGCTTCG
CCACCTACCACACCCCCGCCACCGACTGCAGCGACGGCAACTACAACAGGAACGCCAGCCTGAACAGC
TTCAAGGAGTACTTCAACCTGAGGAACTGCACCTTCATGTACACCTACAACATCACCGAGGACGAGATC
CTGGAGTGGTTCGGCATCACCCAGACCGCCCAGGGCGTGCACCTGTTCAGCAGCAGGTACGTGGACCTG
TACGGCGGCAACATGTTCCAGTTCGCCACCCTGCCCGTGTACGACACCATCAAGTACTACAGCATCATC
CCCCACAGCATCAGGAGCATCCAGAGCGACAGGAAGGCCTGGGCCGCCTTCTACGTGTACAAGCTGCA
GCCCCTGACCTTCCTGCTGGACTTCAGCGTGGACGGCTACATCAGGAGGGCCATCGACTGCGGCTTCAA
CGACCTGAGCCAGCTGCACTGCAGCTACGAGAGCTTCGACGTGGAGAGCGGCGTGTACAGCGTGAGCA
GCTTCGAGGCCAAGCCCAGCGGCAGCGTGGTGGAGCAGGCCGAGGGCGTGGAGTGCGACTTCAGCCCC
CTGCTGAGCGGCACCCCCCCCCAGGTGTACAACTTCAAGAGGCTGGTGTTCACCAACTGCAACTACAAC
CTGACCAAGCTGCTGAGCCTGTTCAGCGTGAACGACTTCACCTGCAGCCAGATCAGCCCCGCCGCCATC
GCCAGCAACTGCTACAGCAGCCTGATCCTGGACTACTTCAGCTACCCCCTGAGCATGAAGAGCGACCTG
AGCGTGAGCAGCGCCGGCCCCATCAGCCAGTTCAACTACAAGCAGAGCTTCAGCAACCCCACCTGCCTG
ATCCTGGCCACCGTGCCCCACAACCTGACCACCATCACCAAGCCCCTGAAGTACAGCTACATCAACAAG
TGCAGCAGGTTCCTGAGCGACGACAGGACCGAGGTGCCCCAGCTGGTGAACGCCAACCAGTACAGCCC
CTGCGTGAGCATCGTGCCCAGCACCGTGTGGGAGGACGGCGACTACTACAGGAAGCAGCTGAGCCCCCT
GGAGGGCGGCGGCTGGCTGGTGGCCAGCGGCAGCACCGTGGCCATGACCGAGCAGCTGCAGATGGGCT
TCGGCATCACCGTGCAGTACGGCACCGACACCAACAGCGTGTGCCCCAAGCTGGAGTTCGCCAACGACA
CCAAGATCGCCAGCCAGCTGGGCAACTGCGTGGAGTACAGCCTGTACGGCGTGAGCGGCAGGGGCGTG
TTCCAGAACTGCACCGCCGTGGGCGTGAGGCAGCAGAGGTTCGTGTACGACGCCTACCAGAACCTGGTG
GGCTACTACAGCGACGACGGCAACTACTACTGCCTGAGGGCCTGCGTGAGCGTGCCCGTGAGCGTGATC
TACGACAAGGAGACCAAGACCCACGCCACCCTGTTCGGCAGCGTGGCCTGCGAGCACATCAGCAGCAC
CATGAGCCAGTACAGCAGGAGCACCAGGAGCATGCTGAAGAGGAGGGACAGCACCTACGGCCCCCTGC
AGACCCCCGTGGGCTGCGTGCTGGGCCTGGTGAACAGCAGCCTGTTCGTGGAGGACTGCAAGCTGCCCC
TGGGCCAGAGCCTGTGCGCCCTGCCCGACACCCCCAGCACCCTGACCCCCAGGAGCGTGAGGAGCGTGC
CCGGCGAGATGAGGCTGGCCAGCATCGCCTTCAACCACCCCATCCAGGTGGACCAGCTGAACAGCAGCT
ACTTCAAGCTGAGCATCCCCACCAACTTCAGCTTCGGCGTGACCCAGGAGTACATCCAGACCACCATCC
AGAAGGTGACCGTGGACTGCAAGCAGTACGTGTGCAACGGCTTCCAGAAGTGCGAGCAGCTGCTGAGG
GAGTACGGCCAGTTCTGCAGCAAGATCAACCAGGCCCTGCACGGCGCCAACCTGAGGCAGGACGACAG
CGTGAGGAACCTGTTCGCCAGCGTGAAGAGCAGCCAGAGCAGCCCCATCATCCCCGGCTTCGGCGGCGA
CTTCAACCTGACCCTGCTGGAGCCCGTGAGCATCAGCACCGGCAGCAGGAGCGCCAGGAGCGCCATCG
AGGACCTGCTGTTCGACAAGGTGACCATCGCCGACCCCGGCTACATGCAGGGCTACGACGACTGCATGC
AGCAGGGCCCCGCCAGCGCCAGGGACCTGATCTGCGCCCAGTACGTGGCCGGCTACAAGGTGCTGCCCC
CCCTGATGGACGTGAACATGGAGGCCGCCTACACCAGCAGCCTGCTGGGCAGCATCGCCGGCGTGGGCT
GGACCGCCGGCCTGAGCAGCTTCGCCGCCATCCCCTTCGCCCAGAGCATCTTCTACAGGCTGAACGGCG
TGGGCATCACCCAGCAGGTGCTGAGCGAGAACCAGAAGCTGATCGCCAACAAGTTCAACCAGGCCCTG
GGCGCCATGCAGACCGGCTTCACCACCACCAACGAGGCCTTCCACAAGGTGCAGGACGCCGTGAACAA
CAACGCCCAGGCCCTGAGCAAGCTGGCCAGCGAGCTGAGCAACACCTTCGGCGCCATCAGCGCCAGCA
TCGGCGACATCATCCAGAGGCTGGACGTGCTGGAGCAGGACGCCCAGATCGACAGGCTGATCAACGGC
AGGCTGACCACCCTGAACGCCTTCGTGGCCCAGCAGCTGGTGAGGAGCGAGAGCGCCGCCCTGAGCGC
CCAGCTGGCCAAGGACAAGGTGAACGAGTGCGTGAAGGCCCAGAGCAAGAGGAGCGGCTTCTGCGGCC
AGGGCACCCACATCGTGAGCTTCGTGGTGAACGCCCCCAACGGCCTGTACTTCATGCACGTGGGCTACT
ACCCCAGCAACCACATCGAGGTGGTGAGCGCCTACGGCCTGTGCGACGCCGCCAACCCCACCAACTGCA
TCGCCCCCGTGAACGGCTACTTCATCAAGACCAACAACACCAGGATCGTGGACGAGTGGAGCTACACCG
GCAGCAGCTTCTACGCCCCCGAGCCCATCACCAGCCTGAACACCAAGTACGTGGCCCCCCAGGTGACCT
ACCAGAACATCAGCACCAACCTGCCCCCCCCCCTGCTGGGCAACAGCACCGGCATCGACTTCCAGGACG
AGCTGGACGAGTTCTTCAAGAACGTGAGCACCAGCATCCCCAACTTCGGCAGCCTGACCCAGATCAACA
CCACCCTGCTGGACCTGACCTACGAGATGCTGAGCCTGCAGCAGGTGGTGAAGGCCCTGAACGAGAGCT
ACATCGACCTGAAGGAGCTGGGCAACTACACCTACTACAACAAGTGGCCCTGGTACATCTGGCTGGGCT
TCATCGCCGGCCTGGTGGCCCTGGCCCTGTGCGTGTTCTTCATCCTGTGCTGCACCGGCTGCGGCACCAA
CTGCATGGGCAAGCTGAAGTGCAACAGGTGCTGCGACAGGTACGAGGAGTACGACCTGGAGCCCCACA
AGGTGCACGTGCAC
5. LS3_MERS-COV2 S RBD
MDWTWILFLVAAATRVHSLRFGIVASRANHALVGGSGGSGGSGGSGGGEAKPSGSVVEQ
AEGVECDFSPLLSGTPPQVYNFKRLVFTNCNYNLTKLLSLFSVNDFTCSQISPAAIASN
CYSSLILDYFSYPLSMKSDLSVSSAGPISQFNYKQSFSNPTCLILATVPHNLTTITKPLK
YSYINKCSRFLSDDRTEVPQLVNANQYSPCVSIVPSTVWEDGDYYRKQLSPLEGGGW
LVASGSTVAMTEQLQMGFGITVQYGTDTNSVCPKLEFANDTKIASQLGNCVEY
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCGAGGCCAAGCCCAG
CGGCAGCGTGGTGGAGCAGGCCGAGGGCGTGGAGTGCGACTTCAGCCCCCTGCTGAGCGGCACCCCCCCCCAGGTGTAC
AACTTCAAGAGGCTGGTGTTCACCAACTGCAACTACAACCTGACCAAGCTGCTGAGCCTGTTCAGCGTGAACGACTTCA
CCTGCAGCCAGATCAGCCCCGCCGCCATCGCCAGCAACTGCTACAGCAGCCTGATCCTGGACTACTTCAGCTACCCCCT
GAGCATGAAGAGCGACCTGAGCGTGAGCAGCGCCGGCCCCATCAGCCAGTTCAACTACAAGCAGAGCTTCAGCAACCCC
ACCTGCCTGATCCTGGCCACCGTGCCCCACAACCTGACCACCATCACCAAGCCCCTGAAGTACAGCTACATCAACAAGT
GCAGCAGGTTCCTGAGCGACGACAGGACCGAGGTGCCCCAGCTGGTGAACGCCAACCAGTACAGCCCCTGCGTGAGCAT
CGTGCCCAGCACCGTGTGGGAGGACGGCGACTACTACAGGAAGCAGCTGAGCCCCCTGGAGGGCGGCGGCTGGCTGGTG
GCCAGCGGCAGCACCGTGGCCATGACCGAGCAGCTGCAGATGGGCTTCGGCATCACCGTGCAGTACGGCACCGACACCA
ACAGCGTGTGCCCCAAGCTGGAGTTCGCCAACGACACCAAGATCGCCAGCCAGCTGGGCAACTGCGTGGAGTAC
6. LS3_RSV F
MDWTWILFLVAAATRVHSLRFGIVASRANHALVGGSGGSGGSGGSGGGNITEEFYQSTCS
AVSKGYLSALRTGWYTSVITIELSNIKENKCNGTDAKVKLIKQELDKYKNAVTELQL
LMQSTPPTNNRARRELPRFMNYTLNNAKKTNVTLSKKRKRRFLGFLLGVGSAIASGV
AVSKVLHLEGEVNKIKSALLSTNKAVVSLSNGVSVLTSKVLDLKNYIDKQLLPIVNK
QSCSISNIETVIEFQQKNNRLLEITREFSVNAGVTTPVSTYMLTNSELLSLINDMPITND
QKKLMSNNVQIVRQQSYSIMSIIKEEVLAYVVQLPLYGVIDTPCWKLHTSPLCTTNTK
EGSNICLTRTDRGWYCDNAGSVSFFPQAETCKVQSNRVFCDTMNSLTLPSEINLCNV
DIFNPKYDCKIMTSKTDVSSSVITSLGAIVSCYGKTKCTASNKNRGIIKTFSNGCDYVS
NKGMDTVSVGNTLYYVNKQEGKSLYVKGEPIINFYDPLVFPSDEFDASISQVNEKIN
QSLAFIRKSDELLHNVNAGKSTTNIMITT
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCAACATCACCGAGGA
GTTCTACCAGAGCACCTGCAGCGCCGTGAGCAAGGGCTACCTGAGCGCCCTGAGGACCGGCTGGTACACCAGCGTGATC
ACCATCGAGCTGAGCAACATCAAGGAGAACAAGTGCAACGGCACCGACGCCAAGGTGAAGCTGATCAAGCAGGAGCTGG
ACAAGTACAAGAACGCCGTGACCGAGCTGCAGCTGCTGATGCAGAGCACCCCCCCCACCAACAACAGGGCCAGGAGGGA
GCTGCCCAGGTTCATGAACTACACCCTGAACAACGCCAAGAAGACCAACGTGACCCTGAGCAAGAAGAGGAAGAGGAGG
TTCCTGGGCTTCCTGCTGGGCGTGGGCAGCGCCATCGCCAGCGGCGTGGCCGTGAGCAAGGTGCTGCACCTGGAGGGCG
AGGTGAACAAGATCAAGAGCGCCCTGCTGAGCACCAACAAGGCCGTGGTGAGCCTGAGCAACGGCGTGAGCGTGCTGAC
CAGCAAGGTGCTGGACCTGAAGAACTACATCGACAAGCAGCTGCTGCCCATCGTGAACAAGCAGAGCTGCAGCATCAGC
AACATCGAGACCGTGATCGAGTTCCAGCAGAAGAACAACAGGCTGCTGGAGATCACCAGGGAGTTCAGCGTGAACGCCG
GCGTGACCACCCCCGTGAGCACCTACATGCTGACCAACAGCGAGCTGCTGAGCCTGATCAACGACATGCCCATCACCAA
CGACCAGAAGAAGCTGATGAGCAACAACGTGCAGATCGTGAGGCAGCAGAGCTACAGCATCATGAGCATCATCAAGGAG
GAGGTGCTGGCCTACGTGGTGCAGCTGCCCCTGTACGGCGTGATCGACACCCCCTGCTGGAAGCTGCACACCAGCCCCC
TGTGCACCACCAACACCAAGGAGGGCAGCAACATCTGCCTGACCAGGACCGACAGGGGCTGGTACTGCGACAACGCCGG
CAGCGTGAGCTTCTTCCCCCAGGCCGAGACCTGCAAGGTGCAGAGCAACAGGGTGTTCTGCGACACCATGAACAGCCTG
ACCCTGCCCAGCGAGATCAACCTGTGCAACGTGGACATCTTCAACCCCAAGTACGACTGCAAGATCATGACCAGCAAGA
CCGACGTGAGCAGCAGCGTGATCACCAGCCTGGGCGCCATCGTGAGCTGCTACGGCAAGACCAAGTGCACCGCCAGCAA
CAAGAACAGGGGCATCATCAAGACCTTCAGCAACGGCTGCGACTACGTGAGCAACAAGGGCATGGACACCGTGAGCGTG
GGCAACACCCTGTACTACGTGAACAAGCAGGAGGGCAAGAGCCTGTACGTGAAGGGCGAGCCCATCATCAACTTCTACG
ACCCCCTGGTGTTCCCCAGCGACGAGTTCGACGCCAGCATCAGCCAGGTGAACGAGAAGATCAACCAGAGCCTGGCCTT
CATCAGGAAGAGCGACGAGCTGCTGCACAACGTGAACGCCGGCAAGAGCACCACCAACATCATGATCACCACC
7. LS3_H3-Kansas-2017_HA
MDWTWILFLVAAATRVHSLRFGIVASRANHALVGGSGGSGGSGGSGGGMKTIIALSCILC
LVFAQKIPGNDNSTATLCLGHHAVPNGTIVKTITNDRIEVTNATELVQNSSIGEICDSP
HQILDGENCTLIDALLGDPQCDGFQNKKWDLFVERNKAYSNCYPYDVPDYASLRSL
VASSGTLEFNNESFNWAGVTQNGTSSSCIRGSKSSFFSRLNWLTHLNSKYPALNVTM
PNNEQFDKLYIWGVHHPGTDKNQISLYAQSSGRITVSTKRSQQAVIPNIGSRPRIRDIP
SRISIYWTIVKPGDILLINSTGNLIAPRGYFKIRSGKSSIMRSDAPIGKCKSECITPNGSIP
NDKPFQNVNRITYGACPRYVKQSTLKLATGMRNVPERQTRGIFGAIAGFIENGWEG
MVDGWYGFRHQNSEGRGQAADLKSTQAAIDQINGKLNRLIGKTNEKFHQIEKEFSE
VEGRIQDLEKYVEDTKIDLWSYNAELLVALENQHTIDLTDSEMNKLFEKTKKQLREN
AEDMGNGCFKIYHKCDNACMGSIRNGTYDHNVYRDEALNNRFQIK
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATGAAGACCATCAT
CGCCCTGAGCTGCATCCTGTGCCTGGTGTTCGCCCAGAAGATCCCCGGCAACGACAACAGCACCGCCACCCTGTGCCTG
GGCCACCACGCCGTGCCCAACGGCACCATCGTGAAGACCATCACCAACGACAGGATCGAGGTGACCAACGCCACCGAGC
TGGTGCAGAACAGCAGCATCGGCGAGATCTGCGACAGCCCCCACCAGATCCTGGACGGCGAGAACTGCACCCTGATCGA
CGCCCTGCTGGGCGACCCCCAGTGCGACGGCTTCCAGAACAAGAAGTGGGACCTGTTCGTGGAGAGGAACAAGGCCTAC
AGCAACTGCTACCCCTACGACGTGCCCGACTACGCCAGCCTGAGGAGCCTGGTGGCCAGCAGCGGCACCCTGGAGTTCA
ACAACGAGAGCTTCAACTGGGCCGGCGTGACCCAGAACGGCACCAGCAGCAGCTGCATCAGGGGCAGCAAGAGCAGCTT
CTTCAGCAGGCTGAACTGGCTGACCCACCTGAACAGCAAGTACCCCGCCCTGAACGTGACCATGCCCAACAACGAGCAG
TTCGACAAGCTGTACATCTGGGGCGTGCACCACCCCGGCACCGACAAGAACCAGATCAGCCTGTACGCCCAGAGCAGCG
GCAGGATCACCGTGAGCACCAAGAGGAGCCAGCAGGCCGTGATCCCCAACATCGGCAGCAGGCCCAGGATCAGGGACAT
CCCCAGCAGGATCAGCATCTACTGGACCATCGTGAAGCCCGGCGACATCCTGCTGATCAACAGCACCGGCAACCTGATC
GCCCCCAGGGGCTACTTCAAGATCAGGAGCGGCAAGAGCAGCATCATGAGGAGCGACGCCCCCATCGGCAAGTGCAAGA
GCGAGTGCATCACCCCCAACGGCAGCATCCCCAACGACAAGCCCTTCCAGAACGTGAACAGGATCACCTACGGCGCCTG
CCCCAGGTACGTGAAGCAGAGCACCCTGAAGCTGGCCACCGGCATGAGGAACGTGCCCGAGAGGCAGACCAGGGGCATC
TTCGGCGCCATCGCCGGCTTCATCGAGAACGGCTGGGAGGGCATGGTGGACGGCTGGTACGGCTTCAGGCACCAGAACA
GCGAGGGCAGGGGCCAGGCCGCCGACCTGAAGAGCACCCAGGCCGCCATCGACCAGATCAACGGCAAGCTGAACAGGCT
GATCGGCAAGACCAACGAGAAGTTCCACCAGATCGAGAAGGAGTTCAGCGAGGTGGAGGGCAGGATCCAGGACCTGGAG
AAGTACGTGGAGGACACCAAGATCGACCTGTGGAGCTACAACGCCGAGCTGCTGGTGGCCCTGGAGAACCAGCACACCA
TCGACCTGACCGACAGCGAGATGAACAAGCTGTTCGAGAAGACCAAGAAGCAGCTGAGGGAGAACGCCGAGGACATGGG
CAACGGCTGCTTCAAGATCTACCACAAGTGCGACAACGCCTGCATGGGCAGCATCAGGAACGGCACCTACGACCACAAC
GTGTACAGGGACGAGGCCCTGAACAACAGGTTCCAGATCAAG
8. LS3_H1-Brisbane-2018_HA
MDWTWILFLVAAATRVHSLRFGIVASRANHALVGGSGGSGGSGGSGGGMKAILVVLLYT
FTTANADTLCIGYHANNSTDTVDTVLEKNVTVTHSVNLLEDKHNGKLCKLGGVAPL
HLGKCNIAGWILGNPECESLSTARSWSYIVETSNSDNGTCYPGDFINYEELREQLSSV
SSFERFEIFPKTSSWPNHDSNKGVTAACPHAGAKSFYKNLIWLVKKGNSYPKLNQTY
INDKGKEVLVLWGIHHPPTTADQQSLYQNADAYVFVGTSRYSKKFKPEIATRPKVRD
REGRMNYYWTLVEPGDKITFEATGNLVVPRYAFTMERNAGSGIIISDTPVHDCNTTC
QTAEGAINTSLPFQNVHPVTIGKCPKYVKSTKLRLATGLRNVPSIQSRGLFGAIAGFIE
GGWTGMVDGWYGYHHQNEQGSGYAADLKSTQNAIDKITNKVNSVIEKMNTQFTA
VGKEFNHLEKRIENLNKKVDDGFLDIWTYNAELLVLLENERTLDYHDSNVKNLYEK
VRNQLKNNAKEIGNGCFEFYHKCDNTCMESVKNGTYDYPKYSEEAKLNREKID
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATGAAGGCCATCCT
GGTGGTGCTGCTGTACACCTTCACCACCGCCAACGCCGACACCCTGTGCATCGGCTACCACGCCAACAACAGCACCGAC
ACCGTGGACACCGTGCTGGAGAAGAACGTGACCGTGACCCACAGCGTGAACCTGCTGGAGGACAAGCACAACGGCAAGC
TGTGCAAGCTGGGCGGCGTGGCCCCCCTGCACCTGGGCAAGTGCAACATCGCCGGCTGGATCCTGGGCAACCCCGAGTG
CGAGAGCCTGAGCACCGCCAGGAGCTGGAGCTACATCGTGGAGACCAGCAACAGCGACAACGGCACCTGCTACCCCGGC
GACTTCATCAACTACGAGGAGCTGAGGGAGCAGCTGAGCAGCGTGAGCAGCTTCGAGAGGTTCGAGATCTTCCCCAAGA
CCAGCAGCTGGCCCAACCACGACAGCAACAAGGGCGTGACCGCCGCCTGCCCCCACGCCGGCGCCAAGAGCTTCTACAA
GAACCTGATCTGGCTGGTGAAGAAGGGCAACAGCTACCCCAAGCTGAACCAGACCTACATCAACGACAAGGGCAAGGAG
GTGCTGGTGCTGTGGGGCATCCACCACCCCCCCACCACCGCCGACCAGCAGAGCCTGTACCAGAACGCCGACGCCTACG
TGTTCGTGGGCACCAGCAGGTACAGCAAGAAGTTCAAGCCCGAGATCGCCACCAGGCCCAAGGTGAGGGACAGGGAGGG
CAGGATGAACTACTACTGGACCCTGGTGGAGCCCGGCGACAAGATCACCTTCGAGGCCACCGGCAACCTGGTGGTGCCC
AGGTACGCCTTCACCATGGAGAGGAACGCCGGCAGCGGCATCATCATCAGCGACACCCCCGTGCACGACTGCAACACCA
CCTGCCAGACCGCCGAGGGCGCCATCAACACCAGCCTGCCCTTCCAGAACGTGCACCCCGTGACCATCGGCAAGTGCCC
CAAGTACGTGAAGAGCACCAAGCTGAGGCTGGCCACCGGCCTGAGGAACGTGCCCAGCATCCAGAGCAGGGGCCTGTTC
GGCGCCATCGCCGGCTTCATCGAGGGCGGCTGGACCGGCATGGTGGACGGCTGGTACGGCTACCACCACCAGAACGAGC
AGGGCAGCGGCTACGCCGCCGACCTGAAGAGCACCCAGAACGCCATCGACAAGATCACCAACAAGGTGAACAGCGTGAT
CGAGAAGATGAACACCCAGTTCACCGCCGTGGGCAAGGAGTTCAACCACCTGGAGAAGAGGATCGAGAACCTGAACAAG
AAGGTGGACGACGGCTTCCTGGACATCTGGACCTACAACGCCGAGCTGCTGGTGCTGCTGGAGAACGAGAGGACCCTGG
ACTACCACGACAGCAACGTGAAGAACCTGTACGAGAAGGTGAGGAACCAGCTGAAGAACAACGCCAAGGAGATCGGCAA
CGGCTGCTTCGAGTTCTACCACAAGTGCGACAACACCTGCATGGAGAGCGTGAAGAACGGCACCTACGACTACCCCAAG
TACAGCGAGGAGGCCAAGCTGAACAGGGAGAAGATCGAC
9. LS3_B-Colorado-2017_HA
MDWTWILFLVAAATRVHSLRFGIVASRANHALVGGSGGSGGSGGSGGGMKAIIVLLMVV
TSSADRICTGITSSNSPHVVKTATQGEVNVTGVIPLTTTPTKSHFANLKGTETRGKLCP
KCLNCTDLDVALGRPKCTGKIPSARVSILHEVRPVTSGCFPIMHDRTKIRQLPNLLRG
YEHVRLSTHNVINAEGAPGGPYKIGTSGSCPNITNGNGFFATMAWAVPDKNKTATNP
LTIEVPYVCTEGEDQITVWGFHSDTETQMAKLYGDSKPQKFTSSANGVTTHYVSQIG
GFPNQTEDGGLPQSGRIVVDYMVQKSGKTGTITYQRGILLPQKVWCASGRSKVIKGS
LPLIGEADCLHEKYGGLNKSKPYYTGEHAKAIGNCPIWVKTPLKLANGTKYRPPAKL
LKERGFFGAIAGFLEGGWEGMIAGWHGYTSHGAHGVAVAADLKSTQEAINKITKNL
NSLSELEVKNLQRLSGAMDELHNEILELDEKVDDLRADTISSQIELAVLLSNEGIINSE
DEHLLALERKLKKMLGPSAVEIGNGCFETKHKCNQTCLDKIAAGTFDAGEFSLPTFD
SLNIT
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATGAAGGCCATCAT
CGTGCTGCTGATGGTGGTGACCAGCAGCGCCGACAGGATCTGCACCGGCATCACCAGCAGCAACAGCCCCCACGTGGTG
AAGACCGCCACCCAGGGCGAGGTGAACGTGACCGGCGTGATCCCCCTGACCACCACCCCCACCAAGAGCCACTTCGCCA
ACCTGAAGGGCACCGAGACCAGGGGCAAGCTGTGCCCCAAGTGCCTGAACTGCACCGACCTGGACGTGGCCCTGGGCAG
GCCCAAGTGCACCGGCAAGATCCCCAGCGCCAGGGTGAGCATCCTGCACGAGGTGAGGCCCGTGACCAGCGGCTGCTTC
CCCATCATGCACGACAGGACCAAGATCAGGCAGCTGCCCAACCTGCTGAGGGGCTACGAGCACGTGAGGCTGAGCACCC
ACAACGTGATCAACGCCGAGGGCGCCCCCGGCGGCCCCTACAAGATCGGCACCAGCGGCAGCTGCCCCAACATCACCAA
CGGCAACGGCTTCTTCGCCACCATGGCCTGGGCCGTGCCCGACAAGAACAAGACCGCCACCAACCCCCTGACCATCGAG
GTGCCCTACGTGTGCACCGAGGGCGAGGACCAGATCACCGTGTGGGGCTTCCACAGCGACACCGAGACCCAGATGGCCA
AGCTGTACGGCGACAGCAAGCCCCAGAAGTTCACCAGCAGCGCCAACGGCGTGACCACCCACTACGTGAGCCAGATCGG
CGGCTTCCCCAACCAGACCGAGGACGGCGGCCTGCCCCAGAGCGGCAGGATCGTGGTGGACTACATGGTGCAGAAGAGC
GGCAAGACCGGCACCATCACCTACCAGAGGGGCATCCTGCTGCCCCAGAAGGTGTGGTGCGCCAGCGGCAGGAGCAAGG
TGATCAAGGGCAGCCTGCCCCTGATCGGCGAGGCCGACTGCCTGCACGAGAAGTACGGCGGCCTGAACAAGAGCAAGCC
CTACTACACCGGCGAGCACGCCAAGGCCATCGGCAACTGCCCCATCTGGGTGAAGACCCCCCTGAAGCTGGCCAACGGC
ACCAAGTACAGGCCCCCCGCCAAGCTGCTGAAGGAGAGGGGCTTCTTCGGCGCCATCGCCGGCTTCCTGGAGGGCGGCT
GGGAGGGCATGATCGCCGGCTGGCACGGCTACACCAGCCACGGCGCCCACGGCGTGGCCGTGGCCGCCGACCTGAAGAG
CACCCAGGAGGCCATCAACAAGATCACCAAGAACCTGAACAGCCTGAGCGAGCTGGAGGTGAAGAACCTGCAGAGGCTG
AGCGGCGCCATGGACGAGCTGCACAACGAGATCCTGGAGCTGGACGAGAAGGTGGACGACCTGAGGGCCGACACCATCA
GCAGCCAGATCGAGCTGGCCGTGCTGCTGAGCAACGAGGGCATCATCAACAGCGAGGACGAGCACCTGCTGGCCCTGGA
GAGGAAGCTGAAGAAGATGCTGGGCCCCAGCGCCGTGGAGATCGGCAACGGCTGCTTCGAGACCAAGCACAAGTGCAAC
CAGACCTGCCTGGACAAGATCGCCGCCGGCACCTTCGACGCCGGCGAGTTCAGCCTGCCCACCTTCGACAGCCTGAACA
TCACC
10. LS3_B-Phuket-2013_HA
MDWTWILFLVAAATRVHSLRFGIVASRANHALVGGSGGSGGSGGSGGGMKAIIVLLMVV
TSNADRICTGITSSNSPHVVKTATQGEVNVTGVIPLTTTPTKSYFANLKGTRTRGKLC
PDCLNCTDLDVALGRPMCVGTTPSAKASILHEVRPVTSGCFPIMHDRTKIRQLPNLLR
GYEKIRLSTQNVIDAEKAPGGPYRLGTSGSCPNATSKIGFFATMAWAVPKDNYKNAT
NPLTVEVPYICTEGEDQITVWGFHSDDKTQMKSLYGDSNPQKFTSSANGVTTHYVSQ
IGDFPDQTEDGGLPQSGRIVVDYMMQKPGKTGTIVYQRGVLLPQKVWCASGRSKVI
KGSLPLIGEADCLHEEYGGLNKSKPYYTGKHAKAIGNCPIWVKTPLKLANGTKYRPP
AKLLKERGFFGAIAGFLEGGWEGMIAGWHGYTSHGAHGVAVAADLKSTQEAINKIT
KNLNSLSELEVKNLQRLSGAMDELHNEILELDEKVDDLRADTISSQIELAVLLSNEGII
NSEDEHLLALERKLKKMLGPSAVDIGNGCFETKHKCNQTCLDRIAAGTFNAGEFSLP
TFDSLNIT
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATGAAGGCCATCAT
CGTGCTGCTGATGGTGGTGACCAGCAACGCCGACAGGATCTGCACCGGCATCACCAGCAGCAACAGCCCCCACGTGGTG
AAGACCGCCACCCAGGGCGAGGTGAACGTGACCGGCGTGATCCCCCTGACCACCACCCCCACCAAGAGCTACTTCGCCA
ACCTGAAGGGCACCAGGACCAGGGGCAAGCTGTGCCCCGACTGCCTGAACTGCACCGACCTGGACGTGGCCCTGGGCAG
GCCCATGTGCGTGGGCACCACCCCCAGCGCCAAGGCCAGCATCCTGCACGAGGTGAGGCCCGTGACCAGCGGCTGCTTC
CCCATCATGCACGACAGGACCAAGATCAGGCAGCTGCCCAACCTGCTGAGGGGCTACGAGAAGATCAGGCTGAGCACCC
AGAACGTGATCGACGCCGAGAAGGCCCCCGGCGGCCCCTACAGGCTGGGCACCAGCGGCAGCTGCCCCAACGCCACCAG
CAAGATCGGCTTCTTCGCCACCATGGCCTGGGCCGTGCCCAAGGACAACTACAAGAACGCCACCAACCCCCTGACCGTG
GAGGTGCCCTACATCTGCACCGAGGGCGAGGACCAGATCACCGTGTGGGGCTTCCACAGCGACGACAAGACCCAGATGA
AGAGCCTGTACGGCGACAGCAACCCCCAGAAGTTCACCAGCAGCGCCAACGGCGTGACCACCCACTACGTGAGCCAGAT
CGGCGACTTCCCCGACCAGACCGAGGACGGCGGCCTGCCCCAGAGCGGCAGGATCGTGGTGGACTACATGATGCAGAAG
CCCGGCAAGACCGGCACCATCGTGTACCAGAGGGGCGTGCTGCTGCCCCAGAAGGTGTGGTGCGCCAGCGGCAGGAGCA
AGGTGATCAAGGGCAGCCTGCCCCTGATCGGCGAGGCCGACTGCCTGCACGAGGAGTACGGCGGCCTGAACAAGAGCAA
GCCCTACTACACCGGCAAGCACGCCAAGGCCATCGGCAACTGCCCCATCTGGGTGAAGACCCCCCTGAAGCTGGCCAAC
GGCACCAAGTACAGGCCCCCCGCCAAGCTGCTGAAGGAGAGGGGCTTCTTCGGCGCCATCGCCGGCTTCCTGGAGGGCG
GCTGGGAGGGCATGATCGCCGGCTGGCACGGCTACACCAGCCACGGCGCCCACGGCGTGGCCGTGGCCGCCGACCTGAA
GAGCACCCAGGAGGCCATCAACAAGATCACCAAGAACCTGAACAGCCTGAGCGAGCTGGAGGTGAAGAACCTGCAGAGG
CTGAGCGGCGCCATGGACGAGCTGCACAACGAGATCCTGGAGCTGGACGAGAAGGTGGACGACCTGAGGGCCGACACCA
TCAGCAGCCAGATCGAGCTGGCCGTGCTGCTGAGCAACGAGGGCATCATCAACAGCGAGGACGAGCACCTGCTGGCCCT
GGAGAGGAAGCTGAAGAAGATGCTGGGCCCCAGCGCCGTGGACATCGGCAACGGCTGCTTCGAGACCAAGCACAAGTGC
AACCAGACCTGCCTGGACAGGATCGCCGCCGGCACCTTCAACGCCGGCGAGTTCAGCCTGCCCACCTTCGACAGCCTGA
ACATCACC
11. LS3_CA09_RBD_HA
MDWTWILFLVAAATRVHSLRFGIVASRANHALVGGSGGSGGSGGSGGGAPLHLGKCNIAGW
ILGNPECESLSTASSWSYIVETPSSDNGTCEPGDFIDYEELREQLSSVSSFERFEIFPKTSSWPNHDSNKGVT
AACPHAGAKSFYKNLIWLVKKGNSYPKLSKSYINDKGKEVLVLWGIHHPSTSADQQSLYQNADTYVFVGS
SRYSKKFKPEIAIRPKVRDQEGRMNYYWTLVEPGDKITFEATGNLVVPRYAFAMERN
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCGCCCCCCTGCACCT
GGGCAAGTGCAACATCGCCGGCTGGATCCTGGGCAACCCCGAGTGCGAGAGCCTGAGCACCGCCAGCAGCTGGAGCTAC
ATCGTGGAGACCCCCAGCAGCGACAACGGCACCTGCTTCCCCGGCGACTTCATCGACTACGAGGAGCTGAGGGAGCAGC
TGAGCAGCGTGAGCAGCTTCGAGAGGTTCGAGATCTTCCCCAAGACCAGCAGCTGGCCCAACCACGACAGCAACAAGGG
CGTGACCGCCGCCTGCCCCCACGCCGGCGCCAAGAGCTTCTACAAGAACCTGATCTGGCTGGTGAAGAAGGGCAACAGC
TACCCCAAGCTGAGCAAGAGCTACATCAACGACAAGGGCAAGGAGGTGCTGGTGCTGTGGGGCATCCACCACCCCAGCA
CCAGCGCCGACCAGCAGAGCCTGTACCAGAACGCCGACACCTACGTGTTCGTGGGCAGCAGCAGGTACAGCAAGAAGTT
CAAGCCCGAGATCGCCATCAGGCCCAAGGTGAGGGACCAGGAGGGCAGGATGAACTACTACTGGACCCTGGTGGAGCCC
GGCGACAAGATCACCTTCGAGGCCACCGGCAACCTGGTGGTGCCCAGGTACGCCTTCGCCATGGAGAGGAAC
12. LS3_MD39
MDWTWILFLVAAATRVHSLRFGIVASRANHALVGGSGGSGGSGGSGGGAE
NLWVTVYYGV PVWKDAETTL FCASDAKAYE TEKHNVWATH ACVPTDPNPQ
EIHLENVTEE FNMWKNNMVE QMHEDIISLW DQSLKPCVKL TPLCVTLQCT
NVTNNITDDM RGELKNCSFN MTTELRDKKQ KVYSLFYRLD VVQINENQGN
RSNNSNKEYR LINCNTSAIT QACPKVSFEP IPIHYCAPAG FAILKCKDKK
FNGTGPCPSV STVQCTHGIK PVVSTQLLLN GSLAEEEVII RSENITNNAK
NILVQLNTPV QINCTRPNNN TVKSIRIGPG QAFYYTGDII GDIRQAHCNV
SKATWNETLG KVVKQLRKHF GNNTIIRFAQ SSGGDLEVTT HSFNCGGEFF
YCNTSGLFNS TWISNTSVQG SNSTGSNDSI TLPCRIKQII NMWQRIGQAM
YAPPIQGVIR CVSNITGLIL TRDGGSTNST TETFRPGGGD MRDNWRSELY
KYKVVKIEPL GVAPTRCKRR VVGRRRRRRA VGIGAVSLGF LGAAGSTMGA
ASMTLTVQAR NLLSGIVQQQ SNLLRAPEPQ QHLLKDTHWG IKQLQARVLA
VEHYLRDQQL LGIWGCSGKL ICCTNVPWNS SWSNRNLSEI WDNMTWLQWD
KEISNYTQII YGLLEESQNQ QEKNEQDLLA LD
DNA Sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCGCCGAGAACCTGTG
GGTGACCGTGTACTACGGCGTGCCCGTGTGGAAGGACGCCGAGACCACCCTGTTCTGCGCCAGCGACGCCAAGGCCTAC
GAGACCGAGAAGCACAACGTGTGGGCCACCCACGCCTGCGTGCCCACCGACCCCAACCCCCAGGAGATCCACCTGGAGA
ACGTGACCGAGGAGTTCAACATGTGGAAGAACAACATGGTGGAGCAGATGCACGAGGACATCATCAGCCTGTGGGACCA
GAGCCTGAAGCCCTGCGTGAAGCTGACCCCCCTGTGCGTGACCCTGCAGTGCACCAACGTGACCAACAACATCACCGAC
GACATGAGGGGCGAGCTGAAGAACTGCAGCTTCAACATGACCACCGAGCTGAGGGACAAGAAGCAGAAGGTGTACAGCC
TGTTCTACAGGCTGGACGTGGTGCAGATCAACGAGAACCAGGGCAACAGGAGCAACAACAGCAACAAGGAGTACAGGCT
GATCAACTGCAACACCAGCGCCATCACCCAGGCCTGCCCCAAGGTGAGCTTCGAGCCCATCCCCATCCACTACTGCGCC
CCCGCCGGCTTCGCCATCCTGAAGTGCAAGGACAAGAAGTTCAACGGCACCGGCCCCTGCCCCAGCGTGAGCACCGTGC
AGTGCACCCACGGCATCAAGCCCGTGGTGAGCACCCAGCTGCTGCTGAACGGCAGCCTGGCCGAGGAGGAGGTGATCAT
CAGGAGCGAGAACATCACCAACAACGCCAAGAACATCCTGGTGCAGCTGAACACCCCCGTGCAGATCAACTGCACCAGG
CCCAACAACAACACCGTGAAGAGCATCAGGATCGGCCCCGGCCAGGCCTTCTACTACACCGGCGACATCATCGGCGACA
TCAGGCAGGCCCACTGCAACGTGAGCAAGGCCACCTGGAACGAGACCCTGGGCAAGGTGGTGAAGCAGCTGAGGAAGCA
CTTCGGCAACAACACCATCATCAGGTTCGCCCAGAGCAGCGGCGGCGACCTGGAGGTGACCACCCACAGCTTCAACTGC
GGCGGCGAGTTCTTCTACTGCAACACCAGCGGCCTGTTCAACAGCACCTGGATCAGCAACACCAGCGTGCAGGGCAGCA
ACAGCACCGGCAGCAACGACAGCATCACCCTGCCCTGCAGGATCAAGCAGATCATCAACATGTGGCAGAGGATCGGCCA
GGCCATGTACGCCCCCCCCATCCAGGGCGTGATCAGGTGCGTGAGCAACATCACCGGCCTGATCCTGACCAGGGACGGC
GGCAGCACCAACAGCACCACCGAGACCTTCAGGCCCGGCGGCGGCGACATGAGGGACAACTGGAGGAGCGAGCTGTACA
AGTACAAGGTGGTGAAGATCGAGCCCCTGGGCGTGGCCCCCACCAGGTGCAAGAGGAGGGTGGTGGGCAGGAGGAGGAG
GAGGAGGGCCGTGGGCATCGGCGCCGTGAGCCTGGGCTTCCTGGGCGCCGCCGGCAGCACCATGGGCGCCGCCAGCATG
ACCCTGACCGTGCAGGCCAGGAACCTGCTGAGCGGCATCGTGCAGCAGCAGAGCAACCTGCTGAGGGCCCCCGAGCCCC
AGCAGCACCTGCTGAAGGACACCCACTGGGGCATCAAGCAGCTGCAGGCCAGGGTGCTGGCCGTGGAGCACTACCTGAG
GGACCAGCAGCTGCTGGGCATCTGGGGCTGCAGCGGCAAGCTGATCTGCTGCACCAACGTGCCCTGGAACAGCAGCTGG
AGCAACAGGAACCTGAGCGAGATCTGGGACAACATGACCTGGCTGCAGTGGGACAAGGAGATCAGCAACTACACCCAGA
TCATCTACGGCCTGCTGGAGGAGAGCCAGAACCAGCAGGAGAAGAACGAGCAGGACCTGCTGGCCCTGGAC
13. F_LS3_E2p_pVax
MDWTWILFLVAAATRVHSMGQIVTFFQEVPHVIEEVMNIVLIALSVLAVLKGLYNFATCGLVGLV
TFLLLCGRSCTTSLYKGVYELQTLELNMETLNMTMPLSCTKNNSHHYIMVGNETGLELTLTNT
SIINHKFCNLSDAHKKNLYDHALMSIISTFHLSIPNFNQYEAMSCDENGGKISVQYNLSHSYAGD
AANHCGTVANGVLOTFMRMAWGGSYIALDSGRGNWDCIMTSYQYLIIQNTTWEDHCQFSRP
SPIGYLGLLSORTRDIYISRRLLGTFTWTLSDSEGKDTPGGYCLTRWMLIEAELKCFGNTAVAK
CNEKHDEEFCDMLRLFDFNKQAIQRLKAEAQMSIQLINKAVNALINDQLIMKNHLRDIMGIPY
CNYSKYWYLNHTTTGRTSLPKCWLVSNGSYLNETHFSDDIEQQADNMITEMLOKEYMENQS
GGSGGLRFGIVASRANHALVGGSGGAAAKPATTEGEFPETREKMSGIRRAIAKAMVHSKHTAP
HVTLMDEADVTKLVAHRKKFKAIAAEKGIKLTFLPYVVKALVSALREYPVLNTSIDDETEEIIQ
KHYYNIGIAADTDRGLLVPVIKHADRKPIFALAQEINELAEKARDGKLTPGEMKGASCTITNIGS
AGGQWFTPVINHPEVAILGIGRIAEKPIVRDGEIVAAPMLALSLSFDHRMIDGATAQKALNHIK
RLLSDPELLLM**
DNA sequence
ATGGACTGGACCTGGATCCTGTTCCTGGTGGCCGCCGCCACCAGGGTGCACAGCATGGGCCAGATCGTGACCTTCTTCC
AGGAGGTGCCCCACGTGATCGAGGAGGTGATGAACATCGTGCTGATCGCCCTGAGCGTGCTGGCCGTGCTGAAGGGCCT
GTACAACTTCGCCACCTGCGGCCTGGTGGGCCTGGTGACCTTCCTGCTGCTGTGCGGCAGGAGCTGCACCACCAGCCTG
TACAAGGGCGTGTACGAGCTGCAGACCCTGGAGCTGAACATGGAGACCCTGAACATGACCATGCCCCTGAGCTGCACCA
AGAACAACAGCCACCACTACATCATGGTGGGCAACGAGACCGGCCTGGAGCTGACCCTGACCAACACCAGCATCATCAA
CCACAAGTTCTGCAACCTGAGCGACGCCCACAAGAAGAACCTGTACGACCACGCCCTGATGAGCATCATCAGCACCTTC
CACCTGAGCATCCCCAACTTCAACCAGTACGAGGCCATGAGCTGCGACTTCAACGGCGGCAAGATCAGCGTGCAGTACA
ACCTGAGCCACAGCTACGCCGGCGACGCCGCCAACCACTGCGGCACCGTGGCCAACGGCGTGCTGCAGACCTTCATGAG
GATGGCCTGGGGCGGCAGCTACATCGCCCTGGACAGCGGCAGGGGCAACTGGGACTGCATCATGACCAGCTACCAGTAC
CTGATCATCCAGAACACCACCTGGGAGGACCACTGCCAGTTCAGCAGGCCCAGCCCCATCGGCTACCTGGGCCTGCTGA
GCCAGAGGACCAGGGACATCTACATCAGCAGGAGGCTGCTGGGCACCTTCACCTGGACCCTGAGCGACAGCGAGGGCAA
GGACACCCCCGGCGGCTACTGCCTGACCAGGTGGATGCTGATCGAGGCCGAGCTGAAGTGCTTCGGCAACACCGCCGTG
GCCAAGTGCAACGAGAAGCACGACGAGGAGTTCTGCGACATGCTGAGGCTGTTCGACTTCAACAAGCAGGCCATCCAGA
GGCTGAAGGCCGAGGCCCAGATGAGCATCCAGCTGATCAACAAGGCCGTGAACGCCCTGATCAACGACCAGCTGATCAT
GAAGAACCACCTGAGGGACATCATGGGCATCCCCTACTGCAACTACAGCAAGTACTGGTACCTGAACCACACCACCACC
GGCAGGACCAGCCTGCCCAAGTGCTGGCTGGTGAGCAACGGCAGCTACCTGAACGAGACCCACTTCAGCGACGACATCG
AGCAGCAGGCCGACAACATGATCACCGAGATGCTGCAGAAGGAGTACATGGAGAACCAGAGCGGCGGCAGCGGCGGCCT
GAGGTTCGGCATCGTGGCCAGCAGGGCCAACCACGCCCTGGTGGGCGGCAGCGGCGGCGCCGCCGCCAAGCCCGCCACC
ACCGAGGGCGAGTTCCCCGAGACCAGGGAGAAGATGAGCGGCATCAGGAGGGCCATCGCCAAGGCCATGGTGCACAGCA
AGCACACCGCCCCCCACGTGACCCTGATGGACGAGGCCGACGTGACCAAGCTGGTGGCCCACAGGAAGAAGTTCAAGGC
CATCGCCGCCGAGAAGGGCATCAAGCTGACCTTCCTGCCCTACGTGGTGAAGGCCCTGGTGAGCGCCCTGAGGGAGTAC
CCCGTGCTGAACACCAGCATCGACGACGAGACCGAGGAGATCATCCAGAAGCACTACTACAACATCGGCATCGCCGCCG
ACACCGACAGGGGCCTGCTGGTGCCCGTGATCAAGCACGCCGACAGGAAGCCCATCTTCGCCCTGGCCCAGGAGATCAA
CGAGCTGGCCGAGAAGGCCAGGGACGGCAAGCTGACCCCCGGCGAGATGAAGGGCGCCAGCTGCACCATCACCAACATC
GGCAGCGCCGGCGGCCAGTGGTTCACCCCCGTGATCAACCACCCCGAGGTGGCCATCCTGGGCATCGGCAGGATCGCCG
AGAAGCCCATCGTGAGGGACGGCGAGATCGTGGCCGCCCCCATGCTGGCCCTGAGCCTGAGCTTCGACCACAGGATGAT
CGACGGCGCCACCGCCCAGAAGGCCCTGAACCACATCAAGAGGCTGCTGAGCGACCCCGAGCTGCTGCTGATG
Underlined: antigen sequence
E2P nanoparticle scaffold plus contiguous cancer antigens
Cancer constructs below:
14. LS3_Gp100
MDWTWILFLVAAATRVHSLRFGIVASRANHALVGGSGGSGGSGGSGGGMDLVLKRCLL
HLAVIGALLAVGATKVPRNQDWLGVSRQLRTKAWNRQLYPEWTEAQRLDCWRGG
QVSLKVSNDGPTLIGANASFSIALNFPGSQKVLPDGQVIWVNNTIINGSQVWGGQPV
YPQETDDACIFPDGGPCPSGSWSQKRSFVYVWKTWGQYWQVLGGPVSGLSIGTGRA
MLGTHTMEVTVYHRRGSRSYVPLAHSSSAFTITDQVPFSVSVSQLRALDGGNKHFLR
NQPLTFALQLHDPSGYLAEADLSYTWDFGDSSGTLISRALVVTHTYLEPGPVTAQVV
LQAAIPLTSCGSSPVPGTTDGHRPTAEAPNTTAGQVPTTEVVGTTPGQAPTAEPSGTT
SVQVPTTEVISTAPVQMPTAESTGMTPEKVPVSEVMGTTLAEMSTPEATGMTPAEVS
IVVLSGTTAAQVTTTEWVETTARELPIPEPEGPDASSIMSTESITGSLGPLLDGTATLRL
VKR
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATGGACCTGGTGCT
GAAGAGGTGCCTGCTGCACCTGGCCGTGATCGGCGCCCTGCTGGCCGTGGGCGCCACCAAGGTGCCCAGGAACCAGGAC
TGGCTGGGCGTGAGCAGGCAGCTGAGGACCAAGGCCTGGAACAGGCAGCTGTACCCCGAGTGGACCGAGGCCCAGAGGC
TGGACTGCTGGAGGGGCGGCCAGGTGAGCCTGAAGGTGAGCAACGACGGCCCCACCCTGATCGGCGCCAACGCCAGCTT
CAGCATCGCCCTGAACTTCCCCGGCAGCCAGAAGGTGCTGCCCGACGGCCAGGTGATCTGGGTGAACAACACCATCATC
AACGGCAGCCAGGTGTGGGGCGGCCAGCCCGTGTACCCCCAGGAGACCGACGACGCCTGCATCTTCCCCGACGGCGGCC
CCTGCCCCAGCGGCAGCTGGAGCCAGAAGAGGAGCTTCGTGTACGTGTGGAAGACCTGGGGCCAGTACTGGCAGGTGCT
GGGCGGCCCCGTGAGCGGCCTGAGCATCGGCACCGGCAGGGCCATGCTGGGCACCCACACCATGGAGGTGACCGTGTAC
CACAGGAGGGGCAGCAGGAGCTACGTGCCCCTGGCCCACAGCAGCAGCGCCTTCACCATCACCGACCAGGTGCCCTTCA
GCGTGAGCGTGAGCCAGCTGAGGGCCCTGGACGGCGGCAACAAGCACTTCCTGAGGAACCAGCCCCTGACCTTCGCCCT
GCAGCTGCACGACCCCAGCGGCTACCTGGCCGAGGCCGACCTGAGCTACACCTGGGACTTCGGCGACAGCAGCGGCACC
CTGATCAGCAGGGCCCTGGTGGTGACCCACACCTACCTGGAGCCCGGCCCCGTGACCGCCCAGGTGGTGCTGCAGGCCG
CCATCCCCCTGACCAGCTGCGGCAGCAGCCCCGTGCCCGGCACCACCGACGGCCACAGGCCCACCGCCGAGGCCCCCAA
CACCACCGCCGGCCAGGTGCCCACCACCGAGGTGGTGGGCACCACCCCCGGCCAGGCCCCCACCGCCGAGCCCAGCGGC
ACCACCAGCGTGCAGGTGCCCACCACCGAGGTGATCAGCACCGCCCCCGTGCAGATGCCCACCGCCGAGAGCACCGGCA
TGACCCCCGAGAAGGTGCCCGTGAGCGAGGTGATGGGCACCACCCTGGCCGAGATGAGCACCCCCGAGGCCACCGGCAT
GACCCCCGCCGAGGTGAGCATCGTGGTGCTGAGCGGCACCACCGCCGCCCAGGTGACCACCACCGAGTGGGTGGAGACC
ACCGCCAGGGAGCTGCCCATCCCCGAGCCCGAGGGCCCCGACGCCAGCAGCATCATGAGCACCGAGAGCATCACCGGCA
GCCTGGGCCCCCTGCTGGACGGCACCGCCACCCTGAGGCTGGTGAAGAGG
15. LS3_PSA
MDWTWILFLVAAATRVHSLRFGIVASRANHALVGGSGGSGGSGGSGGGIVGGWECEKHS
QPWQVLVASRGRAVCGGVLVHPQWVLTAAHCIRNKSVILLGRHSLFHPEDTGQVFQ
VSHSFPHPLYDMSLLKNRFLRPGDDSSHDLMLLRLSEPAELTDAVKVMDLPTQEPAL
GTTCYASGWGSIEPEEFLTPKKLQCVDLHVISNDVCAQVHPQKVTKFMLCAGRWTG
GKSTCSGDSGGPLVCNGVLQGITSWGSEPCALPERPSLYTKVVHYRKWIKDTIVANP
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCATCGTGGGCGGCTG
GGAGTGCGAGAAGCACAGCCAGCCCTGGCAGGTGCTGGTGGCCAGCAGGGGCAGGGCCGTGTGCGGCGGCGTGCTGGTG
CACCCCCAGTGGGTGCTGACCGCCGCCCACTGCATCAGGAACAAGAGCGTGATCCTGCTGGGCAGGCACAGCCTGTTCC
ACCCCGAGGACACCGGCCAGGTGTTCCAGGTGAGCCACAGCTTCCCCCACCCCCTGTACGACATGAGCCTGCTGAAGAA
CAGGTTCCTGAGGCCCGGCGACGACAGCAGCCACGACCTGATGCTGCTGAGGCTGAGCGAGCCCGCCGAGCTGACCGAC
GCCGTGAAGGTGATGGACCTGCCCACCCAGGAGCCCGCCCTGGGCACCACCTGCTACGCCAGCGGCTGGGGCAGCATCG
AGCCCGAGGAGTTCCTGACCCCCAAGAAGCTGCAGTGCGTGGACCTGCACGTGATCAGCAACGACGTGTGCGCCCAGGT
GCACCCCCAGAAGGTGACCAAGTTCATGCTGTGCGCCGGCAGGTGGACCGGCGGCAAGAGCACCTGCAGCGGCGACAGC
GGCGGCCCCCTGGTGTGCAACGGCGTGCTGCAGGGCATCACCAGCTGGGGCAGCGAGCCCTGCGCCCTGCCCGAGAGGC
CCAGCCTGTACACCAAGGTGGTGCACTACAGGAAGTGGATCAAGGACACCATCGTGGCCAACCCC
16. LS3_HER2
MDWTWILFLVAAATRVHSLRFGIVASRANHALVGGSGGSGGSGGSGGGTQVCTGTDMK
LRLPASPETHLDMLRHLYQGCQVVQGNLELTYLPTNASLSFLQDIQEVQGYVLIAHN
QVRQVPLQRLRIVRGTQLFEDNYALAVLDNGDPLNNTTPVTGASPGGLRELQLRSLT
EILKGGVLIQRNPQLCYQDTILWKDIFHKNNQLALTLIDTNRSRACHPCSPMCKGSRC
WGESSEDCQSLTRTVCAGGCARCKGPLPTDCCHEQCAAGCTGPKHSDCLACLHFNH
SGICELHCPALVTYNTDTFESMPNPEGRYTFGASCVTACPYNYLSTDVGSCTLVCPLH
NQEVTAEDGTQRCEKCSKPCARVCYGLGMEHLREVRAVTSANIQEFAGCKKIFGSL
AFLPESFDGDPASNTAPLQPEQLQVFETLEEITGYLYISAWPDSLPDLSVFQNLQVIRG
RILHNGAYSLTLQGLGISWLGLRSLRELGSGLALIHHNTHLCFVHTVPWDQLFRNPH
QALLHTANRPEDECVGEGLACHQLCARGHCWGPGPTQCVNCSQFLRGQECVEECR
VLQGLPREYVNARHCLPCHPECQPQNGSVTCFGPEADQCVACAHYKDPPFCVARCP
SGVKPDLSYMPIWKFPDEEGACQPCPINCTHSCVDLDDKGCPAEQRASPLT
DNA sequence
ATGGACTGGACCTGGATCCTGTTCCTGGTGGCCGCCGCCACCAGGGTGCACAGCCTGAGGTTCGGCATCGTGGCCAGCA
GGGCCAACCACGCCCTGGTGGGCGGCAGCGGCGGCAGCGGCGGCAGCGGCGGCAGCGGCGGCGGCACCCAGGTGTGCAC
CGGCACCGACATGAAGCTGAGGCTGCCCGCCAGCCCCGAGACCCACCTGGACATGCTGAGGCACCTGTACCAGGGCTGC
CAGGTGGTGCAGGGCAACCTGGAGCTGACCTACCTGCCCACCAACGCCAGCCTGAGCTTCCTGCAGGACATCCAGGAGG
TGCAGGGCTACGTGCTGATCGCCCACAACCAGGTGAGGCAGGTGCCCCTGCAGAGGCTGAGGATCGTGAGGGGCACCCA
GCTGTTCGAGGACAACTACGCCCTGGCCGTGCTGGACAACGGCGACCCCCTGAACAACACCACCCCCGTGACCGGCGCC
AGCCCCGGCGGCCTGAGGGAGCTGCAGCTGAGGAGCCTGACCGAGATCCTGAAGGGCGGCGTGCTGATCCAGAGGAACC
CCCAGCTGTGCTACCAGGACACCATCCTGTGGAAGGACATCTTCCACAAGAACAACCAGCTGGCCCTGACCCTGATCGA
CACCAACAGGAGCAGGGCCTGCCACCCCTGCAGCCCCATGTGCAAGGGCAGCAGGTGCTGGGGCGAGAGCAGCGAGGAC
TGCCAGAGCCTGACCAGGACCGTGTGCGCCGGCGGCTGCGCCAGGTGCAAGGGCCCCCTGCCCACCGACTGCTGCCACG
AGCAGTGCGCCGCCGGCTGCACCGGCCCCAAGCACAGCGACTGCCTGGCCTGCCTGCACTTCAACCACAGCGGCATCTG
CGAGCTGCACTGCCCCGCCCTGGTGACCTACAACACCGACACCTTCGAGAGCATGCCCAACCCCGAGGGCAGGTACACC
TTCGGCGCCAGCTGCGTGACCGCCTGCCCCTACAACTACCTGAGCACCGACGTGGGCAGCTGCACCCTGGTGTGCCCCC
TGCACAACCAGGAGGTGACCGCCGAGGACGGCACCCAGAGGTGCGAGAAGTGCAGCAAGCCCTGCGCCAGGGTGTGCTA
CGGCCTGGGCATGGAGCACCTGAGGGAGGTGAGGGCCGTGACCAGCGCCAACATCCAGGAGTTCGCCGGCTGCAAGAAG
ATCTTCGGCAGCCTGGCCTTCCTGCCCGAGAGCTTCGACGGCGACCCCGCCAGCAACACCGCCCCCCTGCAGCCCGAGC
AGCTGCAGGTGTTCGAGACCCTGGAGGAGATCACCGGCTACCTGTACATCAGCGCCTGGCCCGACAGCCTGCCCGACCT
GAGCGTGTTCCAGAACCTGCAGGTGATCAGGGGCAGGATCCTGCACAACGGCGCCTACAGCCTGACCCTGCAGGGCCTG
GGCATCAGCTGGCTGGGCCTGAGGAGCCTGAGGGAGCTGGGCAGCGGCCTGGCCCTGATCCACCACAACACCCACCTGT
GCTTCGTGCACACCGTGCCCTGGGACCAGCTGTTCAGGAACCCCCACCAGGCCCTGCTGCACACCGCCAACAGGCCCGA
GGACGAGTGCGTGGGCGAGGGCCTGGCCTGCCACCAGCTGTGCGCCAGGGGCCACTGCTGGGGCCCCGGCCCCACCCAG
TGCGTGAACTGCAGCCAGTTCCTGAGGGGCCAGGAGTGCGTGGAGGAGTGCAGGGTGCTGCAGGGCCTGCCCAGGGAGT
ACGTGAACGCCAGGCACTGCCTGCCCTGCCACCCCGAGTGCCAGCCCCAGAACGGCAGCGTGACCTGCTTCGGCCCCGA
GGCCGACCAGTGCGTGGCCTGCGCCCACTACAAGGACCCCCCCTTCTGCGTGGCCAGGTGCCCCAGCGGCGTGAAGCCC
GACCTGAGCTACATGCCCATCTGGAAGTTCCCCGACGAGGAGGGCGCCTGCCAGCCCTGCCCCATCAACTGCACCCACA
GCTGCGTGGACCTGGACGACAAGGGCTGCCCCGCCGAGCAGAGGGCCAGCCCCCTGACC

### EXAMPLES

### Example 1. Incorporation of a Novel CD4+ Helper Epitope Identified from Aquifex aeolicus Enhances Humoral Responses Induced by DNA and Protein Vaccinations

Synthetic DNA delivery by electroporation was previously used to mediate in vivo assembly of nanoparticle vaccines and it was observed that some nanoparticle scaffolding domains (used to promote self-assembly of scaffolded antigens) could induce CD4+ T-cell responses (Xu et al., 2020). Here, epitope mapping on several bacterial or viral scaffold protein domains was performed and it was determined that lumazine synthase (LS) from Aquifex aeolicus contained very potent CD4-helper epitopes for both BALB/c and C57BL/6 mice. LS can scaffold the assembly of 60 copies of HIV-priming antigen GT8, eOD-GT8-60mer, as well as other antigens (Jardine et al., 2016; Xu et al., 2020). In silico binding analysis determined that the identified C57BL/6 CD4-helper epitope (LS-3) was predicted also to have high binding affinity (<100 nM) to several common human MHC-II alleles (HLA-DRB1*07:01, HLA-DRB1*15:01 and HLA-DRB5*01:01). How this epitope might contribute to humoral immunity was determined by engineering mutations that knocked out binding of this epitope to murine HLA I-Ab (LS3KO) and it was observed that DNA-launched GT8-60mer nanoparticles containing this mutant epitope (DLnano_CD4MutLS_GT8) induced weaker antibody responses than the corresponding DNA-launched wild-type GT8-60mer nanoparticles (DLnano_LS_GT8). Finally, engineered fusion of the identified LS-3 epitope to a different antigen, hemagglutinin (HA) receptor binding domain (RBD) from influenza H1/CA/07/09 (LS3-CA09), improved humoral responses induced to HA by DNA and protein vaccinations. Overall, this study provides a relatively rigorous demonstration that simple fusion of a dominant CD4-helper epitope to a target antigen could improve humoral responses induced by either protein or DNA vaccines in animal models, and additionally describes the identification of a novel CD4-helper epitope from a bacterial enzyme, which may help inform the design of additional protein and DNA vaccines and be of translational importance.

1. Materials and Methods

i. Structure Modeling and Design of CD4Mut_LS_GT8 Nanoparticles

Mutations to the LS-3 peptide were achieved using a structure-guided process. First, positions 14, 19, 22 and 24 in the LS domain were selected for mutation because they were making minimal contacts to the rest of the LS 1HQK crystal structure, which was hypothesized to have less detrimental effect on protein folding. Second, the ‘fixbb’ application of ROSETTA was used to computationally mutate the selected positions to each of the 20 amino acids allowing neighboring residues to change conformation. Mutations were selected which had similar or lower total score relative to the wild-type amino acid and by visual inspection of the resulting structural models. The mutations were R14K, A19G, A22F, A24G.

ii. DNA Design and Plasmid Synthesis

Protein sequences for IgE Leader Sequence and eOD-GT8-60mer were as previously reported (Briney et al., 2016; Xu et al., 2018). DNA encoding protein sequences were codon and RNA optimized as previously described (Xu et al., 2018). The optimized transgenes were synthesized de novo (GenScript) and cloned into a modified pVAX-1 backbone under the control of the human CMV promoter and bovine growth hormone polyadenylation signal.

iii. Production of His-Tagged LS3-CA09, LS3KO-CA09, or PADRE-CA09

Expi293F cells were transfected with pVAX plasmid vector carrying the His-Tagged LS3-CA09, LS3KO-CA09, PADRE-CA09, GT8-monomer, eOD-GT8-60mer or CD4Mut_LS_GT8-60mer transgene with PEI/OPTI-MEM and harvested 6 days post-transfection. Transfection supernatant was first purified with affinity chromatography using the AKTA pure 25 system and an IMAC Nickel column for His-Tagged constructs and gravity flow columns filled with GNL Lectin beads (for nanoparticles). The eluate fractions from the affinity purification were pooled, concentrated and dialyzed into 1×PBS buffer before being loaded onto the SEC column and then purified with size exclusion chromatography with the Superdex 200 10/300 GL column (GE Healthcare) for His-Tagged constructs, and with Superose 6 Increase 10/300 GL column for nanoparticles. Identified eluate fractions were then collected and concentrated to 1 mg/mL in PBS.

iv. Animals

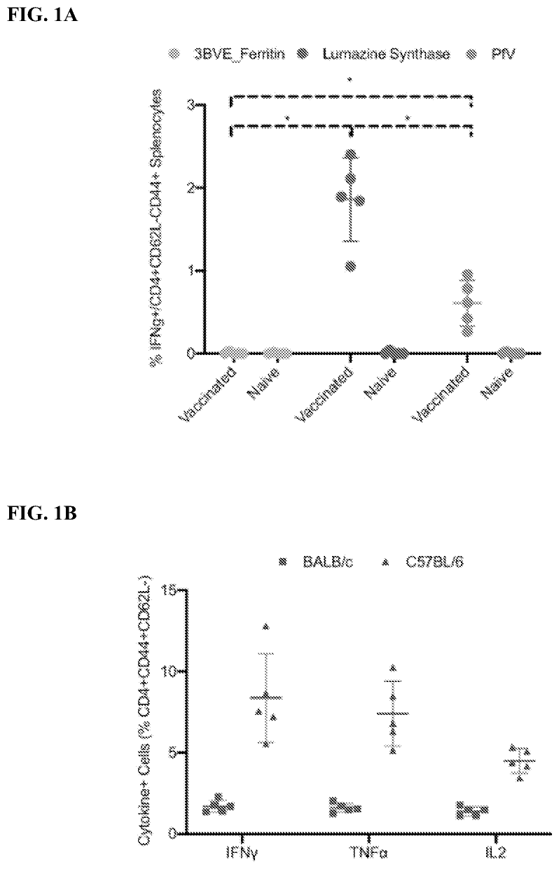

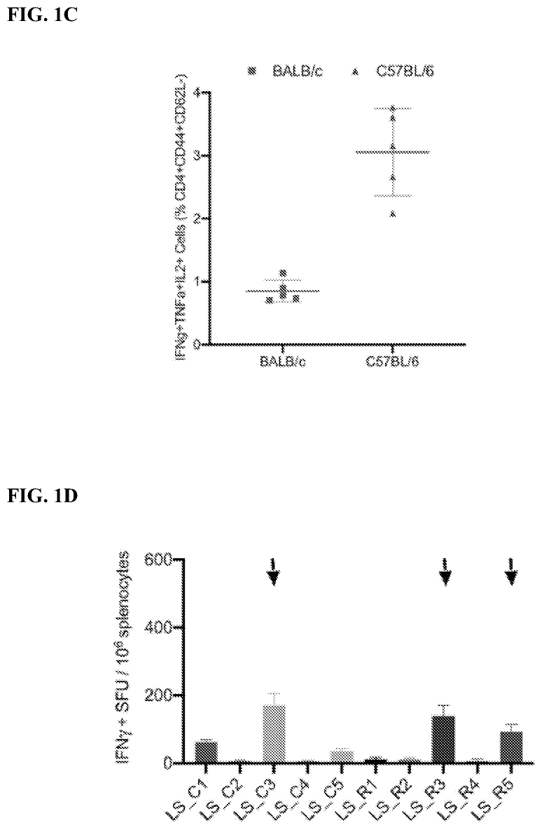

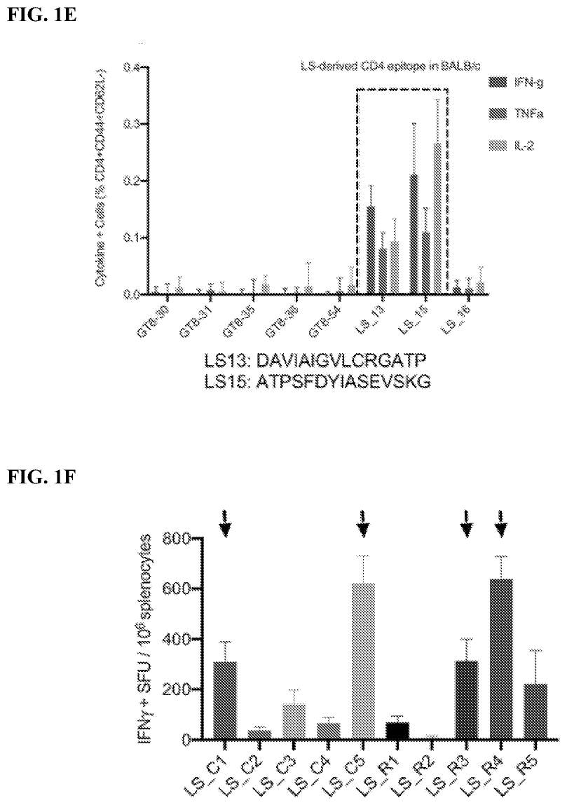

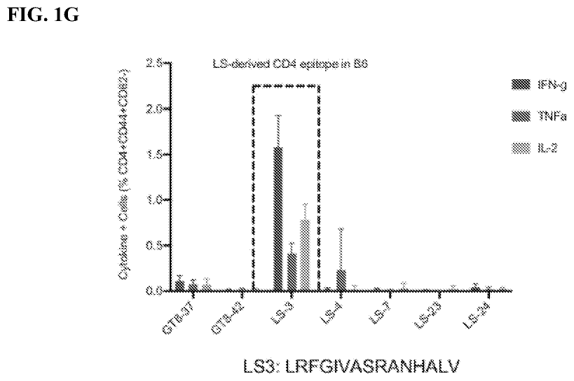

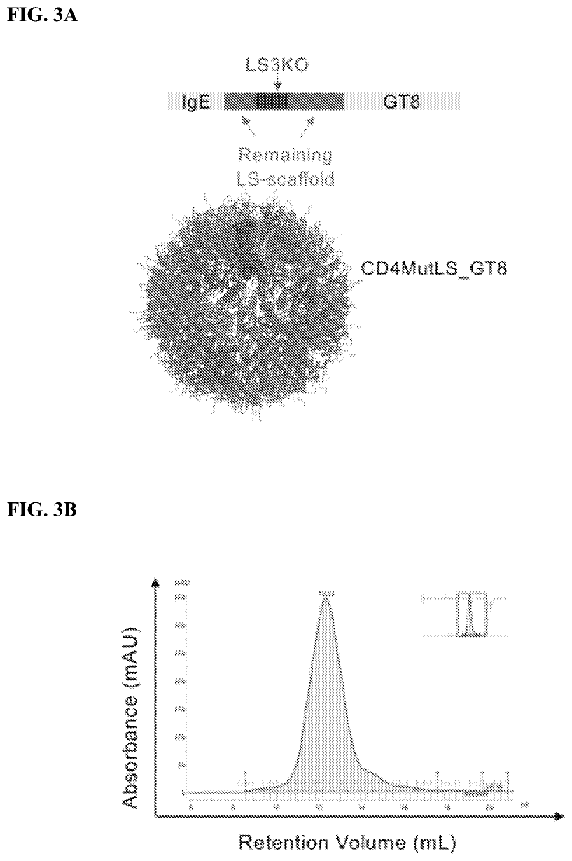

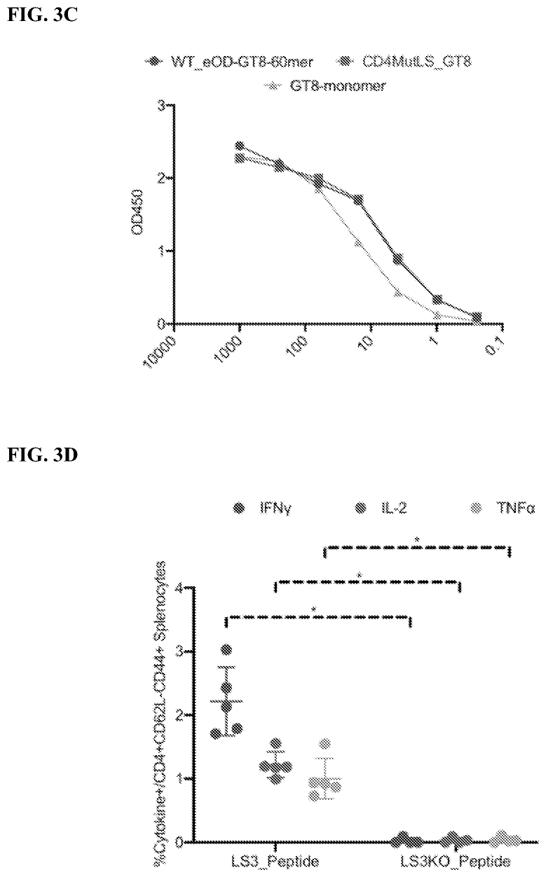

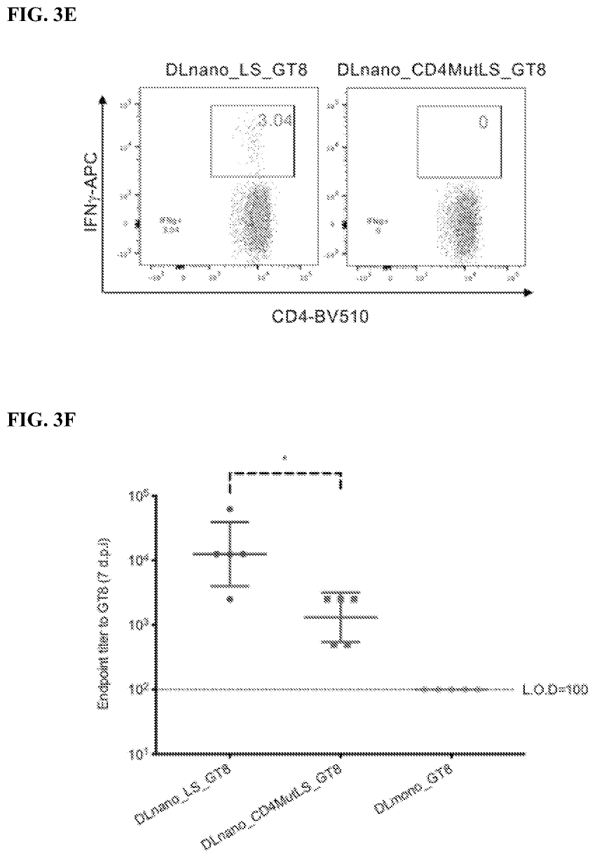

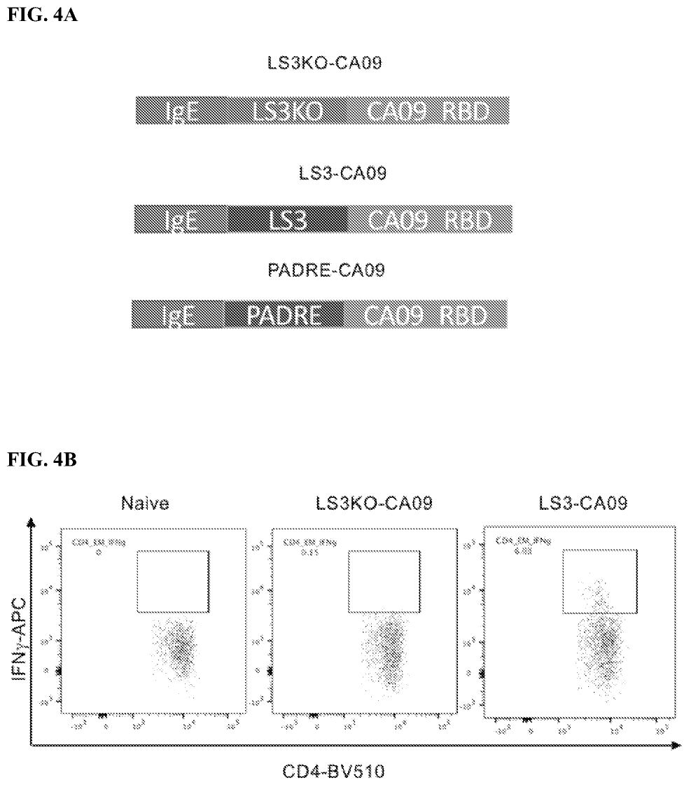

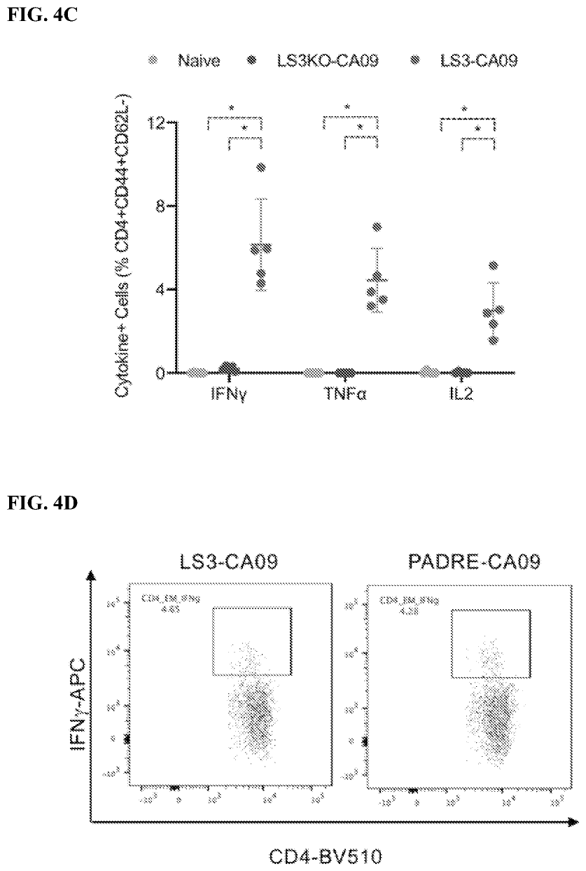

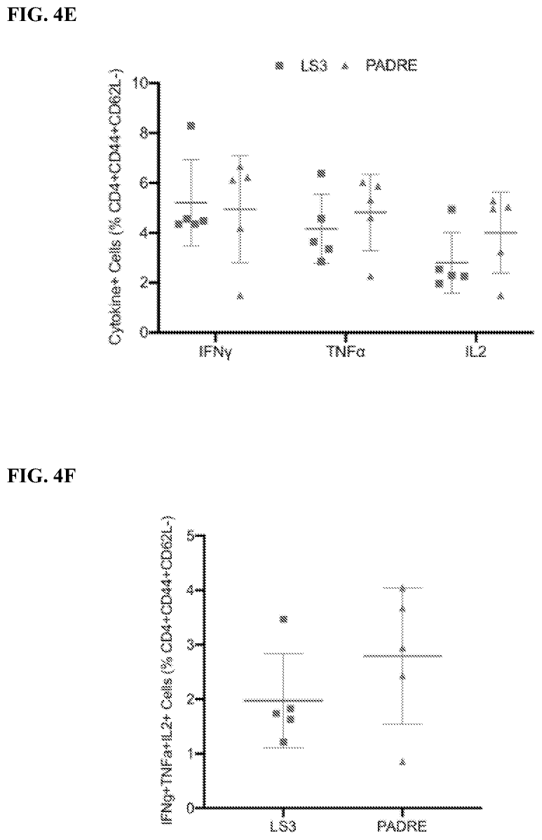

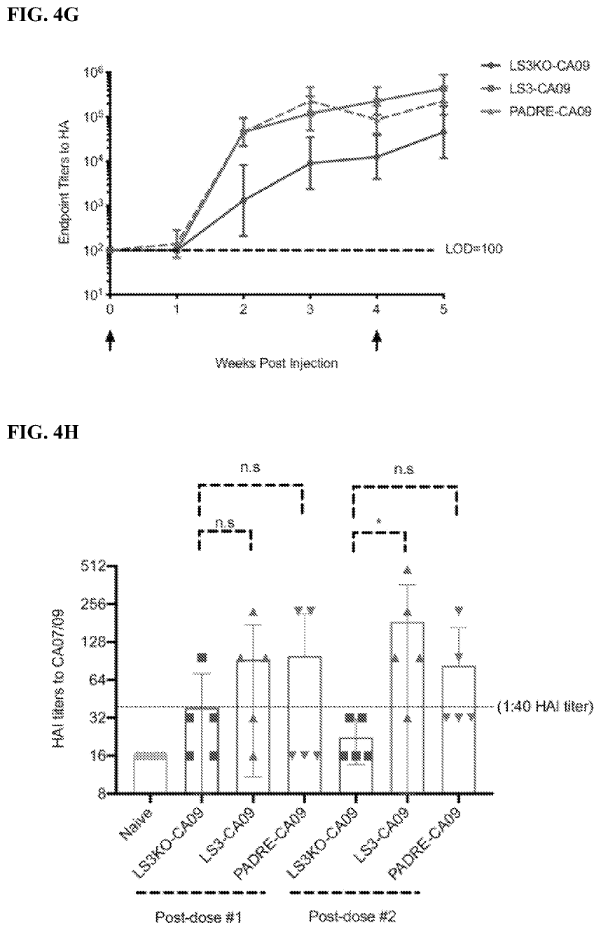

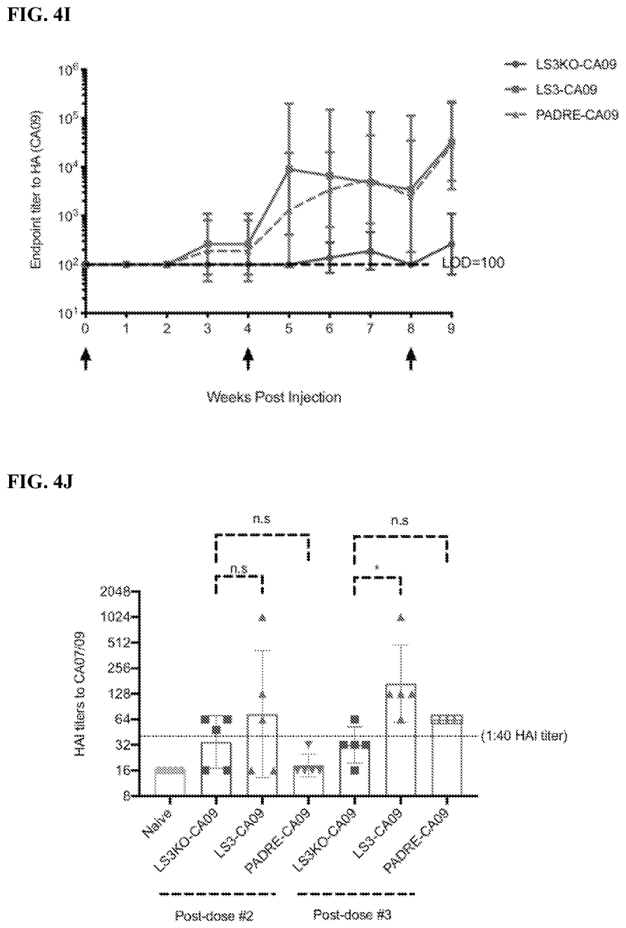

All animal experiments were carried out in accordance with animal protocols 201214 and 201115 approved by the Wistar Institute Institutional Animal Care and Use Committee (IACUC). For DNA-based immunization, 6 to 8 week old female C57BL/6 or BALB/c mice (Jackson Laboratory) were immunized with DNA vaccines via intramuscular injections into the tibialis anterior muscles, coupled with intramuscular EP with the CELLECTRA 3P device (Inovio Pharmaceuticals). In experiments in FIG. 1A-1G and FIG. 3A-3F, mice were immunized twice with 25 μg DNA plasmid three weeks apart and euthanized two weeks post the second vaccination. In experiments in FIG. 4A-4J, mice were immunized twice with 25 μg DNA plasmid twice four weeks apart and euthanized one week post the second vaccination. For vaccinations involving recombinant protein, 6 to 8-week-old female C57BL/6 mice were immunized intramuscularly with 10 μg of recombinant LS3-CA09, LS3KO-CA09 or PADRE-CA09 protein in 15 μL sterile PBS co-formulated with 15 μL Sigma Adjuvant System (SigmaAldrich) in the tibialis anterior muscles three times four weeks apart and were euthanized one week post the third immunization.

v. HA-Binding ELISA

96-well half area plates were coated at 4° C. overnight with 2 μg/mL of recombinant HA(ΔTM)(A/California/04/2009) (Immune Technology), and blocked at room temperature for 2 hours with a solution containing 1×PBS, 5% skim milk, 10% goat serum, 1% BSA, 1% FBS, and 0.2% Tween-20. The plates were subsequently incubated with serially diluted mouse sera at 37° C. for 2 hours, followed by 1-hour incubation with anti-mouse IgG H+L HRP (Bethyl) at 1:20,000 dilution at room temperature and developed with TMB substrate. Absorbance at 450 nm and 570 nm were recorded with BioTEK plate reader.

vi. Antigenic Profile Characterization of Purified eOD-GT8-60Mer and CD4Mut_LS_GT8-60mer

Corning half-area 96-well plates were coated with 2 μg/mL of purified eOD-GT8-60mer or CD4Mut_LS_GT8-60mer at 4° C. overnight. The plates were then blocked with the buffer as described above for 2 hours at room temperature, followed by incubation with serially diluted VRC01 at room temperature for 2 hours. The plates were then incubated with anti-human Fc (cross-adsorbed against rabbits and mice) (Jackson Immunoresearch) at 1:10,000 dilution for 1 hour, followed by addition of TMB substrate for detection. Absorbance at 450 nm and 570 nm were recorded with BioTEK plate reader.

vii. HAI Assay

Mice sera were treated with receptor-destroying enzyme (RDE, 1:3 ratio; SEIKEN) at 37° C. overnight for 18-20 hours followed by complement and enzyme inactivation at 56° C. for 45 minutes. RDE-treated sera were subsequently cross-adsorbed with 10% rooster red blood cells (Lampire Biologicals) in 0.9% saline at 4° C. for 1 hour. The cross-adsorbed sera were then serially diluted with PBS in a 96-well V-bottom microtiter plates (Corning). Four hemagglutinating doses (HAD) of A/California/07/2009 (H1N1)pdm09 (Virapur) were added to each well and the serum-virus mixture was incubated at room temperature for 1 hour. The mixture was then incubated with 50 μl 0.5% v/v rooster red blood cells in 0.9% saline for 30 minutes at room temperature. The HAI antibody titer was scored with the dot method, and the reciprocal of the highest dilution that did not cause agglutination of the rooster red blood cells was recorded.

viii. ELISpot Assay

Spleens from immunized mice were collected and homogenized into single cell suspension with a tissue stomacher in 10% FBS/1% Penicillin-streptomycin in RPMI 1640. Red blood cells were subsequently lysed with ACK lysing buffer (ThermoFisher) and percentage of viable cells were determined with Trypan Blue exclusion using Vi-CELL XR (Beckman Coulter). 200,000 cells were then plated in each well in the mouse IFNγ ELISpot plates (MabTech), followed by addition of peptide pools that span both the lumazine synthase, 3BVE, PfV or GT8 domains, or individual LS-3, LS3KO or PADRE peptides at 5 μg/mL of final concentration for each peptide (GenScript). The cells were then stimulated at 37° C. for 16-18 hours, followed by development according to the manufacturer's instructions. Spots for each well were then imaged and counted with ImmunoSpot Macro Analyzer.

ix. Intracellular Cytokine Staining

Single cell suspension from spleens of immunized animals were prepared as described before and stimulated with 5 μg/mL of peptides (GenScript) for 5 hours at 37° C. in the presence of 1:500 protein transport inhibitor (ThermoFisher). The cells were then incubated with live/dead for 10 minutes at room temperature, surface stains (anti-mouse CD4 BV510, anti-mouse CD8 APC-Cy7, anti-mouse CD44 AF700, anti-mouse CD62L BV771) (BD-Biosciences) at room temperature for 30 minutes. The cells were then fixed and permeabilized according to manufacturer's instructions for BD Cytoperm Cytofix kit and stained with intracellular stains anti-mouse IL-2 PE-Cy7, anti-mouse IFN-γ APC, anti-mouse CD3e PE-Cy5 and anti-mouse TNFa BV605 (BioLegend) at 4° C. for 1 hour. The cells were subsequently analyzed with LSR II 18-color flow cytometer.

x. Epitope Mapping

15-mer peptides spanning the LS and GT8 domains of eOD-GT8-60mer (GenScript) were arranged into row and column pools (each peptide appears exactly once in the row pool and once in the column pool). Splenocytes from BALB/c or C57BL/6 immunized twice with 25 ug DLnano_LS_GT8 were co-incubated with each peptide pool with a final concentration of 5 μg/mL for each peptide overnight in IFNγ ELIspot plates (MabTech). The plates were then developed according to manufacturer's instruction, and peptides that can potentially stimulate T-cell responses were identified based on the combination of row and column pools that induce IFNγ responses. Responses to those peptides were then confirmed with ICS as described in the last section.

xi. Statistics

Power analysis was performed with R based on our preliminary data to determine the smallest sample size that would allow us to achieve a power of 0.9 with a pre-set α-value of 0.05. All statistical analyses were performed with PRISM V 8.2.1 and R V 3.5.1. Each individual data point was sampled independently. Two-tailed Mann Whitney Rank Tests were used to compare differences between groups. Bonferroni corrections were used to adjust for multiple comparisons.

2. Results

i. Identification Novel Murine CD4-Helper Epitopes from the LS Domain of Aquifex aeolicus

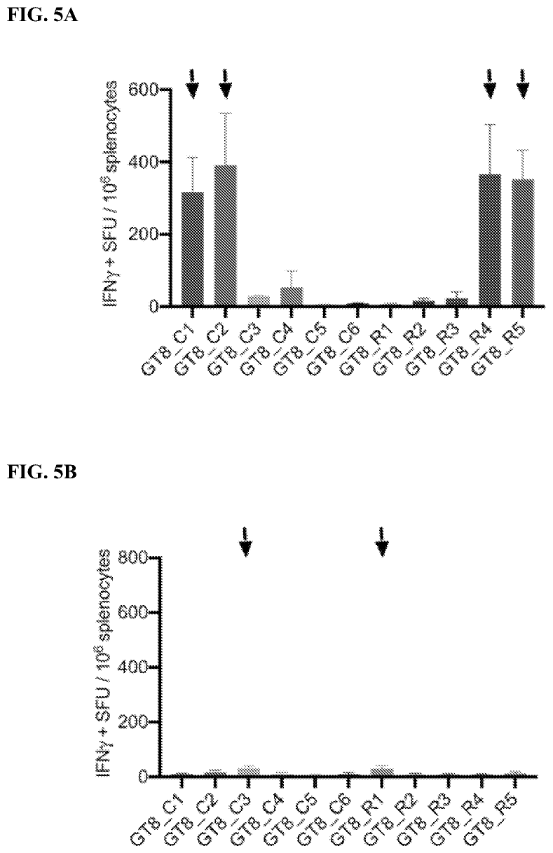

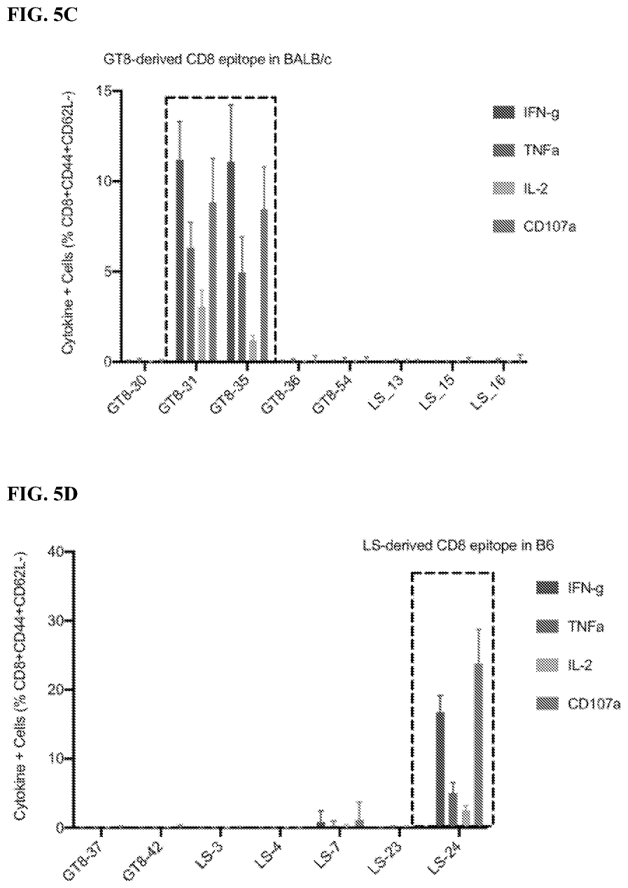

It was previously observed that scaffold domains used to drive in vivo assembly of nanoparticle vaccines could sometimes induce CD4+ T-cell responses (Xu et al., 2020). Here, CD4+ T-cell responses elicited by various nanoparticle scaffolding domains, ferritin from Helicobacter pylori (3BVE), LS from Aquifex aeolicus, and the viral cage of Prototype Foamy Virus (PfV), were compared in BALB/c immunized with DNA-launched GT8 nanoparticle vaccines that incorporate these respective protein domains (DLnano_3BVE_GT8, DLnano_LS_GT8, DLnano_PfV_GT8) (FIG. 1A). All mice in the experiments were immunized twice with 25 μg DNA immunogens three weeks apart and were euthanized two weeks post the second vaccination, at the time point which corresponded to their peak cellular responses. Using intracellular cytokine staining (ICS) to analyze murine splenocytes stimulated with overlapping peptide pools that spanned the respective protein domains, it was determined that the LS domain elicited the most potent CD4+ T-cell responses (approximately 2% of CD3+CD4+CD62L-CD44+ T-cells were observed to IFNγ+ following peptide stimulation), followed by the PfV and the 3BVE domains. Importantly, DLnano_LS_GT8 vaccination elicited even more potent CD4+ T-cell responses to the LS domain in the C57BL/6 mice than in the BALB/c mice, as measured by expression of pro-inflammatory cytokines IFNγ, TNFα and IL-2 upon peptide stimulation (FIG. 1B). LS-specific poly-functional CD4+ T cell responses, as defined by the simultaneous expression of all three cytokines IFNγ, TNFα and IL-2, were induced in both the BALB/c and the C57BL/6 mice, accounting for approximately 1% and 3% of all CD3+CD4+CD62L-CD44+ T cells respectively (FIG. 1C). To identify the exact CD4-helper epitope in both the BABL/c and the C57BL/6 mice, a combination of an IFNγ ELIspot assay for screening and a flow-based ICS assay for confirmation was used. Two predominant non-overlapping CD4+ epitopes in the LS domain were observed for the BALB/c mice (LS-13: DAVIAIGVLCRGATP and LS-15: ATPSFDYIASEVSKG) (FIG. 1D and FIG. 1E), whereas a single dominant CD4+ epitope in the LS domain was observed for the C57BL/6 mice (LS-3: LRFGIVASRANHALV) (FIG. 1F and FIG. 1G). The overall CD4+ T-cell responses measured by ICS were lower in the mapping study than in the previous experiment (FIG. 1B, FIG. 1E and FIG. 1G), likely because while fresh splenocytes were used for ICS analysis previously (FIG. 1B), splenocytes were used 24 hours post-harvest in the mapping experiment due to the time required for the preliminary IFNγ ELIspot screen (FIG. 1E and FIG. 1G). Additional epitopes identified through the preliminary IFNγ ELIspot screen were also characterized (FIG. 5A and FIG. 5B) by ICS, and mapped the CD8+ T-cell responses to two GT8 peptides in the BALB/c mice (FIG. 5C and Table 1) and to one LS peptide in the C57BL/6 mice (FIG. 5D and Table 1).

TABLE 1
Identified CD4+ and CD8+ epitopes in the LS and GT8 domains in the BALB/c
and C57BL/6 mice immunized twice with 25 μg DLnano_LS_GT8 three weeks apart
and sacrificed two weeks post the second vaccination for cellular analysis.

| Strain | Category | Domain | Sequence | Classification |
|---|---|---|---|---|
| BALB/c | CD4 | Lumazine Synthase | DAVIAIGVLCRGATP | WT |
| | | Lumazine Synthase | ATPSFDYIASEVSKG | WT |
| | CD8 | GT8 | TRQGGYSNDNTVIFR | WT |
| | | GT8 | ARCQIAGTVVSTQLF | WT |
| C57BL/6 | CD4 | Lumazine Synthase | LRFGIVASRANHALV | WT |
| | | Lumazine Synthase | LKFGIVGSRFNHGLV | LS3KO |
| | CD8 | Lumazine Synthase | AALCAIEMANLFKSL | WT |

ii. Murine HLA-IAb Epitope was Predicted to have High Binding Affinity for Several Human MHC-II Alleles by in Silico Analysis

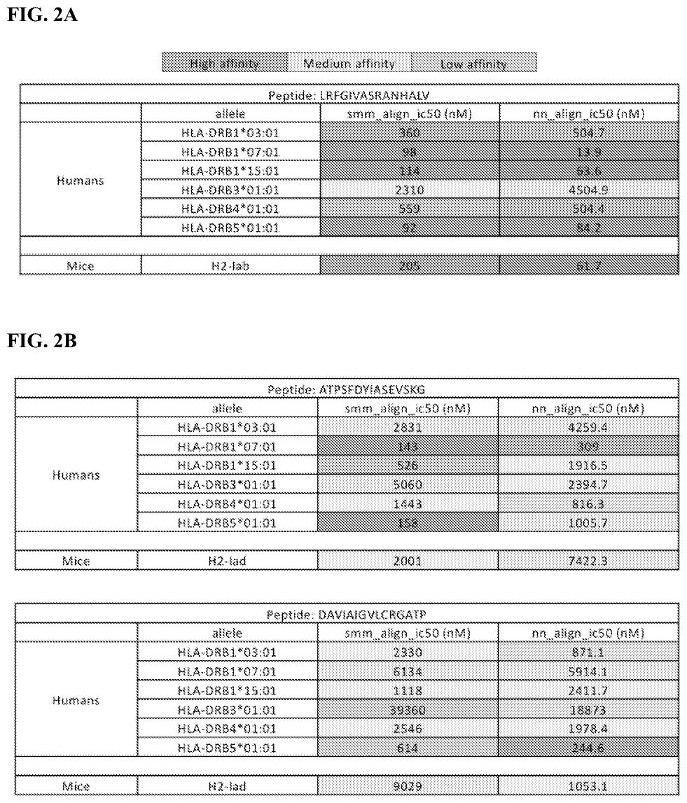

As the identified murine LS CD4-helper epitopes may or may not be conserved in humans, in silico analysis was used to predict the binding affinities of the identified LS-3, LS-13 and LS-15 epitopes to common human MHC-II alleles. Using a stabilization matrix method (SMM-align) and an artificial neural network-based method (NN-align) for alignment (Nielsen and Lund, 2009; Nielsen et al., 2007), the mapped murine C57BL/6 HLA-IAb epitope LS-3 demonstrated high binding affinity (<100 nM) for HLA-DRB1*07:01, HLA-DRB1*15:01 and HLA-DRB5*01:01, which correspond to human allele frequencies of 6.98%, 7.86%, and 14.6% respectively (Louthrenoo et al., 2013; Solberg et al., 2008), and moderate binding affinity (<1000 nM) for HLA-DRB1*03:01 and HLA-DRB4*01:01, which correspond to human allele frequencies of 6.76% and 35% respectively (Geng et al., 1995; Solberg et al., 2008). Low-to-moderate binding affinity (<5000 nM) was observed for LS-3 to the human allele HLA-DRB3*01:01 (FIG. 2A). Of note, both the NN-align and the SMM-align correctly predicted high binding affinities of the LS-3 epitope to murine HLA-IAb. In contrast, the identified murine BALB/c HLA-IAd epitopes LS-13 and LS-15 were predicted to have lower binding affinities to either human or murine HLA alleles than the LS-3 epitope (FIG. 2B). As such, since the LS-3 epitope was more likely to be conserved in humans, it was decided to further characterize the HLA-IAb LS-3 epitope rather than the HLA-IAd LS-13/LS-15 epitopes in the downstream experiments.

iii. Identified Murine LS-3 CD4+ Helper Epitope Supported the Induction of Potent Immune Responses by DLnano_LS_GT8

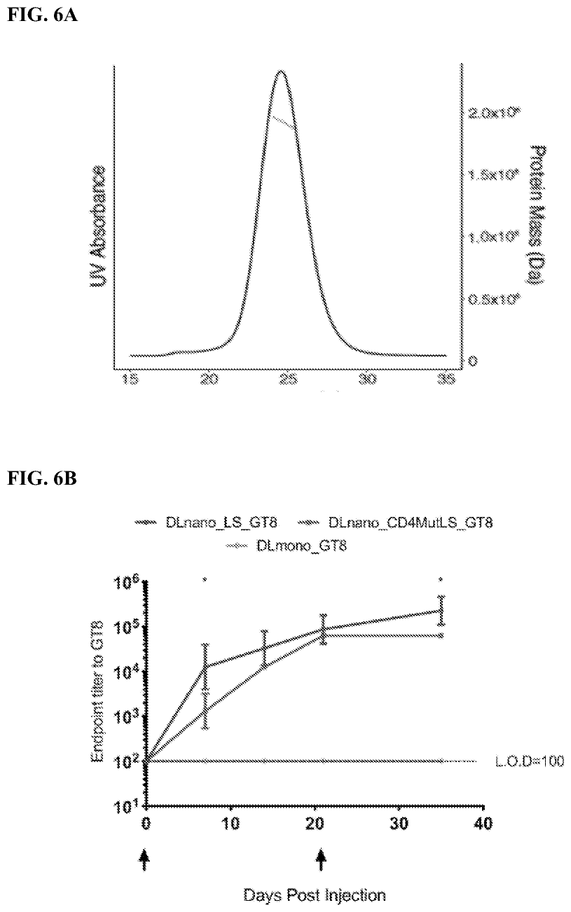

To determine whether CD4+ T-cell help provided by the identified LS-3 epitope can contribute to the induction of humoral immunity by DLnano_LS_GT8, a GT8 nanoparticle variant (DLnano_CD4MutLS_GT8) was engineered through a structure-guided design process in which the LS-3 epitope was selectively mutated to ablate its binding to HLA-IAb (as informed by the NN-align and the SMM-align based binding analysis). Care was taken, simultaneously, to avoid mutations that may disrupt nanoparticle assembly. 27% residues in the LS-3 epitope (4/15 residues) were mutated and the corresponding knockout epitope LS3-KO was generated (FIG. 3A and Table. 1), resulting in reduction of HLA-IAb binding affinity from 205 nM to 4261 nM by the SMM-align and 61.7 nM to 7668 nM by the NN-align. Whether the engineered variant DLnano_CD4MutLS_GT8 incorporating the LS3-KO epitope could still assemble homogenously was verified by expressing this new construct in vitro and performing size exclusion chromatography (SEC) of the lectin-column purified DLnano_CD4MutLS_GT8 transfection supernatant. SEC showed CD4MutLS_GT8 assembled homogenously into 60-mer (single peak observed on the SEC trace centering at 12.33 mL retention volume) similar to what we previously observed for the wildtype eOD-GT8-60mer (FIG. 3B) (Xu et al., 2020). Additionally, Size Exclusion Chromatography Multi Angle Light Scattering (SEC-MALs) analysis determined the molecular weight of CD4MutLS_GT8 to be around 2 Mda, close to the observed molecular weight of eOD-GT8-60mer (FIG. 6A) (Xu et al., 2020). The antigenic profiles of the engineered immunogens were examined and equivalent binding to VRC01, an HIV-1 broadly neutralizing antibody, was observed for eOD-GT8_60mer and CD4MutLS_GT8_60mer (FIG. 3C). ICS analyses of mice immunized with respective DNA-encoded constructs confirmed complete knockout of the LS-3 CD4+ helper epitope in the CD4MutLS_GT8 construct (FIG. 3D and FIG. 3E). Sera from animals seven d.p.i demonstrated significantly attenuated responses to GT8 in animals immunized with DLnano_CD4MutLS_GT8, though they still had stronger responses than those immunized with DNA-encoded GT8-monomer (FIG. 3F). Differences in humoral immunity induced by DLnano_LS_GT8 and DLnano_CD4MutLS_GT8 waned overtime; however, repeat vaccination of DLnano_LS_GT8 but not DLnano_CD4MutLS_GT8 at 21 d.p.i boosted the humoral immunity in mice (FIG. 6B). Taken together, this experiment suggests that the identified LS-3 CD4-helper epitope contributes to the overall antibody responses induced, as partial attenuation was observed when binding of this epitope to HLA IAb was knocked out.

iv. Engineered Fusion of LS-3 CD4+ Helper Epitope to CA09 HA-RBD Enhanced Anti-HA Antibody Responses Induced by DNA or Protein Vaccines

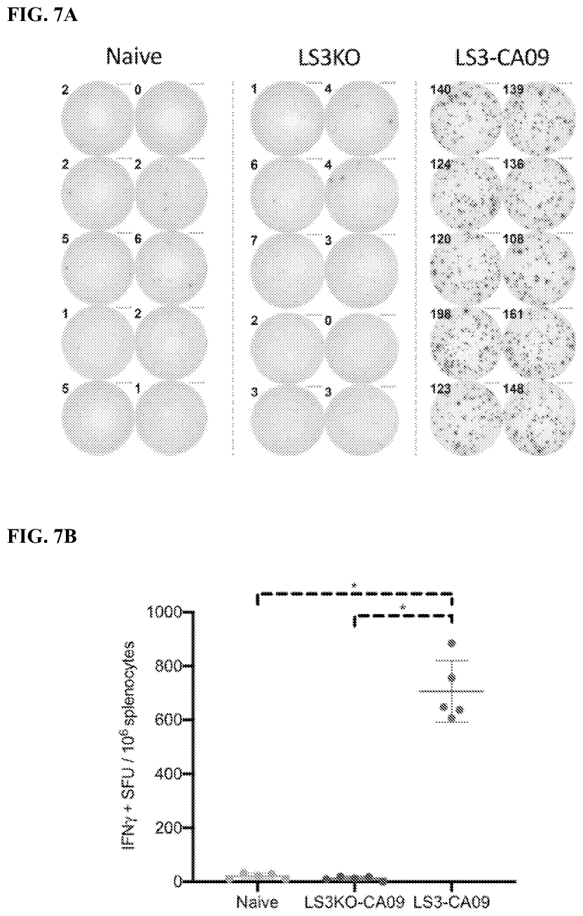

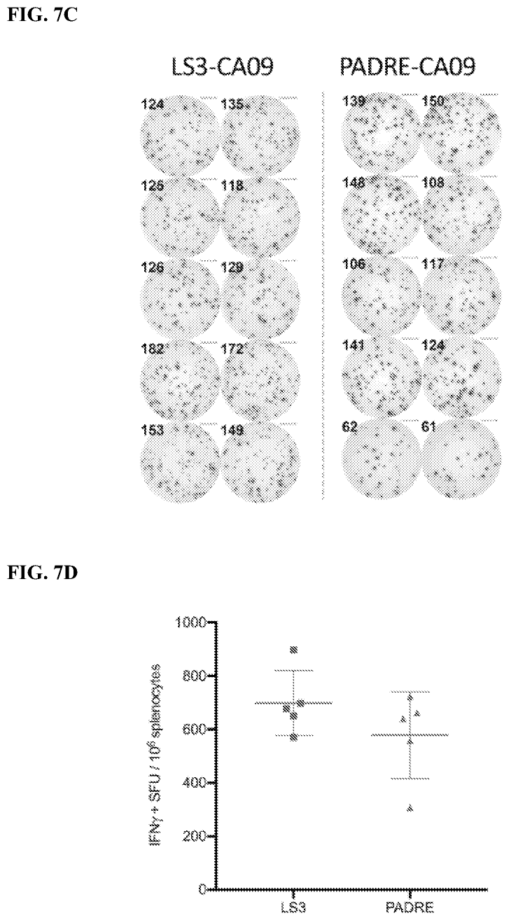

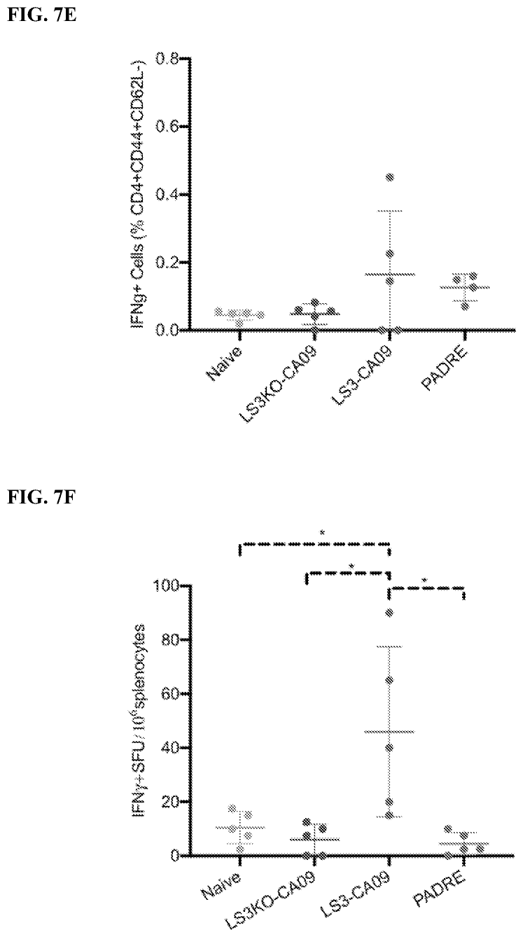

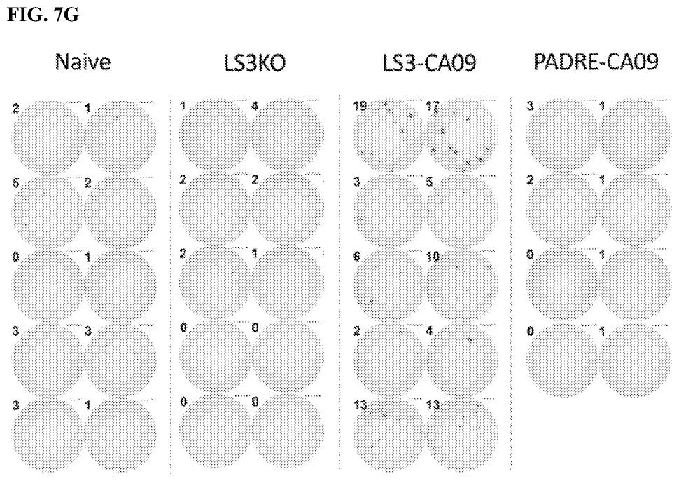

As it has determined that CD4+ T-cell help provided by the LS-3 epitope could contribute to the overall humoral responses, the next step is to determine if it can serve as a “molecular adjuvant” to enhance induced antibody responses by engineering fusion of the epitope with a different model antigen, CA09 HA-RBD. Either an LS3KO epitope, an LS3 epitope, or a PADRE epitope (AKFVAAWTLKAAA) was incorporated on the N-terminus of CA09 HA-RBD, downstream of the IgE leader sequence, which served as a secretion tag for the antigen (FIG. 4A). LS3KO-CA09 served as a better control to which responses induced by LS3-CA09 and PADRE-CA09 would be compared, as the impact of N-terminal peptide fusion on the immunogenicity of an antigen would be considered (protein sequences of LS3KO-CA09 and LS3-CA09 only differed at four residues). First, it was confirmed that DNA-encoded LS3-CA09 could induce CD4+ T-cell responses to the incorporated LS-3 epitope. Indeed, by ICS analysis, C57BL/6 mice immunized with DNA-encoded LS3-CA09 but not those immunized with LS3KO-CA09 were capable of mounting CD4+ T-cell responses to their respective incorporated epitope (FIG. 4B and FIG. 4C). The finding was similarly validated by IFNγ ELIspot analysis (FIG. 7A and FIG. 7B). Next, CD4+ T-cell responses induced by DNA-encoded LS3-CA09 and PADRE-CA09 were compared to those induced by the LS3 and PADRE epitopes, respectively. Both LS-3 and PADRE elicited potent CD4+ T-cell responses upon vaccination of DNA-encoded LS3-CA09 and PADRE-CA09 in C57BL/6 mice, with similar levels of cytokine responses induced as determined by ICS (FIG. 4D and FIG. 4E) and IFNγ ELIspot assays (FIG. 7C and FIG. 7D). Additionally, by ICS analysis, epitope-specific polyfunctional T-cell responses were also similar between the LS-3 and PADRE epitopes (FIG. 4F).

Next, the humoral responses induced by two vaccinations of DNA-encoded LS3KO-CA09, LS3-CA09 and PADRE-CA09 were compared to the humoral responses induced by CA09 HA over time. By ELISA analysis, both DNA-encoded LS3-CA09 and PADRE-CA09 improved induced binding antibody responses to HA as compared to DNA-encoded LS3KO-CA09 prior to and after the boost, with approximately 9.5-fold and 5-fold improvements observed for DNA-encoded LS3-CA09 and PADRE-CA09 respectively (FIG. 4G). Most importantly, it was observed that functional antibody responses, as measured by the hemagglutination inhibition (HAI) titers, were significantly improved for DNA-encoded LS3-CA09 relative to LS3KO-CA09 after the second vaccination (with a mean titer of 126 versus 21, respectively) (FIG. 4H). DNA-encoded PADRE-CA09, on the other hand, did not significantly improve the HAI titers relative to DNA-encoded LS3KO-CA09 after the first or the second vaccination (FIG. 4H).

Whether the observed phenomenon can be generalized to other routes of vaccination, such as protein vaccines, was further determined. C-terminal His-tagged LS3KO-CA09, LS3-CA09 and PADRE-CA09 were expressed in vitro and purified from Expi293F cell transfection supernatant with nickel column. C57BL/6 mice were subsequently immunized with 10 μg recombinant protein LS3KO-CA09, LS3-CA09 or PADRE-CA09 co-formulated with RIBI each time. Humoral and cellular responses induced by protein vaccination were observed to be considerably lower than those induced by DNA vaccinations (FIG. 4G and FIG. 4I), such that three protein vaccinations at Weeks 0, 4 and 8 were required to observed robust humoral responses. Epitope-specific CD4+ T-cell responses induced by protein vaccinations were considerably lower than that by DNA vaccines. However, CD4+ T-cell responses directed at the LS-3 epitope could still be observed by ICS (FIG. 7E) and by IFNγ ELIspot (FIG. 7F and FIG. 7G). CD4+ T-cell responses to PADRE were not observed (FIG. 7E, FIG. 7F and FIG. 7G), likely as a result of the sensitivity of detection of the assays. Regardless, it was observed that HA-binding antibody titers induced by both protein LS3-CA09 and PADRE-CA09 vaccinations were significantly higher (100% sero-conversion in both groups) relative to protein LS3KO-CA09 vaccination, for which 20% sero-conversion (1/5 mice) was observed (FIG. 4I). Lastly, similar to what was observed for DNA-vaccinations, protein LS3-CA09 vaccination induced significantly improved HAI titers post-dose 3 relative to protein LS3KO-CA09 vaccination (with a mean titer of 169 versus 32, respectively). Protein PADRE-CA09 vaccination, on the other hand, was not observed to induce significantly improved HAI titers (FIG. 4J). Taken together, the data suggest that the engineered fusion of the identified LS-3 CD4+ helper epitope to a model antigen can significantly enhance humoral responses to that antigen. Additionally, the incorporation of the LS-3 epitope performed as well as, if not better than, the incorporation of the PADRE epitope in terms of adjuvating humoral responses.

3. Discussion

The importance of CD4+ T-cell help in facilitating antibody maturation and class-switching is well-established (Crum-Cianflone and Wallace, 2014). AIDS patients with low CD4+ T-cell count cannot mount effective antibody responses with vaccination. Similarly, laboratory animals that receive transient CD4+ T-cell depletion also cannot develop strong antibody responses to a foreign gene or an antigen (Duperret et al., 2018; Wise et al., 2020). Both secreted soluble cytokine factors as well as surface-displayed ligands from Tfh cells are indispensable to the survival, AID-dependent somatic hypermutation and proliferation of GCB cells, and are necessary for the generation of antibody-secreting plasma cells and long-lived memory B cells (Crotty, 2015).

While larger antigenic protein domains likely harbor CD4+ T-cell epitopes that can be restricted by the host HLA alleles for the induction of Tfh responses, carbohydrate and peptide vaccines are intrinsically minimalistic and unlikely to contain potent CD4+ T-cell help epitopes (Astronomo and Burton, 2010). Additionally, domain minimization has now become an increasingly important approach in protein engineering and vaccinology, as researchers begin to appreciate the importance of focusing elicited B-cell responses to certain target epitopes by designing protein mini-domain devoid of distracting immunodominant surfaces (van der Lubbe et al., 2018; Yassine et al., 2015). However, these mini-proteins contain fewer overlapping peptides, and therefore statistically will be less likely to harbor potent HLA-restricted CD4+ helper epitopes. As such, several studies have explored conjugation of these carbohydrate, peptide, or mini-protein vaccines to carrier proteins, including but not limited to KLH, tetanus toxin and HbsAg. Induction of more potent antibody responses was observed in many cases (Jin et al., 2017; Marini et al., 2019). However, this approach may undermine the core motivations behind domain minimization by introducing a host of immunodominant distracting surfaces which may skew induced humoral responses.

Conjugation of the antigen with a shorter conserved CD4+ T-cell epitope may offer a promising alternative to conjugation with a whole protein carrier. As the CD4+ T-cell epitope is intrinsically shorter (12-16 amino acid long), it will less represent a distracting immunodominant surface (Hemmer et al., 2000). Additionally, they may alternatively be used as short linker to connect different protein domains, such as to cross-link a nanoparticle protein scaffold with a target antigen to promote vaccine antigen self-assembly (He et al., 2018). Fusing antigen with the PADRE epitope has been demonstrated to improve antibody responses in several animal studies and has also been explored in the clinic (clinical trials NCT01972737 and NCT02264236) (Rosa et al., 2004). Additional CD4+ helper epitopes have also been mapped and explored. For example, a recent study reported that co-delivery of MPER antigen with a Leishmania major derived HLA I-Ad helper CD4+ T-cell epitope (LACK) in liposomes can improve induced anti-MPER antibody responses (Elbahnasawy et al., 2018).

This study reported that the identification and characterization of a novel HLA I-Ab epitope LS-3 from the LS domain of Aquifex aeolicus, which is also the protein domain that can be used to scaffold the assembly of a 60-mer nanoparticle. In silico analysis predicted the LS-3 epitope to have high binding affinity to several common human HLA alleles, particularly HLA-DRB1*07:01, HLA-DRB1*15:01 and HLA-DRB5*01:01. Epitope knockout experiment demonstrated that CD4+ T-cell help provided by this epitope could indeed contribute to the overall antibody responses. Finally, engineered genetic fusion of the LS-3 epitope with a different target antigen CA09 HA-RBD (LS3-CA09) significantly increased binding and HAI antibody titers elicited by protein and DNA vaccinations to HA as compared to the control antigen, LS3KO-CA09. The study demonstrates the potential utility in this epitope as a “molecular adjuvant” to increase vaccine-induced antibody responses, in both preclinical murine studies as well as possibly in translational vaccine trials.

### REFERENCES

1. Alexander, J., del Guercio, M. F., Maewal, A., Qiao, L., Fikes, J., Chesnut, R. W., Paulson, J., Bundle, D. R., DeFrees, S., and Sette, A. (2000). Linear PADRE T helper epitope and carbohydrate B cell epitope conjugates induce specific high titer IgG antibody responses. J Immunol 164, 1625-1633.
2. Andreatta, M., Karosiene, E., Rasmussen, M., Stryhn, A., Buus, S., and Nielsen, M. (2015). Accurate pan-specific prediction of peptide-MHC class II binding affinity with improved binding core identification. Immunogenetics 67, 641-650.
3. Astronomo, R. D., and Burton, D. R. (2010). Carbohydrate vaccines: developing sweet solutions to sticky situations? Nat Rev Drug Discov 9, 308-324.
4. Briney, B., Sok, D., Jardine, J. G., Kulp, D. W., Skog, P., Menis, S., Jacak, R., Kalyuzhniy, O., de Val, N., Sesterhenn, F., et al. (2016). Tailored Immunogens Direct Affinity Maturation toward HIV Neutralizing Antibodies. Cell 166, 1459-1470 e1411.
5. Burton, D. R., and Hangartner, L. (2016). Broadly Neutralizing Antibodies to HIV and Their Role in Vaccine Design. Annu Rev Immunol 34, 635-659.
6. Carrasco, Y. R., Fleire, S. J., Cameron, T., Dustin, M. L., and Batista, F. D. (2004). LFA-1/ICAM-1 interaction lowers the threshold of B cell activation by facilitating B cell adhesion and synapse formation. Immunity 20, 589-599.
7. Clem, A. S. (2011). Fundamentals of vaccine immunology. J Glob Infect Dis 3, 73-78.
8. Collins, K. A., Snaith, R., Cottingham, M. G., Gilbert, S. C., and Hill, A. V. S. (2017). Enhancing protective immunity to malaria with a highly immunogenic virus-like particle vaccine. Sci Rep 7, 46621.
9. Corti, D., and Lanzavecchia, A. (2013). Broadly neutralizing antiviral antibodies. Annu Rev Immunol 31, 705-742.
10. Crotty, S. (2014). T follicular helper cell differentiation, function, and roles in disease. Immunity 41, 529-542.
11. Crotty, S. (2015). A brief history of T cell help to B cells. Nat Rev Immunol 15, 185-189.
12. Crum-Cianflone, N. F., and Wallace, M. R. (2014). Vaccination in HIV-infected adults. AIDS Patient Care STDS 28, 397-410.
13. Diethelm-Okita, B. M., Okita, D. K., Banaszak, L., and Conti-Fine, B. M. (2000). Universal epitopes for human CD4+ cells on tetanus and diphtheria toxins. J Infect Dis 181, 1001-1009.
14. Duperret, E. K., Trautz, A., Stoltz, R., Patel, A., Wise, M. C., Perales-Puchalt, A., Smith, T., Broderick, K. E., Masteller, E., Kim, J. J., et al. (2018). Synthetic DNA-Encoded Monoclonal Antibody Delivery of Anti-CTLA-4 Antibodies Induces Tumor Shrinkage In Vivo. Cancer Res 78, 6363-6370.
15. Elbahnasawy, M. A., Donius, L. R., Reinherz, E. L., and Kim, M. (2018). Co-delivery of a CD4 T cell helper epitope via covalent liposome attachment with a surface-arrayed B cell target antigen fosters higher affinity antibody responses. Vaccine 36, 6191-6201.
16. Elgueta, R., Benson, M. J., de Vries, V. C., Wasiuk, A., Guo, Y., and Noelle, R. J. (2009). Molecular mechanism and function of CD40/CD40L engagement in the immune system. Immunol Rev 229, 152-172.
17. Falugi, F., Petracca, R., Mariani, M., Luzzi, E., Mancianti, S., Carinci, V., Melli, M. L., Finco, O., Wack, A., Di Tommaso, A., et al. (2001). Rationally designed strings of promiscuous CD4(+) T cell epitopes provide help to Haemophilus influenzae type b oligosaccharide: a model for new conjugate vaccines. Eur J Immunol 31, 3816-3824.
18. Flynn, S., Toellner, K. M., Raykundalia, C., Goodall, M., and Lane, P. (1998). CD4 T cell cytokine differentiation: the B cell activation molecule, OX40 ligand, instructs CD4 T cells to express interleukin 4 and upregulates expression of the chemokine receptor, Blr-1. J Exp Med 188, 297-304.
19. Geng, L., Imanishi, T., Tokunaga, K., Zhu, D., Mizuki, N., Xu, S., Geng, Z., Gojobori, T., Tsuji, K., and Inoko, H. (1995). Determination of HLA class II alleles by genotyping in a Manchu population in the northern part of China and its relationship with Han and Japanese populations. Tissue Antigens 46, 111-116.
20. Ghaffari-Nazari, H., Tavakkol-Afshari, J., Jaafari, M. R., Tahaghoghi-Hajghorbani, S., Masoumi, E., and Jalali, S. A. (2015). Improving Multi-Epitope Long Peptide Vaccine Potency by Using a Strategy that Enhances CD4+T Help in BALB/c Mice. PLoS One 10, e0142563.
21. Ghosh, M., Solanki, A. K., Roy, K., Dhoke, R. R., Ashish, and Roy, S. (2013). Carrier protein influences immunodominance of a known epitope: implication in peptide vaccine design. Vaccine 31, 4682-4688.
22. He, L., Kumar, S., Allen, J. D., Huang, D., Lin, X., Mann, C. J., Saye-Francisco, K. L., Copps, J., Sarkar, A., Blizard, G. S., et al. (2018). HIV-1 vaccine design through minimizing envelope metastability. Sci Adv 4, eaau6769.
23. Hemmer, B., Kondo, T., Gran, B., Pinilla, C., Cortese, I., Pascal, J., Tzou, A., McFarland, H. F., Houghten, R., and Martin, R. (2000). Minimal peptide length requirements for CD4(+) T cell clones—implications for molecular mimicry and T cell survival. Int Immunol 12, 375-383.
24. Jardine, J. G., Kulp, D. W., Havenar-Daughton, C., Sarkar, A., Briney, B., Sok, D., Sesterhenn, F., Ereno-Orbea, J., Kalyuzhniy, O., Deresa, I., et al. (2016). HIV-1 broadly neutralizing antibody precursor B cells revealed by germline-targeting immunogen. Science 351, 1458-1463.
25. Jiang, S., Hillyer, C., and Du, L. (2020). Neutralizing Antibodies against SARS-CoV-2 and Other Human Coronaviruses. Trends Immunol.
26. Jin, C., Gibani, M. M., Moore, M., Juel, H. B., Jones, E., Meiring, J., Harris, V., Gardner, J., Nebykova, A., Kerridge, S. A., et al. (2017). Efficacy and immunogenicity of a Vi-tetanus toxoid conjugate vaccine in the prevention of typhoid fever using a controlled human infection model of Salmonella Typhi: a randomised controlled, phase 2b trial. Lancet 390, 2472-2480.
27. Kageyama, R., Cannons, J. L., Zhao, F., Yusuf, I., Lao, C., Locci, M., Schwartzberg, P. L., and Crotty, S. (2012). The receptor Ly108 functions as a SAP adaptor-dependent on-off switch for T cell help to B cells and NKT cell development. Immunity 36, 986-1002.
28. Kurdi, A. T., Glavey, S. V., Bezman, N. A., Jhatakia, A., Guerriero, J. L., Manier, S., Moschetta, M., Mishima, Y., Roccaro, A., Detappe, A., et al. (2018). Antibody-Dependent Cellular Phagocytosis by Macrophages is a Novel Mechanism of Action of Elotuzumab. Mol Cancer Ther 17, 1454-1463.
29. Kwong, P. D., Mascola, J. R., and Nabel, G. J. (2013). Broadly neutralizing antibodies and the search for an HIV-1 vaccine: the end of the beginning. Nat Rev Immunol 13, 693-701.
30. Laursen, N. S., Friesen, R. H. E., Zhu, X., Jongeneelen, M., Blokland, S., Vermond, J., van Eijgen, A., Tang, C., van Diepen, H., Obmolova, G., et al. (2018). Universal protection against influenza infection by a multidomain antibody to influenza hemagglutinin. Science 362, 598-602.
31. Louthrenoo, W., Kasitanon, N., Wichainun, R., Wangkaew, S., Sukitawut, W., Ohnogi, Y., Hong, G. H., Kuwata, S., and Takeuchi, F. (2013). The genetic contribution of HLA-DRB5*01:01 to systemic lupus erythematosus in Thailand. Int J Immunogenet 40, 126-130.
32. Marini, A., Zhou, Y., Li, Y., Taylor, I. J., Leneghan, D. B., Jin, J., Zaric, M., Mekhaiel, D., Long, C. A., Miura, K., et al. (2019). A Universal Plug-and-Display Vaccine Carrier Based on HBsAg VLP to Maximize Effective Antibody Response. Front Immunol 10, 2931.
33. Nielsen, M., and Lund, O. (2009). NN-align. An artificial neural network-based alignment algorithm for MHC class II peptide binding prediction. BMC Bioinformatics 10, 296.
34. Nielsen, M., Lundegaard, C., and Lund, O. (2007). Prediction of MHC class II binding affinity using SMM-align, a novel stabilization matrix alignment method. BMC Bioinformatics 8, 238.
35. Pichichero, M. E. (2013). Protein carriers of conjugate vaccines: characteristics, development, and clinical trials. Hum Vaccin Immunother 9, 2505-2523.
36. Ragupathi, G., Cappello, S., Yi, S. S., Canter, D., Spassova, M., Bommann, W. G., Danishefsky, S. J., and Livingston, P. O. (2002). Comparison of antibody titers after immunization with monovalent or tetravalent KLH conjugate vaccines. Vaccine 20, 1030-1038.
37. Rosa, D. S., Tzelepis, F., Cunha, M. G., Soares, I. S., and Rodrigues, M. M. (2004). The pan HLA DR-binding epitope improves adjuvant-assisted immunization with a recombinant protein containing a malaria vaccine candidate. Immunol Lett 92, 259-268.
38. Seidel, U. J., Schlegel, P., and Lang, P. (2013). Natural killer cell mediated antibody-dependent cellular cytotoxicity in tumor immunotherapy with therapeutic antibodies. Front Immunol 4, 76.
39. Snook, A. E., Baybutt, T. R., Xiang, B., Abraham, T. S., Flickinger, J. C., Jr., Hyslop, T., Zhan, T., Kraft, W. K., Sato, T., and Waldman, S. A. (2019). Split tolerance permits safe Ad5-GUCY2C-PADRE vaccine-induced T-cell responses in colon cancer patients. J Immunother Cancer 7, 104.
40. Solberg, O. D., Mack, S. J., Lancaster, A. K., Single, R. M., Tsai, Y., Sanchez-Mazas, A., and Thomson, G. (2008). Balancing selection and heterogeneity across the classical human leukocyte antigen loci: a meta-analytic review of 497 population studies. Hum Immunol 69, 443-464.
41. Valea, I., Adjei, S., Usuf, E., Traore, O., Ansong, D., Tinto, H., Owusu Boateng, H., Leach, A., Mwinessobaonfou Some, A., Buabeng, P., et al. (2018). Immune response to the hepatitis B antigen in the RTS,S/AS01 malaria vaccine, and co-administration with pneumococcal conjugate and rotavirus vaccines in African children: A randomized controlled trial. Hum Vaccin Immunother 14, 1489-1500.
42. van der Lubbe, J. E. M., Verspuij, J. W. A., Huizingh, J., Schmit-Tillemans, S. P. R., Tolboom, J., Dekking, L., Kwaks, T., Brandenburg, B., Meijberg, W., Zahn, R. C., et al. (2018). Mini-HA Is Superior to Full Length Hemagglutinin Immunization in Inducing Stem-Specific Antibodies and Protection Against Group 1 Influenza Virus Challenges in Mice. Front Immunol 9, 2350.
43. van Erp, E. A., Luytjes, W., Ferwerda, G., and van Kasteren, P. B. (2019). Fc-Mediated Antibody Effector Functions During Respiratory Syncytial Virus Infection and Disease. Front Immunol 10, 548.
44. Wise, M. C., Xu, Z., Tello-Ruiz, E., Beck, C., Trautz, A., Patel, A., Elliott, S. T., Chokkalingam, N., Kim, S., Kerkau, M. G., et al. (2020). In vivo delivery of synthetic DNA-encoded antibodies induces broad HIV-1-neutralizing activity. J Clin Invest.
45. Xu, Z., and Kulp, D. W. (2019). Protein engineering and particulate display of B-cell epitopes to facilitate development of novel vaccines. Curr Opin Immunol 59, 49-56.
46. Xu, Z., Wise, M. C., Choi, H., Perales-Puchalt, A., Patel, A., Tello-Ruiz, E., Chu, J. D., Muthumani, K., and Weiner, D. B. (2018). Synthetic DNA delivery by electroporation promotes robust in vivo sulfation of broadly neutralizing anti-HIV immunoadhesin eCD4-Ig. EBioMedicine 35, 97-105.
47. Xu, Z., Wise, M. C., Chokkalingam, N., Walker, S., Tello-Ruiz, E., Elliott, S. T. C., Perales-Puchalt, A., Xiao, P., Zhu, X., Pumroy, R. A., et al. (2020). In Vivo Assembly of Nanoparticles Achieved through Synergy of Structure-Based Protein Engineering and Synthetic DNA Generates Enhanced Adaptive Immunity. Adv Sci (Weinh) 7, 1902802.
48. Yassine, H. M., Boyington, J. C., McTamney, P. M., Wei, C. J., Kanekiyo, M., Kong, W. P., Gallagher, J. R., Wang, L., Zhang, Y., Joyce, M. G., et al. (2015). Hemagglutinin-stem nanoparticles generate heterosubtypic influenza protection. Nat Med 21, 1065-1070.
49. Zhang, X., Ing, S., Fraser, A., Chen, M., Khan, O., Zakem, J., Davis, W., and Quinet, R. (2013). Follicular helper T cells: new insights into mechanisms of autoimmune diseases. Ochsner J 13, 131-139.

1. A composition comprising: (i) an expressible nucleic acid sequence encoding an adjuvant peptide comprising an HLA I-Ab epitope from Aquifex aeolicus or a functional fragment thereof; or (ii) an amino acid sequence comprising an adjuvant peptide comprising an HLA I-Ab epitope from Aquifex aeolicus or a functional fragment thereof; wherein the adjuvant peptide is no more than about 1000 amino acids.

2. The composition of claim 1, wherein the HLA I-Ab epitope is no more than about 15 amino acids.

3. The composition of claim 1, wherein the adjuvant peptide is capable of binding HLA-DRB1*07:01, HLA-DRB1*15:01 and HLA-DRB5*01:01.

4. (canceled)

5. The composition of claim 1, wherein the nucleic acid sequence encoding the HLA I-Ab epitope comprises at least about 70% sequence identity to SEQ ID NO:2.

6. The composition of claim 1, wherein the expressible nucleic acid comprises a nucleic acid sequence that encodes a viral antigen or a cancer antigen, or wherein the amino acid comprises a viral antigen or a cancer antigen.

7. The composition of claim 6, wherein the viral antigen comprises a Coronaviridae antigen, Respiratory syncytial virus (RSV) antigen, Influenza antigen.

8.-19. (canceled)

20. The composition of claim 1, wherein the expressible nucleic acid sequence encodes an amino acid sequence comprising, from amino terminal to carboxy terminal orientation, the adjuvant peptide, a linker domain, and a viral and/or cancer antigen.

21. A pharmaceutical composition comprising: (i) a therapeutically or prophylactically effective amount of the expressible nucleic acid sequence of claim 1; or a therapeutically or prophylactically effective amount of the amino acid sequence of claim 1; and (ii) a pharmaceutically acceptable carrier.

22. A method of inducing an immune response in a subject comprising administering to the subject the pharmaceutical composition of claim 21.

23. A method of treating a viral infection or cancer in a subject in need thereof comprising administering to the subject the pharmaceutical composition of claim 21.

24. (canceled)

25. A method of activating and/or improving the antigen-specific immune response of a vaccine in a subject in need thereof comprising administering a therapeutically effective amount of the pharmaceutical composition of claim 21 to the subject.

26. The method of claim 23, wherein the viral infection is a Coronaviridae infection, RSV infection, or Influenza infection.

27. The method of claim 23, wherein the cancer is skin cancer, breast cancer or prostate cancer.

28. The method of claim 23, wherein the viral infection is COVID-19 infection.

29. The method of claim 23, wherein the cancer is melanoma.

30. The method of claim 23, wherein the cancer is HER2-breast cancer.

31. The method of claim 23, wherein the cancer is prostate cancer characterized by elevated PSA numbers as compared to a control subject.

32. A nucleic acid vaccine comprising an expressible nucleic acid sequence comprising SEQ ID NO:2 or a functional fragment or variant thereof that comprises at least 70% sequence identity to SEQ ID NO:2.

33. The nucleic acid vaccine of claim 32, wherein the expressible nucleic acid sequence further comprises, a linker, a leader sequence and a nucleic acid sequence encoding an antigen.
